SEQUENCE LISTING (3 sequences)

SEQ ID NO: 1 - protein, 20 aa, Artificial Sequence
  Activating Peptide 1
  MOD_RES (3)..(3): PHOSPHORYLATED TYROSINE
  MOD_RES (9)..(10): Pegylated (dPEG8) between residues 9 and 10
  MOD_RES (13)..(13): PHOSPHORYLATED TYROSINE
  MOD_RES (20)..(20): AMIDATED Lysine
LNYIDLDLVLSTYASINFQK

SEQ ID NO: 2 - protein, 19 aa, Artificial Sequence
  Activating Peptide 2
  MOD_RES (3)..(3): PHOSPHORYLATED TYROSINE
  MOD_RES (9)..(10): Pegylated (dPEG8) between residues 9 and 10
  MOD_RES (13)..(13): PHOSPHORYLATED TYROSINE
  MOD_RES (19)..(19): AMIDATED Phenylalanine
LNYAQLWHALTIYATIRRF

SEQ ID NO: 3 - protein, 593 aa, Homo sapiens [sequence omitted]

(19) United States
(12) Patent Application Publication
(10) Pub. No.: US 2023/0070338 A1
(43) Pub. Date: Mar. 9, 2023

(54) SHP2 Inhibitor Dosing and Methods of Treating Cancer

(71) Applicant: Revolution Medicines, Inc., Redwood City, CA (US)

(72) Inventors: Steve Kelsey, Redwood City, CA (US); Mallika Singh, Redwood City, CA (US); Xiaolin Wang, Redwood City, CA (US); Zhengping Wang, Redwood City, CA (US)

(73) Assignee: Revolution Medicines, Inc., Redwood City, CA (US)

(21) Appl. No.: 17/854,721

(22) Filed: Jun. 30, 2022

Related U.S. Application Data
(63) Continuation of application No. PCT/US2021/012361, filed on Jan. 6, 2021.
(60) Provisional application No. 63/105,148, filed on Oct. 23, 2020.
(60) Provisional application No. 63/041,090, filed on Jun. 18, 2020.
(60) Provisional application No. 62/959,783, filed on Jan. 10, 2020.
(60) Provisional application No. 62/958,260, filed on Jan. 7, 2020.

(51) Int. Cl. A61K 31/497; A61K 45/06; A61P 35/00

(52) U.S. Cl. CPC A61K 31/497; A61K 45/06; A61P 35/00

(57) ABSTRACT

Disclosed are SHP2 inhibitor compositions and methods of treating diseases or disorders using an intermittent dosing schedule.

### RELATED APPLICATIONS

This application is continuation of International Application No. PCT/US2021/012361, filed Jan. 6, 2021, which claims benefit of, and priority to, U.S. Application Nos. 62/958,260 filed Jan. 7, 2020, 62/959,783 filed Jan. 10, 2020, 63/041,090 filed Jun. 18, 2020 and 63/105,148 filed Oct. 23, 2020, the entire contents of which are incorporated herein by reference.

### SEQUENCE LISTING

This application is filed with a Sequence Listing in electronic format. The Sequence Listing is provided as a file entitled “2022-06-29_01183-0197-00 PCT-REV_Sequence_Listing_ST25.txt”, created Jun. 29, 2022, which is 6,768 bytes in size. The information in the electronic format of the sequence listing is incorporated herein by reference in its entirety.

### FIELD OF THE DISCLOSURE

The disclosure relates to methods for the treatment of diseases or disorders (e.g., cancer) with inhibitors of the protein tyrosine phosphatase SHP2. Specifically, disclosed herein are methods of treating diseases or disorders (such as cancer) in subjects using an intermittent dosing schedule of a SHP2 inhibitor alone or in combination with one or more additional therapeutic agents.

### BACKGROUND

Cancer remains one of the most deadly threats to human health. There remains a long-felt and unmet need for a therapeutically effective dosing regimen for treatment of cancer using a SHP2 inhibitor alone or in combination with one or more additional therapeutic agents.

### SUMMARY

The disclosure provides a method of treating a disease or disorder, comprising administering to a subject in need thereof a first dose of a first Src homology region 2 (SH2)-containing protein tyrosine phosphatase 2 (SHP2) inhibitor and a second dose of a second SHP2 inhibitor, wherein the first dose and the second dose are administered on an intermittent schedule. In some embodiments, the first SHP2 inhibitor and the second SHP2 inhibitor are identical. In some embodiments, the first SHP2 inhibitor and the second SHP2 inhibitor are not identical. In some embodiments, the first dose is administered on a first day (D1) of the intermittent schedule and the second dose is administered on a fourth day (D4) of the intermittent schedule. In some embodiments, the first dose is administered on a first day (D1) of the intermittent schedule and the second dose is administered on an eighth day (D8) of the intermittent schedule.

In some embodiments of the disclosure, the SHP2 inhibitor comprises or consists of RMC-4630. In some embodiments, RMC-4630 has the following structure:

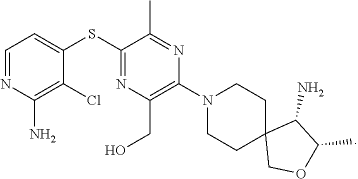 [SMILES: Cc1nc(N2CCC3(CC2)CO[C@@H](C)[C@H]3N)c(CO)nc1Sc1ccnc(N)c1Cl]

As used herein, the term “identical” as it is applied to an inhibitor, including an SHP2 inhibitor of the disclosure, it is meant to describe a small molecule inhibitor having the same structure and/or composition, a nucleic acid having an identical sequence, a protein having an identical sequence or a composition having an active ingredient fulfilling one or more of these criteria. In some embodiments, an identical SHP2 inhibitor is a bioequivalent of the SHP2 inhibitor. In some embodiments, an identical SHP2 inhibitor is a biosimilar of the SHP2 inhibitor.

The disclosure provides a method of treating a disease or disorder, comprising administering to a subject in need thereof a first dose of a first Src homology region 2 (SH2)-containing protein tyrosine phosphatase 2 (SHP2) inhibitor and a second dose of a second SHP2 inhibitor, wherein the subject has a mutation of SHP2 and wherein the first dose and the second dose are administered on an intermittent schedule. In some embodiments, the first SHP2 inhibitor and the second SHP2 inhibitor are identical. In some embodiments, the first SHP2 inhibitor and the second SHP2 inhibitor are not identical. In some embodiments, the first dose is administered on a first day (D1) of the intermittent schedule and the second dose is administered on a fourth day (D4) of the intermittent schedule. In some embodiments, the first dose is administered on a first day (D1) of the intermittent schedule and the second dose is administered on an eighth day (D8) of the intermittent schedule.

In some embodiments of the methods of the disclosure, the first dose is administered on a first day (D1) of the intermittent schedule and the second dose is administered on a second day (D2) of the intermittent schedule. In some embodiments, the method further comprises administering a third dose of a third SHP2 inhibitor on a third day (D3) of the intermittent schedule and a fourth dose of a fourth SHP2 inhibitor on a fourth day (D4) of the intermittent schedule. In some embodiments, at least two of the first SHP2 inhibitor, the second SHP2 inhibitor, the third SHP2 inhibitor and the fourth SHP2 inhibitor are identical. In some embodiments, at least three of the first SHP2 inhibitor, the second SHP2 inhibitor, the third SHP2 inhibitor and the fourth SHP2 inhibitor are identical. In some embodiments, the first SHP2 inhibitor, the second SHP2 inhibitor, the third SHP2 inhibitor and the fourth SHP2 inhibitor are identical. In some embodiments, the first SHP2 inhibitor, the second SHP2 inhibitor, the third SHP2 inhibitor and the fourth SHP2 inhibitor are not identical.

In some embodiments of the methods of the disclosure, the first dose is administered on a first day (D1) of the intermittent schedule and the method further comprises determining a plasma concentration value of the first SHP2 inhibitor of the subject on each subsequent day of the intermittent schedule. In some embodiments, the second dose is administered the day after a plasma concentration value is less than an EC₅₀ value of a phosphorylated extracellular signal-regulated kinase (ERK) (pERK) of the subject. In some embodiments, the EC₅₀ value of the pERK is a predetermined value or a measured value. In some embodiments, the second dose is administered on the fourth day (D4) of the intermittent schedule. In some embodiments, the second dose is administered on the eighth day (D8) of the intermittent schedule. In some embodiments, a complete iteration of the intermittent schedule is 7 days. In some embodiments, a complete iteration of the intermittent schedule consists of 7 days.

In some embodiments of the methods of the disclosure, the first dose is administered on the first day (D1) of the intermittent schedule, wherein the second dose is administered on a second day (D2) of the intermittent schedule, wherein the method further comprises determining a first plasma concentration value of the first SHP2 inhibitor and a second plasma concentration value the second SHP2 inhibitor of the subject on each subsequent day of the intermittent schedule, and wherein a subsequent dose of a subsequent SHP2 inhibitor is administered the day after the first plasma concentration value or the second plasma concentration value is less than an EC₅₀ value of pERK of the subject. In some embodiments, the subsequent dose of the subsequent SHP2 inhibitor is administered the day after the first plasma concentration value and the second plasma concentration value are each less than an EC₅₀ value of pERK of the subject. In some embodiments, the method further comprises administering a third dose of a third SHP2 inhibitor on a third day (D3) of the intermittent schedule and a fourth dose of a fourth SHP2 inhibitor on a fourth day (D4) of the intermittent schedule, and determining a third plasma concentration value of the third SHP2 inhibitor and a fourth plasma concentration value of the fourth SHP2 inhibitor of the subject on each subsequent day of the intermittent schedule, wherein the subsequent dose of the subsequent SHP2 inhibitor is administered the day after the first plasma concentration value, the second plasma concentration value, the third plasma concentration value, or the fourth plasma concentration value, is less than an EC₅₀ value of pERK of the subject. In some embodiments, the subsequent dose of the subsequent SHP2 inhibitor is administered the day after the first plasma concentration value, the second plasma concentration value, the third plasma concentration value, and the fourth plasma concentration value, are each less than an EC₅₀ value of pERK of the subject. In some embodiments, the EC₅₀ value of pERK is a predetermined value or a measured value. In some embodiments, a complete iteration of the intermittent schedule is 7 days. In some embodiments, a complete iteration of the intermittent schedule consists of 7 days. In some embodiments, the subsequent dose is administered on an eighth day (D8). In some embodiments, D8 is the first day of a second or subsequent iteration. In some embodiments, two or more of the first SHP2 inhibitor, the second SHP2 inhibitor, the third SHP2 inhibitor, the fourth SHP2 inhibitor and the subsequent SHP2 inhibitor are identical. In some embodiments, three or more of the first SHP2 inhibitor, the second SHP2 inhibitor, the third SHP2 inhibitor, the fourth SHP2 inhibitor and the subsequent SHP2 inhibitor are identical. In some embodiments, four or more of the first SHP2 inhibitor, the second SHP2 inhibitor, the third SHP2 inhibitor, the fourth SHP2 inhibitor and the subsequent SHP2 inhibitor are identical. In some embodiments, the first SHP2 inhibitor, the second SHP2 inhibitor, the third SHP2 inhibitor, the fourth SHP2 inhibitor and the subsequent SHP2 inhibitor are identical. In some embodiments, the first SHP2 inhibitor, the second SHP2 inhibitor, the third SHP2 inhibitor, the fourth SHP2 inhibitor and the subsequent SHP2 inhibitor are not identical. In some embodiments, a first iteration comprises the first dose and the second dose and the subsequent dose is the first dose of a second or subsequent iteration. In some embodiments, a first iteration comprises the first dose, the second dose, the third dose and the fourth dose, and the subsequent dose is the first dose of a second or subsequent iteration.

In some embodiments of the methods of the disclosure, the method comprises administering at least one complete iteration of the intermittent schedule.

In some embodiments of the methods of the disclosure, the method comprises administering at least 2, 3, 4, 5, 6, 7, 8, 9, or 10 complete iterations of the intermittent schedule.

In some embodiments of the methods of the disclosure, the method further comprises administering a second therapeutic agent. In some embodiments, the method further comprises administering a third or subsequent therapeutic agent. In some embodiments, the method further comprises administering a fourth or subsequent therapeutic agent. A second, third, fourth or subsequent therapeutic agent of the disclosure may comprise one or more of the therapeutic agents known in the art or described herein.

In some embodiments of the methods of the disclosure, the second therapeutic agent comprises a second cell proliferation inhibitor. In some embodiments, the second, third, fourth or subsequent therapeutic agent comprises a second cell proliferation inhibitor. In some embodiments, the second therapeutic agent comprises a mitogen-activated protein kinase kinase (MEK) inhibitor. In some embodiments, the second, third, fourth or subsequent therapeutic agent comprises a mitogen-activated protein kinase kinase (MEK) inhibitor. In some embodiments, the second therapeutic agent comprises cobimetinib. In some embodiments, the second, third, fourth or subsequent therapeutic agent comprises cobimetinib.

In some embodiments of the methods of the disclosure, the second therapeutic agent comprises a second cell proliferation inhibitor. In some embodiments, the second, third, fourth or subsequent therapeutic agent comprises a second cell proliferation inhibitor. In some embodiments, the second therapeutic agent comprises a rat sarcoma (RAS) inhibitor. In some embodiments, the second, third, fourth or subsequent therapeutic agent comprises a rat sarcoma (RAS) inhibitor. In some embodiments, the RAS inhibitor inhibits one or more of Kristen rat sarcoma (KRAS), neuroblastoma RAS (NRAS) and Harvey rat sarcoma (HRAS). In some embodiments, the RAS inhibitor inhibits Kristen rat sarcoma (KRAS), neuroblastoma RAS (NRAS) and Harvey rat sarcoma (HRAS). In some embodiments, the second therapeutic agent comprises a KRAS inhibitor. In some embodiments, the second, third, fourth or subsequent therapeutic agent comprises a KRAS inhibitor. In some embodiments, the RAS inhibitor is a non-covalent inhibitor. In some embodiments, the RAS inhibitor is a covalent inhibitor. In some embodiments, the RAS inhibitor inhibits an activated or guanine triphosphate (GTP)-bound form of RAS. In some embodiments, the RAS inhibitor inhibits an inactivated or guanine diphosphate (GDP)-bound form of RAS. In some embodiments, the second therapeutic agent comprises a KRAS^{G12C} inhibitor. In some embodiments, the second, third, fourth or subsequent therapeutic agent comprises a KRAS^{G12C} inhibitor. In some embodiments, the second, third, fourth or subsequent therapeutic agent comprises

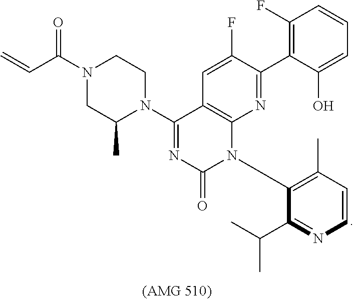

In some embodiments, the second, third, fourth or subsequent therapeutic agent comprises

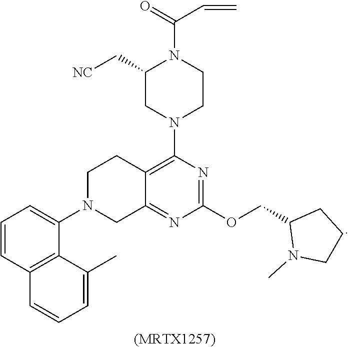

In some embodiments, the second, third, fourth or subsequent therapeutic agent comprises

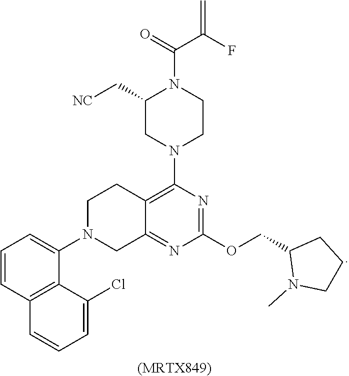

In some embodiments, the second, third, fourth or subsequent therapeutic agent comprises ARS 3248 or JNJ-74699157.

In some embodiments, the second, third, fourth or subsequent therapeutic agent comprises

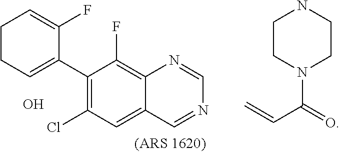

In some embodiments of the methods of the disclosure, the method comprises administering a first dose of the second therapeutic agent and a second dose of the second therapeutic agent, wherein the first dose of the second therapeutic agent and the second dose of the second therapeutic agent are administered on an intermittent schedule. In some embodiments, one or more of the first SHP2 inhibitor, the second SHP2 inhibitor, the third SHP2 inhibitor, the fourth SHP2 inhibitor and the subsequent SHP2 inhibitor, and the second therapeutic agent are administered simultaneously. In some embodiments, one or more of the first SHP2 inhibitor, the second SHP2 inhibitor, the third SHP2 inhibitor, the fourth SHP2 inhibitor and the subsequent SHP2 inhibitor, and the second therapeutic agent are not administered simultaneously.

In some embodiments of the methods of the disclosure, the method comprises administering a first dose of the second therapeutic agent and a second dose of the second therapeutic agent, wherein the first dose of the second therapeutic agent and the second dose of the second therapeutic agent are administered on an intermittent schedule. In some embodiments, the first SHP2 inhibitor or the first dose of a SHP2 inhibitor and the second therapeutic agent are administered simultaneously. In some embodiments, the first SHP2 inhibitor or the first dose of a SHP2 inhibitor and the second therapeutic agent are not administered simultaneously. In some embodiments, the second SHP2 inhibitor or the second dose of a SHP2 inhibitor and the second therapeutic agent are administered simultaneously. In some embodiments, the second SHP2 inhibitor or the second dose of a SHP2 inhibitor and the second therapeutic agent are not administered simultaneously. In some embodiments, the third SHP2 inhibitor or the third dose of a SHP2 inhibitor; and the second therapeutic agent are administered simultaneously. In some embodiments, the third SHP2 inhibitor or the third dose of a SHP2 inhibitor and the second therapeutic agent are not administered simultaneously. In some embodiments, the fourth SHP2 inhibitor or the fourth dose of a SHP2 inhibitor and the second therapeutic agent are administered simultaneously. In some embodiments, the fourth SHP2 inhibitor or the fourth dose of a SHP2 inhibitor; and the second therapeutic agent are not administered simultaneously. In some embodiments, the subsequent SHP2 inhibitor or the subsequent dose of a SHP2 inhibitor and the second therapeutic agent are administered simultaneously. In some embodiments, the subsequent SHP2 inhibitor or the subsequent dose of a SHP2 inhibitor and the second therapeutic agent are not administered simultaneously.

In some embodiments of the methods of the disclosure, the method comprises administering a first dose of the second therapeutic agent and a second dose of the second therapeutic agent, wherein the first dose of the second therapeutic agent and the second dose of the second therapeutic agent are administered on an intermittent schedule. In some embodiments, one or more of the first SHP2 inhibitor, the second SHP2 inhibitor, the third SHP2 inhibitor, the fourth SHP2 inhibitor and the subsequent SHP2 inhibitor, and the second therapeutic agent are administered sequentially. In some embodiments, the first SHP2 inhibitor or the first dose of a SHP2 inhibitor is administered before the second therapeutic agent. In some embodiments, the second therapeutic agent is administered before the first SHP2 inhibitor or the first dose of a SHP2 inhibitor. In some embodiments, the second SHP2 inhibitor or the second dose of a SHP2 inhibitor is administered before the second therapeutic agent. In some embodiments, the second therapeutic agent is administered before the second SHP2 inhibitor or the second dose of a SHP2 inhibitor. In some embodiments, the third SHP2 inhibitor or the third dose of a SHP2 inhibitor is administered before the second therapeutic agent. In some embodiments, the second therapeutic agent is administered before the third SHP2 inhibitor or the third dose of a SHP2 inhibitor. In some embodiments, the fourth SHP2 inhibitor or the fourth dose of a SHP2 inhibitor is administered before the second therapeutic agent. In some embodiments, the second therapeutic agent is administered before the fourth SHP2 inhibitor or the fourth dose of a SHP2 inhibitor. In some embodiments, the subsequent SHP2 inhibitor or the subsequent dose of a SHP2 inhibitor is administered before the second therapeutic agent. In some embodiments, the second therapeutic agent is administered before the subsequent SHP2 inhibitor or the subsequent dose of a SHP2 inhibitor.

In some embodiments of the methods of the disclosure, the first dose of the first SHP2 inhibitor and a first dose of the second therapeutic agent are administered on D1 of the intermittent schedule and the second dose of the second SHP2 inhibitor and a second dose of the second therapeutic agent are administered on different days of the intermittent schedule. In some embodiments, the first SHP2 inhibitor and the second SHP2 inhibitor are identical. In some embodiments, the first SHP2 inhibitor and the second SHP2 inhibitor are not identical. In some embodiments, a complete iteration of the intermittent schedule is 7 days. In some embodiments, a complete iteration of the intermittent schedule consists of 7 days. In some embodiments, the method comprises administering at least one complete iteration of the intermittent schedule. In some embodiments, the method comprises administering at least 2, 3, 4, 5, 6, 7, 8, 9, or 10 complete iterations of the intermittent schedule.

In some embodiments of the methods of the disclosure, the first dose of the first SHP2 inhibitor and a first dose of the second therapeutic agent are administered on D1 of the intermittent schedule and the second dose of the second SHP2 inhibitor and a first dose of a third therapeutic agent are administered on different days of the intermittent schedule. In some embodiments, the first SHP2 inhibitor and the second SHP2 inhibitor are identical. In some embodiments, the first SHP2 inhibitor and the second SHP2 inhibitor are not identical. In some embodiments, the second therapeutic agent and the third therapeutic agent are identical. In some embodiments, the second therapeutic agent and the third therapeutic agent are not identical. In some embodiments, a complete iteration of the intermittent schedule is 7 days. In some embodiments, a complete iteration of the intermittent schedule consists of 7 days. In some embodiments, the method comprises administering at least one complete iteration of the intermittent schedule. In some embodiments, the method comprises administering at least 2, 3, 4, 5, 6, 7, 8, 9, or 10 complete iterations of the intermittent schedule.

In some embodiments of the methods of the disclosure, the first dose of the SHP2 inhibitor and a first dose of the second therapeutic agent are administered on different days of the intermittent schedule and the second dose of the second SHP2 inhibitor and a second dose of the second therapeutic agent are administered on the same day of the intermittent schedule. In some embodiments, the first SHP2 inhibitor and the second SHP2 inhibitor are identical. In some embodiments, the first SHP2 inhibitor and the second SHP2 inhibitor are not identical. In some embodiments, a complete iteration of the intermittent schedule is 7 days. In some embodiments, a complete iteration of the intermittent schedule consists of 7 days. In some embodiments, the method comprises administering at least one complete iteration of the intermittent schedule. In some embodiments, the method comprises administering at least 2, 3, 4, 5, 6, 7, 8, 9, or 10 complete iterations of the intermittent schedule.

In some embodiments of the methods of the disclosure, the first dose of the SHP2 inhibitor and a first dose of the second therapeutic agent are administered on different days of the intermittent schedule and wherein the second dose of the second SHP2 inhibitor and a first dose of a third therapeutic agent are administered on the same day of the intermittent schedule. In some embodiments, the first SHP2 inhibitor and the second SHP2 inhibitor are identical. In some embodiments, the first SHP2 inhibitor and the second SHP2 inhibitor are not identical. In some embodiments, the second therapeutic agent and the third therapeutic agent are identical. In some embodiments, the second therapeutic agent and the third therapeutic agent are not identical. In some embodiments, a complete iteration of the intermittent schedule is 7 days. In some embodiments, a complete iteration of the intermittent schedule consists of 7 days. In some embodiments, the method comprises administering at least one iteration of the intermittent schedule. In some embodiments, the method comprises administering at least 2, 3, 4, 5, 6, 7, 8, 9, or 10 iterations of the intermittent schedule.

In some embodiments of the methods of the disclosure, the SHP2 inhibitor is an allosteric SHP2 inhibitor.

In some embodiments of the methods of the disclosure, the SHP2 inhibitor is an allosteric SHP2 inhibitor and the mutation of SHP2 is sensitive to an allosteric SHP2 inhibitor. In some embodiments, the mutation of SHP2 comprises one or more of F285S, L262R, S189A, D61G, E69K, T73I and Q506P. In some embodiments, the mutation of SHP2 comprises one or more of F285S, L262R and S189A. In some embodiments, the mutation of SHP2 comprises D61G. In some embodiments, the mutation of SHP2 comprises one or more of E69K, T73I and Q506P.

In some embodiments of the methods of the disclosure, the subject does not have a mutation of SHP2 resistant to an allosteric SHP2 inhibitor. In some embodiments, the mutation of SHP2 resistant to an allosteric SHP2 inhibitor comprises one or more of E76K, P491S and S502P. In some embodiments, the mutation of SHP2 resistant to an allosteric SHP2 inhibitor comprises E76K or P491S. In some embodiments, the mutation of SHP2 resistant to an allosteric SHP2 inhibitor comprises S502P.

In some embodiments of the methods of the disclosure, the subject has been identified as having the mutation of SHP2 prior to administration of the first dose of a SHP2 inhibitor. In some embodiments, the subject has been identified as being at risk of developing a disease or disorder caused by the mutation of SHP2 prior to administration of the first dose of a SHP2 inhibitor. In some embodiments, the subject has been identified as having a disease or disorder caused by the mutation of SHP2 prior to administration of the first dose of a SHP2 inhibitor. In some embodiments, the SHP2 inhibitor is a first SHP2 inhibitor, a second SHP2 inhibitor, a third SHP2 inhibitor, a fourth SHP2 inhibitor or a subsequent SHP2 inhibitor.

In some embodiments of the methods of the disclosure, including compositions of the disclosure for use in treating a disease or disorder of the disclosure, the subject has been identified as having a relapsed or refractory form of the disease or disorder. In some embodiments, the disease or disorder of the disclosure comprises a tumor, a proliferation or a cancer. In some embodiments, the tumor, the proliferation or the cancer originates (is a primary presentation) or metastasizes (a secondary presentation) to any cell type, tissue or location in the body. In some embodiments, the tumor, the proliferation or the cancer originates (is a primary presentation) or metastasizes (a secondary presentation) to the colon. In some embodiments, the tumor, the proliferation or the cancer is a colon cancer or a subtype thereof. In some embodiments, a relapsed disease or disorder of the disclosure comprises one or more of a (1) disease or disorder treated by a composition or method other than one of the disclosure (including, for example, the established or art-recognized standard of care), which, after an initial period of response, improvement, or remission, the disease or disorder reappears or reduces/reverses its response to the initial treatment; (2) disease or disorder treated by a composition or method of the disclosure, which, after an initial period of response, improvement, or remission, the disease or disorder reappears or reduces/reverses its response to the initial treatment; (3) disease or disorder that, when treated by any known composition or method (including, for example, the established or art-recognized standard of care), demonstrates a lack of sensitivity to the treatment or a refractory response to the treatment; (4) disease or disorder that, in the subject in need of treatment, when treated by any known composition or method (including, for example, the established or art-recognized standard of care), demonstrates a lack of sensitivity to the treatment or a refractory response to the treatment; (5) any combination of (1)-(4). In some embodiments, the standard of care comprises a first-line therapy for the disease or disorder. In some embodiments, the standard of care comprises an approved therapy (e.g. by a government regulatory authority assessing safety and efficacy) for the disease or disorder. In some embodiments, the standard of care comprises a therapy approved for a first disease or disorder by a government regulatory authority assessing safety and efficacy, but which has been repurposed for a disease or disorder of the disclosure.

In some embodiments of the methods of the disclosure, the SHP2 inhibitor comprises (i) SHP099; (ii) an allosteric SHP2 inhibitor compound of any one of Formula I, of Formula II, of Formula III, of Formula 1-VI, of Formula I-V2, of Formula I-W, of Formula i-X, of Formula I-Y, of Formula I-Z, of Formula IV, of Formula V, of Formula VI, of Formula IV-X, of Formula IV-Y, of Formula 1V-Z, of Formula VII, of Formula VIII, of Formula IX, and of Formula X; (iii) TNO155; (iv) JAB-3068; (v) a compound from Table 1, disclosed herein; (vi) a compound from Table 2, disclosed herein; (vii) RLY-1971; or (viii) a combination thereof.

In some embodiments of the methods of the disclosure, the SHP2 inhibitor comprises

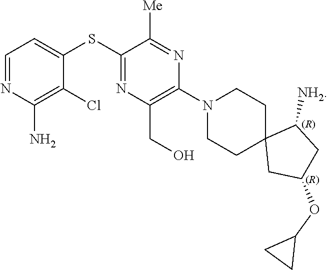

In some embodiments of the methods of the disclosure, the SHP2 inhibitor comprises

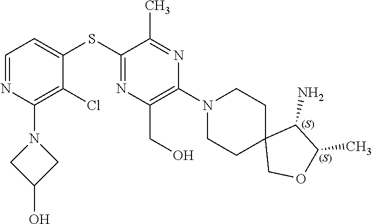

In some embodiments of the methods of the disclosure, the SHP2 inhibitor comprises

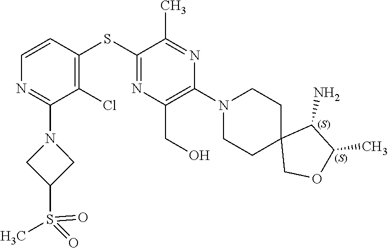

In some embodiments of the methods of the disclosure, the SHP2 inhibitor comprises

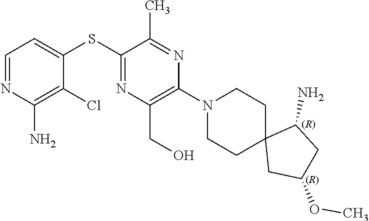

In some embodiments of the methods of the disclosure, the SHP2 inhibitor comprises

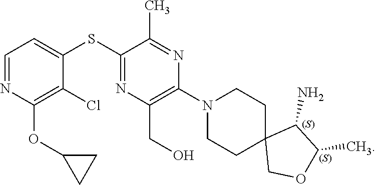

In some embodiments of the methods of the disclosure, the SHP2 inhibitor comprises

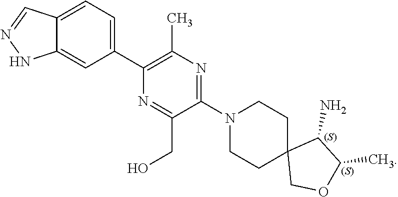

In some embodiments of the methods of the disclosure, the SHP2 inhibitor comprises

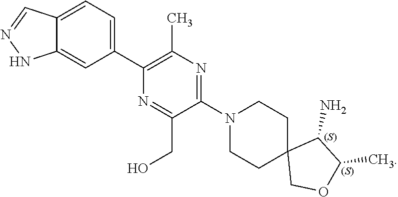

In some embodiments of the methods of the disclosure, the SHP2 inhibitor comprises

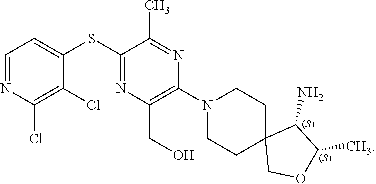

In some embodiments of the methods of the disclosure, the SHP2 inhibitor comprises

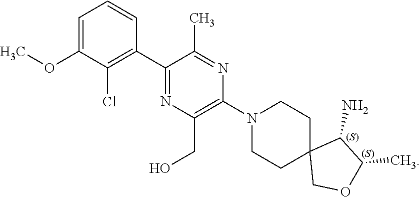

In some embodiments of the methods of the disclosure, the SHP2 inhibitor comprises

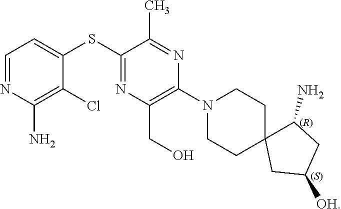

In some embodiments of the methods of the disclosure, the SHP2 inhibitor comprises

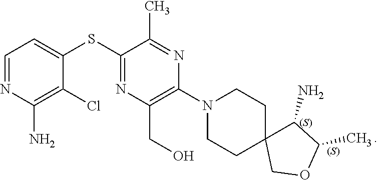

In some embodiments of the methods of the disclosure, the SHP2 inhibitor comprises

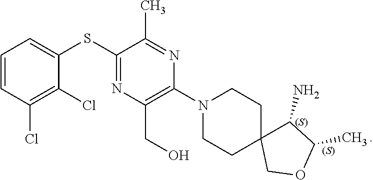

In some embodiments of the methods of the disclosure, the SHP2 inhibitor comprises

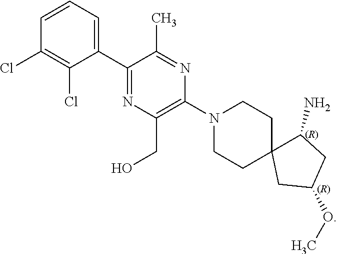

In some embodiments of the methods of the disclosure, the SHP2 inhibitor comprises

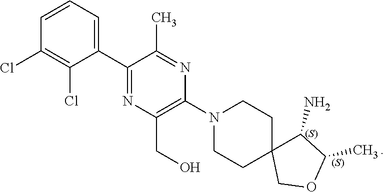

In some embodiments of the methods of the disclosure, the subject further comprises a mutation in a component of a rat sarcoma (RAS) signaling pathway. In some embodiments, the mutation in the component of the RAS signaling pathway occurs in KRAS, neurofibromin 1 (NF1), or serine/threonine-protein kinase B-raf (BRAF). In some embodiments, the mutation in the component of the RAS signaling pathway comprises a substitution of a cysteine (C) for a glycine (G) at position 12 of KRAS (KRAS^{G12C}). In some embodiments, the mutation in the component of the RAS signaling pathway comprises a KRAS amplification (KRAS^{amp}). In some embodiments, the mutation in the component of the RAS signaling pathway comprises a loss of function (LOF) mutation of NF1 (NF1^{LOF}) In some embodiments, the mutation in the component of the RAS signaling pathway comprises a class 3 mutant of BRAF (BRAF^{class3}) In some embodiments, the mutation in the component of the RAS signaling pathway does not comprise a substitution of a glutamic acid (E) for a valine (V) at position 600 of BRAF.

In some embodiments of the methods of the disclosure, the disease or disorder is a tumor. In some embodiments, the tumor is a malignant tumor. In some embodiments, the tumor is a cancer. In some embodiments, the tumor is metastatic. In some embodiments, the cancer is metastatic. In some embodiments, the tumor or the cancer has a primary presentation in one or both lung(s) of the subject. In some embodiments, the tumor or the cancer has a secondary presentation in one or both lung(s) of the subject. In some embodiments, the tumor or the cancer is non-small cell lung cancer. In some embodiments, the tumor or the cancer presents a brain metastasis in the subject.

In some embodiments of the methods of the disclosure, the disease or disorder is a tumor. In some embodiments, the tumor is a malignant tumor. In some embodiments, the tumor is a cancer. In some embodiments, the tumor is metastatic. In some embodiments, the cancer is metastatic. In some embodiments, the tumor or the cancer has a primary presentation in a pancreas of the subject. In some embodiments, the tumor or the cancer has a secondary presentation in a pancreas of the subject.

In some embodiments of the methods of the disclosure, the disease or disorder is a tumor. In some embodiments, the tumor is a malignant tumor. In some embodiments, the tumor is a cancer. In some embodiments, the tumor is metastatic. In some embodiments, the cancer is metastatic. In some embodiments, the tumor or the cancer has a primary presentation in one or more of a large intestine, a small intestine, a stomach, a bladder, a kidney, a colon or a rectum of the subject. In some embodiments, the tumor or the cancer has a secondary presentation in one or more of a large intestine, a small intestine, a stomach, a bladder, a kidney, a colon or a rectum of the subject.

In some embodiments of the methods of the disclosure, the disease or disorder is a tumor. In some embodiments, the tumor is a malignant tumor. In some embodiments, the tumor is a cancer. In some embodiments, the tumor is metastatic. In some embodiments, the cancer is metastatic. In some embodiments, the tumor or the cancer has a primary presentation as a sarcoma in the subject. In some embodiments, the tumor or the cancer has a secondary presentation as a sarcoma in the subject.

In some embodiments of the methods of the disclosure, the subject is human. In some embodiments, the subject is female. In some embodiments, the subject is male.

In some embodiments of the methods of the disclosure, the first dose of the first SHP2 inhibitor or the second dose of the second SHP2 inhibitor comprises a therapeutically effective amount of a SHP2 inhibitor. In some embodiments, the first dose of the SHP2 inhibitor and the second dose of the SHP2 inhibitor each comprises a therapeutically effective amount of the SHP2 inhibitor. In some embodiments, the first dose of the first SHP2 inhibitor or the second dose of the second SHP2 inhibitor reduces tumor burden of the subject. In some embodiments, the first dose of the first SHP2 inhibitor and the second dose of the second SHP2 inhibitor each reduce tumor burden of the subject. In some embodiments, the combination of the first dose of the first SHP2 inhibitor and the second dose of the second SHP2 inhibitor reduces tumor burden of the subject. In some embodiments, the first dose of the first SHP2 inhibitor or the second dose of the second SHP2 inhibitor decreases activation of a component of a RAS signaling pathway in the subject. In some embodiments, the first dose of the first SHP2 inhibitor and the second dose of the second SHP2 inhibitor each decrease activation of a component of a RAS signaling pathway in the subject. In some embodiments, the combination of the first dose of the first SHP2 inhibitor and the second dose of the second SHP2 inhibitor decreases activation of a component of a RAS signaling pathway in the subject.

In some embodiments of the methods of the disclosure, the first dose of the SHP2 inhibitor, the second dose of the SHP2 inhibitor, the third dose of the third SHP2 inhibitor, or the fourth dose of the fourth SHP2 inhibitor comprises a therapeutically effective amount of a SHP2 inhibitor. In some embodiments, the first dose of the SHP2 inhibitor, the second dose of the SHP2 inhibitor, the third dose of the third SHP2 inhibitor, and the fourth dose of the fourth SHP2 inhibitor each comprise a therapeutically effective amount of a SHP2 inhibitor. In some embodiments, the first dose of the first SHP2 inhibitor, the second dose of the second SHP2 inhibitor, the third dose of the SHP2 inhibitor or the fourth dose of the SHP2 inhibitor reduces tumor burden of the subject. In some embodiments, the first dose of the first SHP2 inhibitor, the second dose of the second SHP2 inhibitor, the third dose of the SHP2 inhibitor and the fourth dose of the SHP2 inhibitor each reduce tumor burden of the subject. In some embodiments, the combination of the first dose of the first SHP2 inhibitor, the second dose of the second SHP2 inhibitor, the third dose of the SHP2 inhibitor and the fourth dose of the SHP2 inhibitor reduces tumor burden of the subject. In some embodiments, the first dose of the first SHP2 inhibitor, the second dose of the second SHP2 inhibitor, the third dose of the SHP2 inhibitor or the fourth dose of the SHP2 inhibitor decreases activation of a component of a RAS signaling pathway in the subject. In some embodiments, the first dose of the first SHP2 inhibitor, the second dose of the second SHP2 inhibitor, the third dose of the SHP2 inhibitor and the fourth dose of the SHP2 inhibitor each decrease activation of a component of a RAS signaling pathway in the subject. In some embodiments, the combination of the first dose of the first SHP2 inhibitor, the second dose of the second SHP2 inhibitor, the third dose of the SHP2 inhibitor and the fourth dose of the SHP2 inhibitor decreases activation of a component of a RAS signaling pathway in the subject.

In some embodiments of the methods of the disclosure, treating comprises reducing tumor burden of the subject.

In some embodiments of the methods of the disclosure, treating comprises decreasing activation of a component of a RAS signaling pathway in the subject. In some embodiments, decreasing activation of a component of a RAS signaling pathway comprises decreasing phosphorylation of ERK.

In some embodiments of the methods of the disclosure, the first dose of the first SHP2 inhibitor or the second dose of the second SHP2 inhibitor is administered systemically. In some embodiments, the first dose of the first SHP2 inhibitor or the second dose of the second SHP2 inhibitor is administered orally. In some embodiments of the methods of the disclosure, the first dose of the first SHP2 inhibitor, the second dose of the second SHP2 inhibitor, the third dose of the SHP2 inhibitor or the fourth dose of the SHP2 inhibitor is administered systemically. In some embodiments, the first dose of the first SHP2 inhibitor, the second dose of the second SHP2 inhibitor, the third dose of the SHP2 inhibitor or the fourth dose of the SHP2 inhibitor is administered orally. In some embodiments, the first dose of the first SHP2 inhibitor, the second dose of the second SHP2 inhibitor, the third dose of the third SHP2 inhibitor, the fourth dose of the fourth SHP2 inhibitor or the subsequent dose of the subsequent SHP2 inhibitor is at least 10 milligrams (mg), 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg or at least any number of mg in between. In some embodiments, the first dose of the first SHP2 inhibitor, the second dose of the second SHP2 inhibitor, the third dose of the third SHP2 inhibitor, the fourth dose of the fourth SHP2 inhibitor or the subsequent dose of the subsequent SHP2 inhibitor is between 20 mg and 300 mg, inclusive of the endpoints. In some embodiments, the first dose of the first SHP2 inhibitor, the second dose of the second SHP2 inhibitor, the third dose of the third SHP2 inhibitor, the fourth dose of the fourth SHP2 inhibitor or the subsequent dose of the subsequent SHP2 inhibitor is at least 80 mg. In some embodiments, the first dose of the first SHP2 inhibitor, the second dose of the second SHP2 inhibitor, the third dose of the third SHP2 inhibitor, the fourth dose of the fourth SHP2 inhibitor or the subsequent dose of the subsequent SHP2 inhibitor is about 80 mg. In some embodiments, the first dose of the first SHP2 inhibitor, the second dose of the second SHP2 inhibitor, the third dose of the third SHP2 inhibitor, the fourth dose of the fourth SHP2 inhibitor or the subsequent dose of the subsequent SHP2 inhibitor is 80 mg. In some embodiments, the first dose of the first SHP2 inhibitor, the second dose of the second SHP2 inhibitor, the third dose of the third SHP2 inhibitor, the fourth dose of the fourth SHP2 inhibitor or the subsequent dose of the subsequent SHP2 inhibitor is at least 140 mg. In some embodiments, the first dose of the first SHP2 inhibitor, the second dose of the second SHP2 inhibitor, the third dose of the third SHP2 inhibitor, the fourth dose of the fourth SHP2 inhibitor or the subsequent dose of the subsequent SHP2 inhibitor is about 140 mg. In some embodiments, the first dose of the first SHP2 inhibitor, the second dose of the second SHP2 inhibitor, the third dose of the third SHP2 inhibitor, the fourth dose of the fourth SHP2 inhibitor or the subsequent dose of the subsequent SHP2 inhibitor is 140 mg. In some embodiments, the first dose of the first SHP2 inhibitor, the second dose of the second SHP2 inhibitor, the third dose of the third SHP2 inhibitor, the fourth dose of the fourth SHP2 inhibitor or the subsequent dose of the subsequent SHP2 inhibitor is at least 200 mg. In some embodiments, the first dose of the first SHP2 inhibitor, the second dose of the second SHP2 inhibitor, the third dose of the third SHP2 inhibitor, the fourth dose of the fourth SHP2 inhibitor or the subsequent dose of the subsequent SHP2 inhibitor is about 200 mg. In some embodiments, the first dose of the first SHP2 inhibitor, the second dose of the second SHP2 inhibitor, the third dose of the third SHP2 inhibitor, the fourth dose of the fourth SHP2 inhibitor or the subsequent dose of the subsequent SHP2 inhibitor is 200 mg.

In some embodiments of the methods of the disclosure, the second, third or subsequent therapeutic agent is administered at a dose of at least 10 milligrams (mg), 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg or at least any number of mg in between. In some embodiments, the second, third or subsequent therapeutic agent is administered at a dose of between 10 mg and 300 mg, inclusive of the endpoints. In some embodiments, the second, third or subsequent therapeutic agent is administered at a dose of at least 20 mg, 40 mg, 60 mg, 80 mg or at least any number of mg in between. In some embodiments, the second, third or subsequent therapeutic agent is administered at a dose of about 20 mg, 40 mg, 60 mg or 80 mg. In some embodiments, the second, third or subsequent therapeutic agent is administered at a dose of 20 mg, 40 mg, 60 mg or 80 mg. In some embodiments, the second, third or subsequent therapeutic agent is administered at a dose of between 20 mg and 80 mg, inclusive of the endpoints. In some embodiments, the second, third or subsequent therapeutic agent is administered at a dose of 20 mg. In some embodiments, the second, third or subsequent therapeutic agent is administered at a dose of 40 mg. In some embodiments, the second, third or subsequent therapeutic agent is administered at a dose of 60 mg.

### BRIEF DESCRIPTION OF THE DRAWINGS

FIG. 1 is a schematic drawing depicting a SHP2-mediated signaling pathway (see Nichols et al, Nat Cell Biol, 2018). RAS signaling is frequently dysregulated in human cancers. Treatment options are limited for patients with tumors harboring RAS, NF1, or BRAF mutations other than BRAF^{V600E}. RMC-4630 is a potent, selective, orally bioavailable, allosteric inhibitor of SHP2. The RMC-4630 clinical program tests the emerging hypothesis of semi-autonomous, SHP2-dependent, RAS signaling mutations such as KRAS^{G12C}, NF1^{LOF}, BRAF^{class3} and others (e.g. KRAS^{amp}). In some embodiments, RMC-4630 has the following structure:

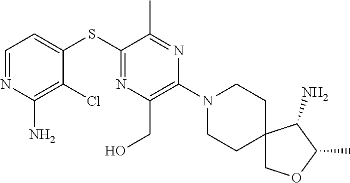 [SMILES: Cc1nc(N2CCC3(CC2)CO[C@@H](C)[C@H]3N)c(CO)nc1Sc1ccnc(N)c1Cl]

FIG. 2 is a pair of graphs demonstrating RMC-4630 induces status and regression in a preclinical mouse model of non-small cell lung cancer (NSCLC) having a mutation in KRAS (KRAS^{G12C}). In this study, RMC-4630 was administered daily at either 10 mg/kg or 30 mg/kg.

FIG. 3 is a pair of schematic drawings depicting the experimental design of the first-in-human study for RMC-4630.

FIG. 4 is a pair of tables providing baseline characteristics of patients enrolled in the first-in-human study depicted in FIG. 3.

FIG. 5 is a table providing initial data of adverse events reported by patients enrolled in the first-in-human study depicted in FIG. 3.

FIG. 6 is a graph depicting plasma concentrations sustained above pERK EC50 for KRAS G12C tumors following administration of RMC-4630 on either a single dose schedule (at one of 20 mg, 40 mg, 60 mg or 80 mg) or an intermittent schedule (140 mg or 200 mg provided at D1 or D4 of a 7 day iteration).

FIG. 7A is a graph depicting the H-Score for nuclear and cytoplasmic ERK phosphorylation in cells obtained from each of four patients following treatment with RMC-4630 on a daily dosing schedule provided in FIG. 7C. H score is the product of percentage of tumor cells staining positive for pERK and the intensity of staining per cell. Both nuclear and cytoplasmic pERK are shown.

FIG. 7B is a photograph of tissue obtained from patients 1 and 3 following treatment with RMC-4630 on a daily dosing schedule provided in FIG. 7C. Tissue staining reveals the degree of inhibition of ERK as pERK stains brown. Panel B shows the immunohistochemistry sections from which the H score is estimated. pERK stains brown.

FIG. 7C is a table providing disease characteristics and treatment regimen for each patent of the study from which data was extracted for FIGS. 7A and 7B. The table (panel C) provides information for each patient on whom paired biopsies were obtained.

FIG. 8 is a graph depicting the change in tumor burden of patients having NSCLC and a KRAS mutation (G12C, G12D or G12V), following treatment with RMC-4630.

FIG. 9 is a series of photographs depicting radiologic responses of a patient diagnosed with KRAS^{G12C} NSCLC following treatment with RMC-4630.

FIG. 10 is a table providing demographics and disease characteristics of patients receiving RMC4630 as part of the RMC-4630-01, phase 1 study, in accordance with an intermittent dosing schedule.

FIG. 11 is a table providing a list of related adverse events (AEs) occurring in more than 15% of patients dosed with RMC-4630 as part of the RMC-4630-01, phase 1 study, in accordance with an intermittent dosing schedule. The occurrence of AEs is presented by grade.

FIG. 12 is a table providing the pharmacokinetics of RMC-4630 action following administration by an intermittent dosing schedule in a mouse study and in the RMC-4630-01, phase 1 study.

FIG. 13 is a pair of graphs depicting the pharmacokinetics of RMC-4630 action following administration by an intermittent dosing schedule in the RMC-4630-01, phase 1 study. Pharmacokinetic profile of RMC-4630 dosed at either 140 mg or 200 mg on D1 and D4 of each week. Steady state is considered to be day 15 of iteration 1. EC₅₀/fu and EC₇₅/fu are the total estimated plasma concentrations in humans that correspond to 50% and 75% inhibition of pERK in KRAS^{G12C} tumor models.

FIG. 14 is a table providing demographics and disease characteristics of patients receiving RMC4630 as part of the RMC-4630-01, phase 1 study, in accordance with a daily dosing schedule.

FIG. 15 is a table providing a list of related adverse events (AEs) occurring in patients dosed with RMC4630 as part of the RMC-4630-01, phase 1 study, in accordance with a daily dosing schedule. The occurrence of AEs is presented by grade.

FIG. 16 is a table providing a list of severe adverse events (SAEs) occurring in patients dosed with RMC4630 as part of the RMC-4630-01, phase 1 study, in accordance with a daily dosing schedule. The occurrence of SAEs is presented by grade.

FIG. 17 is a table providing the pharmacokinetics of RMC-4630 action following administration by a daily dosing schedule in a mouse study and in the RMC-4630-01, phase 1 study.

FIG. 18 is a pair of graphs depicting the pharmacokinetics of RMC-4630 action following administration by a daily dosing schedule in the RMC-4630-01, phase 1 study. Pharmacokinetic profile of RMC-4630 dosed at either 20 mg, 40 mg, 60 mg or 80 mg daily. Steady state is considered to be day 22 of iteration 1. EC₅₀/fu and EC₇₅/fu are the total estimated plasma concentrations in humans that correspond to 50% and 75% inhibition of pERK in KRAS^{G12C} tumor models.

FIG. 19 is a table providing circulating KRAS^{G12C} allele frequency in patients with KRAS^{G12C} Tumors.

FIG. 20 is a graph depicting the best change in tumor burden from baseline in KRAS^{G12C} NSCLC. Waterfall plot of best tumor response for five patients with KRAS^{G12C} NSCLC who had baseline target lesions assessed and at least one radiologic follow-up assessment of target lesion size. Percentage (Y axis) represents the percentage change from baseline in the Sum of Longest Diameters of target lesions using RECIST 1.1. Colors represent different dose levels.

FIG. 21 is a graph depicting the best change in tumor burden from baseline in NSCLC for any KRAS mutation (including G12C, G12D, G12V, and G12S). Waterfall plot of best tumor response for fourteen patients with KRAS mutant NSCLC, including KRAS^{G12C}, who had baseline target lesions assessed and at least one radiologic follow-up assessment of target lesion size. Percentage (Y axis) represents the percentage change from baseline in the Sum of Longest Diameters of target lesions using RECIST 1.1. Colors represent different KRAS mutations.

FIG. 22 is a table providing demographics and disease characteristics of patients receiving RMC-4630 and cobimetinib as part of the RMC-4630-02, phase 1b/2 study.

FIG. 23 is a table providing related AEs attributed to RMC-4630 in patients receiving RMC-4630 and cobimetinib as part of the RMC-4630-02, phase 1b/2 study. The occurrence of AEs is presented by grade.

FIG. 24 is a table providing related AEs attributed to cobimetinib in patients receiving RMC-4630 and cobimetinib as part of the RMC-4630-02, phase 1b/2 study. The occurrence of AEs is presented by grade.

FIG. 25 is a table providing the pharmacokinetics in the RMC-4630-02, phase 1b/2 study.

FIG. 26 is a pair of graphs depicting the pharmacokinetics of RMC-4630 as part of the RMC-4630-02, phase 1b/2 study. Pharmacokinetic profile of RMC-4630 dosed at 80 mg D1, D4 and cobimetinib dosed 20 mg daily in the RMC-4630-02 study. Steady state is considered to be day 15 of iteration 1. EC₅₀/fu and EC₇₅/fu are the total estimated plasma concentrations of RMC-4630 in humans that correspond to 50% and 75% inhibition of pERK in KRAS^{G12C} tumor models.

FIG. 27A is a graph depicting plasma concentration over time profiles. RMC-4630 was dosed daily at 60 mg or intermittent twice weekly at 140 mg (D1, D4) or 200 mg (D1, D2). For 60 mg daily dosing, plasma concentration profile was from Cycle 1 Day 22 (steady state). For 140 mg (D1, D4) and 200 mg (D1, D2) schedules, plasma concentration profiles from week 1 were presented. No accumulation was observed following twice weekly dosing. The dotted lines on the plot indicate the cytostatic and apoptotic thresholds and represent the approximate plasma concentrations required to inhibit RAS pathway activity in tumor xenograft models in mice in vivo by 50% (EC₅₀) and 75% (EC₇₅) respectively. These thresholds are based on the preclinical anti-tumor activity of RMC-4630 in vivo in the NCI-H358 KRASG12C xenograft model. Lower doses of RMC-4630 (10 mg/kg daily) produced durable coverage (12-16 hr) over the EC₅₀ but did not exceed the EC₇₅ and were associated with tumor growth inhibition (cytostatic threshold) but not regressions. Tumor regressions (apoptotic threshold) were observed for higher doses (30 mg/kg daily) at which the plasma exposures exceeded the EC₇₅ for 4-6 hr and the EC₅₀ for the entire dosing interval. A single dose of 30 mg/kg of RMC-4630 has been shown to induce apoptosis in vivo in the KRASG12C pancreatic tumor cell line MIA PaCa-2. The actual plasma concentration at which cell death (apoptosis) may occur may vary from tumor to tumor. It should be noted also that in in vitro studies the induction of apoptosis in KRASG12C tumor cell lines is both concentration and time-dependent. Characterization of RAS pathway activation has not been performed for normal tissue. However, in in vivo rodent studies, lower trough plasma concentrations (below EC₅₀) have been associated with improved tolerability. PK sampled at: ¹C1D22, ²Post-C1D1 dosing and C1D8 trough (˜168 h), ³Post-C1D1 and C1D2 dosing and C1D8 trough (˜168 h).

FIG. 27B is a Schematic representation of RMC-4630 pharmacokinetics at three tolerated dose schedules with peak and trough concentrations of RMC-4630 derived from the data from FIG. 27A and Table 3. Schematic depiction of the pharmacokinetic profiles in humans of three tolerated dosing regimens; daily at 60 mg, intermittent twice weekly at 140 mg (D1, D4) and intermittent twice weekly at 200 mg (D1, D2). Blue bars indicate the Cmax and Trough plasma concentrations for the respective dose regimens (see also Table 3 and FIG. 27A). Pharmacokinetic profiles for the 60 mg daily group were available from N=11. The cytostatic and apoptotic thresholds are defined in the legend to FIG. 27A.

FIG. 28 is a waterfall plot of patients with NSCLC or gynecologic tumors harboring NF1 LOF treated with RMC-4630. Data are presented for the efficacy evaluable population (N=6) defined as participants with baseline and at least one post-baseline scan or who died or had clinical progression prior to first post-baseline scan. One patient (NSCLC) with death due to clinical PD prior to first scan is not represented in this figure. NF1 LOF is loss, or significant reduction, in neurofibromin protein function is presumed from nature of mutation.

FIG. 29 is a schematic diagram depicting the phase 1b dose escalation design.

FIG. 30 is a pair of tables providing patient baseline characteristics for the phase 1b study depicted in FIG. 29.

FIG. 31 is a table providing common adverse events related to either RMC-4630 or cobimetinib. As used in the study depicted in this figure, the term “reported” in the context of AEs, is meant to describe a confidential relay of communication from a clinician to the sponsor. * Includes platelet count decrease; ** Company-defined MedDRA Query (CMQ) includes eyelid edema, face edema, generalized edema, lip edema, edema, edema peripheral, periorbital edema, and peripheral swelling. *** Includes rash, rash maculo-papular, and rash pustular; **** Includes hemoglobin decrease; ***** Includes symptoms associated with MEKi retinopathy including vision blurred and visual impairment; ^{£} RMC-4630 doses tested with daily cobimetinib: 80 mg D1D4 (n=14) and 140 mg D1D4 (n=19); ^{Σ} RMC-4630 dose tested with intermittent cobimetinib: 140 mg D1D2.

FIG. 32 is a table providing data for acceptable tolerability with RMC-4630 140 mg D1D2+Cobimetinib 40 mg D1D2. ^{£} RMC-4630 and cobimetinib doses included: RMC-4630 80 mg D1D4+cobimetinib 20 mg 21/7 (n=8), ^{Σ} RMC-4630 80 mg D1D4+cobimetinib 40 mg 21/7 (n=6), RMC-4630 140 mg D1D4+cobimetinib 20 mg 21/7 (n=12), and RMC-4630 140 mg D1D2+cobimetinib 20 mg 21/7 (n=7). ^{§} Related to either RMC-4630 or cobimetinib; ^{¥} Dose interruption, reduction, or discontinuation of either RMC-4630 or cobimetinib.

FIG. 33 is a pair of graphs demonstrating that intermittent dosing (D1D2) of RMC-4630 and Cobimetinib exceeds target plasma exposures.

FIG. 34 is a graph and corresponding table demonstrating the best change in tumor burden from baseline in KRAS^{MUT} colorectal cancer. ^{¥} Data presented for the 7 patients with KRAS mutant colorectal cancer treated with RMC-4630 140 mg twice weekly and varying cobimetinib dose and schedules (table below) out of the efficacy evaluable population (N=8) defined as patients with baseline scan and at least one post-baseline scan, or who died, or had clinical progression prior to first post-baseline scan. PD (progressive disease); SD (stable disease; PR (partial response).

FIG. 35 is a pair of tumor images for 53-year-old white female patient with KRAS^{G12D} colon cancer. Patient received two therapies: 1) FOLFOX+Avastin® and 2) FOLFIRI+Avastin®, prior to administration of RMC-4630 140 mg D1D2+cobimetinib 60 mg D1D2. Images depict a 30% reduction in tumor burden at end of cycle 2; 25% reduction at end of cycle 4—unconfirmed partial response (PR). Progressive disease (PD) measurement at 6 months.

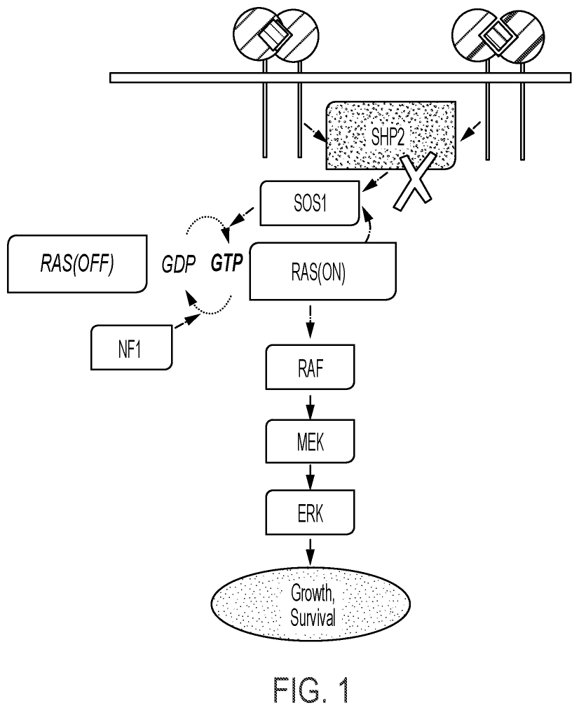

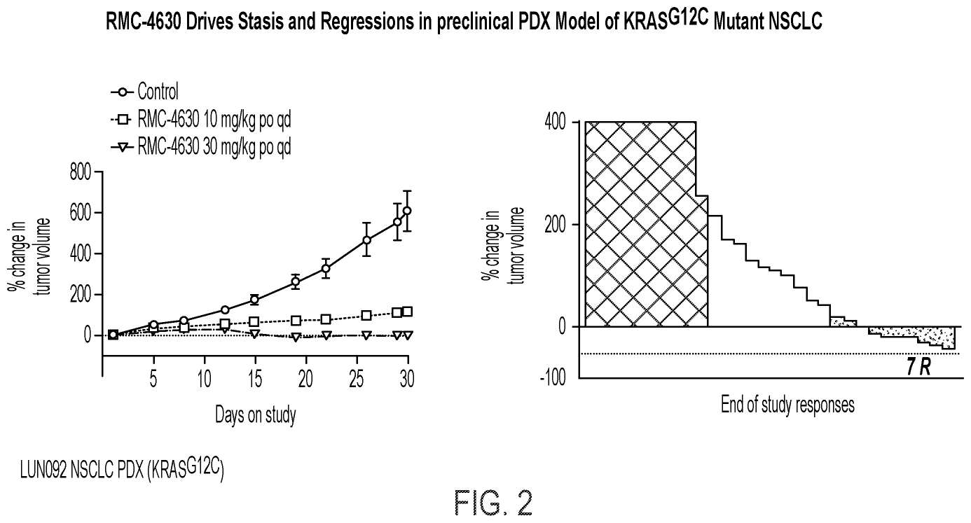

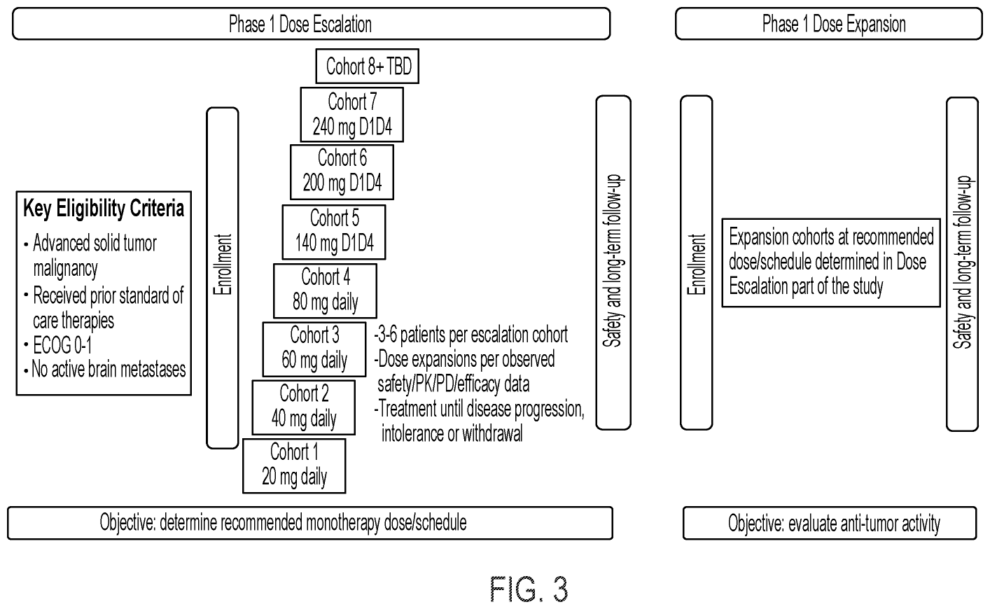

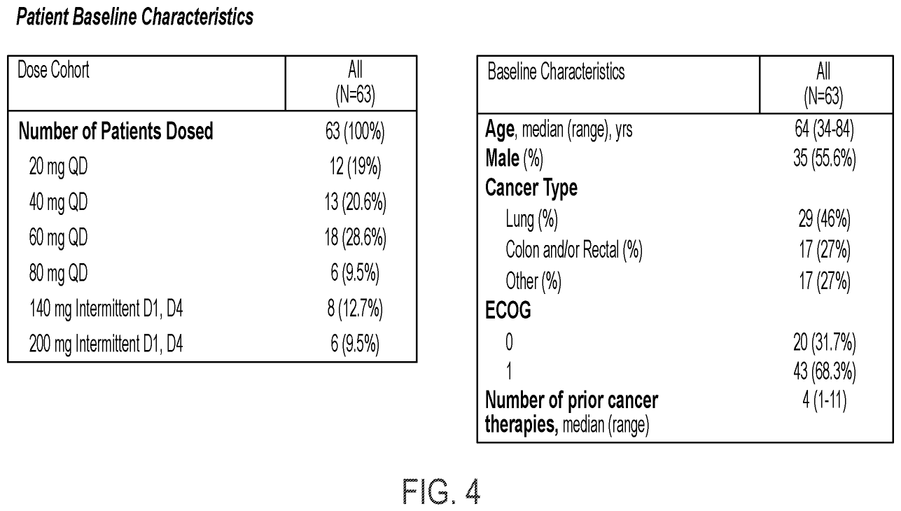

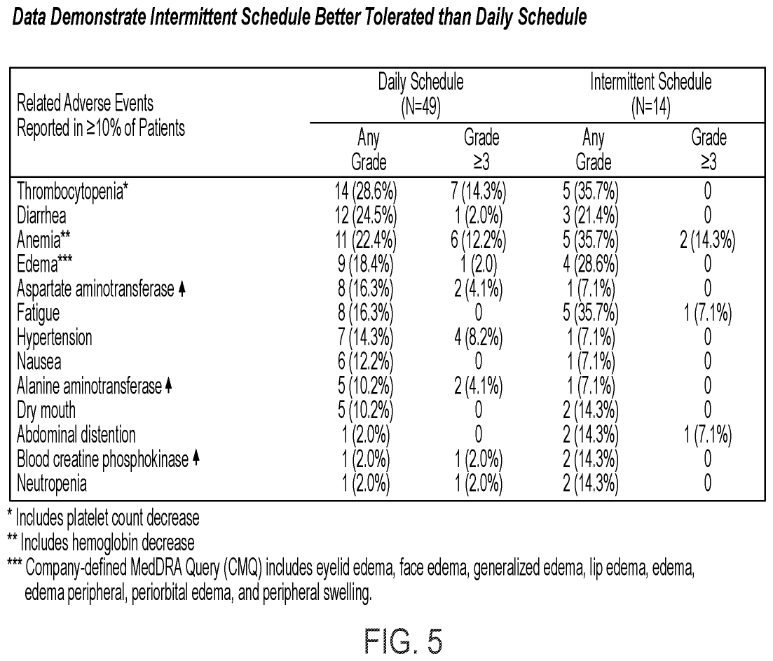

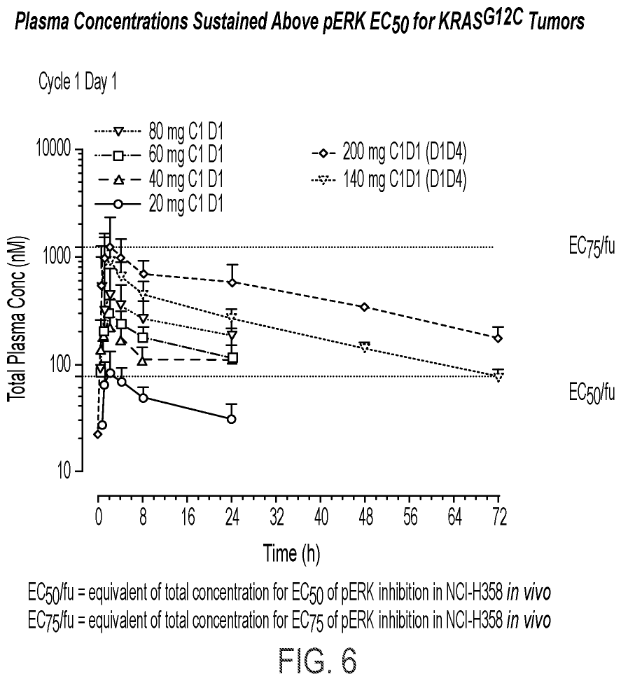

### DETAILED DESCRIPTION

Disclosed are SHP2 inhibitor compositions and methods of treatment of diseases and disorders comprising administering a SHP2 inhibitor composition of the disclosure according to an intermittent dosing schedule. Without being bound by theory, the intermittent dosing schedule provides superior treatment efficacy as either a monotherapy or a combination therapy comprising a SHP2 inhibitor when compared to a daily administration schedule at least in part because the intermittent schedule may permit healthy cells to recover between intermittent doses (e.g. a D1D4 or a D1D8 schedule). Alternatively, or in addition, an intermittent schedule in which a series of doses are provided in close succession followed by a series of resting days may increase the tumor cell killing efficacy of the target cells by inducing the target diseased cells to enter apoptosis while this blocked intermittent schedule permits a sufficient period of time for healthy cells to recover before another series of doses with a SHP2 inhibitor (e.g. a D1D2 or D1D2D3D4 schedule in a 7 day iteration).

In some embodiments, a period of time sufficient to allow healthy cells to recover may be determined by relative levels of a determination of a plasma concentration of the SHP2 inhibitor and a predetermined or measured value of an EC50 for inhibition of ERK phosphorylation following administration of the SHP2 inhibitor. In some embodiments, the predetermined or measured value of an EC50 for inhibition of ERK phosphorylation may be predetermined or measured in an in vitro or ex vivo assay or from a prior study including a sufficient number of study subjects, optionally of character-matched healthy individuals, to lead statistical power to provide a value of the EC50 for inhibition of ERK phosphorylation in the subject under treatment following the dose of the SHP2 inhibitor.

A particular treatment outcome measure is tumor burden. As used in the disclosure, the term “tumor burden” is meant to describe, without limitation, one or more of a number of cancer cells in a tumor, a number of cancer cells in a biopsy, a number of cancer cells in a structure (e.g. a lymph node or an organ), a number of cells in the circulating blood of the subject or a number of cells in the subject's body; a size of a tumor; a volume of a tumor; a circumference or diameter of a tumor, or the amount of cancer in the body. The term tumor burden is meant to be synonymous with the term “tumor load”.

A particular treatment outcome measure is inhibition of ERK phosphorylation.

A particular treatment outcome measure is reduction or elimination of a sign or a symptom of the disease or disorder. A sign of a disease or disorder is presented by the subject as an objectively detectable characteristic, regardless of the subject's awareness of the sign or a change in the sign (e.g. tumor burden). A symptom of a disease or disorder is a subjective experience of the disease or disorder felt by the patient (e.g. pain).

A particular treatment outcome measure is induction of remission of the disease or disorder. Alternatively or in addition, a particular treatment outcome measure is prevention of relapse of the disease or disorder.

A particular treatment outcome measure is elimination of the disease or disorder, also referred to as a cure.

Methods of the disclosure comprise administration of a SHP2 inhibitor. While any SHP2 inhibitor is contemplated, a particular SHP2 inhibitor is RMC-4630. SHP2 inhibitors of the disclosure may be administered as monotherapies or as combination therapies with any other therapeutic agent. Particular second or additional therapeutic agents for use in a combination therapy include proliferation inhibitors. Exemplary proliferation inhibitors include, but are not limited to RAS inhibitors and MEK inhibitors. A particular second or additional therapeutic agent comprises cobimetinib. A particular second or additional therapeutic agent is a PD-L1 or PD-1 inhibitor. A particular second or additional therapeutic agent is a CDK4/6 inhibitor. In particular embodiments, SHP2 inhibitors of the disclosure, including RMC-4630, are administered according to an intermittent schedule. When provided as a combination therapy, SHP2 inhibitors of the disclosure, including RMC-4630, are administered according to an intermittent schedule. Optionally, when provided as a combination therapy, the second or additional therapeutic agent is provided on an intermittent schedule. Alternatively, the second or additional therapeutic agent may be provided on a continuous, daily, weekly, or monthly schedule.

### Clinical Data Using RMC-4630

The RMC-4630 phase 1/2 program includes two clinical trials. RMC-4630-01, a phase 1 dose escalation study of RMC-4630 as a single agent RMC-4630-02, a phase 1b/2 study of RMC-4630 in combination with the MEK inhibitor cobimetinib (Cotellic®). The disclosure provides clinical data from both the RMC-4630-01 study and RMC-4630-02 study.

RMC-4630-01 Study of Single Agent RMC-4630 Inpatients with Advanced Solid Tumors. RMC-4630-01 is a phase 1 dose escalation study in patients with advanced cancers that evaluates the safety, pharmacokinetics and pharmacodynamic effects of RMC-4630 as a single agent under two different dose administration schedules; daily dosing and twice weekly dosing. Anti-tumor activity is also evaluated in patients who have tumors harboring mutations in the RAS-MAPK pathway.

The RMC-4630-01 study was initially designed to evaluate two different schedules: a daily dosing schedule and an intermittent dosing schedule (D1, D4 of every week). The intermittent schedule was intended to achieve intermittent target coverage, which, in preclinical models, was associated with similar or superior activity and better tolerability.

At the latest data cut-off, 63 patients had received study drug and were evaluable for safety: 14 with the intermittent schedule and 49 with the daily schedule. Dose escalation has been completed for the daily dosing schedule. Dose escalation continues using the intermittent schedule. Preliminary data suggest that the intermittent schedule is a particular schedule for RMC-4630. Safety, tolerability and PK data for patients treated with the intermittent schedule are provided here separately from patients treated with the daily schedule.

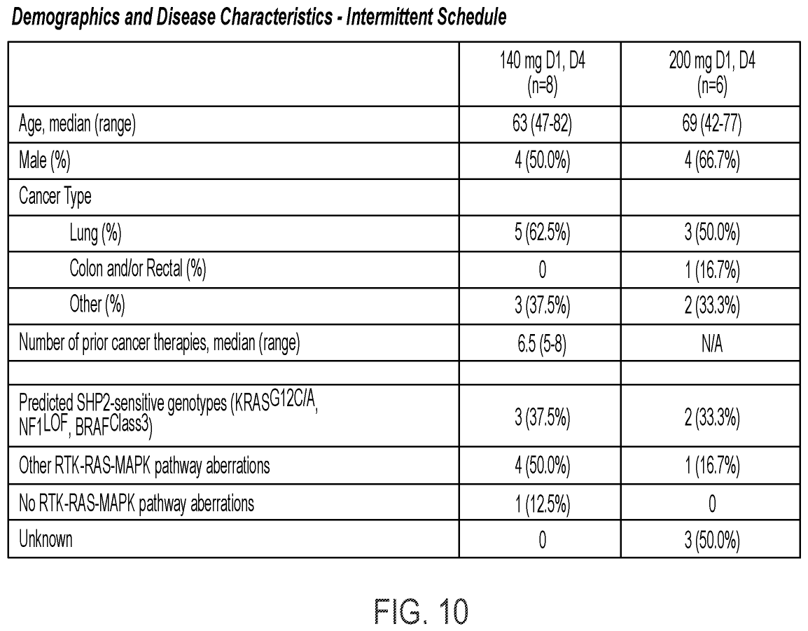

RMC 6430 Interim safety and tolerability of an intermittent schedule. Fourteen patients dosed with the D1, D4 schedule have been evaluated for safety after a median follow-up of 2 months. Demographic information is shown in FIG. 10.

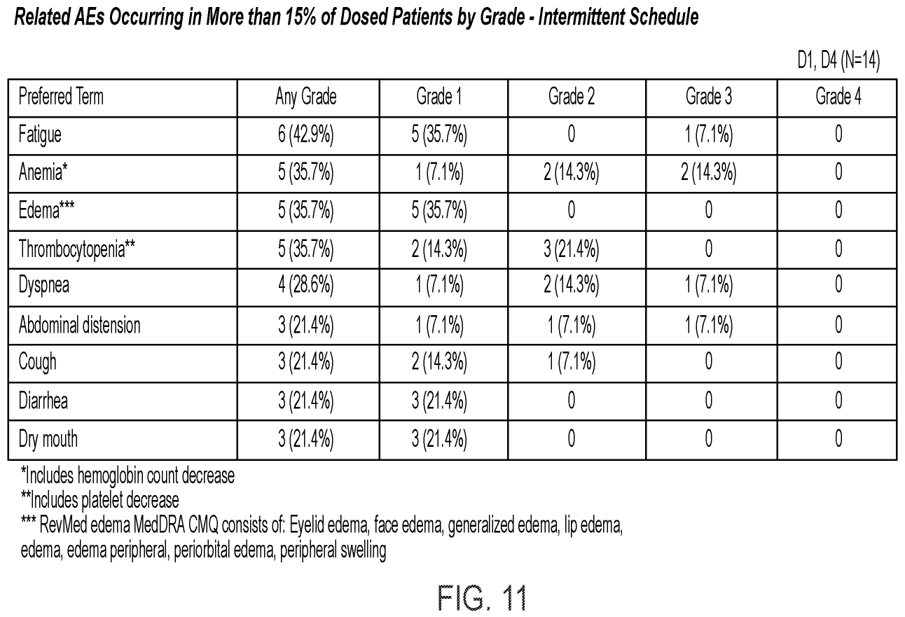

The emerging safety profile is consistent with the mechanistic effects of the drug candidate on SHP2 and hence the RAS signaling cascade, including edema, reduced red cell production (low hemoglobin concentration and worsening of pre-existing anemia), reduced platelet production (thrombocytopenia), hypertension and fatigue. This safety profile was largely predictable from non-clinical studies and clinical studies of other well-known inhibitors of this pathway. Treatment-related and emergent adverse events (AEs) occurring in greater than 15% of patients are provided in FIG. 11. No related grade 4 or grade 5 AEs have been reported for this schedule. One related SAE has been reported in a patient with pancreatic cancer receiving 200 mg twice weekly who was hospitalized with grade 3 abdominal distension; the AE was unresolved at the time the patient withdrew from the study to transfer to hospice care.

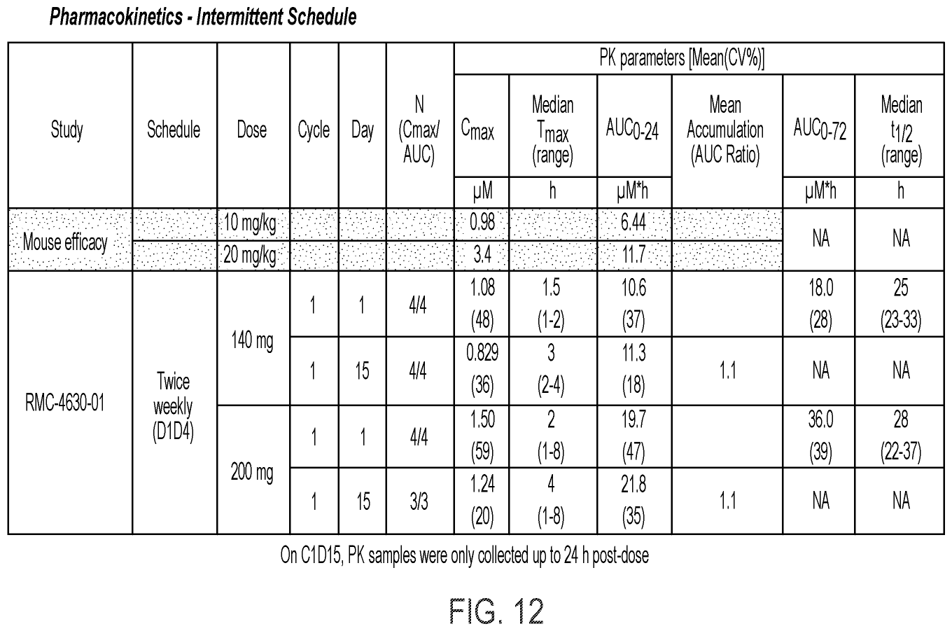

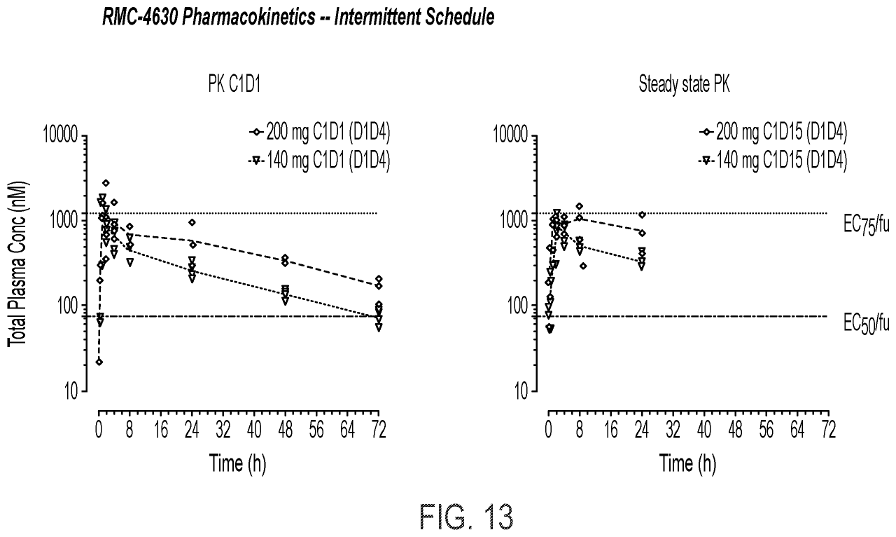

RMC-4630 Pharmacokinetics with Intermittent Schedule. The pharmacokinetic profile of RMC-4630 after dosing on D1, D4 schedule is shown in FIGS. 12 and 13. Plasma levels of RMC-4630 after oral administration to patients were similar to those predicted from preclinical studies in rats and dogs. No accumulation from day 1 to day 15 was observed. Plasma exposure at both dose levels was within the range anticipated to be biologically active from preclinical models. After a single dose of 140 mg the plasma concentration of RMC-4630 remains above the in vivo EC₅₀ for pERK for 72 hours. The half-life of RMC-4630 is estimated to be 25 hrs.

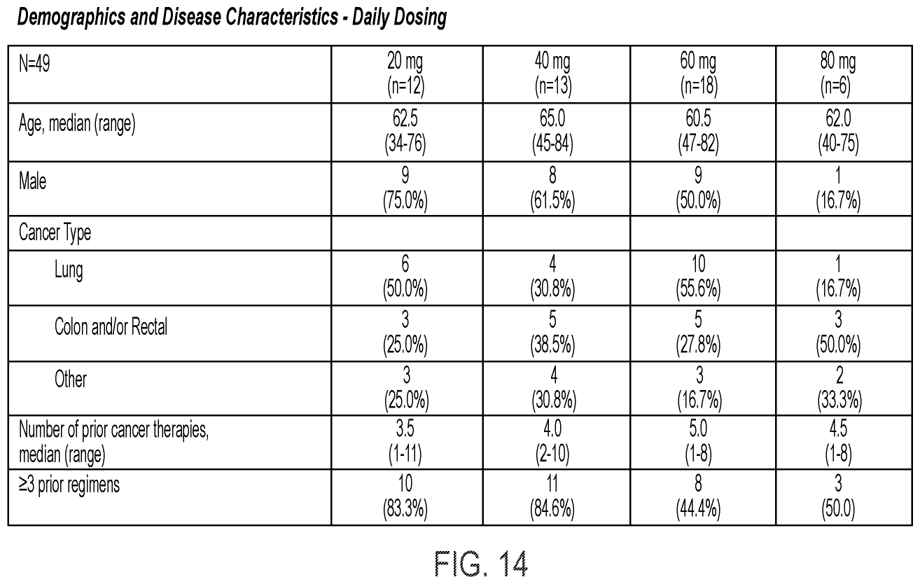

Interim safety and tolerability of RMC-4630 by a daily schedule. Forty-nine patients have been treated with the daily schedule. Median follow-up is 2 months (range 1-14 m). Demographic information is shown in FIG. 14.

Daily dosing has been associated with more frequent and severe AEs than the intermittent schedule. As with the intermittent dosing schedule, the emerging safety profile from the daily dosing schedule is consistent with the mechanistic effects of the drug on SHP2 and the RAS signaling pathways. The maximal tolerated dose (MTD) for daily dosing has not been formally determined, although dose escalation will not continue beyond the 80 mg daily level already evaluated. Were further development with this schedule to be pursued, the recommended phase 2 dose for this daily schedule would be in the range of 60 mg.

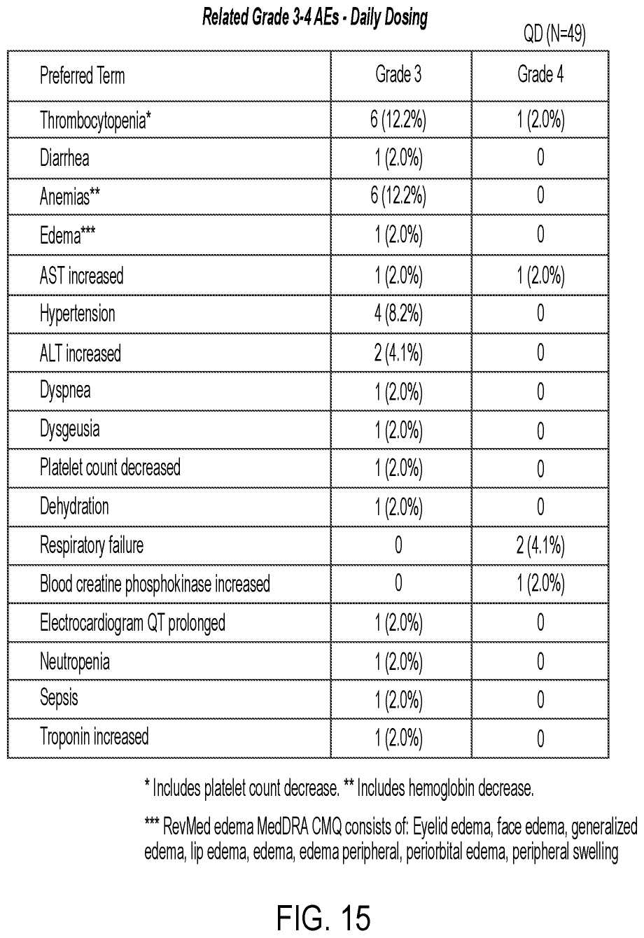

Related grade 3 and grade 4 AEs are shown in FIG. 15. No toxicities consistent with ‘off-target’ effects have been reported. No deaths (grade 5 AEs) have been ascribed to daily administration of RMC-4630. Increases in liver enzymes such as alanine transaminase and aspartate transaminase have been observed at all grades. These have been attributed, wholly or in part, to RMC-4630 in 10% or 16% of patients treated with the daily schedule respectively. In two patients (4%) the increase in alanine transaminase or aspartate transaminase was either grade 3 or grade 4.

Eight patients (16%) treated with the daily schedule have experienced toxicities involving the lungs or respiratory system that were attributed by the treating investigator in part to RMC-4630. These were generally moderate or mild. Two additional cases of grade 4 respiratory failure are discussed in more detail below in the description of serious adverse events (SAEs). There has been little evidence of systemic activation of the immune system in subjects treated with RMC-4630. There have been no reports of pneumonitis. Related adverse events involving other important organs such as the heart, brain, kidneys have been either uncommon and mild to moderate in severity, or not reported.

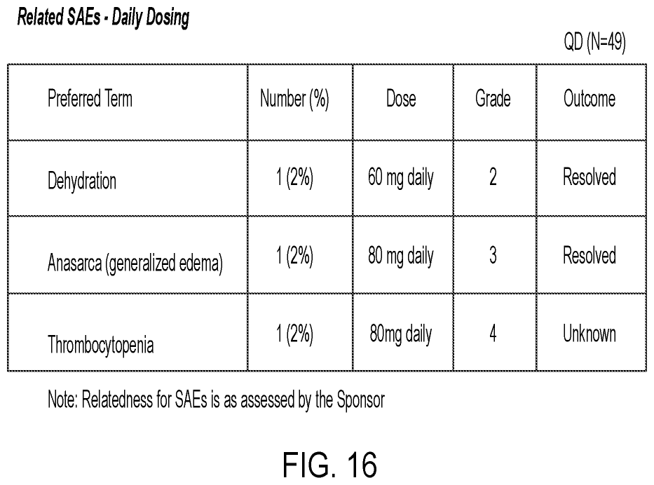

There have been three (6%) serious adverse events thought to be possibly or probably related to study drug as assessed by the Sponsor (FIG. 16). Three additional SAEs have occurred in which the investigator was unable to rule out an association with study drug, but where the evidence for causality by RMC-4630 was absent or considered unlikely by the Sponsor. One patient with extensive metastases of tumor in the lungs developed grade 4 shortness of breath and was hospitalized and treated with oxygen. The adverse event was ongoing when the patient was withdrawn from the study. A second patient with fever and radiologic evidence of infectious pneumonia developed grade 4 respiratory failure and was treated with oxygen, systemic antibiotics and corticosteroids. The event was ongoing when the patient died due to progression of underlying cancer. A third patient developed a single reading of grade 3 prolongation of QTc. This patient had been receiving 60 mg daily of RMC-4630 but had not received any dose for three days at the time of the reading. The patient had a previous history of prolonged QTc, underlying systemic lupus, and was taking ondansetron. QTc was prolonged (grade 1) at baseline. Five hours after the prolonged QTc reading the patient had two follow-up ECGs that showed normal QTc interval.

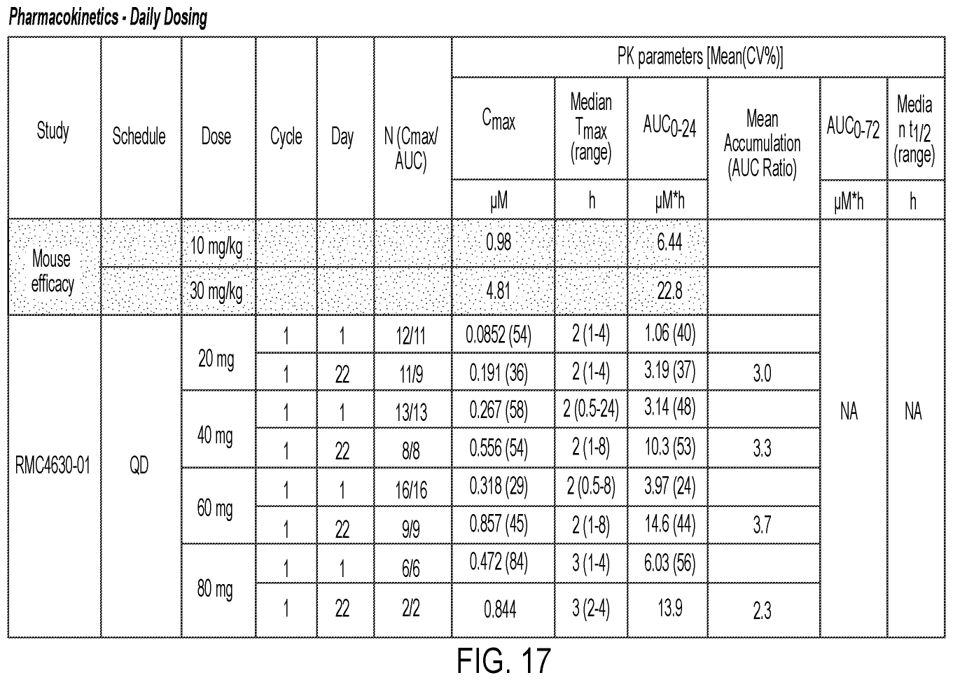

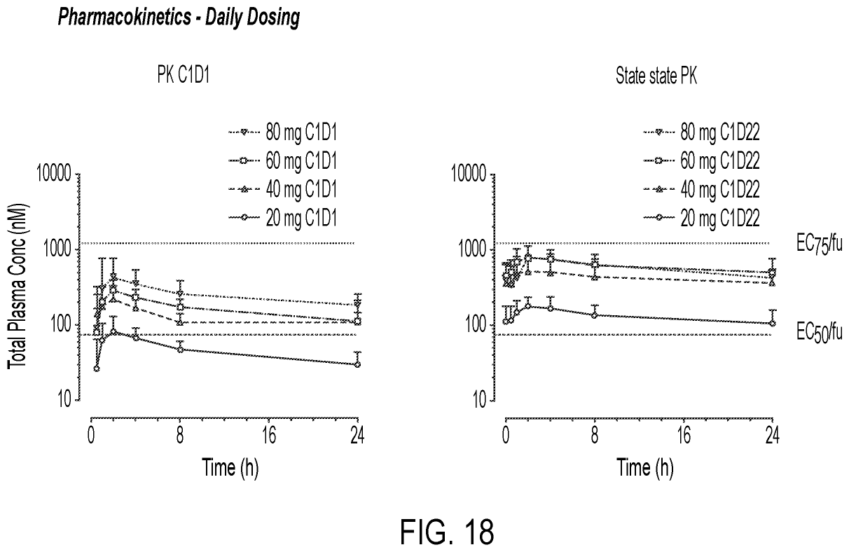

Pharmacokinetics of RMC-4630 with daily schedule. With daily dosing plasma concentrations of RMC-4630 reached a steady state by day 22 (FIGS. 17 and 18). Plasma concentrations of RMC-4630 in the blood at all daily dose levels were consistently higher than the in vivo EC₅₀ for pERK in tumor models. Exposure increased approximately proportionally with increasing dose. The total exposure to RMC-4630 over a 24 hour period at the putative MTD of 60 mg daily was 14.6 uM·hr. This is more than twice the exposure that is required to see anti-tumor effects, particularly tumor stasis, in animal models (6.44 uM·hr).

Pharmacodynamic effects of RMC-4630, comparison of daily and intermittent schedules. Activation of the protein ERK, which is an important protein in the RAS signaling pathway and a substrate for MEK, is a good surrogate for the inhibition of pathway activity by a SHP2 inhibitor. The pharmacodynamic effects of RMC-4630 on activation of ERK were studied in the blood cells of patients being treated with RMC-4630. Despite considerable assay variability and inter-patient variability, which is common for these types of dynamic assays in patients, there was a trend in favor of inhibition of activated ERK in peripheral blood cells at all dose levels tested. These effects are consistent with engagement and inhibition of the SHP2 target and downstream RAS signaling by RMC-4630.

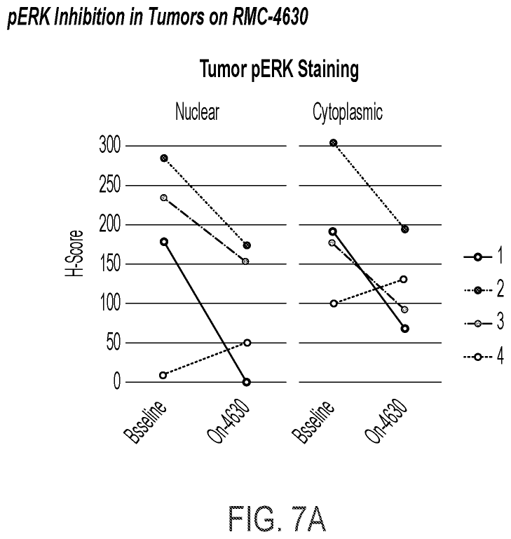

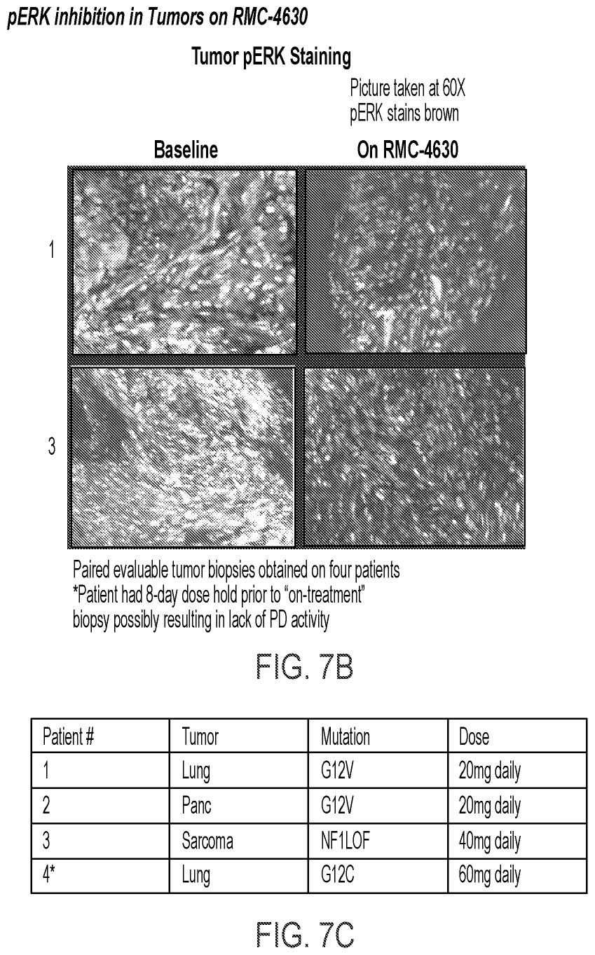

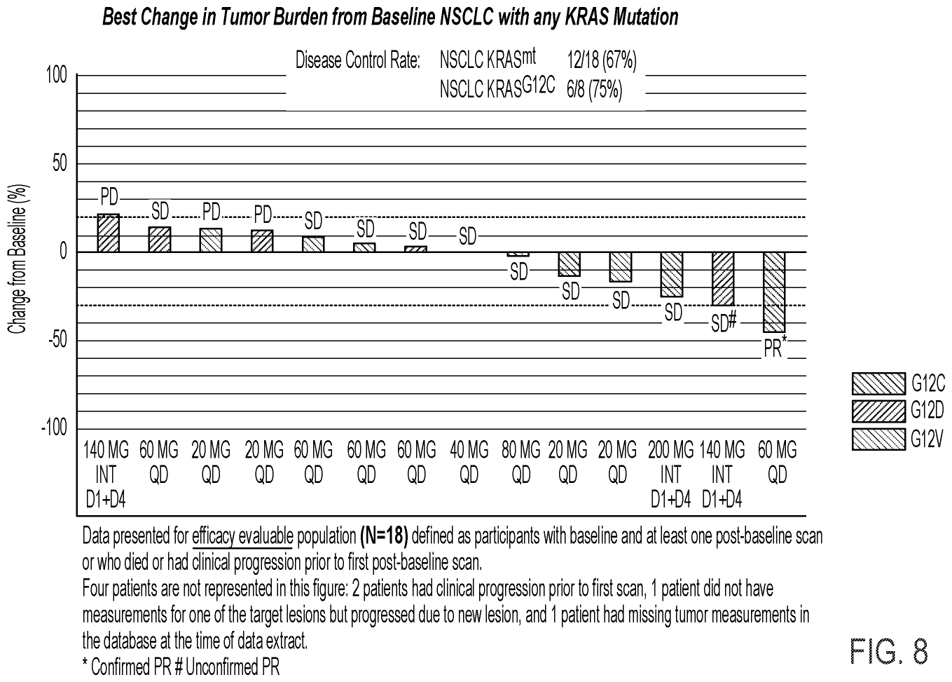

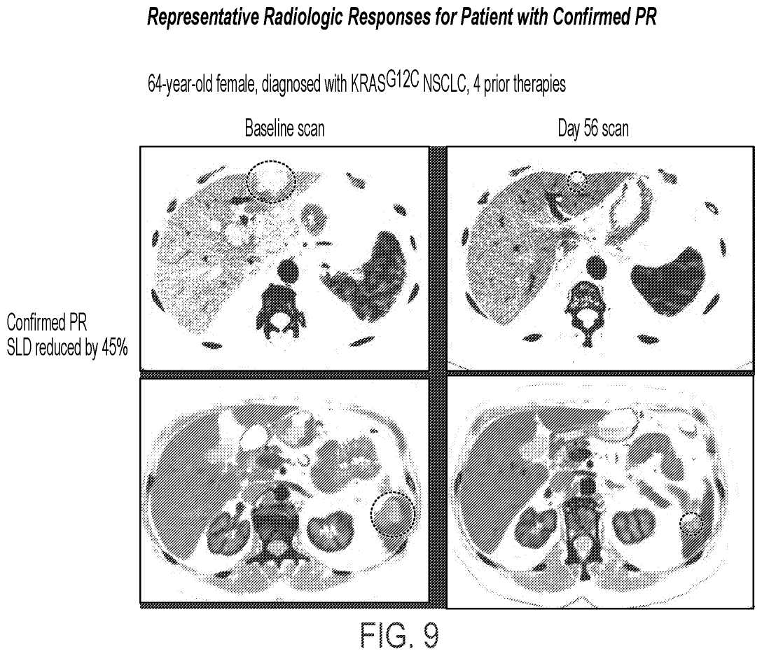

Phosphorylation of ERK has been assessed in tumor before, and while receiving, RMC-4630 (FIG. 7). In three cases there was a reduction in phosphorylation of cytoplasmic and nuclear ERK in the tumor while RMC-4630 was at steady state. One patient's tumor showed no reduction in tumor pERK, but this tumor showed very little phosphorylation in the pre-treatment sample and had not received any RMC-4630 for eight days prior to the second tumor biopsy.

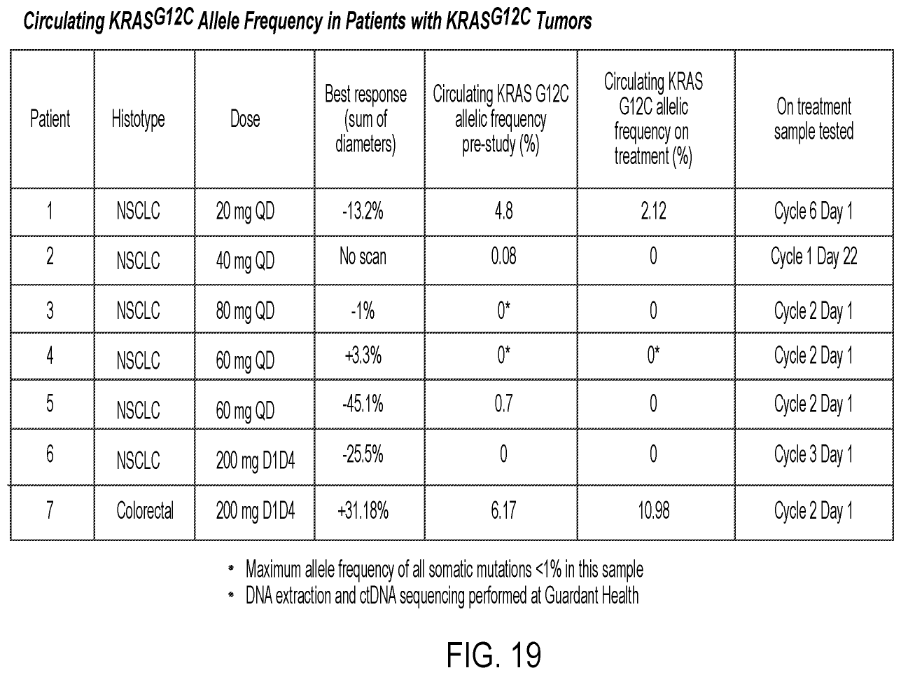

Allelic burden of circulating KRAS^{G12C} tumor DNA (ctDNA) has been assessed prior to study and at least once on study in seven patients with tumors harboring KRAS^{G12C} (FIG. 19). KRAS^{G12C} DNA was detected in four of seven patients prior to study. In three patients with NSCLC and either PR or SD as best response there was a reduction in circulating KRAS^{G12C} In one patient with colon cancer who had PD the allelic frequency of KRAS^{G12C} increased.

Interim evidence of clinical activity of RMC-4630 on daily and intermittent schedules. There is preliminary evidence that RMC-4630 has single agent anti-tumor activity in KRAS mutant NSCLC. One patient with KRAS^{G12C} NSCLC treated at 60 mg daily had a confirmed PR, with a 49% reduction in tumor volume as measured by CT imaging. A second NSCLC patient with KRAS^{G12D}+SHP2^{V428M} treated with 140 mg D1, D4 had an unconfirmed PR. Disease control rate (DCR, the sum of best response of PR and SD cases) for patients with KRAS^{G12C} NSCLC thus far is 6/8 (75%).

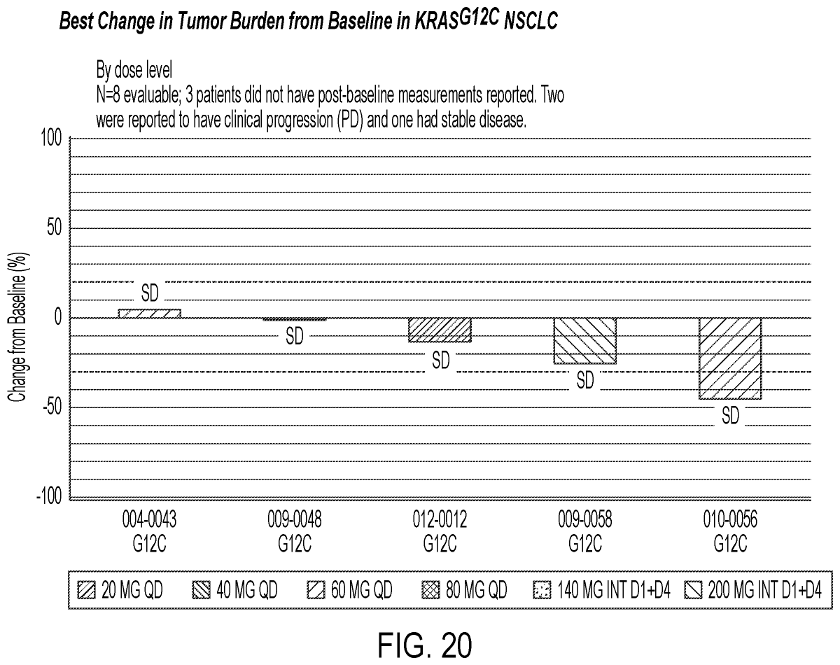

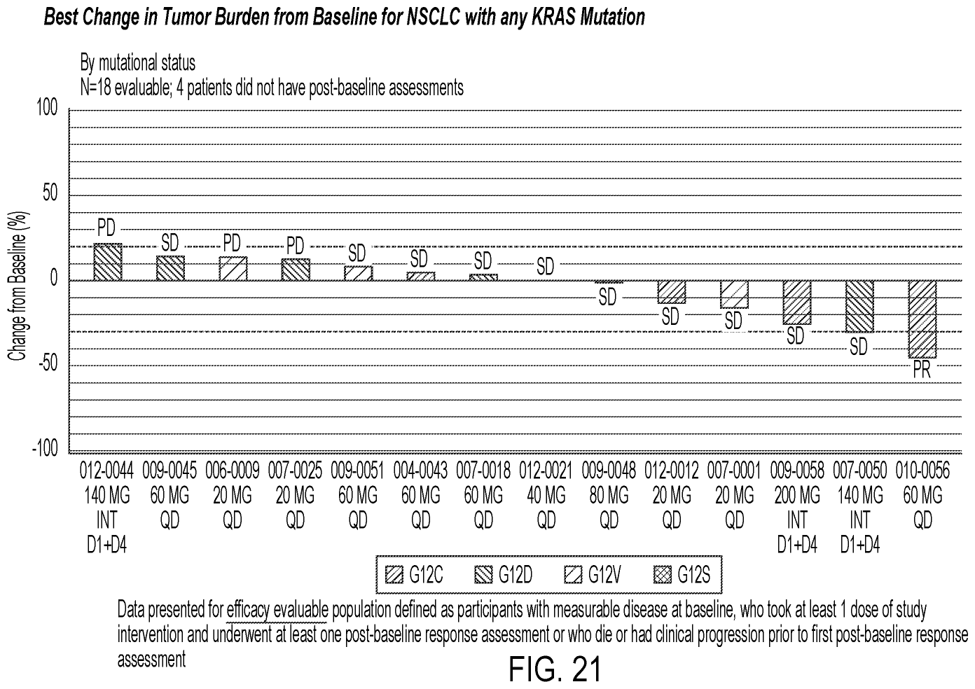

Five patients with KRAS^{G12C} NSCLC have had follow-up CT scans of target lesions and have had either PR or SD (FIG. 20); three patients have not reported follow-up measurements of target lesions, of which one has been recorded as best response of SD and two of PD. For all patients with KRAS mutant NSCLC disease, DCR thus far is 12/18 (67%) (FIG. 21). One patient with KRAS^{G12V} NSCLC has been on treatment for over 14 months with stable disease (˜15% reduction in tumor volume). In histotypes other than NSCLC the best response thus far has been SD.

RMC-4630-02 study of RMC-4630 in combination with cobimetinib (Cotellic®) patients with advanced solid tumors. RMC-4630-02 is a phase 1b/2 dose escalation study of RMC-4630 in combination with the MEK inhibitor cobimetinib in patients with advanced cancers that harbor mutations in the RAS signaling pathway. The study evaluates the safety, tolerability and pharmacokinetics of RMC-4630 and cobimetinib under two different dose administration schedules in order to determine a recommended phase 2 dose and schedule for further clinical testing. Initially the study assesses twice weekly RMC-4630 (D1, D4) with daily cobimetinib (21 days on, 7 off). In the second schedule, both RMC-4630 and cobimetinib are dosed intermittently. A preliminary evaluation of anti-tumor activity is also being made.

At the latest data cut-off, eight patients had received study medication at the first dose level and were evaluable for safety. Dose escalation to the next highest dose level has occurred and enrollment is ongoing.

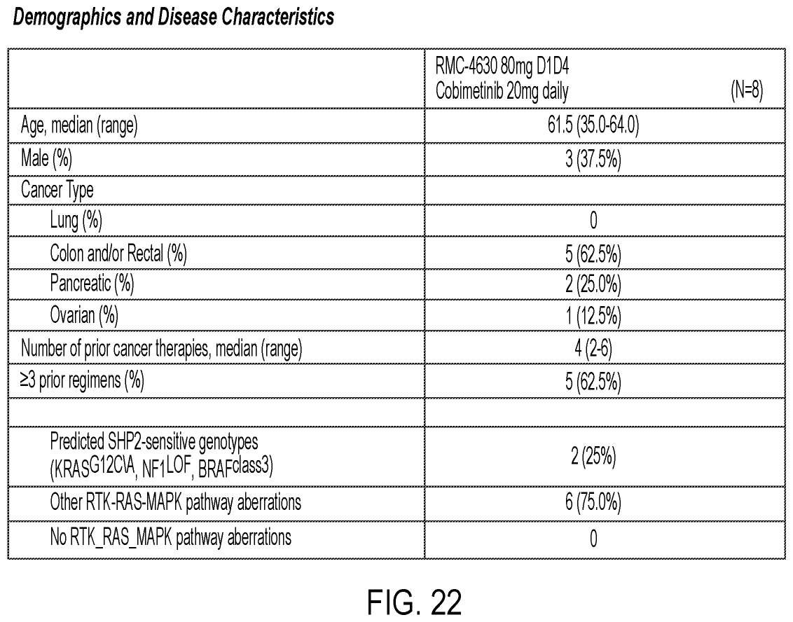

Interim safety and tolerability. Eight patients have been evaluated for safety after a median follow-up of less than 2 months. Demographic information is shown in FIG. 22.

The emerging safety profile is consistent with the mechanistic effects of both SHP2 inhibition and MEK inhibition, including edema, diarrhea and other gastrointestinal toxicity, anemia and rash. This safety profile was largely predictable from single agent clinical studies of both agents.

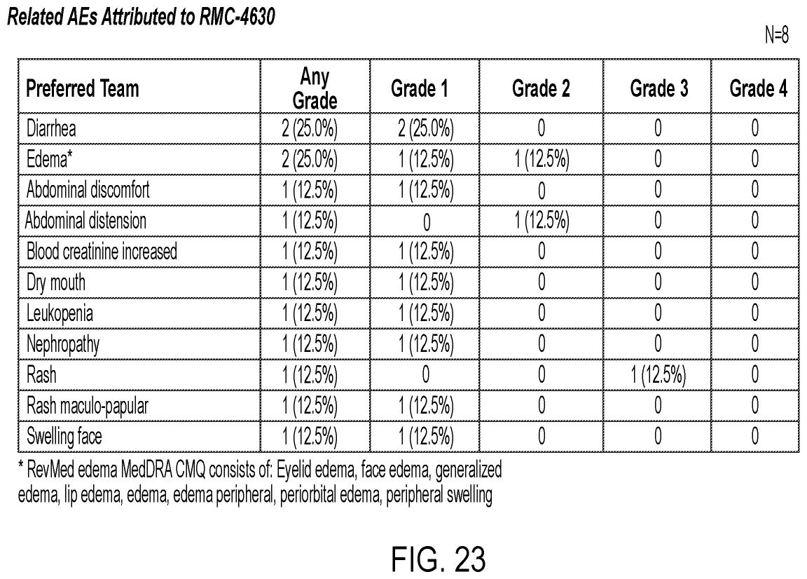

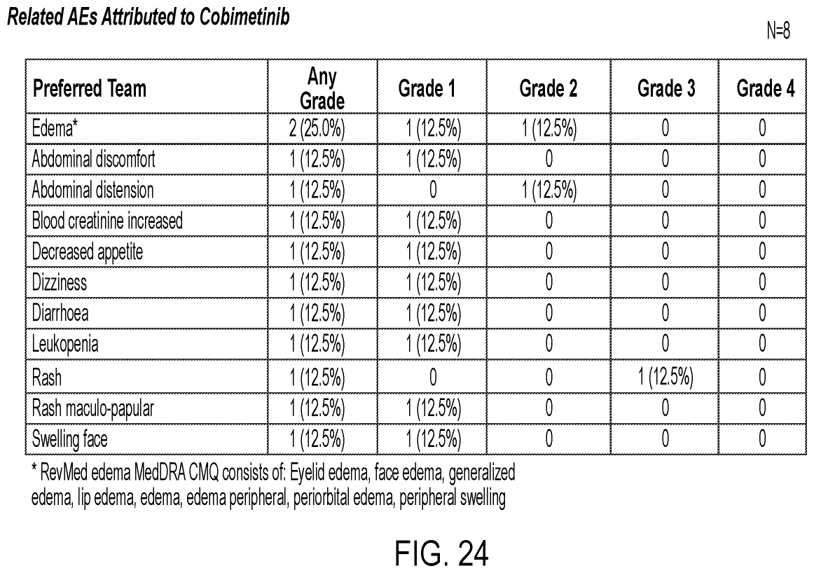

Treatment-related and emergent adverse events (AEs) are listed in FIGS. 23 and 24. There have been no grade 4 or grade 5 AEs or related serious AEs (SAEs) reported.

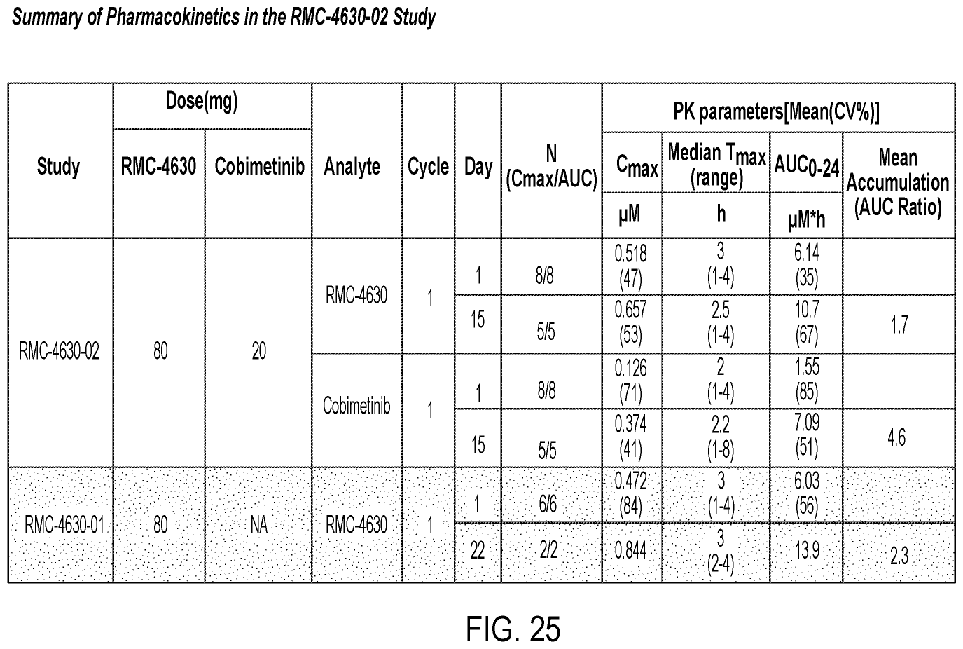

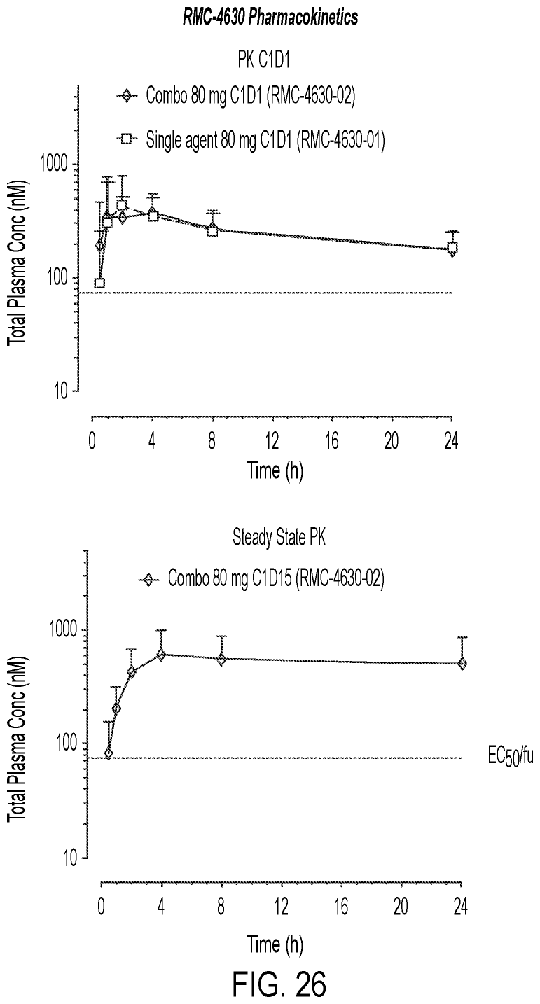

Pharmacokinetics. The pharmacokinetic profiles of RMC-4630 and cobimetinib are shown in FIGS. 25 and 26. Plasma levels of RMC-4630 are continuously greater than the predicted EC₅₀ for pERK inhibition in preclinical tumor models.

PD and Clinical activity. Only three patients have been evaluated for efficacy in this study. No efficacy data or ctDNA data are available in the electronic database at the time of reporting.

### Combination Therapy

The methods of the invention may include a compound of the invention used alone or in combination with one or more additional therapies (e.g., non-drug treatments or therapeutic agents). The dosages of one or more of the additional therapies (e.g., non-drug treatments or therapeutic agents) may be reduced from standard dosages when administered alone. For example, doses may be determined empirically from drug combinations and permutations or may be deduced by isobolographic analysis (e.g., Black et al., Neurology 65:S3-S6 (2005)).

A compound of the present invention may be administered before, after, or concurrently with one or more of such additional therapies. When combined, dosages of a compound of the invention and dosages of the one or more additional therapies (e.g., non-drug treatment or therapeutic agent) provide a therapeutic effect (e.g., synergistic or additive therapeutic effect). A compound of the present invention and an additional therapy, such as an anti-cancer agent, may be administered together, such as in a unitary pharmaceutical composition, or separately and, when administered separately, this may occur simultaneously or sequentially. Such sequential administration may be close or remote in time.

In some embodiments, the additional therapy is the administration of side-effect limiting agents (e.g., agents intended to lessen the occurrence or severity of side effects of treatment). For example, in some embodiments, the compounds of the present invention can also be used in combination with a therapeutic agent that treats nausea. Examples of agents that can be used to treat nausea include: dronabinol, granisetron, metoclopramide, ondansetron, and prochlorperazine, or pharmaceutically acceptable salts thereof.

In some embodiments, the one or more additional therapies includes a non-drug treatment (e.g., surgery or radiation therapy). In some embodiments, the one or more additional therapies includes a therapeutic agent (e.g., a compound or biologic that is an anti-angiogenic agent, signal transduction inhibitor, antiproliferative agent, glycolysis inhibitor, or autophagy inhibitor). In some embodiments, the one or more additional therapies includes a non-drug treatment (e.g., surgery or radiation therapy) and a therapeutic agent (e.g., a compound or biologic that is an anti-angiogenic agent, signal transduction inhibitor, antiproliferative agent, glycolysis inhibitor, or autophagy inhibitor). In other embodiments, the one or more additional therapies includes two therapeutic agents. In still other embodiments, the one or more additional therapies includes three therapeutic agents. In some embodiments, the one or more additional therapies includes four or more therapeutic agents.

### Non-Drug Therapies

Examples of non-drug treatments include, but are not limited to, radiation therapy, cryotherapy, hyperthermia, surgery (e.g., surgical excision of tumor tissue), and T cell adoptive transfer (ACT) therapy.

In some embodiments, the compounds of the invention may be used as an adjuvant therapy after surgery. In some embodiments, the compounds of the invention may be used as a neo-adjuvant therapy prior to surgery.

Radiation therapy may be used for inhibiting abnormal cell growth or treating a hyperproliferative disorder, such as cancer, in a subject (e.g., mammal (e.g., human)). Techniques for administering radiation therapy are known in the art. Radiation therapy can be administered through one of several methods, or a combination of methods, including, without limitation, external-beam therapy, internal radiation therapy, implant radiation, stereotactic radiosurgery, systemic radiation therapy, radiotherapy, and permanent or temporary interstitial brachy therapy. The term “brachy therapy,” as used herein, refers to radiation therapy delivered by a spatially confined radioactive material inserted into the body at or near a tumor or other proliferative tissue disease site. The term is intended, without limitation, to include exposure to radioactive isotopes (e.g., At-211, I-131, I-125, Y-90, Re-186, Re-188, Sm-153, Bi-212, P-32, and radioactive isotopes of Lu). Suitable radiation sources for use as a cell conditioner of the present invention include both solids and liquids. By way of non-limiting example, the radiation source can be a radionuclide, such as I-125, I-131, Yb-169, Ir-192 as a solid source, I-125 as a solid source, or other radionuclides that emit photons, beta particles, gamma radiation, or other therapeutic rays. The radioactive material can also be a fluid made from any solution of radionuclide(s), e.g., a solution of I-125 or I-131, or a radioactive fluid can be produced using a slurry of a suitable fluid containing small particles of solid radionuclides, such as Au-198, or Y-90, Moreover, the radionuclide(s) can be embodied in a gel or radioactive micro spheres.

In some embodiments, the compounds of the present invention can render abnormal cells more sensitive to treatment with radiation for purposes of killing or inhibiting the growth of such cells. Accordingly, this invention further relates to a method for sensitizing abnormal cells in a mammal to treatment with radiation that comprises administering to the mammal an amount of a compound of the present invention, which amount is effective to sensitize abnormal cells to treatment with radiation. The amount of the compound in this method can be determined according to the means for ascertaining effective amounts of such compounds described herein. In some embodiments, the compounds of the present invention may be used as an adjuvant therapy after radiation therapy or as a neo-adjuvant therapy prior to radiation therapy.

In some embodiments, the non-drug treatment is a T cell adoptive transfer (ACT) therapy. In some embodiments, the T cell is an activated T cell. The T cell may be modified to express a chimeric antigen receptor (CAR). CAR modified T (CAR-T) cells can be generated by any method known in the art. For example, the CAR-T cells can be generated by introducing a suitable expression vector encoding the CAR to a T cell. Prior to expansion and genetic modification of the T cells, a source of T cells is obtained from a subject. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain embodiments of the present invention, any number of T cell lines available in the art may be used. In some embodiments, the T cell is an autologous T cell. Whether prior to or after genetic modification of the T cells to express a desirable protein (e.g., a CAR), the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 7,572,631; 5,883,223; 6,905,874; 6,797,514; and 6,867,041.

### Therapeutic Agents

A therapeutic agent may be a compound used in the treatment of cancer or symptoms associated therewith.

For example, a therapeutic agent may be a steroid. Accordingly, in some embodiments, the one or more additional therapies includes a steroid. Suitable steroids may include, but are not limited to, 21-acetoxypregnenolone, alclometasone, algestone, amcinonide, beclomethasone, betamethasone, budesonide, chloroprednisone, clobetasol, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, hydrocortisone, loteprednol etabonate, mazipredone, medrysone, meprednisone, methylprednisolone, mometasone furoate, paramethasone, prednicarbate, prednisolone, prednisolone 25-diethylaminoacetate, prednisolone sodium phosphate, prednisone, prednival, prednylidene, rimexolone, tixocortol, triamcinolone, triamcinolone acetonide, triamcinolone benetonide, triamcinolone hexacetonide, and salts or derivatives thereof.

Further examples of therapeutic agents that may be used in combination therapy with a compound of the present invention include compounds described in the following patents: U.S. Pat. Nos. 6,258,812, 6,630,500, 6,515,004, 6,713,485, 5,521,184, 5,770,599, 5,747,498, 5,990,141, 6,235,764, and 8,623,885, and International Patent Applications WO01/37820, WO01/32651, WO02/68406, WO02/66470, WO02/55501, WO04/05279, WO04/07481, WO04/07458, WO04/09784, WO02/59110, WO99/45009, WO00/59509, WO99/61422, WO00/12089, and WO00/02871.

A therapeutic agent may be a biologic (e.g., cytokine (e.g., interferon or an interleukin such as IL-2)) used in treatment of cancer or symptoms associated therewith. In some embodiments, the biologic is an immunoglobulin-based biologic, e.g., a monoclonal antibody (e.g., a humanized antibody, a fully human antibody, an Fc fusion protein, or a functional fragment thereof) that agonizes a target to stimulate an anti-cancer response or antagonizes an antigen important for cancer. Also included are antibody-drug conjugates.

A therapeutic agent may be a T-cell checkpoint inhibitor. In one embodiment, the checkpoint inhibitor is an inhibitory antibody (e.g., a monospecific antibody such as a monoclonal antibody). The antibody may be, e.g., humanized or fully human. In some embodiments, the checkpoint inhibitor is a fusion protein, e.g., an Fc-receptor fusion protein. In some embodiments, the checkpoint inhibitor is an agent, such as an antibody, that interacts with a checkpoint protein. In some embodiments, the checkpoint inhibitor is an agent, such as an antibody, that interacts with the ligand of a checkpoint protein. In some embodiments, the checkpoint inhibitor is an inhibitor (e.g., an inhibitory antibody or small molecule inhibitor) of CTLA-4 (e.g., an anti-CTLA-4 antibody or fusion a protein). In some embodiments, the checkpoint inhibitor is an inhibitor or antagonist (e.g., an inhibitory antibody or small molecule inhibitor) of PD-1. In some embodiments, the checkpoint inhibitor is an inhibitor or antagonist (e.g., an inhibitory antibody or small molecule inhibitor) of PDL-1. In some embodiments, the checkpoint inhibitor is an inhibitor or antagonist (e.g., an inhibitory antibody or Fc fusion or small molecule inhibitor) of PDL-2 (e.g., a PDL-2/Ig fusion protein). In some embodiments, the checkpoint inhibitor is an inhibitor or antagonist (e.g., an inhibitory antibody or small molecule inhibitor) of B7-H3, B7-H4, BTLA, HVEM, TIM3, GAL9, LAG3, VISTA, KIR, 2B4, CD160, CGEN-15049, CHK 1, CHK2, A2aR, B-7 family ligands, or a combination thereof. In some embodiments, the checkpoint inhibitor is pembrolizumab, nivolumab, PDR001 (NVS), a PD-L1 antibody such as, e.g., avelumab, durvalumab, atezolizumab, pidilizumab, JNJ-63723283 (JNJ), BGB-A317 (BeiGene & Celgene) or a checkpoint inhibitor disclosed in Preusser, M. et al. (2015) Nat. Rev. Neurol., including, without limitation, ipilimumab, tremelimumab, nivolumab, pembrolizumab, AMP224, AMP514/MEDI0680, BMS936559, MED14736, MPDL3280A, MSB0010718C, BMS986016, IMP321, lirilumab, IPH2101, 1-7F9, and KW-6002.

A therapeutic agent may be an anti-TIGIT antibody, such as MBSA43, BMS-986207, MK-7684, COM902, AB154, MTIG7192A or OMP-313M32 (etigilimab).

A therapeutic agent may be an agent that treats cancer or symptoms associated therewith (e.g., a cytotoxic agent, non-peptide small molecules, or other compound useful in the treatment of cancer or symptoms associated therewith, collectively, an “anti-cancer agent”). Anti-cancer agents can be, e.g., chemotherapeutics or targeted therapy agents.

Anti-cancer agents include mitotic inhibitors, intercalating antibiotics, growth factor inhibitors, cell cycle inhibitors, enzymes, topoisomerase inhibitors, biological response modifiers, alkylating agents, antimetabolites, folic acid analogs, pyrimidine analogs, purine analogs and related inhibitors, vinca alkaloids, epipodophyllotoxins, antibiotics, L-Asparaginase, topoisomerase inhibitors, interferons, platinum coordination complexes, anthracenedione substituted urea, methyl hydrazine derivatives, adrenocortical suppressant, adrenocorticosteroids, progestins, estrogens, antiestrogen, androgens, antiandrogen, and gonadotropin-releasing hormone analog. Further anti-cancer agents include leucovorin (LV), irinotecan, oxaliplatin, capecitabine, paclitaxel, and doxetaxel. In some embodiments, the one or more additional therapies includes two or more anti-cancer agents. The two or more anti-cancer agents can be used in a cocktail to be administered in combination or administered separately. Suitable dosing regimens of combination anti-cancer agents are known in the art and described in, for example, Saltz et al., Proc. Am. Soc. Clin. Oncol. 18:233a (1999), and Douillard et al., Lancet 355(9209):1041-1047 (2000).

Other non-limiting examples of anti-cancer agents include Gleevec® (Imatinib Mesylate); Kyprolis® (carfilzomib); Velcade® (bortezomib); Casodex (bicalutamide); Iressa® (gefitinib); alkylating agents such as thiotepa and cyclosphosphamide; alkyl sulfonates such as busulfan, improsulfan and piposulfan; aziridines such as benzodopa, carboquone, meturedopa, and uredepa; ethylenimines and methylamelamines including altretamine, triethylenemelamine, triethylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; sarcodictyin A; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimustine; antibiotics such as the enediyne antibiotics (e.g., calicheamicin, such as calicheamicin gammall and calicheamicin omegall (see, e.g., Agnew, Chem. Intl. Ed Engl. 33:183-186 (1994)); dynemicin such as dynemicin A; bisphosphonates such as clodronate; an esperamicin; neocarzinostatin chromophore and related chromoprotein enediyne antibiotic chromophores, aclacinomysins, actinomycin, authramycin, azaserine, bleomycins, cactinomycin, calicheamicin, carabicin, caminomycin, carminomycin, carzinophilin, chromomycins, dactinomycin, daunorubicin, detorubicin, 6-diazo-5-oxo-L-norleucine, adriamycin (doxorubicin), morpholino-doxorubicin, cyanomorpholino-doxorubicin, 2-pyrrolino-doxorubicin, deoxydoxorubicin, epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenishers such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone such as epothilone B; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes such as T-2 toxin, verracurin A, roridin A and anguidine; urethane; vindesine; dacarbazine; mannomustine; mitobronitol; mitolactol; pipobroman; gacytosine; arabinoside (“Ara-C”); cyclophosphamide; thiotepa; taxoids, e.g., Taxol® (paclitaxel), Abraxane® (cremophor-free, albumin-engineered nanoparticle formulation of paclitaxel), and Taxotere® (doxetaxel); chloranbucil; tamoxifen (Nolvadex™); raloxifene; aromatase inhibiting 4(5)-imidazoles; 4-hydroxytamoxifen; trioxifene; keoxifene; LY 117018; onapristone; toremifene (Fareston®); flutamide, nilutamide, bicalutamide, leuprolide, goserelin; chlorambucil; Gemzar® gemcitabine; 6-thioguanine; mercaptopurine; platinum coordination complexes such as cisplatin, oxaliplatin and carboplatin; vinblastine; platinum; etoposide (VP-16); ifosfamide; mitoxantrone; vincristine; Navelbine® (vinorelbine); novantrone; teniposide; edatrexate; daunomycin; aminopterin; ibandronate; irinotecan (e.g., CPT-11); topoisomerase inhibitor RFS 2000; difluoromethylornithine (DMFO); retinoids such as retinoic acid; esperamicins; capecitabine (e.g., Xeloda®); and pharmaceutically acceptable salts of any of the above.

Additional non-limiting examples of anti-cancer agents include trastuzumab (Herceptin®), bevacizumab (Avastin®), cetuximab (Erbitux®), rituximab (Rituxan®), Taxol®, Arimidex®, ABVD, avicine, abagovomab, acridine carboxamide, adecatumumab, 17-N-allylamino-17-demethoxygeldanamycin, alphaprodine, 3-aminopyridine-2-carboxaldehyde thiosemicarbazone, amonafide, anthracenedione, anti-CD22 immunotoxins, antineoplastics (e.g., cell-cycle nonspecific antineoplastic agents, and other antineoplastics described herein), antitumorigenic herbs, apaziquone, atiprimod, azathioprine, belotecan, bendamustine, BIBW 2992, biricodar, brostallicin, bryostatin, buthionine sulfoximine, CBV (chemotherapy), calyculin, dichloroacetic acid, discodermolide, elsamitrucin, enocitabine, eribulin, exatecan, exisulind, ferruginol, forodesine, fosfestrol, ICE chemotherapy regimen, IT-101, imexon, imiquimod, indolocarbazole, irofulven, laniquidar, larotaxel, lenalidomide, lucanthone, lurtotecan, mafosfamide, mitozolomide, nafoxidine, nedaplatin, olaparib, ortataxel, PAC-1, pawpaw, pixantrone, proteasome inhibitors, rebeccamycin, resiquimod, rubitecan, SN-38, salinosporamide A, sapacitabine, Stanford V, swainsonine, talaporfin, tariquidar, tegafur-uracil, temodar, tesetaxel, triplatin tetranitrate, tris(2-chloroethyl)amine, troxacitabine, uramustine, vadimezan, vinflunine, ZD6126, and zosuquidar.

Further non-limiting examples of anti-cancer agents include natural products such as vinca alkaloids (e.g., vinblastine, vincristine, and vinorelbine), epidipodophyllotoxins (e.g., etoposide and teniposide), antibiotics (e.g., dactinomycin (actinomycin D), daunorubicin, and idarubicin), anthracyclines, mitoxantrone, bleomycins, plicamycin (mithramycin), mitomycin, enzymes (e.g., L-asparaginase which systemically metabolizes L-asparagine and deprives cells which do not have the capacity to synthesize their own asparagine), antiplatelet agents, antiproliferative/antimitotic alkylating agents such as nitrogen mustards (e.g., mechlorethamine, cyclophosphamide and analogs, melphalan, and chlorambucil), ethylenimines and methylmelamines (e.g., hexamethylmelamine and thiotepa), CDK inhibitors (e.g., a CDK4/6 inhibitor such as palbociclib; seliciclib, UCN-01, P1446A-05, PD-0332991, dinaciclib, P27-00, AT-7519, RGB286638, and SCH727965), alkyl sulfonates (e.g., busulfan), nitrosoureas (e.g., carmustine (BCNU) and analogs, and streptozocin), trazenes-dacarbazinine (DTIC), antiproliferative/antimitotic antimetabolites such as folic acid analogs, pyrimidine analogs (e.g., fluorouracil, floxuridine, and cytarabine), purine analogs and related inhibitors (e.g., mercaptopurine, thioguanine, pentostatin, and 2-chlorodeoxyadenosine), aromatase inhibitors (e.g., anastrozole, exemestane, and letrozole), and platinum coordination complexes (e.g., cisplatin and carboplatin), procarbazine, hydroxyurea, mitotane, aminoglutethimide, histone deacetylase (HDAC) inhibitors (e.g., trichostatin, sodium butyrate, apicidan, suberoyl anilide hydroamic acid, vorinostat, LBH 589, romidepsin, ACY-1215, and panobinostat), mTOR inhibitors (e.g., vistusertib, temsirolimus, everolimus, ridaforolimus, and sirolimus), KSP (Eg5) inhibitors (e.g., Array 520), DNA binding agents (e.g., Zalypsis®), PI3K inhibitors such as PI3K delta inhibitor (e.g., GS-1101 and TGR-1202), PI3K delta and gamma inhibitor (e.g., CAL-130), copanlisib, alpelisib and idelalisib; multi-kinase inhibitor (e.g., TG02 and sorafenib), hormones (e.g., estrogen) and hormone agonists such as leutinizing hormone releasing hormone (LHRH) agonists (e.g., goserelin, leuprolide and triptorelin), BAFF-neutralizing antibody (e.g., LY2127399), IKK inhibitors, p38 MAPK inhibitors, anti-IL-6 (e.g., CNT0328), telomerase inhibitors (e.g., GRN 163L), aurora kinase inhibitors (e.g., MHLN8237), cell surface monoclonal antibodies (e.g., anti-CD38 (HUMAX-CD38), anti-CSl (e.g., elotuzumab), HSP90 inhibitors (e.g., 17 AAG and KOS 953), P13K/Akt inhibitors (e.g., perifosine), Akt inhibitors (e.g., GSK-2141795), PKC inhibitors (e.g., enzastaurin), FTIs (e.g., Zarnestra™), anti-CD138 (e.g., BT062), Torc1/2 specific kinase inhibitors (e.g., INK128), ER/UPR targeting agents (e.g., MKC-3946), cFMS inhibitors (e.g., ARRY-382), JAK1/2 inhibitors (e.g., CYT387), PARP inhibitors (e.g., olaparib and veliparib (ABT-888)), and BCL-2 antagonists.

In some embodiments, an anti-cancer agent is selected from mechlorethamine, camptothecin, ifosfamide, tamoxifen, raloxifene, gemcitabine, Navelbine®, sorafenib, or any analog or derivative variant of the foregoing.

In some embodiments, the anti-cancer agent is a HER2 inhibitor. Non-limiting examples of HER2 inhibitors include monoclonal antibodies such as trastuzumab (Herceptin®) and pertuzumab (Perjeta®); small molecule tyrosine kinase inhibitors such as gefitinib (Iressa®), erlotinib (Tarceva®), osimertinib (TAGRISSO®), pelitinib, CP-654577, CP-724714, canertinib (CI 1033), HKI-272, lapatinib (GW-572016; Tykerb®), PKI-166, AEE788, BMS-599626, HKI-357, BIBW 2992, ARRY-334543, and JNJ-26483327.

In some embodiments, an anti-cancer agent is an ALK inhibitor. Non-limiting examples of ALK inhibitors include ceritinib, TAE-684 (NVP-TAE694), PF02341066 (crizotinib or 1066), alectinib; brigatinib; entrectinib; ensartinib (X-396); lorlatinib; ASP3026; CEP-37440; 4SC-203; TL-398; PLB1003; TSR-011; CT-707; TPX-0005, and AP26113. Additional examples of ALK kinase inhibitors are described in examples 3-39 of WO05016894.

In some embodiments, an anti-cancer agent is an inhibitor of a member downstream of a Receptor Tyrosine Kinase (RTK)/Growth Factor Receptor (e.g., a SHP2 inhibitor (e.g., SHP099, TNO155, RMC-4550, RMC-4630, JAB-3068, RLY-1971), a SOS1 inhibitor (e.g., BI-1701963, BI-3406), a Raf inhibitor, a MEK inhibitor, an ERK inhibitor, a PI3K inhibitor, a PTEN inhibitor, an AKT inhibitor, or an mTOR inhibitor (e.g., mTORC1 inhibitor or mTORC2 inhibitor). In some embodiments, the anti-cancer agent is JAB-3312. In some embodiments, an anti-cancer agent is a Ras inhibitor (e.g., AMG 510, MRTX1257, MRTX849, MRTX1133, JNJ-74699157 (ARS-3248), LY3499446, or ARS-1620), or a Ras vaccine, or another therapeutic modality designed to directly or indirectly decrease the oncogenic activity of Ras.

In some embodiments, the present disclosure provides for method for treating a disease or disorder, e.g., a cancer, with a combination therapy comprising a SHP2 inhibitor in combination with an inhibitor of RAS, such as AMG 510, BI-2852, or ARS-3248. In some embodiments, an inhibitor of RAS is an inhibitor of a mutant RAS selected from:

(a) the following K-Ras mutants: G12D, G12V, G12C, G13D, G12R, G12A, Q61H, G12S, A146T, G13C, Q61L, Q61R, K117N, A146V, G12F, Q61K, L19F, Q22K, V14I, A59T, A146P, G13R, G12L, or G13V, and combinations thereof,

(b) the following H-Ras mutants: Q61R, G13R, Q61K, G12S, Q61L, G12D, G13V, G13D, G12C, K117N, A59T, G12V, G13C, Q61H, G13S, A18V, D119N, G13N, A146T, A66T, G12A, A146V, G12N, or G12R, and combinations thereof, and

(c) the following N-Ras mutants: Q61R, Q61K, G12D, Q61L, Q61H, G13R, G13D, G12S, G12C, G12V, G12A, G13V, G12R, P185S, G13C, A146T, G60E, Q61P, A59D, E132K, E49K, T50I, A146V, or A59T, and combinations thereof, or a combination of any of the foregoing.

In some embodiments, a therapeutic agent that may be combined with a compound of the present invention is an inhibitor of the MAP kinase (MAPK) pathway (or “MAPK inhibitor”). MAPK inhibitors include, but are not limited to, one or more MAPK inhibitor described in Cancers (Basel) 2015 September; 7(3): 1758-1784. For example, the MAPK inhibitor may be selected from one or more of trametinib, binimetinib, selumetinib, cobimetinib, LErafAON (NeoPharm), ISIS 5132; vemurafenib, pimasertib, TAK733, RO4987655 (CH4987655); CI-1040; PD-0325901; CH5126766; MAP855; AZD6244; refametinib (RDEA 119/BAY 86-9766); GDC-0973/XL581; AZD8330 (ARRY-424704/ARRY-704); RO5126766 (Roche, described in PLoS One. 2014 Nov. 25; 9 (11)); and GSK1120212 (or JTP-74057, described in Clin Cancer Res. 2011 Mar. 1; 17(5):989-1000). The MAPK inhibitor may be PLX8394, LXH254, GDC-5573, or LY3009120.

In some embodiments, an anti-cancer agent is a disrupter or inhibitor of the RAS-RAF-ERK or PI3K-AKT-TOR or PI3K-AKT signaling pathways. The PI3K/AKT inhibitor may include, but is not limited to, one or more PI3K/AKT inhibitor described in Cancers (Basel) 2015 September; 7(3): 1758-1784. For example, the PI3K/AKT inhibitor may be selected from one or more of NVP-BEZ235; BGT226; SF1126; GDC-0980; PI-103; PF-04691502; PKI-587; GSK2126458.

In some embodiments, an anti-cancer agent is a PD-1 or PD-L1 antagonist.

In some embodiments, additional therapeutic agents include ALK inhibitors, HER2 inhibitors, EGFR inhibitors, IGF-1R inhibitors, MEK inhibitors, PI3K inhibitors, AKT inhibitors, TOR inhibitors, MCL-1 inhibitors, BCL-2 inhibitors, SHP2 inhibitors, proteasome inhibitors, and immune therapies. In some embodiments, a therapeutic agent may be a pan-RTK inhibitor, such as afatinib.

IGF-1R inhibitors include linsitinib, or a pharmaceutically acceptable salt thereof.

EGFR inhibitors include, but are not limited to, small molecule antagonists, antibody inhibitors, or specific antisense nucleotide or siRNA. Useful antibody inhibitors of EGFR include cetuximab (Erbitux®), panitumumab (Vectibix®), zalutumumab, nimotuzumab, and matuzumab. Further antibody-based EGFR inhibitors include any anti-EGFR antibody or antibody fragment that can partially or completely block EGFR activation by its natural ligand. Non-limiting examples of antibody-based EGFR inhibitors include those described in Modjtahedi et al., Br. J. Cancer 1993, 67:247-253; Teramoto et al., Cancer 1996, 77:639-645; Goldstein et al., Clin. Cancer Res. 1995, 1:1311-1318; Huang et al., 1999, Cancer Res. 15:59(8):1935-40; and Yang et al., Cancer Res. 1999, 59:1236-1243. The EGFR inhibitor can be monoclonal antibody Mab E7.6.3 (Yang, 1999 supra), or Mab C225 (ATCC Accession No. HB-8508), or an antibody or antibody fragment having the binding specificity thereof.

Small molecule antagonists of EGFR include almonertinib (Ameile®), gefitinib (Iressa®), erlotinib (Tarceva®), osimertinib (TAGRISSO®) and lapatinib (TykerB®). See, e.g., Yan et al., Pharmacogenetics and Pharmacogenomics In Oncology Therapeutic Antibody Development, BioTechniques 2005, 39(4):565-8; and Paez et al., EGFR Mutations In Lung Cancer Correlation With Clinical Response To Gefitinib Therapy, Science 2004, 304(5676):1497-500. Further non-limiting examples of small molecule EGFR inhibitors include any of the EGFR inhibitors described in the following patent publications, and all pharmaceutically acceptable salts of such EGFR inhibitors: EP 0520722; EP 0566226; WO96/33980; U.S. Pat. No. 5,747,498; WO96/30347; EP 0787772; WO97/30034; WO97/30044; WO97/38994; WO97/49688; EP 837063; WO98/02434; WO97/38983; WO95/19774; WO95/19970; WO97/13771; WO98/02437; WO98/02438; WO97/32881; DE 19629652; WO98/33798; WO97/32880; WO97/32880; EP 682027; WO97/02266; WO97/27199; WO98/07726; WO97/34895; WO96/31510; WO98/14449; WO98/14450; WO98/14451; WO95/09847; WO97/19065; WO98/17662; U.S. Pat. Nos. 5,789,427; 5,650,415; 5,656,643; WO99/35146; WO99/35132; WO99/07701; and WO92/20642. Additional non-limiting examples of small molecule EGFR inhibitors include any of the EGFR inhibitors described in Traxler et al., Exp. Opin. Ther. Patents 1998, 8(12):1599-1625.

MEK inhibitors include, but are not limited to, pimasertib, selumetinib, cobimetinib (Cotellic®), trametinib (Mekinist®), and binimetinib (Mektovi®). In some embodiments, a MEK inhibitor targets a MEK mutation that is a Class I MEK1 mutation selected from D67N; P124L; P124S; and L177V. In some embodiments, the MEK mutation is a Class II MEK1 mutation selected from AE51-Q58; AF53-Q58; E203K; L177M; C121S; F53L; K57E; Q56P; and K57N.

PI3K inhibitors include, but are not limited to, wortmannin; 17-hydroxywortmannin analogs described in WO06/044453; 4-[2-(1H-Indazol-4-yl)-6-[[4-(methylsulfonyl)piperazin-1-yl]methyl]thieno[3,2-d]pyrimidin-4-yl]morpholine (also known as pictilisib or GDC-0941 and described in WO09/036082 and WO09/055730); 2-methyl-2-[4-[3-methyl-2-oxo-8-(quinolin-3-yl)-2,3-dihydroimidazo[4,5-c]quinolin-1-yl]phenyl]propionitrile (also known as BEZ 235 or NVP-BEZ 235, and described in WO06/122806); (S)-1-(4-((2-(2-aminopyrimidin-5-yl)-7-methyl-4-morpholinothieno[3,2-d]pyrimidin-6-yl)methyl)piperazin-1-yl)-2-hydroxypropan-1-one (described in WO08/070740); LY294002 (2-(4-morpholinyl)-8-phenyl-4H-1-benzopyran-4-one (available from Axon Medchem); PI 103 hydrochloride (3-[4-(4-morpholinylpyrido-[3′,2′:4,5]furo[3,2-d]pyrimidin-2-yl]phenol hydrochloride (available from Axon Medchem); PIK 75 (2-methyl-5-nitro-2-[(6-bromoimidazo[1,2-a]pyridin-3-yl)methylene]-1-methylhydrazide-benzenesulfonic acid, monohydrochloride) (available from Axon Medchem); PIK 90 (N-(7,8-dimethoxy-2,3-dihydro-imidazo[1,2-c]quinazolin-5-yl)-nicotinamide (available from Axon Medchem); AS-252424 (5-[1-[5-(4-fluoro-2-hydroxy-phenyl)-furan-2-yl]-meth-(Z)-ylidene]-thiazolidine-2,4-dione (available from Axon Medchem); TGX-221 (7-methyl-2-(4-morpholinyl)-9-[1-(phenylamino)ethyl]-4H-pyrido-[1,2-a]pyrimidin-4-one (available from Axon Medchem); XL-765; and XL-147. Other PI3K inhibitors include demethoxyviridin, perifosine, CAL101, PX-866, BEZ235, SF1126, INK1117, IPI-145, BKM120, XL147, Palomid 529, GSK1059615, ZSTK474, PWT33597, IC87114, TGI 00-115, CAL263, PI-103, GNE-477, CUDC-907, and AEZS-136.

AKT inhibitors include, but are not limited to, Akt-1-1 (inhibits Akt1) (Barnett et al., Biochem. J. 2005, 385 (Pt. 2): 399-408); Akt-1-1,2 (inhibits Ak1 and 2) (Barnett et al., Biochem. J. 2005, 385 (Pt. 2): 399-408); API-59CJ-Ome (e.g., Jin et al., Br. J. Cancer 2004, 91:1808-12); 1-H-imidazo[4,5-c]pyridinyl compounds (e.g., WO 05/011700); indole-3-carbinol and derivatives thereof (e.g., U.S. Pat. No. 6,656,963; Sarkar and Li J Nutr. 2004, 134 (12 Suppl):34935-34985); perifosine (e.g., interferes with Akt membrane localization; Dasmahapatra et al. Clin. Cancer Res. 2004, 10(15):5242-52); phosphatidylinositol ether lipid analogues (e.g., Gills and Dennis Expert. Opin. Investig. Drugs 2004, 13:787-97); and triciribine (TCN or API-2 or NCI identifier: NSC 154020; Yang et al., Cancer Res. 2004, 64:4394-9).

mTOR inhibitors include, but are not limited to, ATP-competitive mTORC1/mTORC2 inhibitors, e.g., PI-103, PP242, PP30; Torin 1; FKBP12 enhancers; 4H-1-benzopyran-4-one derivatives; and rapamycin (also known as sirolimus) and derivatives thereof, including: temsirolimus (Torisel®); everolimus (Afinitor®; WO94/09010); ridaforolimus (also known as deforolimus or AP23573); rapalogs, e.g., as disclosed in WO98/02441 and WO01/14387, e.g. AP23464 and AP23841; 40-(2-hydroxyethyl)rapamycin; 40-[3-hydroxy(hydroxymethyl)methylpropanoate]-rapamycin (also known as CC1779); 40-epi-(tetrazolyt)-rapamycin (also called ABT578); 32-deoxorapamycin; 16-pentynyloxy-32 (S)-dihydrorapanycin; derivatives disclosed in WO05/005434; derivatives disclosed in U.S. Pat. Nos. 5,258,389, 5,118,677, 5,118,678, 5,100,883, 5,151,413, 5,120,842, and 5,256,790, and in WO94/090101, WO92/05179, WO93/111130, WO94/02136, WO94/02485, WO95/14023, WO94/02136, WO95/16691, WO96/41807, WO96/41807, and WO2018204416; and phosphorus-containing rapamycin derivatives (e.g., WO05/016252). In some embodiments, the mTOR inhibitor is a bisteric inhibitor, such as RMC-5552.

BRAF inhibitors that may be used in combination with compounds of the invention include, for example, vemurafenib, dabrafenib, and encorafenib. A BRAF may comprise a Class 3 BRAF mutation. In some embodiments, the Class 3 BRAF mutation is selected from one or more of the following amino acid substitutions in human BRAF: D287H; P367R; V459L; G466V; G466E; G466A; S467L; G469E; N581S; N5811; D594N; D594G; D594A; D594H; F595L; G596D; G596R and A762E.

MCL-1 inhibitors include, but are not limited to, AMG-176, MIK665, and S63845. The myeloid cell leukemia-1 (MCL-1) protein is one of the key anti-apoptotic members of the B-cell lymphoma-2 (BCL-2) protein family. Over-expression of MCL-1 has been closely related to tumor progression as well as to resistance, not only to traditional chemotherapies but also to targeted therapeutics including BCL-2 inhibitors such as ABT-263.

In some embodiments, the additional therapeutic agent is selected from the group consisting of a HER2 inhibitor, a CDK4/6 inhibitor, an mTOR inhibitor, a SOS1 inhibitor, or a PD-L1 inhibitor. See, e.g., Hallin et al., Cancer Discovery, DOI: 10.1158/2159-8290 (Oct. 28, 2019) and Canon et al., Nature, 575:217 (2019).

Proteasome inhibitors include, but are not limited to, carfilzomib (Kyprolis®), bortezomib (Velcade®), and oprozomib.

Immune therapies include, but are not limited to, monoclonal antibodies, immunomodulatory imides (IMiDs), GITR agonists, genetically engineered T-cells (e.g., CAR-T cells), bispecific antibodies (e.g., BiTEs), and anti-PD-1, anti-PDL-1, anti-CTLA4, anti-LAG1, and anti-OX40 agents).

Immunomodulatory agents (IMiDs) are a class of immunomodulatory drugs (drugs that adjust immune responses) containing an imide group. The IMiD class includes thalidomide and its analogues (lenalidomide, pomalidomide, and apremilast).

Exemplary anti-PD-1 antibodies and methods for their use are described by Goldberg et al., Blood 2007, 110(1):186-192; Thompson et al., Clin. Cancer Res. 2007, 13(6):1757-1761; and WO06/121168 A1), as well as described elsewhere herein.

GITR agonists include, but are not limited to, GITR fusion proteins and anti-GITR antibodies (e.g., bivalent anti-GITR antibodies), such as, a GITR fusion protein described in U.S. Pat. Nos. 6,111,090, 8,586,023, WO2010/003118 and WO2011/090754; or an anti-GITR antibody described, e.g., in U.S. Pat. No. 7,025,962, EP 1947183, U.S. Pat. Nos. 7,812,135, 8,388,967, 8,591,886, 7,618,632, EP 1866339, and WO2011/028683, WO2013/039954, WO05/007190, WO07/133822, WO05/055808, WO99/40196, WO01/03720, WO99/20758, WO06/083289, WO05/115451, and WO2011/051726.

Another example of a therapeutic agent that may be used in combination with the compounds of the invention is an anti-angiogenic agent. Anti-angiogenic agents are inclusive of, but not limited to, in vitro synthetically prepared chemical compositions, antibodies, antigen binding regions, radionuclides, and combinations and conjugates thereof. An anti-angiogenic agent can be an agonist, antagonist, allosteric modulator, toxin or, more generally, may act to inhibit or stimulate its target (e.g., receptor or enzyme activation or inhibition), and thereby promote cell death or arrest cell growth. In some embodiments, the one or more additional therapies include an anti-angiogenic agent.

Anti-angiogenic agents can be MMP-2 (matrix-metalloproteinase 2) inhibitors, MMP-9 (matrix-metalloproteinase 9) inhibitors, and COX-II (cyclooxygenase 11) inhibitors. Non-limiting examples of anti-angiogenic agents include rapamycin, temsirolimus (CCI-779), everolimus (RAD001), sorafenib, sunitinib, and bevacizumab. Examples of useful COX-II inhibitors include alecoxib, valdecoxib, and rofecoxib. Examples of useful matrix metalloproteinase inhibitors are described in WO96/33172, WO96/27583, WO98/07697, WO98/03516, WO98/34918, WO98/34915, WO98/33768, WO98/30566, WO90/05719, WO99/52910, WO99/52889, WO99/29667, WO99007675, EP0606046, EP0780386, EP1786785, EP1181017, EP0818442, EP1004578, and US20090012085, and U.S. Pat. Nos. 5,863,949 and 5,861,510. Particular MMP-2 and MMP-9 inhibitors are those that have little or no activity inhibiting MMP-1. More particular, are those that selectively inhibit MMP-2 or AMP-9 relative to the other matrix-metalloproteinases (i.e., MAP-1, MMP-3, MMP-4, MMP-5, MMP-6, MMP-7, MMP-8, MMP-10, MMP-11, MMP-12, and MMP-13). Some specific examples of MMP inhibitors are AG-3340, RO 32-3555, and RS 13-0830.

Further exemplary anti-angiogenic agents include KDR (kinase domain receptor) inhibitory agents (e.g., antibodies and antigen binding regions that specifically bind to the kinase domain receptor), anti-VEGF agents (e.g., antibodies or antigen binding regions that specifically bind VEGF (e.g., bevacizumab), or soluble VEGF receptors or a ligand binding region thereof) such as VEGF-TRAP™, and anti-VEGF receptor agents (e.g., antibodies or antigen binding regions that specifically bind thereto), EGFR inhibitory agents (e.g., antibodies or antigen binding regions that specifically bind thereto) such as Vectibix® (panitumumab), erlotinib (Tarceva®), osimertinib (TAGRISSO®), anti-Ang1 and anti-Ang2 agents (e.g., antibodies or antigen binding regions specifically binding thereto or to their receptors, e.g., Tie2/Tek), and anti-Tie2 kinase inhibitory agents (e.g., antibodies or antigen binding regions that specifically bind thereto). Other anti-angiogenic agents include Campath, IL-8, B-FGF, Tek antagonists (US2003/0162712; U.S. Pat. No. 6,413,932), anti-TWEAK agents (e.g., specifically binding antibodies or antigen binding regions, or soluble TWEAK receptor antagonists; see U.S. Pat. No. 6,727,225), ADAM distintegrin domain to antagonize the binding of integrin to its ligands (U.S. 2002/0042368), specifically binding anti-eph receptor or anti-ephrin antibodies or antigen binding regions (U.S. Pat. Nos. 5,981,245; 5,728,813; 5,969,110; 6,596,852; 6,232,447; 6,057,124 and patent family members thereof), and anti-PDGF-BB antagonists (e.g., specifically binding antibodies or antigen binding regions) as well as antibodies or antigen binding regions specifically binding to PDGF-BB ligands, and PDGFR kinase inhibitory agents (e.g., antibodies or antigen binding regions that specifically bind thereto). Additional anti-angiogenic agents include: SD-7784 (Pfizer, USA); cilengitide (Merck KGaA, Germany, EPO 0770622); pegaptanib octasodium, (Gilead Sciences, USA); Alphastatin, (BioActa, UK); M-PGA, (Celgene, USA, U.S. Pat. No. 5,712,291); ilomastat, (Arriva, USA, U.S. Pat. No. 5,892,112); emaxanib, (Pfizer, USA, U.S. Pat. No. 5,792,783); vatalanib, (Novartis, Switzerland); 2-methoxyestradiol (EntreMed, USA); TLC ELL-12 (Elan, Ireland); anecortave acetate (Alcon, USA); alpha-D148 Mab (Amgen, USA); CEP-7055 (Cephalon, USA); anti-Vn Mab (Crucell, Netherlands), DACantiangiogenic (ConjuChem, Canada); Angiocidin (InKine Pharmaceutical, USA); KM-2550 (Kyowa Hakko, Japan); SU-0879 (Pfizer, USA); CGP-79787 (Novartis, Switzerland, EP 0970070); ARGENT technology (Ariad, USA); YIGSR-Stealth (Johnson & Johnson, USA); fibrinogen-E fragment (BioActa, UK); angiogenic inhibitor (Trigen, UK); TBC-1635 (Encysive Pharmaceuticals, USA); SC-236 (Pfizer, USA); ABT-567 (Abbott, USA); Metastatin (EntreMed, USA); maspin (Sosei, Japan); 2-methoxyestradiol (Oncology Sciences Corporation, USA); ER-68203-00 (IV AX, USA); BeneFin (Lane Labs, USA); Tz-93 (Tsumura, Japan); TAN-1120 (Takeda, Japan); FR-111142 (Fujisawa, Japan, JP 02233610); platelet factor 4 (RepliGen, USA, EP 407122); vascular endothelial growth factor antagonist (Borean, Denmark); bevacizumab (pINN) (Genentech, USA); angiogenic inhibitors (SUGEN, USA); XL 784 (Exelixis, USA); XL 647 (Exelixis, USA); MAb, alpha5 beta3 integrin, second generation (Applied Molecular Evolution, USA and MedImmune, USA); enzastaurin hydrochloride (Lilly, USA); CEP 7055 (Cephalon, USA and Sanofi-Synthelabo, France); BC 1 (Genoa Institute of Cancer Research, Italy); rBPI 21 and BPI-derived antiangiogenic (XOMA, USA); PI 88 (Progen, Australia); cilengitide (Merck KGaA, German; Munich Technical University, Germany, Scripps Clinic and Research Foundation, USA); AVE 8062 (Ajinomoto, Japan); AS 1404 (Cancer Research Laboratory, New Zealand); SG 292, (Telios, USA); Endostatin (Boston Childrens Hospital, USA); ATN 161 (Attenuon, USA); 2-methoxyestradiol (Boston Childrens Hospital, USA); ZD 6474, (AstraZeneca, UK); ZD 6126, (Angiogene Pharmaceuticals, UK); PPI 2458, (Praecis, USA); AZD 9935, (AstraZeneca, UK); AZD 2171, (AstraZeneca, UK); vatalanib (pINN), (Novartis, Switzerland and Schering AG, Germany); tissue factor pathway inhibitors, (EntreMed, USA); pegaptanib (Pinn), (Gilead Sciences, USA); xanthorrhizol, (Yonsei University, South Korea); vaccine, gene-based, VEGF-2, (Scripps Clinic and Research Foundation, USA); SPV5.2, (Supratek, Canada); SDX 103, (University of California at San Diego, USA); PX 478, (Pro1X, USA); METASTATIN, (EntreMed, USA); troponin I, (Harvard University, USA); SU 6668, (SUGEN, USA); OXI 4503, (OXiGENE, USA); o-guanidines, (Dimensional Pharmaceuticals, USA); motuporamine C, (British Columbia University, Canada); CDP 791, (Celltech Group, UK); atiprimod (pINN), (GlaxoSmithKline, UK); E 7820, (Eisai, Japan); CYC 381, (Harvard University, USA); AE 941, (Aeterna, Canada); vaccine, angiogenic, (EntreMed, USA); urokinase plasminogen activator inhibitor, (Dendreon, USA); oglufanide (pINN), (Melmotte, USA); HIF-1 alfa inhibitors, (Xenova, UK); CEP 5214, (Cephalon, USA); BAY RES 2622, (Bayer, Germany); Angiocidin, (InKine, USA); A6, (Angstrom, USA); KR 31372, (Korea Research Institute of Chemical Technology, South Korea); GW 2286, (GlaxoSmithKline, UK); EHT 0101, (ExonHit, France); CP 868596, (Pfizer, USA); CP 564959, (OSI, USA); CP 547632, (Pfizer, USA); 786034, (GlaxoSmithKline, UK); KRN 633, (Kirin Brewery, Japan); drug delivery system, intraocular, 2-methoxyestradiol; anginex (Maastricht University, Netherlands, and Minnesota University, USA); ABT 510 (Abbott, USA); AAL 993 (Novartis, Switzerland); VEGI (ProteomTech, USA); tumor necrosis factor-alpha inhibitors; SU 11248 (Pfizer, USA and SUGEN USA); ABT 518, (Abbott, USA); YH16 (Yantai Rongchang, China); S-3 APG (Boston Childrens Hospital, USA and EntreMed, USA); MAb, KDR (ImClone Systems, USA); MAb, alpha5 beta (Protein Design, USA); KDR kinase inhibitor (Celltech Group, UK, and Johnson & Johnson, USA); GFB 116 (South Florida University, USA and Yale University, USA); CS 706 (Sankyo, Japan); combretastatin A4 prodrug (Arizona State University, USA); chondroitinase AC (IBEX, Canada); BAY RES 2690 (Bayer, Germany); AGM 1470 (Harvard University, USA, Takeda, Japan, and TAP, USA); AG 13925 (Agouron, USA); Tetrathiomolybdate (University of Michigan, USA); GCS 100 (Wayne State University, USA) CV 247 (Ivy Medical, UK); CKD 732 (Chong Kun Dang, South Korea); irsogladine, (Nippon Shinyaku, Japan); RG 13577; WX 360 (Wilex, Germany); squalamine, (Genaera, USA); RPI 4610 (Sirna, USA); heparanase inhibitors (InSight, Israel); KL 3106 (Kolon, South Korea); Honokiol (Emory University, USA); ZK CDK (Schering AG, Germany); ZK Angio (Schering AG, Germany); ZK 229561 (Novartis, Switzerland, and Schering AG, Germany); XMP 300 (XOMA, USA); VGA 1102 (Taisho, Japan); VE-cadherin-2 antagonists (ImClone Systems, USA); Vasostatin (National Institutes of Health, USA); Flk-1 (ImClone Systems, USA); TZ 93 (Tsumura, Japan); TumStatin (Beth Israel Hospital, USA); truncated soluble FLT 1 (vascular endothelial growth factor receptor 1) (Merck & Co, USA); Tie-2 ligands (Regeneron, USA); and thrombospondin 1 inhibitor (Allegheny Health, Education and Research Foundation, USA).

Further examples of therapeutic agents that may be used in combination with compounds of the invention include agents (e.g., antibodies, antigen binding regions, or soluble receptors) that specifically bind and inhibit the activity of growth factors, such as antagonists of hepatocyte growth factor (HGF, also known as Scatter Factor), and antibodies or antigen binding regions that specifically bind its receptor, c-Met.

Another example of a therapeutic agent that may be used in combination with compounds of the invention is an autophagy inhibitor. Autophagy inhibitors include, but are not limited to chloroquine, 3-methyladenine, hydroxychloroquine (Plaquenil™), bafilomycin A1, 5-amino-4-imidazole carboxamide riboside (AICAR), okadaic acid, autophagy-suppressive algal toxins which inhibit protein phosphatases of type 2A or type 1, analogues of cAMP, and drugs which elevate cAMP levels such as adenosine, LY204002, N6-mercaptopurine riboside, and vinblastine. In addition, antisense or siRNA that inhibits expression of proteins including but not limited to ATG5 (which are implicated in autophagy), may also be used. In some embodiments, the one or more additional therapies include an autophagy inhibitor.

Another example of a therapeutic agent that may be used in combination with compounds of the invention is an anti-neoplastic agent. In some embodiments, the one or more additional therapies include an anti-neoplastic agent. Non-limiting examples of anti-neoplastic agents include acemannan, aclarubicin, aldesleukin, alemtuzumab, alitretinoin, altretamine, amifostine, aminolevulinic acid, amrubicin, amsacrine, anagrelide, anastrozole, ancer, ancestim, arglabin, arsenic trioxide, BAM-002 (Novelos), bexarotene, bicalutamide, broxuridine, capecitabine, celmoleukin, cetrorelix, cladribine, clotrimazole, cytarabine ocfosfate, DA 3030 (Dong-A), daclizumab, denileukin diftitox, deslorelin, dexrazoxane, dilazep, docetaxel, docosanol, doxercalciferol, doxifluridine, doxorubicin, bromocriptine, carmustine, cytarabine, fluorouracil, HIT diclofenac, interferon alfa, daunorubicin, doxorubicin, tretinoin, edelfosine, edrecolomab, eflornithine, emitefur, epirubicin, epoetin beta, etoposide phosphate, exemestane, exisulind, fadrozole, filgrastim, finasteride, fludarabine phosphate, formestane, fotemustine, gallium nitrate, gemcitabine, gemtuzumab zogamicin, gimeracil/oteracil/tegafur combination, glycopine, goserelin, heptaplatin, human chorionic gonadotropin, human fetal alpha fetoprotein, ibandronic acid, idarubicin, (imiquimod, interferon alfa, interferon alfa, natural, interferon alfa-2, interferon alfa-2a, interferon alfa-2b, interferon alfa-N1, interferon alfa-n3, interferon alfacon-1, interferon alpha, natural, interferon beta, interferon beta-1a, interferon beta-1b, interferon gamma, natural interferon gamma-1a, interferon gamma-1b, interleukin-1 beta, iobenguane, irinotecan, irsogladine, lanreotide, LC 9018 (Yakult), leflunomide, lenograstim, lentinan sulfate, letrozole, leukocyte alpha interferon, leuprorelin, levamisole+fluorouracil, liarozole, lobaplatin, lonidamine, lovastatin, masoprocol, melarsoprol, metoclopramide, mifepristone, miltefosine, mirimostim, mismatched double stranded RNA, mitoguazone, mitolactol, mitoxantrone, molgramostim, nafarelin, naloxone+pentazocine, nartograstim, nedaplatin, nilutamide, noscapine, novel erythropoiesis stimulating protein, NSC 631570 octreotide, oprelvekin, osaterone, oxaliplatin, paclitaxel, pamidronic acid, pegaspargase, peginterferon alfa-2b, pentosan polysulfate sodium, pentostatin, picibanil, pirarubicin, rabbit antithymocyte polyclonal antibody, polyethylene glycol interferon alfa-2a, porfimer sodium, raloxifene, raltitrexed, rasburiembodiment, rhenium Re 186 etidronate, RII retinamide, rituximab, romurtide, samarium (153 Sm) lexidronam, sargramostim, sizofiran, sobuzoxane, sonermin, strontium-89 chloride, suramin, tasonermin, tazarotene, tegafur, temoporfin, temozolomide, teniposide, tetrachlorodecaoxide, thalidomide, thymalfasin, thyrotropin alfa, topotecan, toremifene, tositumomab-iodine 131, trastuzumab, treosulfan, tretinoin, trilostane, trimetrexate, triptorelin, tumor necrosis factor alpha, natural, ubenimex, bladder cancer vaccine, Maruyama vaccine, melanoma lysate vaccine, valrubicin, verteporfin, vinorelbine, virulizin, zinostatin stimalamer, or zoledronic acid; abarelix; AE 941 (Aeterna), ambamustine, antisense oligonucleotide, bcl-2 (Genta), APC 8015 (Dendreon), decitabine, dexaminoglutethimide, diaziquone, EL 532 (Elan), EM 800 (Endorecherche), eniluracil, etanidazole, fenretinide, filgrastim SD01 (Amgen), fulvestrant, galocitabine, gastrin 17 immunogen, HLA-B7 gene therapy (Vical), granulocyte macrophage colony stimulating factor, histamine dihydrochloride, ibritumomab tiuxetan, ilomastat, IM 862 (Cytran), interleukin-2, iproxifene, LDI 200 (Milkhaus), leridistim, lintuzumab, CA 125 MAb (Biomira), cancer MAb (Japan Pharmaceutical Development), HER-2 and Fc MAb (Medarex), idiotypic 105AD7 MAb (CRC Technology), idiotypic CEA MAb (Trilex), LYM-1-iodine 131 MAb (Techni clone), polymorphic epithelial mucin-yttrium 90 MAb (Antisoma), marimastat, menogaril, mitumomab, motexafin gadolinium, MX 6 (Galderma), nelarabine, nolatrexed, P 30 protein, pegvisomant, pemetrexed, porfiromycin, prinomastat, RL 0903 (Shire), rubitecan, satraplatin, sodium phenylacetate, sparfosic acid, SRL 172 (SR Pharma), SU 5416 (SUGEN), TA 077 (Tanabe), tetrathiomolybdate, thaliblastine, thrombopoietin, tin ethyl etiopurpurin, tirapazamine, cancer vaccine (Biomira), melanoma vaccine (New York University), melanoma vaccine (Sloan Kettering Institute), melanoma oncolysate vaccine (New York Medical College), viral melanoma cell lysates vaccine (Royal Newcastle Hospital), or valspodar.

Additional examples of therapeutic agents that may be used in combination with compounds of the invention include ipilimumab (Yervoy®); tremelimumab; galiximab; nivolumab, also known as BMS-936558 (Opdivo®); pembrolizumab (Keytruda®); avelumab (Bavencio®); AMP224; BMS-936559; MPDL3280A, also known as RG7446; MEDI-570; AMG557; MGA271; IMP321; BMS-663513; PF-05082566; CDX-1127; anti-OX40 (Providence Health Services); huMAbOX40L; atacicept; CP-870893; lucatumumab; dacetuzumab; muromonab-CD3; ipilimumab; MEDI4736 (Imfinzi®); MSB0010718C; AMP 224; adalimumab (Humira®); ado-trastuzumab emtansine (Kadcyla®); alemtuzumab (Campath®); basiliximab (Simulect®); belimumab (Benlysta®); basiliximab (Simulect®); belimumab (Benlysta®); brentuximab vedotin (Adcetris®); canakinumab (Ilaris®); certolizumab pegol (Cimzia®); daclizumab (Zenapax®); daratumumab (Darzalex®); denosumab (Prolia®); eculizumab (Soliris®); efalizumab (Raptiva®); gemtuzumab ozogamicin (Mylotarg®); golimumab (Simponi®); ibritumomab tiuxetan (Zevalin®); infliximab (Remicade®); motavizumab (Numax®); natalizumab (Tysabri®); obinutuzumab (Gazyva®); ofatumumab (Arzerra®); omalizumab (Xolair®); palivizumab (Synagis®); pertuzumab (Perjeta®); pertuzumab (Perjeta®); ranibizumab (Lucentis®); raxibacumab (Abthrax®); tocilizumab (Actemra®); tositumomab; tositumomab-i-131; tositumomab and tositumomab-i-131 (Bexxar®); ustekinumab (Stelara®); AMG 102; AMG 386; AMG 479; AMG 655; AMG 706; AMG 745; and AMG 951.

### Diseases and Disorders

The methods of the disclosure may be used to treat any proliferative disease or disorder. In some embodiments of the methods of the disclosure, the proliferative disorder is cancer.

The methods of the disclosure may be used to treat any proliferative disease or disorder associated with an oncogenic RTK fusion that activates MAPK. In some embodiments, the oncogenic RTK fusion that activates MAPK sensitizes the mutated cell to allosteric inhibitors of SHP2. Several such diseases or conditions that may be treatable according to the instant disclosure are known in the art. For example, in certain embodiments, the present disclosure provides methods for treating a disease or condition selected from, but not limited to, tumors of hemopoietic and lymphoid system including myeloproliferative syndromes, myelodysplastic syndromes, and leukemia, e.g., acute myeloid leukemia, and juvenile myelomonocytic leukemias; esophageal cancer; breast cancer; lung cancer; colon cancer; gastric cancer, neuroblastoma, bladder cancer, prostate cancer; glioblastoma; urothelial carcinoma, uterine carcinoma, adenoid and ovarian serous cystadenocarcinoma, paraganglioma, pheochromocytoma, pancreatic cancer, adrenocortical carcinoma, stomach adenocarcinoma, sarcoma, rhabdomyosarcoma, lymphoma, head and neck cancer, skin cancer, peritoneum cancer, intestinal cancer (small and large intestine), thyroid cancer, endometrial cancer, cancer of the biliary tract, soft tissue cancer, ovarian cancer, central nervous system cancer (e.g., primary CNS lymphoma), stomach cancer, pituitary cancer, genital tract cancer, urinary tract cancer, salivary gland cancer, cervical cancer, liver cancer, eye cancer, cancer of the adrenal gland, cancer of autonomic ganglia, cancer of the upper aerodigestive tract, bone cancer, testicular cancer, pleura cancer, kidney cancer, penis cancer, parathyroid cancer, cancer of the meninges, vulvar cancer and melanoma comprising a method disclosed herein, such as, e.g., a monotherapy or combination therapy disclosed herein comprising a SHP2 inhibitor.

In some embodiments of the methods of the disclosure, administration of a SHP2 inhibitor to a subject having a cancer, for example, that comprises a MAPK-activating RTK fusion may result in improvements in efficacy that are more than additive over administration of the SHP2 inhibitor to the general population of subjects with the cancer. For example, in certain aspects, the present disclosure provides for patient stratification for treatment with a SHP2 inhibitor based on the presence or absence of a MAPK-activating RTK fusion in a cancer cell of a subject, wherein administering a SHP2 inhibitor to the patient that has been determined to have a such a MAPK-activating RTK fusion results in a synergistic treatment of the cancer as compared to the treatment that would be expected to result from administration of the SHP2 inhibitor to the general population of patients with the cancer. The effectiveness of the treatment may be based on any detectable readout. For example, in some instances, the synergistic treatment is based on reductions in tumor burden. In some instances, the synergistic treatment is based on SHP2-inhibitor induced tumor killing.

In some embodiments of the methods of the disclosure, administration of a SHP2 inhibitor to a subject having a cancer, for example, a gynecological cancer. In some exemplary by nonlimiting embodiments of the disclosure, a gynecological cancer comprises one or more of a uterine cancer, an endometrial cancer, an ovarian cancer, a cervical cancer, a vaginal cancer, a vulvar cancer and any subtype or variant form of a cancer thereof. In some exemplary by nonlimiting embodiments of the disclosure, a gynecological cancer comprises a metastasis of one or more of a uterine cancer, an endometrial cancer, an ovarian cancer, a cervical cancer, a vaginal cancer, a vulvar cancer and any subtype or variant form of a cancer thereof.

In some embodiments of the methods of the disclosure, the cancer is a uterine cancer, a subtype or variant form of a uterine cancer or a metastasis of a uterine cancer. Uterine cancer of the disclosure may comprise endometrial cancer, endometrial adenocarcinoma, adenosquamous carcinoma, papillary serous carcinoma, and/or uterine sarcoma. Endometrial adenocarcinoma may be localized to the glands of the endometrium or may metastasize from the glands of the endometrium. Adenosquamous carcinoma may comprise squamous cells and/or gland-like cells.

Papillary serous carcinomacinoma may be characterized as aggressive cancer or aggressive subtype of uterine cancer that tends to return even when caught early. Uterine sarcoma may be localized to the uterine muscle wall (myometrium) or may metastasize from the uterine muscle wall (myometrium). Uterine sarcoma may be characterized as a rapidly spreading cancer or subtype of uterine cancer that spreads more quickly than endometrial cancer. In some embodiments, a uterine cancer of the disclosure metastasizes to one or both lungs. In some embodiments, a uterine sarcoma of the disclosure metastasizes to one or both lungs.

In some embodiments of the methods of the disclosure, the cancer is an ovarian cancer, a subtype or variant form of an ovarian cancer or a metastasis of an ovarian cancer. Ovarian cancer of the disclosure may comprise a type I carcinoma or a type II carcinoma. Type I carcinomas may be characterized as slow-growing, indolent neoplasms and may arise from a precursor lesion. Exemplary forms of a type I carcinoma include, but are not limited to, endometrioid carcinoma, clear cell carcinoma and low-grade serous carcinoma. Type II carcinomas may be characterized as clinically aggressive neoplasms that can develop de novo from serous tubal intraepithelial carcinomas (STIC) and/or ovarian surface epithelium. Exemplary forms of a type II carcinoma include, but are not limited to, high-grade serous carcinoma. In some embodiments of the disclosure, a subject characterized as having an ovarian cancer may have a precursor lesion.

In some embodiments of the methods of the disclosure, a subject has a cancer, for example, a gynecological cancer and exhibits a sign or symptom of the gynecological cancer, including, but not limited to, fatigue, pain (local or referred pain to an area outside local site of cancer), localized itching sensation, localized burning sensation, changes to bathroom habits (constipation, diarrhea, increased frequency of urination, blood in stool or blood in urine), bloating, unusual bleeding or discharge, difficulty eating, a feeling of being full to quickly while eating (especially for ovarian cancer), unexplained weight loss and/or changes to the skin texture, color or appearance of rash, sores or warts on the vulva. With respect to pain, in a subject having an ovarian cancer, the pain may present within the subject's back and/or abdominal areas. With respect to pain, in a subject having a uterine or an endometrial cancer, the pain may present within the subject's pelvis or may present as pressure in the pelvis.

Activation of the MAPK pathway may be determined using any suitable method known in the art or described herein. For example, activation of the MAPK pathway may be determined by immunoblot; immunofluorescence; or ELISA; e.g., utilizing antibodies that are specific for phosphorylated versions of MAPK signaling molecules.

Many suitable genotyping methods are known in the art, discussed below, and are suitable for use in the present invention. These may include, e.g., sequencing approaches, microarray approaches, mass spectrometry, high-throughput sequencing approaches, e.g., at a single molecule level.

For example, but not to be limited in anyway, in some aspects, a biological sample from a patient (e.g., a cell such as a tumor cell) may be genotyped using a hybridization detection method to determine whether the cell contains an oncogenic RTK fusion (e.g., an oncogenic RTK fusion that is known to activate the MAPK pathway).

Hybridization detection methods are based on the formation of specific hybrids between complementary nucleic acid sequences that serve to detect nucleic acid sequence mutation(s). Such methods include, e.g., microarray analysis and real time PCR. Hybridization methods, such as Southern analysis, Northern analysis, or in situ hybridizations, may also be used (see Current Protocols in Molecular Biology, Ausubel et al., eds., John Wiley & Sons 2003, incorporated by reference in its entirety).

Other suitable methods for genotyping a cell (e.g., a tumor cell) to determine whether it contains an RTK fusion (e.g., an oncogenic RTK fusion that is known to activate the MAPK pathway) include direct manual sequencing (Church and Gilbert, Proc. Natl. Acad. Sci. USA 81:1991-1995 (1988); Sanger et al., Proc. Natl. Acad. Sci. USA 74:5463-5467 (1977); Beavis et al. U.S. Pat. No. 5,288,644, each incorporated by reference in its entirety for all purposes); automated fluorescent sequencing; single-stranded conformation polymorphism assays (SSCP); clamped denaturing gel electrophoresis (CDGE); two-dimensional gel electrophoresis (2 DGE or TDGE); conformational sensitive gel electrophoresis (CSGE); denaturing gradient gel electrophoresis (DGGE) (Sheffield et al., Proc. Natl. Acad. Sci. USA 86:232-236 (1989)), mobility shift analysis (Orita et al., Proc. Natl. Acad. Sci. USA 86:2766-2770 (1989), incorporated by reference in its entirety), restriction enzyme analysis (Flavell et al., Cell 15:25 (1978); Geever et al., Proc. Natl. Acad. Sci. USA 78:5081 (1981), incorporated by reference in its entirety); quantitative real-time PCR (Raca et al., Genet Test 8(4):387-94 (2004), incorporated by reference in its entirety); heteroduplex analysis; chemical mismatch cleavage (CMC) (Cotton et al., Proc. Natl. Acad. Sci. USA 85:4397-4401 (1985), incorporated by reference in its entirety); RNase protection assays (Myers et al., Science 230:1242 (1985), incorporated by reference in its entirety); use of polypeptides that recognize nucleotide mismatches, e.g., E. coli mutS protein; allele-specific PCR, for example. See, e.g., U.S. Patent Publication No. 2004/0014095, which is incorporated herein by reference in its entirety.

In one embodiment, genomic DNA (gDNA) or a fragment (“region”) thereof containing the site of an RTK fusion present in the sample obtained from the subject, is first amplified. The RTK fusion gDNA, in one embodiment, is one or more of the oncogenic RTK fusions described herein. Such regions can be amplified and isolated by PCR using oligonucleotide primers designed based on genomic and/or cDNA sequences that flank the site. See e.g., PCR Primer: A Laboratory Manual, Dieffenbach and Dveksler, (Eds.); McPherson et al., PCR Basics: From Background to Bench (Springer Verlag, 2000, incorporated by reference in its entirety); Mattila et al., Nucleic Acids Res., 19:4967 (1991), incorporated by reference in its entirety; Eckert et al., PCR Methods and Applications, 1:17 (1991), incorporated by reference in its entirety; PCR (eds. McPherson et al., IRL Press, Oxford), incorporated by reference in its entirety; and U.S. Pat. No. 4,683,202, incorporated by reference in its entirety. Other amplification methods that may be employed include the ligase chain reaction (LCR) (Wu and Wallace, Genomics, 4:560 (1989), Landegren et al., Science, 241:1077 (1988), transcription amplification (Kwoh et al., Proc. Natl. Acad. Sci. USA, 86:1173 (1989)), self-sustained sequence replication (Guatelli et al., Proc. Nat. Acad. Sci. USA, 87:1874 (1990)), incorporated by reference in its entirety, and nucleic acid based sequence amplification (NASBA). Guidelines for selecting primers for PCR amplification are known to those of ordinary skill in the art. See, e.g., McPherson et al., PCR Basics: From Background to Bench, Springer-Verlag, 2000, incorporated by reference in its entirety.

In one example, a sample (e.g., a sample comprising genomic DNA), is obtained from a subject. The DNA in the sample is then examined to determine its RTK fusion profile and as described herein. The term “RTK fusion profile” refers to presence or absence of any one or more known RTK fusion mutations (including, e.g., an oncogenic RTK fusion described herein). The profile is determined by any method described herein, e.g., by sequencing or by hybridization of the gene in the genomic DNA, RNA, or cDNA to a nucleic acid probe, e.g., a DNA probe (which includes cDNA and oligonucleotide probes) or an RNA probe. The nucleic acid probe can be designed to specifically or preferentially hybridize with a gDNA region on the RTK fusion.

In some embodiments, restriction digest analysis can be used to detect the existence of an RTK fusion, if alternate RTK fusion result in the creation or elimination of a restriction site. A sample containing genomic DNA is obtained from the individual. Polymerase chain reaction (PCR) can be used to amplify a region comprising the RTK fusion site (e.g., the C-terminus of the protein fused to the RTK and the N-terminus of the RTK protein), and restriction fragment length analysis s conducted (see Current Protocols in Molecular Biology, Ausubel et al., eds., John Wiley & Sons 2003, incorporated by reference in its entirety). The digestion pattern of the relevant DNA fragment indicates the presence or absence of a particular RTK fusion and is therefore indicative of the presence or absence of susceptibility to treatment with a SHP2 inhibitor.

Sequence analysis can also be used to detect the one or more RTK fusions (e.g., an oncogenic RTK fusion described herein). A sample comprising DNA or RNA is obtained from the subject. PCR or other appropriate methods can be used to amplify a portion encompassing the RTK fusion site, if desired. The sequence is then ascertained, using any standard method, and the presence of an RTK fusion is determined.

Allele-specific oligonucleotides can also be used to detect the presence of an RTK fusion, e.g., through the use of dot-blot hybridization of amplified oligonucleotides with allele-specific oligonucleotide (ASO) probes (see, for example, Saiki et al., Nature (London) 324:163-166 (1986)). An “allele-specific oligonucleotide” (also referred to herein as an “allele-specific oligonucleotide probe”) is typically an oligonucleotide of approximately 10-50 base pairs, preferably approximately 15-30 base pairs, that specifically hybridizes to a nucleic acid region that contains an RTK fusion. An allele-specific oligonucleotide probe that is specific for a particular RTK fusion can be prepared using standard methods (see Current Protocols in Molecular Biology, Ausubel et al., eds., John Wiley & Sons 2003, incorporated by reference in its entirety).

In some embodiments, to determine which of RTK fusions are present in a subject, a sample comprising DNA may be obtained from the subject. PCR or another amplification procedure may be used to amplify a portion encompassing the RTK fusion site.

Real-time pyrophosphate DNA sequencing is yet another approach to detection of RTK fusions (Alderborn et al., (2000) Genome Research, 10(8):1249-1258, incorporated by reference in its entirety). Additional methods include, for example, PCR amplification in combination with denaturing high performance liquid chromatography (dHPLC) (Underhill et al., Genome Research, Vol. 7, No. 10, pp. 996-1005, 1997, incorporated by reference in its entirety for all purposes).

High throughput sequencing, or next-generation sequencing can also be employed to detect one or more of the RTK fusions described herein. Such methods are known in the art (see e.g., Zhang et al., J Genet Genomics. 2011 Mar. 20; 38(3):95-109, incorporated by reference in its entirety for all purposes; Metzker, Nat Rev Genet. 2010 January; 11(1):31-46, incorporated by reference in its entirety for all purposes) and include, but are not limited to, technologies such as ABI SOLiD sequencing technology (now owned by Life Technologies, Carlsbad, Calif.); Roche 454 FLX which uses sequencing by synthesis technology known as pyrosequencing (Roche, Basel Switzerland); Illumina Genome Analyzer (Illumina, San Diego, Calif.); Dover Systems Polonator G.007 (Salem, N.H.); Helicos (Helicos BioSciences Corporation, Cambridge Mass., USA), and Sanger. In one embodiment, DNA sequencing may be performed using methods well known in the art including mass spectrometry technology and whole genome sequencing technologies, single molecule sequencing, etc.

In one embodiment, nucleic acid, for example, genomic DNA is sequenced using nanopore sequencing, to determine the presence of the one or more RTK fusions described herein (e.g., as described in Soni et al. (2007). Clin Chem 53, pp. 1996-2001, incorporated by reference in its entirety for all purposes). Nanopore sequencing is a single-molecule sequencing technology whereby a single molecule of DNA is sequenced directly as it passes through a nanopore. A nanopore is a small hole, of the order of 1 nanometer in diameter. Immersion of a nanopore in a conducting fluid and application of a potential (voltage) across it results in a slight electrical current due to conduction of ions through the nanopore. The amount of current that flows is sensitive to the size and shape of the nanopore. As a DNA molecule passes through a nanopore, each nucleotide on the DNA molecule obstructs the nanopore to a different degree, changing the magnitude of the current through the nanopore in different degrees. Thus, this change in the current as the DNA molecule passes through the nanopore represents a reading of the DNA sequence. Nanopore sequencing technology as disclosed in U.S. Pat. Nos. 5,795,782, 6,015,714, 6,627,067, 7,238,485 and 7,258,838 and U.S. Patent Application Publication Nos. 2006/003171 and 2009/0029477, each incorporated by reference in its entirety for all purposes, is amenable for use with the methods described herein.

### RTK Fusions

In some embodiments of the disclosure, an RTK fusion may be an oncogenic RTK fusion. RTK fusions may induce, enhance, or propagate oncogenesis. Exemplary RTK fusions include, but are not limited to, an ALK fusion, a ROS1, fusion, a RET fusion, and an NTRK fusion (e.g., NTRK1). Alternatively or in addition, the NTRK fusion may include an NTRK2 or an NTRK3 fusion. The RTK fusion may comprise the RTK and at least a portion of SDC4, SLC34A2, FIG, LRIG3, EZR, TPM3, CD74, GOPC, KDELR3, CCDC6, or EML4. For example, the RTK fusion may comprise SDC4, SLC34A2, FIG, LRIG3, EZR, TPM3, CD74, GOPC, KDELR3, CCDC6, or EML4 fused to a ALK, ROS1, RET, NTRK1. The RTK fusion may comprise SDC4, SLC34A2, FIG, LRIG3, EZR, TPM3, or EML4 fused to the N-terminus of ALK, ROS1, RET, NTRK1. In some embodiments, exemplary RTK fusions include, but are not limited to SDC4-ROS1, SLC34A2-ROS1, FIG-ROS1, LRIG3-ROS1, EZR-ROS1, TPM3-ROS1, CD74-ROS1, GOPC-ROS1, KDELR3v, CCDC6-ROS1. In particular embodiments, the RTK fusion may include an SDC4-ROS1 fusion or an SLC34A2-ROS1 fusion. In particular embodiments, the RTK fusion may include a FIG-ROS1 fusion; a LRIG3-ROS1 fusion; an EZR-ROS1 fusion, and a TPM3-ROS1 fusion. In particular embodiments, the RTK fusion may include an EML4-ALK fusion. In particular embodiments, the RTK fusion may include an ETV6-NTRK3 fusion; a TPM3-NTRK1 fusion, a MPRIP-NTRK1 fusion, a CD74-NTRK1 fusion. In particular embodiments, the RTK fusion may include MPRIP; CD74; RABGAP1L; TPM3; TPR; TFG; PPL; CHTOP; ARHGEF2; NFASC; BCAN; LMNA; TP53; QKI; NACC2; VCL; AGBL4; TRIM24; AFAP1; SQSTM1; ETV6; BTB1; LYN; RBPMS fused to an RTK (e.g., to an NTRK). In particular embodiments, the RTK fusion may include MPRIP-NTRK1; CD74-NTRK1; RABGAP1L-NTRK1; TPM3-NTRK1; TPR-NTRK1; TFG-NTRK1; PPL-NTRK1; CHTOP-NTRK1; ARHGEF2-NTRK1; NFASC-NTRK1; BCAN-NTRK1; LMNA-NTRK1; TP53-NTRK1; QKI-NTRK2; NACC2-NTRK2; VCL-NTRK2; AGBL4-NTRK2; TRIM24-NTRK2; AFAP1-NTRK2; SQSTM1-NTRK2; ETV6-NTRK3; BTB1-NTRK3; LYN-NTRK3; RBPMS-NTRK3. In some embodiments, one or more of the particular or contemplated RTK fusions activates the MAPK pathway.

### SHP2 Inhibitors

In some embodiments of the disclosure, the compositions and methods disclosed herein, e.g., the methods for treating such diseases or disorders discussed herein (e.g., cancer), involve administering to a subject an effective amount of a SHP2 inhibitor or a composition (e.g., a pharmaceutical composition) comprising a SHP2 inhibitor. The terms “SHP2 inhibitor” and an “inhibitor of SHP2” are used interchangeably herein to refer to any compound or substance that is capable of inhibiting SHP2. These terms include, without limitation “allosteric SHP2 inhibitors” described herein, as well as other SHP2 inhibitors. Any such compound or substance capable of inhibiting SHP2 may be utilized in application with the present disclosure to inhibit SHP2.

In some embodiments, the compositions and methods described herein may comprise one or more SHP2 inhibitor(s) provided on Table 1.

In some embodiments, the compositions and methods described herein may comprise one or more SHP2 inhibitor(s) provided on Table 2.

In some embodiments, the compositions and methods described herein may comprise,

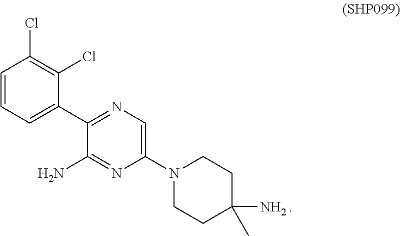 [SMILES: CC1(N)CCN(c2cnc(-c3cccc(Cl)c3Cl)c(N)n2)CC1]

The compositions and methods described herein may utilize one or more SHP2 inhibitor selected from, but not limited to any SHP2 inhibitor disclosed in any one of PCT applications PCT/US2017/041577 (WO2018013597); PCT/US2018/013018 (WO 2018136264); and PCT/US2018/013023 (WO 2018136265), each of which is incorporated herein by reference in its entirety. The compositions and methods described herein may utilize one or more SHP2 inhibitor selected from, but not limited to any SHP2 inhibitor disclosed in PCT applications PCT/IB2015/050343 (WO2015107493); PCT/IB2015/050344 (WO2015107494); PCT/IB2015/050345 (WO201507495); PCT/IB2016/053548 (WO2016/203404); PCT/IB2016/053549 (WO2016203405); PCT/IB2016/053550 (WO2016203406); PCT/US2010/045817 (WO2011022440); PCT/US2017/021784 (WO2017156397); PCT/US2016/060787 (WO2017079723); and PCT/CN2017/087471 (WO 2017211303), each of which is incorporated herein by reference in its entirety.

In some embodiments, the compositions and methods described herein may comprise,

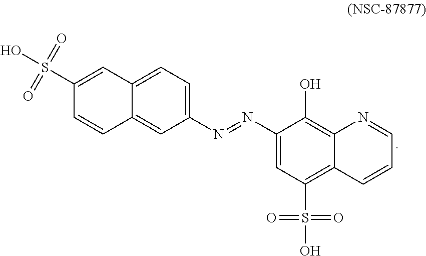 [SMILES: O=S(=O)(O)c1ccc2cc(/N=N/c3cc(S(=O)(=O)O)c4cccnc4c3O)ccc2c1]

In some embodiments, the compositions and methods described herein may comprise TNO155 (see also ClinicalTrials.gov Identifier: NCT03114319, available at world wide web address: clinicaltrials.gov/ct2/show/NCT03114319, incorporated herein by reference in its entirety).

In some embodiments, the compositions and methods described herein may comprise RLY-1971 (see also ClinicalTrials.gov Identifier: NCT04252339, available at world wide web address: clinicaltrials.gov/ct2/show/NCT04252339, incorporated herein by reference in its entirety).

In some embodiments, the compositions and methods described herein may comprise a SHP2 inhibitor compound of any one of Formula I, Formula II, Formula III, Formula I-V1, Formula I-V2, Formula I-W, Formula I-X, Formula I-Y, Formula I-Z, Formula IV, Formula V, Formula VI, Formula IV-X, Formula IV-Y, Formula IV-Z, Formula VII, Formula VIII, Formula IX, and Formula X, disclosed herein.

In some embodiments, the compositions and methods described herein may comprise the SHP2 inhibitor Compound RMC-4550.

In some embodiments, the compositions and methods described herein may comprise the SHP2 inhibitor Compound RMC-3943.

In some embodiments, the compositions and methods described herein may comprise the SHP2 inhibitor Compound RMC-4630. In some embodiments, Compound RMC-4630 has the following structure:

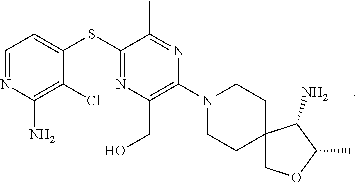 [SMILES: Cc1nc(N2CCC3(CC2)CO[C@@H](C)[C@H]3N)c(CO)nc1Sc1ccnc(N)c1Cl]

The disclosure provides compounds of Formula I:

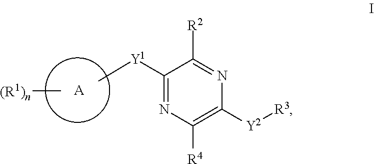

and pharmaceutically acceptable salts, prodrugs, solvates, hydrates, tautomers, or isomers thereof, wherein:
A is a 5- to 12-membered monocyclic or polycyclic cycloalkyl, heterocycloalkyl, aryl, or heteroaryl;
Y¹ is —S— or a direct bond;
Y² is —NR^{a}—, —(CR^{a}₂)ₘ—, —C(O)—, —C(R^{a})₂NH—, —(CR^{a}₂)ₘO—, —C(O)N(R^{a})—, —N(R^{a})C(O)—, —S(O)₂N(R^{a})—, —N(R^{a})S(O)₂—, —N(R^{a})C(O)N(R^{a})—, —N(R^{a})C(S)N(R^{a})—, —C(O)O—, —OC(O)—, —OC(O)N(R^{a})—, —N(R^{a})C(O)O—, —C(O)N(R^{a})O—, —N(R^{a})C(S)—, —C(S)N(R^{a})—, or —OC(O)O—; wherein the bond on the left side of Y², as drawn, is bound to the pyrazine ring and the bond on the right side of the Y² moiety is bound to R³;
R¹ is independently, at each occurrence, —H, -D, —C₁-C₆ alkyl, —C₂-C₆ alkenyl, —C₄-C₈ cycloalkenyl, —C₂-C₆ alkynyl, —C₃-C₈ cycloalkyl, —OH, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)R⁵, or —CO₂R⁵, wherein each alkyl, alkenyl, cycloalkenyl, alkynyl, or cycloalkyl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;
R² is independently —OR^{b}, —CN, —C₁-C₆ alkyl, —C₂-C₆ alkenyl, —C₄-C₈ cycloalkenyl, —C₂-C₆ alkynyl, —C₃-C₈ cycloalkyl, aryl, heterocyclyl containing 1-5 heteroatoms selected from the group consisting of N, S, P, and O, or heteroaryl containing 1-5 heteroatoms selected from the group consisting of N, S, P, and O; wherein each alkyl, alkenyl, cycloalkenyl, alkynyl, cycloalkyl, heterocyclyl, aryl, or heteroaryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; and wherein the heterocyclyl or heteroaryl is not attached via a nitrogen atom;
R^{a} is independently, at each occurrence, —H, -D, —OH, —C₃-C₈ cycloalkyl, or —C₁-C₆ alkyl, wherein each alkyl or cycloalkyl is optionally substituted with one or more —NH₂, wherein 2 R^{a}, together with the carbon atom to which they are both attached, can combine to form a 3- to 8-membered cycloalkyl;
R^{b} is independently, at each occurrence, —H, -D, —C₁-C₆ alkyl, —C₃-C₈ cycloalkyl, —C₂-C₆ alkenyl, or heterocyclyl containing 1-5 heteroatoms selected from the group consisting of N, S, P, and O;
wherein each alkyl, cycloalkyl, alkenyl, or heterocycle is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;
R³ is independently —C₁-C₆ alkyl or a 3- to 12-membered monocyclic or polycyclic heterocycle, wherein each alkyl or heterocycle is optionally substituted with one or more —C₁-C₆ alkyl, —OH, or —NH₂; or
R³ can combine with R^{a} to form a 3- to 12-membered monocyclic or polycyclic heterocycle or a 5- to 12-membered spiroheterocycle, wherein each heterocycle or spiroheterocycle is optionally substituted with one or more —C₁-C₆ alkyl, —OH, or —NH₂;
R⁴ is independently —H, -D, or —C₁-C₆ alkyl, wherein each alkyl is optionally substituted with one or more —OH, —NH₂, halogen, or oxo; or
R^{a} and R⁴, together with the atom or atoms to which they are attached, can combine to form a monocyclic or polycyclic C₃-C₁₂ cycloalkyl or a monocyclic or polycyclic 3- to 12-membered heterocycle, wherein the cycloalkyl or heterocycle is optionally substituted with oxo;
R⁵ and R⁶ are independently, at each occurrence, —H, -D, —C₁-C₆ alkyl, —C₂-C₆ alkenyl, —C₄-C₈ cycloalkenyl, —C₂-C₆ alkynyl, —C₃-C₈ cycloalkyl, a monocyclic or polycyclic 3- to 12-membered heterocycle, —OR⁷, —SR⁷, halogen, —NR⁷R⁸, —NO₂, or —CN;
R⁷ and R⁸ are independently, at each occurrence, —H, -D, —C₁-C₆ alkyl, —C₂-C₆ alkenyl, —C₄-C₈ cycloalkenyl, —C₂-C₆ alkynyl, —C₃-C₈ cycloalkyl, or a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN;
m is independently, at each occurrence, 1, 2, 3, 4, 5 or 6; and
n is independently, at each occurrence, 0, 1, 2, 3, 4, 5, 6, 7, 8, 9, or 10.

The disclosure provides compounds of Formula II:

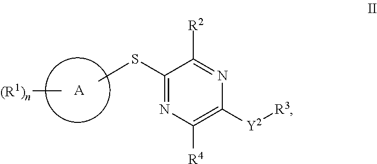

and pharmaceutically acceptable salts, prodrugs, solvates, hydrates, tautomers, or isomers thereof, wherein:
A is a 5- to 12-membered monocyclic or polycyclic cycloalkyl, heterocycloalkyl, aryl, or heteroaryl;
Y² is —NR^{a}—, —(CR^{a}₂)ₘ—, —C(O)—, —C(R^{a})₂NH—, —(CR^{a}₂)ₘO—, —C(O)N(R^{a})—, —N(R^{a})C(O)—, —S(O)₂N(R^{a})—, —N(R^{a})S(O)₂—, —N(R^{a})C(O)N(R^{a})—, —N(R^{a})C(S)N(R^{a})—, —C(O)O—, —OC(O)—, —OC(O)N(R^{a})—, —N(R^{a})C(O)O—, —C(O)N(R^{a})O—, —N(R^{a})C(S)—, —C(S)N(R^{a})—, or —OC(O)O—; wherein the bond on the left side of Y², as drawn, is bound to the pyrazine ring and the bond on the right side of the Y² moiety is bound to R³;
R¹ is independently, at each occurrence, —H, -D, —C₁-C₆ alkyl, —C₂-C₆ alkenyl, —C₄-C₈ cycloalkenyl, —C₂-C₆ alkynyl, —C₃-C₈ cycloalkyl, —OH, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)R⁵, or —CO₂R⁵, wherein each alkyl, alkenyl, cycloalkenyl, alkynyl, or cycloalkyl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;
R² is independently —OR^{b}, —CN, —C₁-C₆ alkyl, —C₂-C₆ alkenyl, —C₄-C₈ cycloalkenyl, —C₂-C₆ alkynyl, —C₃-C₈ cycloalkyl, aryl, heterocyclyl containing 1-5 heteroatoms selected from the group consisting of N, S, P, and O, or heteroaryl containing 1-5 heteroatoms selected from the group consisting of N, S, P, and O; wherein each alkyl, alkenyl, cycloalkenyl, alkynyl, cycloalkyl, heterocyclyl, aryl, or heteroaryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; and wherein the heterocyclyl or heteroaryl is not attached via a nitrogen atom;
R^{a} is independently, at each occurrence, —H, -D, —OH, —C₃-C₈ cycloalkyl, or —C₁-C₆ alkyl, wherein each alkyl or cycloalkyl is optionally substituted with one or more —NH₂, wherein 2 R^{a}, together with the carbon atom to which they are both attached, can combine to form a 3- to 8-membered cycloalkyl;
R^{b} is independently, at each occurrence, —H, -D, —C₁-C₆ alkyl, —C₃-C₈ cycloalkyl, —C₂-C₆ alkenyl, or heterocyclyl containing 1-5 heteroatoms selected from the group consisting of N, S, P, and O; wherein each alkyl, cycloalkyl, alkenyl, or heterocycle is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;
R³ is independently —C₁-C₆ alkyl or a 3- to 12-membered monocyclic or polycyclic heterocycle, wherein each alkyl or heterocycle is optionally substituted with one or more —C₁-C₆ alkyl, —OH, or —NH₂; or
R³ can combine with R^{a} to form a 3- to 12-membered monocyclic or polycyclic heterocycle or a 5- to 12-membered spiroheterocycle, wherein each heterocycle or spiroheterocycle is optionally substituted with one or more —C₁-C₆ alkyl, —OH, or —NH₂;
R⁴ is independently —H, -D, or —C₁-C₆ alkyl, wherein each alkyl is optionally substituted with one or more —OH, —NH₂, halogen, or oxo; or R^{a} and R⁴, together with the atom or atoms to which they are attached, can combine to form a monocyclic or polycyclic C₃-C₁₂ cycloalkyl or a monocyclic or polycyclic 3- to 12-membered heterocycle, wherein the cycloalkyl or heterocycle is optionally substituted with oxo;
R⁵ and R⁶ are independently, at each occurrence, —H, -D, —C₁-C₆ alkyl, —C₂-C₆ alkenyl, —C₄-C₈ cycloalkenyl, —C₂-C₆ alkynyl, —C₃-C₈ cycloalkyl, a monocyclic or polycyclic 3- to 12-membered heterocycle, —OR⁷, —SR⁷, halogen, —NR⁷R⁸, —NO₂, or —CN;
R⁷ and R⁸ are independently, at each occurrence, —H, -D, —C₁-C₆ alkyl, —C₂-C₆ alkenyl, —C₄-C₈ cycloalkenyl, —C₂-C₆ alkynyl, —C₃-C₈ cycloalkyl, or a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN;
m is independently, at each occurrence, 1, 2, 3, 4, 5 or 6; and
n is independently, at each occurrence, 0, 1, 2, 3, 4, 5, 6, 7, 8, 9, or 10.

The disclosure provides compounds of Formula III:

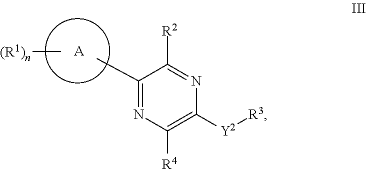

and pharmaceutically acceptable salts, prodrugs, solvates, hydrates, tautomers, or isomers thereof, wherein:
A is a 5- to 12-membered monocyclic or polycyclic cycloalkyl, heterocycloalkyl, aryl, or heteroaryl;
Y² is —NR^{a}—, —(CR^{a}₂)ₘ—, —C(O)—, —C(R^{a})₂NH—, —(CR^{a}₂)ₘO—, —C(O)N(R^{a})—, —N(R^{a})C(O)—, —S(O)₂N(R^{a})—, —N(R^{a})S(O)₂—, —N(R^{a})C(O)N(R^{a})—, —N(R^{a})C(S)N(R^{a})—, —C(O)O—, —OC(O)—, —OC(O)N(R^{a})—, —N(R^{a})C(O)O—, —C(O)N(R^{a})O—, —N(R^{a})C(S)—, —C(S)N(R^{a})—, or —OC(O)O—; wherein the bond on the left side of Y², as drawn, is bound to the pyrazine ring and the bond on the right side of the Y² moiety is bound to R³;
R¹ is independently, at each occurrence, —H, -D, —C₁-C₆ alkyl, —C₂-C₆ alkenyl, —C₄-C₈ cycloalkenyl, —C₂-C₆ alkynyl, —C₃-C₈ cycloalkyl, —OH, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)R⁵, or —CO₂R⁵, wherein each alkyl, alkenyl, cycloalkenyl, alkynyl, or cycloalkyl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;
R² is independently —OR^{b}, —CN, —C₁-C₆ alkyl, —C₂-C₆ alkenyl, —C₄-C₈ cycloalkenyl, —C₂-C₆ alkynyl, —C₃-C₈ cycloalkyl, aryl, heterocyclyl containing 1-5 heteroatoms selected from the group consisting of N, S, P, and O, or heteroaryl containing 1-5 heteroatoms selected from the group consisting of N, S, P, and O; wherein each alkyl, alkenyl, cycloalkenyl, alkynyl, cycloalkyl, heterocyclyl, aryl, or heteroaryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; and wherein the heterocyclyl or heteroaryl is not attached via a nitrogen atom;
R^{a} is independently, at each occurrence, —H, -D, —OH, —C₃-C₈ cycloalkyl, or —C₁-C₆ alkyl, wherein each alkyl or cycloalkyl is optionally substituted with one or more —NH₂, wherein 2 R^{a}, together with the carbon atom to which they are both attached, can combine to form a 3- to 8-membered cycloalkyl;
R^{b} is independently, at each occurrence, —H, -D, —C₁-C₆ alkyl, —C₃-C₈ cycloalkyl, —C₂-C₆ alkenyl, or heterocyclyl containing 1-5 heteroatoms selected from the group consisting of N, S, P, and O; wherein each alkyl, cycloalkyl, alkenyl, or heterocycle is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;
R³ is independently —C₁-C₆ alkyl or a 3- to 12-membered monocyclic or polycyclic heterocycle, wherein each alkyl or heterocycle is optionally substituted with one or more —C₁-C₆ alkyl, —OH, or —NH₂; or
R³ can combine with R^{a} to form a 3- to 12-membered monocyclic or polycyclic heterocycle or a 5- to 12-membered spiroheterocycle, wherein each heterocycle or spiroheterocycle is optionally substituted with one or more —C₁-C₆ alkyl, —OH, or —NH₂;
R⁴ is independently —H, -D, or —C₁-C₆ alkyl, wherein each alkyl is optionally substituted with one or more —OH, —NH₂, halogen, or oxo; or
R^{a} and R⁴, together with the atom or atoms to which they are attached, can combine to form a monocyclic or polycyclic C₃-C₁₂ cycloalkyl or a monocyclic or polycyclic 3- to 12-membered heterocycle, wherein the cycloalkyl or heterocycle is optionally substituted with oxo;
R⁵ and R⁶ are independently, at each occurrence, —H, -D, —C₁-C₆ alkyl, —C₂-C₆ alkenyl, —C₄-C₈ cycloalkenyl, —C₂-C₆ alkynyl, —C₃-C₈ cycloalkyl, a monocyclic or polycyclic 3- to 12-membered heterocycle, —OR⁷, —SR⁷, halogen, —NR⁷R⁸, —NO₂, or —CN;
R⁷ and R⁸ are independently, at each occurrence, —H, -D, —C₁-C₆ alkyl, —C₂-C₆ alkenyl, —C₄-C₈ cycloalkenyl, —C₂-C₆ alkynyl, —C₃-C₈ cycloalkyl, or a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN;
m is independently, at each occurrence, 1, 2, 3, 4, 5 or 6; and
n is independently, at each occurrence, 0, 1, 2, 3, 4, 5, 6, 7, 8, 9, or 10.

The disclosure provides compounds of Formula I-V1:

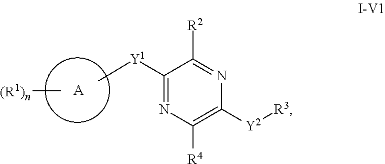

and pharmaceutically acceptable salts, prodrugs, solvates, hydrates, tautomers, or isomers thereof, wherein:
A is cycloalkyl, heterocycloalkyl, aryl, or heteroaryl, wherein cycloalkyl, heterocycloalkyl, aryl, and heteroaryl are 5- to 12-membered monocyclic or 5- to 12-membered polycyclic; Y¹ is —S—, a direct bond, —NH—, —S(O)₂—, —S(O)₂—NH—, —C(═CH₂)—, —CH—, or —S(O)—;
Y² is —NR^{a}—, wherein the bond on the left side of Y², as drawn, is bound to the pyrazine ring and the bond on the right side of the Y² moiety, as drawn, is bound to R³;
R^{a} and R⁴, together with the atom or atoms to which they are attached, are combined to form a monocyclic or polycyclic C₃-C₁₂ cycloalkyl or a monocyclic or polycyclic 3- to 12-membered heterocycle, wherein the cycloalkyl or heterocycle is optionally substituted with oxo; wherein the heterocycle optionally comprises —S(O)₂— in the heterocycle;
R¹ is independently, at each occurrence, —H, -D, —C₁-C₆ alkyl, —C₂-C₆ alkenyl, —C₄-C₈ cycloalkenyl, —C₂-C₆ alkynyl, —C₃-C₈ cycloalkyl, —OH, —OR⁶, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)R⁵, —CO₂R⁵, —C(O)NR⁵R⁶, —NR⁵C(O)R⁶, monocyclic or polycyclic heterocyclyl, spiroheterocyclyl, heteroaryl, or oxo, wherein each alkyl, alkenyl, cycloalkenyl, alkynyl, cycloalkyl, heterocyclyl, spiroheterocyclyl, or heteroaryl is optionally substituted with one or more —OH, halogen, —NO₂, oxo, ═O, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;
R² is independently —NH₂, —OR^{b}, —CN, —C₁-C₆ alkyl, —C₂-C₆ alkenyl, —C₄-C₈ cycloalkenyl, —C₂-C₆ alkynyl, halogen, —C(O)OR^{b}, —C₃-C₈ cycloalkyl, aryl, heterocyclyl containing 1-5 heteroatoms selected from the group consisting of N, S, P, and O, or heteroaryl containing 1-5 heteroatoms selected from the group consisting of N, S, P, and O; wherein each alkyl, alkenyl, cycloalkenyl, alkynyl, cycloalkyl, heterocyclyl, aryl, or heteroaryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; and wherein the heterocyclyl or heteroaryl is not attached via a nitrogen atom;
R^{b} is independently, at each occurrence, —H, -D, —OH, —C₁-C₆ alkyl, —C₃-C₈ cycloalkyl, —C₂-C₆ alkenyl, —(CH₂)ₙ-aryl, heterocyclyl containing 1-5 heteroatoms selected from the group consisting of N, S, P, and O, or heteroaryl containing 1-5 heteroatoms selected from the group consisting of N, S, P, and O; wherein each alkyl, cycloalkyl, alkenyl, heterocycle, heteroaryl, or —(CH₂)ₙ-aryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)NR⁵R⁶, —NR⁵C(O)R⁶, heterocycle, aryl, heteroaryl, —(CH₂)ₙOH, —C₁-C₆ alkyl, —CF₃, —CHF₂, or —CH₂F;
R³ is independently —H, —C₁-C₆ alkyl, a 3- to 12-membered monocyclic or polycyclic heterocycle, a 5- to 12-membered spiroheterocycle, C₃-C₈ cycloalkyl, or —(CH₂)ₙ—R^{b}, wherein each alkyl, spiroheterocycle, heterocycle, or cycloalkyl is optionally substituted with one or more —C₁-C₆ alkyl, —OH, —NH₂, —OR^{b}, —NHR^{b}, —(CH₂)ₙOH, heterocyclyl, or spiroheterocyclyl;
R⁵ and R⁶ are independently, at each occurrence, —H, -D, —C₁-C₆ alkyl, —C₂-C₆ alkenyl, —C₄-C₈ cycloalkenyl, —C₂-C₆ alkynyl, —C₃-C₈ cycloalkyl, a monocyclic or polycyclic 3- to 12-membered heterocycle, —OR⁷, —SR⁷, halogen, —NR⁷R⁸, —NO₂, —CF₃, or —CN;
R⁷ and R⁸ are independently, at each occurrence, —H, -D, —C₁-C₆ alkyl, —C₂-C₆ alkenyl, —C₄-C₈ cycloalkenyl, —C₂-C₆ alkynyl, —C₃-C₈ cycloalkyl, —OR^{b}, or a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN; and
n is independently, at each occurrence, 0, 1, 2, 3, 4, 5, 6, 7, 8, 9, or 10.

The disclosure provides compounds of Formula I-V2:

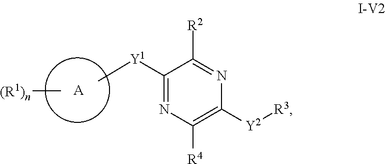

and pharmaceutically acceptable salts, prodrugs, solvates, hydrates, tautomers, and isomers thereof, wherein:
A is cycloalkyl, heterocycloalkyl, aryl, or heteroaryl, wherein cycloalkyl, heterocycloalkyl, aryl, and heteroaryl are 5- to 12-membered monocyclic or 5- to 12-membered polycyclic;
Y¹ is —S—, a direct bond, —NH—, —S(O)₂—, —S(O)₂—NH—, —C(═CH₂)—, —CH—, or —S(O)—;
Y² is —NR^{a}—, wherein the bond on the left side of Y², as drawn, is bound to the pyrazine ring and the bond on the right side of the Y² moiety, as drawn, is bound to R³;
R³ is combined with R^{a} to form a 3- to 12-membered polycyclic heterocycle or a 5- to 12-membered spiroheterocycle, wherein each heterocycle or spiroheterocycle is optionally substituted with one or more —C₁-C₆ alkyl, halogen, —OH, —OR^{b}, —NH₂, —NHR^{b}, heteroaryl, heterocyclyl, —(CH₂)ₙNH₂, —(CH₂)ₙOH, —COOR^{b}, —CON, —CONH(CH₂)ₙCOOR, —NHCOOR^{b}, —CF₃, —CHF₂, —CH₂F, or ═O;
R¹ is independently, at each occurrence, —H, -D, —C₁-C₆ alkyl, —C₂-C₆ alkenyl, —C₄-C₈ cycloalkenyl, —C₂-C₆ alkynyl, —C₃-C₈ cycloalkyl, —OH, —OR⁶, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)R⁵, —CO₂R⁵, —C(O)NR⁵R⁶, —NR⁵C(O)R⁶, monocyclic or polycyclic heterocyclyl, spiroheterocyclyl, heteroaryl, or oxo, wherein each alkyl, alkenyl, cycloalkenyl, alkynyl, cycloalkyl, heterocyclyl, spiroheterocyclyl, or heteroaryl is optionally substituted with one or more —OH, halogen, —NO₂, oxo, ═O, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;
R² is independently —NH₂, —OR^{b}, —CN, —C₁-C₆ alkyl, —C₂-C₆ alkenyl, —C₄-C₈ cycloalkenyl, —C₂-C₆ alkynyl, halogen, —C(O)OR, —C₃-C₈ cycloalkyl, aryl, heterocyclyl containing 1-5 heteroatoms selected from the group consisting of N, S, P, and O, or heteroaryl containing 1-5 heteroatoms selected from the group consisting of N, S, P, and O; wherein each alkyl, alkenyl, cycloalkenyl, alkynyl, cycloalkyl, heterocyclyl, aryl, or heteroaryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; and wherein the heterocyclyl or heteroaryl is not attached via a nitrogen atom;
R^{b} is independently, at each occurrence, —H, -D, —OH, —C₁-C₆ alkyl, —C₃-C₈ cycloalkyl, —C₂-C₆ alkenyl, —(CH₂)ₙ-aryl, heterocyclyl containing 1-5 heteroatoms selected from the group consisting of N, S, P, and O, or heteroaryl containing 1-5 heteroatoms selected from the group consisting of N, S, P, and O; wherein each alkyl, cycloalkyl, alkenyl, heterocycle, heteroaryl, or —(CH₂)ₙ-aryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)NR⁵R⁶, —NR⁵C(O)R⁶, heterocycle, aryl, heteroaryl, —(CH₂)ₙOH, —C₁-C₆ alkyl, —CF₃, —CHF₂, or —CH₂F;
R⁴ is independently —H, -D, —C₁-C₆ alkyl, —C₁-C₆haloalkyl, —C₁-C₆ hydroxyalkyl, —CF₂OH, —CHFOH, —NH—NHR⁵, —NH—OR⁵, —O—NR⁵R⁶, —NHR⁵, —OR⁵, —NHC(O)R⁵, —NHC(O)NHR⁵, —NHS(O)₂R⁵, —NHS(O)₂NHR⁵, —S(O)₂OH, —C(O)OR⁵, —NH(CH₂)ₙOH, —C(O)NH(CH₂)ₙOH, —C(O)NH(CH₂)ₙR^{b}, —C(O)R^{b}, —NH₂, —OH, —CN, —C(O)NR⁵R⁶, —S(O)₂NR⁵R⁶, C₃-C₈ cycloalkyl, aryl, heterocyclyl containing 1-5 heteroatoms selected from the group consisting of N, S, P, and O, or heteroaryl containing 1-5 heteroatoms selected from the group consisting of N, S, P, and O, wherein each alkyl, cycloalkyl, or heterocyclyl is optionally substituted with one or more —OH, —NH₂, —OR^{b}, halogen, or oxo; wherein each aryl or heteroaryl is optionally substituted with one or more —OH, —NH₂, or halogen;
R⁵ and R⁶ are independently, at each occurrence, —H, -D, —C₁-C₆ alkyl, —C₂-C₆ alkenyl, —C₄-C₈ cycloalkenyl, —C₂-C₆ alkynyl, —C₃-C₈ cycloalkyl, a monocyclic or polycyclic 3- to 12-membered heterocycle, —OR⁷, —SR⁷, halogen, —NR⁷R⁸, —NO₂, —CF₃, or —CN;
R⁷ and R⁸ are independently, at each occurrence, —H, -D, —C₁-C₆ alkyl, —C₂-C₆ alkenyl, —C₄-C₈ cycloalkenyl, —C₂-C₆ alkynyl, —C₃-C₈ cycloalkyl, —OR^{b}, or a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN; and
n is independently, at each occurrence, 0, 1, 2, 3, 4, 5, 6, 7, 8, 9, or 10.

The disclosure provides compounds of Formula I-W:

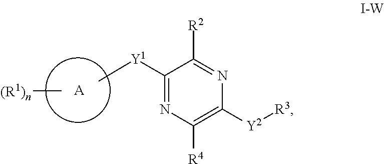

and pharmaceutically acceptable salts, prodrugs, solvates, hydrates, tautomers, and isomers thereof, wherein:
A is cycloalkyl, heterocycloalkyl, aryl, or heteroaryl, wherein cycloalkyl, heterocycloalkyl, aryl, and heteroaryl are 5- to 12-membered monocyclic or 5- to 12-membered polycyclic;
Y¹ is —S—, a direct bond, —NH—, —S(O)₂—, —S(O)₂—NH—, —C(═CH₂)—, —CH—, or —S(O)—;
Y² is —NR^{a}—, —(CR^{a}₂)ₘ—, —C(O)—, —C(R^{a})₂NH—, —(CR^{a}₂)ₘO—, —C(O)N(R^{a})—, —N(R^{a})C(O)—, —S(O)₂N(R^{a})—, —N(R^{a})S(O)₂—, —N(R^{a})C(O)N(R^{a})—, —N(R^{a})C(S)N(R^{a})—, —C(O)O—, —OC(O)—, —OC(O)N(R^{a})—, —N(R^{a})C(O)O—, —C(O)N(R^{a})O—, —N(R^{a})C(S)—, —C(S)N(R^{a})—, or —OC(O)O—; wherein the bond on the left side of Y², as drawn, is bound to the pyrazine ring and the bond on the right side of the Y² moiety, as drawn, is bound to R³;
R¹ is independently, at each occurrence, —H, -D, —C₁-C₆ alkyl, —C₂-C₆ alkenyl, —C₄-C₈ cycloalkenyl, —C₂-C₆ alkynyl, —C₃-C₈ cycloalkyl, —OH, —OR⁶, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)R⁵, —CO₂R⁵, —C(O)NR⁵R⁶, —NR⁵C(O)R⁶, monocyclic or polycyclic heterocyclyl, spiroheterocyclyl, heteroaryl, or oxo, wherein each alkyl, alkenyl, cycloalkenyl, alkynyl, cycloalkyl, heterocyclyl, spiroheterocyclyl, or heteroaryl is optionally substituted with one or more —OH, halogen, —NO₂, oxo, ═O, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; R² is independently —OR^{b}, —CN, —C₁-C₆ alkyl, —C₂-C₆ alkenyl, —C₄-C₈ cycloalkenyl, —C₂-C₆ alkynyl, halogen, —C(O)OR^{b}, —C₃-C₈ cycloalkyl, aryl, heterocyclyl containing 1-5 heteroatoms selected from the group consisting of N, S, P, and O, or heteroaryl containing 1-5 heteroatoms selected from the group consisting of N, S, P, and O; wherein each alkyl, alkenyl, cycloalkenyl, alkynyl, cycloalkyl, heterocyclyl, aryl, or heteroaryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; and wherein the heterocyclyl or heteroaryl is not attached via a nitrogen atom;
R^{a} is independently, at each occurrence, —H, -D, —OH, —C₃-C₈ cycloalkyl, —C₁-C₆ alkyl, 3- to 12-membered heterocyclyl, or —(CH₂)ₙ-aryl, wherein each alkyl or cycloalkyl is optionally substituted with one or more —NH₂, or wherein 2 R^{a}, together with the carbon atom to which they are both attached, can combine to form a 3- to 8-membered cycloalkyl;
R^{b} is independently, at each occurrence, —H, -D, —OH, —C₁-C₆ alkyl, —C₃-C₈ cycloalkyl, —C₂-C₆ alkenyl, —(CH₂)ₙ-aryl, heterocyclyl containing 1-5 heteroatoms selected from the group consisting of N, S, P, and O, or heteroaryl containing 1-5 heteroatoms selected from the group consisting of N, S, P, and O; wherein each alkyl, cycloalkyl, alkenyl, heterocycle, heteroaryl, or —(CH₂)ₙ-aryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)NR⁵R⁶, —NR⁵C(O)R⁶, heterocycle, aryl, heteroaryl, —(CH₂)ₙOH, —C₁-C₆ alkyl, —CF₃, —CHF₂, or —CH₂F;
R³ is independently —H, —C₁-C₆ alkyl, a 3- to 12-membered monocyclic or polycyclic heterocycle, a 5- to 12-membered spiroheterocycle, C₃-C₈ cycloalkyl, or —(CH₂)ₙ—R^{b}, wherein each alkyl, spiroheterocycle, heterocycle, or cycloalkyl is optionally substituted with one or more —C₁-C₆ alkyl, —OH, —NH₂, —OR^{b}, —NHR^{b}, —(CH₂)ₙOH, heterocyclyl, or spiroheterocyclyl; or
R³ can combine with R^{a} to form a 3- to 12-membered monocyclic or polycyclic heterocycle or a 5- to 12-membered spiroheterocycle, wherein each heterocycle or spiroheterocycle is optionally substituted with one or more —C₁-C₆ alkyl, halogen, —OH, —OR^{b}, —NH₂, —NHR^{b}, heteroaryl, heterocyclyl, —(CH₂)ₙNH₂, —(CH₂)ₙOH, —COOR^{b}, —CONHR^{b}, —CONH(CH₂)ₙCOOR^{b}, —NHCOOR^{b}, —CF₃, —CHF₂, —CH₂F, or ═O;
R⁴ is independently —H, -D, —C₁-C₆ alkyl, —C₁-C₆haloalkyl, —C₁-C₆ hydroxyalkyl —CF₂OH, —CHFOH—NH—NHR⁵, —NH—OR⁵, —O—NR⁵R⁶, —NHR⁵, —OR⁵, —NHC(O)R⁵, —NHC(O)NHR⁵, —NHS(O)₂R⁵, —NHS(O)₂NHR⁵, —S(O)₂OH, —C(O)OR⁵, —NH(CH₂)ₙOH, —C(O)NH(CH₂)ₙOH, —C(O)NH(CH₂)ₙR^{b}, —C(O)R^{b}, —NH₂, —OH, —CN, —C(O)NR⁵R⁶, —S(O)₂NR⁵R⁶, C₃-C₈ cycloalkyl, aryl, heterocyclyl containing 1-5 heteroatoms selected from the group consisting of N, S, P, and O, or heteroaryl containing 1-5 heteroatoms selected from the group consisting of N, S, P, and O, wherein each alkyl, cycloalkyl, or heterocyclyl is optionally substituted with one or more —OH, —NH₂, —OR^{b}, halogen, or oxo; wherein each aryl or heteroaryl is optionally substituted with one or more —OH, —NH₂, or halogen; or
R^{a} and R⁴, together with the atom or atoms to which they are attached, can combine to form a monocyclic or polycyclic C₃-C₁₂ cycloalkyl or a monocyclic or polycyclic 3- to 12-membered heterocycle, wherein the cycloalkyl or heterocycle is optionally substituted with oxo; wherein the heterocycle optionally comprises —S(O)₂— in the heterocycle;
R⁵ and R⁶ are independently, at each occurrence, —H, -D, —C₁-C₆ alkyl, —C₂-C₆ alkenyl, —C₄-C₈ cycloalkenyl, —C₂-C₆ alkynyl, —C₃-C₈ cycloalkyl, a monocyclic or polycyclic 3- to 12-membered heterocycle, —OR⁷, —SR⁷, halogen, —NR⁷R⁸, —NO₂, —CF₃, or —CN;
R⁷ and R⁸ are independently, at each occurrence, —H, -D, —C₁-C₆ alkyl, —C₂-C₆ alkenyl, —C₄-C₈ cycloalkenyl, —C₂-C₆ alkynyl, —C₃-C₈ cycloalkyl, —OR^{b}, or a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN;
m is independently, at each occurrence, 1, 2, 3, 4, 5 or 6; and
n is independently, at each occurrence, 0, 1, 2, 3, 4, 5, 6, 7, 8, 9, or 10.

The disclosure provides compounds of Formula I-X:

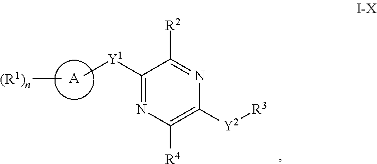

and pharmaceutically acceptable salts, prodrugs, solvates, hydrates, tautomers, or isomers thereof, wherein:
A is a 5- to 12-membered monocyclic or polycyclic cycloalkyl, heterocycloalkyl, aryl, or heteroaryl;
Y¹ is —S— or a direct bond;
Y² is —NR^{a}—, —(CR^{a}₂)ₘ—, —C(O)—, —C(R^{a})₂NH—, —(CR^{a}₂)ₘO—, —C(O)N(R^{a})—, —N(R^{a})C(O)—, —S(O)₂N(R^{a})—, —N(R^{a})S(O)₂—, —N(R^{a})C(O)N(R^{a})—, —N(R^{a})C(S)N(R^{a})—, —C(O)O—, —OC(O)—, —OC(O)N(R^{a})—, —N(R^{a})C(O)O—, —C(O)N(R^{a})O—, —N(R^{a})C(S)—, —C(S)N(R^{a})—, or —OC(O)O—; wherein the bond on the left side of Y², as drawn, is bound to the pyrazine ring and the bond on the right side of the Y² moiety, as drawn, is bound to R³;
R¹ is independently, at each occurrence, —H, -D, —C₁-C₆ alkyl, —C₂-C₆ alkenyl, —C₄-C₈ cycloalkenyl, —C₂-C₆ alkynyl, —C₃-C₈ cycloalkyl, —OH, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)R⁵, or —CO₂R⁵, wherein each alkyl, alkenyl, cycloalkenyl, alkynyl, or cycloalkyl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;
R² is independently —OR^{b}, —CN, —C₁-C₆ alkyl, —C₂-C₆ alkenyl, —C₄-C₈ cycloalkenyl, —C₂-C₆ alkynyl, —C₃-C₈ cycloalkyl, aryl, heterocyclyl containing 1-5 heteroatoms selected from the group consisting of N, S, P, and O, or heteroaryl containing 1-5 heteroatoms selected from the group consisting of N, S, P, and O; wherein each alkyl, alkenyl, cycloalkenyl, alkynyl, cycloalkyl, heterocyclyl, aryl, or heteroaryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; and wherein the heterocyclyl or heteroaryl is not attached via a nitrogen atom;
R^{a} is independently, at each occurrence, —H, -D, —OH, —C₃-C₈ cycloalkyl, or —C₁-C₆ alkyl, wherein each alkyl or cycloalkyl is optionally substituted with one or more —NH₂, wherein 2 R^{a}, together with the carbon atom to which they are both attached, can combine to form a 3- to 8-membered cycloalkyl;
R^{b} is independently, at each occurrence, —H, -D, —C₁-C₆ alkyl, —C₃-C₈ cycloalkyl, —C₂-C₆ alkenyl, or heterocyclyl containing 1-5 heteroatoms selected from the group consisting of N, S, P, and O; wherein each alkyl, cycloalkyl, alkenyl, or heterocycle is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;
R³ is independently —H, —C₁-C₆ alkyl, or a 3- to 12-membered monocyclic or polycyclic heterocycle, wherein each alkyl or heterocycle is optionally substituted with one or more —C₁-C₆ alkyl, —OH, or —NH₂; or
R³ can combine with R^{a} to form a 3- to 12-membered monocyclic or polycyclic heterocycle or a 5- to 12-membered spiroheterocycle, wherein each heterocycle or spiroheterocycle is optionally substituted with one or more —C₁-C₆ alkyl, —OH, or —NH₂;
R⁴ is independently —H, -D, —C₁-C₆ alkyl, —NH—NHR⁵, —NH—OR⁵, —O—NR⁵R⁶, —NHR⁵, —OR⁵, —NHC(O)R⁵, —NHC(O)NHR⁵, —NHS(O)₂R⁵, —NHS(O)₂NHR⁵, —S(O)₂OH, —C(O)OR⁵, —C(O)NR⁵R⁶, —S(O)₂NR⁵R⁶, C₃-C₈ cycloalkyl, aryl, heterocyclyl containing 1-5 heteroatoms selected from the group consisting of N, S, P, and O, or heteroaryl containing 1-5 heteroatoms selected from the group consisting of N, S, P, and O, wherein each alkyl, cycloalkyl, or heterocyclyl is optionally substituted with one or more —OH, —NH₂, halogen, or oxo; wherein each aryl or heteroaryl is optionally substituted with one or more —OH, —NH₂, or halogen; or
R^{a} and R⁴, together with the atom or atoms to which they are attached, can combine to form a monocyclic or polycyclic C₃-C₁₂ cycloalkyl or a monocyclic or polycyclic 3- to 12-membered heterocycle, wherein the cycloalkyl or heterocycle is optionally substituted with oxo; wherein the heterocycle optionally comprises —S(O)₂— in the heterocycle;
R⁵ and R⁶ are independently, at each occurrence, —H, -D, —C₁-C₆ alkyl, —C₂-C₆ alkenyl, —C₄-C₈ cycloalkenyl, —C₂-C₆ alkynyl, —C₃-C₈ cycloalkyl, a monocyclic or polycyclic 3- to 12-membered heterocycle, —OR⁷, —SR⁷, halogen, —NR⁷R⁸, —NO₂, or —CN; R⁷ and R⁸ are independently, at each occurrence, —H, -D, —C₁-C₆ alkyl, —C₂-C₆ alkenyl, —C₄-C₈ cycloalkenyl, —C₂-C₆ alkynyl, —C₃-C₈ cycloalkyl, or a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN;
m is independently, at each occurrence, 1, 2, 3, 4, 5 or 6; and
n is independently, at each occurrence, 0, 1, 2, 3, 4, 5, 6, 7, 8, 9, or 10.

The disclosure provides compounds of Formula I-Y:

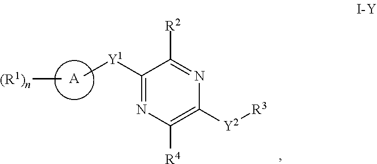

and pharmaceutically acceptable salts, prodrugs, solvates, hydrates, tautomers, or isomers thereof, wherein:
A is a 5- to 12-membered monocyclic or polycyclic cycloalkyl, heterocycloalkyl, aryl, or heteroaryl;
Y¹ is —S— or a direct bond;
Y² is —NR^{a}—, —(CR^{a}₂)ₘ—, —C(O)—, —C(R^{a})₂NH—, —(CR^{a}₂)ₘO—, —C(O)N(R^{a})—, —N(R^{a})C(O)—, —S(O)₂N(R^{a})—, —N(R^{a})S(O)₂—, —N(R^{a})C(O)N(R^{a})—, —N(R^{a})C(S)N(R^{a})—, —C(O)O—, —OC(O)—, —OC(O)N(R^{a})—, —N(R^{a})C(O)O—, —C(O)N(R^{a})O—, —N(R^{a})C(S)—, —C(S)N(R^{a})—, or —OC(O)O—; wherein the bond on the left side of Y², as drawn, is bound to the pyrazine ring and the bond on the right side of the Y² moiety, as drawn, is bound to R³;
R¹ is independently, at each occurrence, —H, -D, —C₁-C₆ alkyl, —C₂-C₆ alkenyl, —C₄-C₈ cycloalkenyl, —C₂-C₆ alkynyl, —C₃-C₈ cycloalkyl, —OH, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)R⁵, or —CO₂R⁵, wherein each alkyl, alkenyl, cycloalkenyl, alkynyl, or cycloalkyl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;
R² is independently —OR^{b}, —CN, —C₁-C₆ alkyl, —C₂-C₆ alkenyl, —C₄-C₈ cycloalkenyl, —C₂-C₆ alkynyl, —C₃-C₈ cycloalkyl, aryl, heterocyclyl containing 1-5 heteroatoms selected from the group consisting of N, S, P, and O, or heteroaryl containing 1-5 heteroatoms selected from the group consisting of N, S, P, and O; wherein each alkyl, alkenyl, cycloalkenyl, alkynyl, cycloalkyl, heterocyclyl, aryl, or heteroaryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; and wherein the heterocyclyl or heteroaryl is not attached via a nitrogen atom;
R^{a} is independently, at each occurrence, —H, -D, —OH, —C₃-C₈ cycloalkyl, or —C₁-C₆ alkyl, wherein each alkyl or cycloalkyl is optionally substituted with one or more —NH₂, wherein 2 R^{a}, together with the carbon atom to which they are both attached, can combine to form a 3- to 8-membered cycloalkyl;
R^{b} is independently, at each occurrence, —H, -D, —C₁-C₆ alkyl, —C₃-C₈ cycloalkyl, —C₂-C₆ alkenyl, or heterocyclyl containing 1-5 heteroatoms selected from the group consisting of N, S, P, and O; wherein each alkyl, cycloalkyl, alkenyl, or heterocycle is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, heteroaryl, —(CH₂)ₙOH, —C₁-C₆ alkyl, —CF₃, —CHF₂, or —CH₂F;
R³ is independently —H, —C₁-C₆ alkyl, a 3- to 12-membered monocyclic or polycyclic heterocycle, C₃-C₈ cycloalkyl, or —(CH₂)ₙ—R^{b}, wherein each alkyl, heterocycle, or cycloalkyl is optionally substituted with one or more —C₁-C₆ alkyl, —OH, —NH₂, —OR^{b}, —NHR^{b}, —(CH₂)ₙOH, heterocyclyl, or spiroheterocyclyl; or
R³ can combine with R^{a} to form a 3- to 12-membered monocyclic or polycyclic heterocycle or a 5- to 12-membered spiroheterocycle, wherein each heterocycle or spiroheterocycle is optionally substituted with one or more —C₁-C₆ alkyl, —OH, —NH₂, heteroaryl, heterocyclyl, —(CH₂)ₙNH₂, —COOR^{b}, —CONHR^{b}, —CONH(CH₂)ₙCOOR^{b}, —NHCOOR^{b}, —CF₃, —CHF₂, or —CH₂F;
R⁴ is independently —H, -D, —C₁-C₆ alkyl, —NH—NHR⁵, —NH—OR⁵, —O—NR⁵R⁶, —NHR⁵, —OR⁵, —NHC(O)R⁵, —NHC(O)NHR⁵, —NHS(O)₂R⁵, —NHS(O)₂NHR⁵, —S(O)₂OH, —C(O)OR⁵, —NH(CH₂)ₙOH, —C(O)NH(CH₂)ₙOH, —C(O)NH(CH₂)ₙR^{b}, —C(O)R^{b}, —NH₂, —OH, —CN, —C(O)NR⁵R⁶, —S(O)₂NR⁵R⁶, C₃-C₈ cycloalkyl, aryl, heterocyclyl containing 1-5 heteroatoms selected from the group consisting of N, S, P, and O, or heteroaryl containing 1-5 heteroatoms selected from the group consisting of N, S, P, and O, wherein each alkyl, cycloalkyl, or heterocyclyl is optionally substituted with one or more —OH, —NH₂, halogen, or oxo; wherein each aryl or heteroaryl is optionally substituted with one or more —OH, —NH₂, or halogen; or
R^{a} and R⁴, together with the atom or atoms to which they are attached, can combine to form a monocyclic or polycyclic C₃-C₁₂ cycloalkyl or a monocyclic or polycyclic 3- to 12-membered heterocycle, wherein the cycloalkyl or heterocycle is optionally substituted with oxo; wherein the heterocycle optionally comprises —S(O)₂— in the heterocycle;
R⁵ and R⁶ are independently, at each occurrence, —H, -D, —C₁-C₆ alkyl, —C₂-C₆ alkenyl, —C₄-C₈ cycloalkenyl, —C₂-C₆ alkynyl, —C₃-C₈ cycloalkyl, a monocyclic or polycyclic 3- to 12-membered heterocycle, —OR⁷, —SR⁷, halogen, —NR⁷R⁸, —NO₂, or —CN;
R⁷ and R⁸ are independently, at each occurrence, —H, -D, —C₁-C₆ alkyl, —C₂-C₆ alkenyl, —C₄-C₈ cycloalkenyl, —C₂-C₆ alkynyl, —C₃-C₈ cycloalkyl, or a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN;
m is independently, at each occurrence, 1, 2, 3, 4, 5 or 6; and
n is independently, at each occurrence, 0, 1, 2, 3, 4, 5, 6, 7, 8, 9, or 10.

The disclosure provides compounds of Formula I-Z:

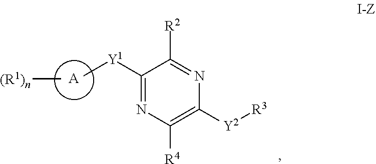

and pharmaceutically acceptable salts, prodrugs, solvates, hydrates, tautomers, or isomers thereof, wherein:
A is a 5- to 12-membered monocyclic or polycyclic cycloalkyl, heterocycloalkyl, aryl, or heteroaryl;
Y¹ is —S—, a direct bond, —NH—, —S(O)₂—, —S(O)₂—NH—, —C(═CH₂)—, —CH—, or —S(O)—;
Y² is —NR^{a}—, —(CR^{a}₂)ₘ—, —C(R^{a})₂NH—, —(CR^{a}₂)ₘO—, —C(O)N(R^{a})—, —N(R^{a})C(O)—, —S(O)₂N(R^{a})—, —N(R^{a})S(O)₂—, —N(R^{a})C(O)N(R^{a})—, —N(R^{a})C(S)N(R^{a})—, —OC(O)N(R^{a})—, —N(R^{a})C(O)O—, —C(O)N(R^{a})O—, —N(R^{a})C(S)—, or —C(S)N(R^{a})—; wherein the bond on the left side of Y², as drawn, is bound to the pyrazine ring and the bond on the right side of the Y² moiety, as drawn, is bound to R³;
R¹ is independently, at each occurrence, —H, -D, —C₁-C₆ alkyl, —C₂-C₆ alkenyl, —C₄-C₈ cycloalkenyl, —C₂-C₆ alkynyl, —C₃-C₈ cycloalkyl, —OH, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)R⁵, or —CO₂R⁵, wherein each alkyl, alkenyl, cycloalkenyl, alkynyl, or cycloalkyl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;
R² is independently —OR^{b}, —NH₂, —CN, —C₁-C₆ alkyl, —C₂-C₆ alkenyl, —C₄-C₈ cycloalkenyl, —C₂-C₆ alkynyl, halogen, —C(O)OR^{b}, —C₃-C₈ cycloalkyl, aryl, heterocyclyl containing 1-5 heteroatoms selected from the group consisting of N, S, P, and O, or heteroaryl containing 1-5 heteroatoms selected from the group consisting of N, S, P, and O; wherein each alkyl, alkenyl, cycloalkenyl, alkynyl, cycloalkyl, heterocyclyl, aryl, or heteroaryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; and wherein the heterocyclyl or heteroaryl is not attached via a nitrogen atom;
R^{a} is independently, at each occurrence —OH, —C₃-C₈ cycloalkyl, or —C₁-C₆ alkyl, wherein each alkyl or cycloalkyl is optionally substituted with one or more —NH₂, wherein 2 R^{a}, together with the carbon atom to which they are both attached, can combine to form a 3- to 8-membered cycloalkyl;
R^{b} is independently, at each occurrence, —H, -D, —C₁-C₆ alkyl, —C₃-C₈ cycloalkyl, —C₂-C₆ alkenyl, or heterocyclyl containing 1-5 heteroatoms selected from the group consisting of N, S, P, and O; wherein each alkyl, cycloalkyl, alkenyl, or heterocycle is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, heteroaryl, —(CH₂)ₙOH, —C₁-C₆ alkyl, —CF₃, —CHF₂, or —CH₂F;
R³ is independently —H, —C₁-C₆ alkyl, a 3- to 12-membered monocyclic or polycyclic heterocycle, C₃-C₈ cycloalkyl, or —(CH₂)ₙ—R^{b}, wherein each alkyl, heterocycle, or cycloalkyl is optionally substituted with one or more —C₁-C₆ alkyl, —OH, —NH₂, —OR^{b}, —NHR^{b}, —(CH₂)ₙOH, heterocyclyl, or spiroheterocyclyl; or
R³ can combine with R^{a} to form a 3- to 12-membered monocyclic or polycyclic heterocycle or a 5- to 12-membered spiroheterocycle, wherein each heterocycle or spiroheterocycle is optionally substituted with one or more —C₁-C₆ alkyl, —OH, —NH₂, heteroaryl, heterocyclyl, —(CH₂)ₙNH₂, —COOR^{b}, —CONHR^{b}, —CONH(CH₂)ₙCOOR, —NHCOOR^{b}, —CF₃, —CHF₂, or —CH₂F; R⁴ is independently —C₁-C₆ alkyl, —NH—NHR⁵, —NH—OR⁵, —O—NR⁵R⁶, —NHR⁵, —OR⁵, —NHC(O)R⁵, —NHC(O)NHR⁵, —NHS(O)₂R⁵, —NHS(O)₂NHR⁵, —S(O)₂OH, —C(O)OR⁵, —NH(CH₂)ₙOH, —C(O)NH(CH₂)ₙOH, —C(O)NH(CH₂)ₙR^{b}, —C(O)R^{b}, —NH₂, —OH, —C(O)NR⁵R⁶, —S(O)₂NR⁵R⁶, C₃-C₈ cycloalkyl, aryl, heterocyclyl containing 1-5 heteroatoms selected from the group consisting of N, S, P, and O, or heteroaryl containing 1-5 heteroatoms selected from the group consisting of N, S, P, and O, wherein each alkyl, cycloalkyl, or heterocyclyl is optionally substituted with one or more —OH, —NH₂, halogen, or oxo; wherein each aryl or heteroaryl is optionally substituted with one or more —OH, —NH₂, or halogen;
R^{a} and R⁴, together with the atom or atoms to which they are attached, are combined to form a monocyclic or polycyclic C₃-C₁₂ cycloalkyl or a monocyclic or polycyclic 3- to 12-membered heterocycle, wherein the cycloalkyl or heterocycle is optionally substituted with oxo; wherein the heterocycle optionally comprises —S(O)₂— in the heterocycle;
R⁵ and R⁶ are independently, at each occurrence, —H, -D, —C₁-C₆ alkyl, —C₂-C₆ alkenyl, —C₄-C₈ cycloalkenyl, —C₂-C₆ alkynyl, —C₃-C₈ cycloalkyl, a monocyclic or polycyclic 3- to 12-membered heterocycle, —OR⁷, —SR⁷, halogen, —NR⁷R⁸, —NO₂, or —CN;
R⁷ and R⁸ are independently, at each occurrence, —H, -D, —C₁-C₆ alkyl, —C₂-C₆ alkenyl, —C₄-C₈ cycloalkenyl, —C₂-C₆ alkynyl, —C₃-C₈ cycloalkyl, or a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN;
m is independently, at each occurrence, 1, 2, 3, 4, 5 or 6; and
n is independently, at each occurrence, 0, 1, 2, 3, 4, 5, 6, 7, 8, 9, or 10.

The disclosure provides compounds of Formula IV:

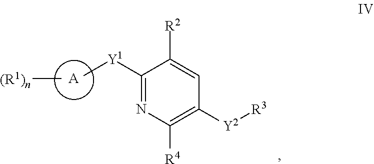

and pharmaceutically acceptable salts, prodrugs, solvates, hydrates, tautomers, or isomers thereof, wherein:
A is selected from the group consisting of 5- to 12-membered monocyclic or polycyclic cycloalkyl, heterocycloalkyl, aryl, or heteroaryl;
Y¹ is —S— or a direct bond;
Y² is selected from the group consisting of: —NR^{a}—, —(CR^{a}₂)ₘ—, —C(O)—, —C(R^{a})₂NH—, —(CR^{a}₂)ₘO—, —C(O)N(R^{a})—, —N(R^{a})C(O)—, —S(O)₂N(R^{a})—, —N(R^{a})S(O)₂—, —N(R^{a})C(O)N(R^{a})—, —N(R^{a})C(S)N(R^{a})—, —C(O)O—, —OC(O)—, —OC(O)N(R^{a})—, —N(R^{a})C(O)O—, —C(O)N(R^{a})O—, —N(R^{a})C(S)—, —C(S)N(R^{a})—, and —OC(O)O—; wherein the bond on the left side of Y², as drawn, is bound to the pyridine ring and the bond on the right side of the Y² moiety is bound to R³;
R¹ is independently, at each occurrence, —H, -D, —C₁-C₆ alkyl, —C₂-C₆ alkenyl, —C₄-C₈ cycloalkenyl, —C₂-C₆ alkynyl, —C₃-C₈ cycloalkyl, —OH, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)R⁵, or —CO₂R⁵, wherein each alkyl, alkenyl, cycloalkenyl, alkynyl, or cycloalkyl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;
R² is independently —OR^{b}, —CN, —C₁-C₆ alkyl, —C₂-C₆ alkenyl, —C₄-C₈ cycloalkenyl, —C₂-C₆ alkynyl, —C₃-C₈ cycloalkyl, aryl, heterocyclyl containing 1-5 heteroatoms selected from the group consisting of N, S, P, or O, or heteroaryl containing 1-5 heteroatoms selected from the group consisting of N, S, P, or O; wherein each alkyl, alkenyl, cycloalkenyl, alkynyl, cycloalkyl, heterocyclyl, aryl, or heteroaryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; and wherein the heterocyclyl or heteroaryl is not attached via a nitrogen atom;
R^{a} is independently, at each occurrence, selected from the group consisting of —H, -D, —OH, —C₃-C₈ cycloalkyl, and —C₁-C₆ alkyl, wherein each alkyl or cycloalkyl is optionally substituted with one or more —NH₂, wherein 2 R^{a}, together with the carbon atom to which they are both attached, can combine to form a 3- to 8-membered cycloalkyl;
R^{b} is independently —H, -D, —C₁-C₆ alkyl, —C₁-C₆ cycloalkyl, —C₂-C₆ alkenyl, or heterocyclyl containing 1-5 heteroatoms selected from the group consisting of N, S, P, or O; wherein each alkyl, cycloalkyl, alkenyl, or heterocycle is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;
R³ is independently, at each occurrence, selected from the group consisting of —C₁-C₆ alkyl, or a 3- to 12-membered monocyclic or polycyclic heterocycle, wherein each alkyl or heterocycle is optionally substituted with one or more —C₁-C₆ alkyl, —OH, or —NH₂; or
R³ can combine with R^{a} to form a 3- to 12-membered monocyclic or polycyclic heterocycle, or a 5- to 12-membered spiroheterocycle, wherein each heterocycle or spiroheterocycle is optionally substituted with —C₁-C₆ alkyl, —OH, or —NH₂;
R⁴ is independently, at each occurrence, —H, -D, or —C₁-C₆ alkyl, wherein each alkyl is optionally substituted with one or more —OH, —NH₂, halogen, or oxo; or
R^{a} and R⁴, together with the atom or atoms to which they are attached, can combine to form a monocyclic or polycyclic C₃-C₁₂ cycloalkyl, or a monocyclic or polycyclic 3- to 12-membered heterocycle, wherein the cycloalkyl or heterocycle is optionally substituted with oxo;
R⁵ and R⁶ are each independently, at each occurrence, selected from the group consisting of —H, -D, —C₁-C₆ alkyl, —C₂-C₆ alkenyl, —C₄-C₈ cycloalkenyl, —C₂-C₆ alkynyl, —C₃-C₈ cycloalkyl, a monocyclic or polycyclic 3- to 12-membered heterocycle, —OR⁷, —SR⁷, halogen, —NR⁷R⁸, —NO₂, and —CN;
R⁷ and R⁸ are independently, at each occurrence, —H, -D, —C₁-C₆ alkyl, —C₂-C₆ alkenyl, —C₄-C₈ cycloalkenyl, —C₂-C₆ alkynyl, —C₃-C₈ cycloalkyl, a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN;
m is independently 1, 2, 3, 4, 5 or 6; and
n is independently 0, 1, 2, 3, 4, 5, 6, 7, 8, 9, or 10.

The disclosure provides compounds of Formula V:

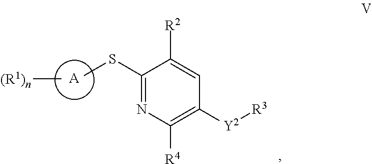

and pharmaceutically acceptable salts, prodrugs, solvates, hydrates, tautomers, or isomers thereof, wherein:
A is selected from the group consisting of 5- to 12-membered monocyclic or polycyclic cycloalkyl, heterocycloalkyl, aryl, or heteroaryl;
Y² is selected from the group consisting of: —NR^{a}—, —(CR^{a}₂)ₘ—, —C(O)—, —C(R^{a})₂NH—, —(CR^{a}₂)ₘO—, —C(O)N(R^{a})—, —N(R^{a})C(O)—, —S(O)₂N(R^{a})—, —N(R^{a})S(O)₂—, —N(R^{a})C(O)N(R^{a})—, —N(R^{a})C(S)N(R^{a})—, —C(O)O—, —OC(O)—, —OC(O)N(R^{a})—, —N(R^{a})C(O)O—, —C(O)N(R^{a})O—, —N(R^{a})C(S)—, —C(S)N(R^{a})—, and —OC(O)O—; wherein the bond on the left side of Y², as drawn, is bound to the pyridine ring and the bond on the right side of the Y² moiety is bound to R³;
R¹ is independently, at each occurrence, —H, -D, —C₁-C₆ alkyl, —C₂-C₆ alkenyl, —C₄-C₈ cycloalkenyl, —C₂-C₆ alkynyl, —C₃-C₈ cycloalkyl, —OH, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)R⁵, or —CO₂R⁵, wherein each alkyl, alkenyl, cycloalkenyl, alkynyl, or cycloalkyl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;
R² is independently —OR^{b}, —CN, —C₁-C₆ alkyl, —C₂-C₆ alkenyl, —C₄-C₈ cycloalkenyl, —C₂-C₆ alkynyl, —C₃-C₈ cycloalkyl, aryl, heterocyclyl containing 1-5 heteroatoms selected from the group consisting of N, S, P, or O, or heteroaryl containing 1-5 heteroatoms selected from the group consisting of N, S, P, or O; wherein each alkyl, alkenyl, cycloalkenyl, alkynyl, cycloalkyl, heterocyclyl, aryl, or heteroaryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; and wherein the heterocyclyl or heteroaryl is not attached via a nitrogen atom;
R^{a} is independently, at each occurrence, selected from the group consisting of —H, -D, —OH, —C₃-C₈ cycloalkyl, and —C₁-C₆ alkyl, wherein each alkyl or cycloalkyl is optionally substituted with one or more —NH₂, wherein 2 R^{a}, together with the carbon atom to which they are both attached, can combine to form a 3- to 8-membered cycloalkyl;
R^{b} is independently —H, -D, —C₁-C₆ alkyl, —C₁-C₆ cycloalkyl, —C₂-C₆ alkenyl, or heterocyclyl containing 1-5 heteroatoms selected from the group consisting of N, S, P, or O; wherein each alkyl, cycloalkyl, alkenyl, or heterocycle is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;
R³ is independently, at each occurrence, selected from the group consisting of —C₁-C₆ alkyl, or a 3- to 12-membered monocyclic or polycyclic heterocycle, wherein each alkyl or heterocycle is optionally substituted with one or more —C₁-C₆ alkyl, —OH, or —NH₂; or
R³ can combine with R^{a} to form a 3- to 12-membered monocyclic or polycyclic heterocycle, or a 5- to 12-membered spiroheterocycle, wherein each heterocycle or spiroheterocycle is optionally substituted with —C₁-C₆ alkyl, —OH, or —NH₂;
R⁴ is independently, at each occurrence, —H, -D, or —C₁-C₆ alkyl, wherein each alkyl is optionally substituted with one or more —OH, —NH₂, halogen, or oxo; or
R^{a} and R⁴, together with the atom or atoms to which they are attached, can combine to form a monocyclic or polycyclic C₃-C₁₂ cycloalkyl, or a monocyclic or polycyclic 3- to 12-membered heterocycle, wherein the cycloalkyl or heterocycle is optionally substituted with oxo;
R⁵ and R⁶ are each independently, at each occurrence, selected from the group consisting of —H, -D, —C₁-C₆ alkyl, —C₂-C₆ alkenyl, —C₄-C₈ cycloalkenyl, —C₂-C₆ alkynyl, —C₃-C₈ cycloalkyl, a monocyclic or polycyclic 3- to 12-membered heterocycle, —OR⁷, —SR⁷, halogen, —NR⁷R⁸, —NO₂, and —CN;
R⁷ and R⁸ are independently, at each occurrence, —H, -D, —C₁-C₆ alkyl, —C₂-C₆ alkenyl, —C₄-C₈ cycloalkenyl, —C₂-C₆ alkynyl, —C₃-C₈ cycloalkyl, a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN;
m is independently 1, 2, 3, 4, 5 or 6; and
n is independently 0, 1, 2, 3, 4, 5, 6, 7, 8, 9, or 10.

The disclosure provides compounds of Formula VI:

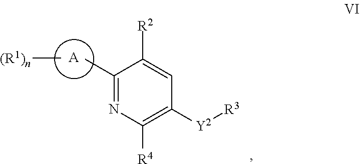

and pharmaceutically acceptable salts, prodrugs, solvates, hydrates, tautomers, or isomers thereof, wherein:
A is selected from the group consisting of 5- to 12-membered monocyclic or polycyclic cycloalkyl, heterocycloalkyl, aryl, or heteroaryl;
Y² is selected from the group consisting of: —NR^{a}—, —(CR^{a}₂)ₘ—, —C(O)—, —C(R^{a})₂NH—, —(CR^{a}₂)ₘO—, —C(O)N(R^{a})—, —N(R^{a})C(O)—, —S(O)₂N(R^{a})—, —N(R^{a})S(O)₂—, —N(R^{a})C(O)N(R^{a})—, —N(R^{a})C(S)N(R^{a})—, —C(O)O—, —OC(O)—, —OC(O)N(R^{a})—, —N(R^{a})C(O)O—, —C(O)N(R^{a})O—, —N(R^{a})C(S)—, —C(S)N(R^{a})—, and —OC(O)O—; wherein the bond on the left side of Y², as drawn, is bound to the pyridine ring and the bond on the right side of the Y² moiety is bound to R³;
R¹ is independently, at each occurrence, —H, -D, —C₁-C₆ alkyl, —C₂-C₆ alkenyl, —C₄-C₈ cycloalkenyl, —C₂-C₆ alkynyl, —C₃-C₈ cycloalkyl, —OH, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)R⁵, or —CO₂R⁵, wherein each alkyl, alkenyl, cycloalkenyl, alkynyl, or cycloalkyl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;
R² is independently —OR^{b}, —CN, —C₁-C₆ alkyl, —C₂-C₆ alkenyl, —C₄-C₈ cycloalkenyl, —C₂-C₆ alkynyl, —C₃-C₈ cycloalkyl, aryl, heterocyclyl containing 1-5 heteroatoms selected from the group consisting of N, S, P, or O, or heteroaryl containing 1-5 heteroatoms selected from the group consisting of N, S, P, or O; wherein each alkyl, alkenyl, cycloalkenyl, alkynyl, cycloalkyl, heterocyclyl, aryl, or heteroaryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; and wherein the heterocyclyl or heteroaryl is not attached via a nitrogen atom;
R^{a} is independently, at each occurrence, selected from the group consisting of —H, -D, —OH, —C₃-C₈ cycloalkyl, and —C₁-C₆ alkyl, wherein each alkyl or cycloalkyl is optionally substituted with one or more —NH₂, wherein 2 R^{a}, together with the carbon atom to which they are both attached, can combine to form a 3- to 8-membered cycloalkyl;
R^{b} is independently —H, -D, —C₁-C₆ alkyl, —C₁-C₆ cycloalkyl, —C₂-C₆ alkenyl, or heterocyclyl containing 1-5 heteroatoms selected from the group consisting of N, S, P, or O; wherein each alkyl, cycloalkyl, alkenyl, or heterocycle is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;
R³ is independently, at each occurrence, selected from the group consisting of —C₁-C₆ alkyl, or a 3- to 12-membered monocyclic or polycyclic heterocycle, wherein each alkyl or heterocycle is optionally substituted with one or more —C₁-C₆ alkyl, —OH, or —NH₂; or
R³ can combine with R^{a} to form a 3- to 12-membered monocyclic or polycyclic heterocycle, or a 5- to 12-membered spiroheterocycle, wherein each heterocycle or spiroheterocycle is optionally substituted with —C₁-C₆ alkyl, —OH, or —NH₂;
R⁴ is independently, at each occurrence, —H, -D, or —C₁-C₆ alkyl, wherein each alkyl is optionally substituted with one or more —OH, —NH₂, halogen, or oxo; or R^{a} and R⁴, together with the atom or atoms to which they are attached, can combine to form a monocyclic or polycyclic C₃-C₁₂ cycloalkyl, or a monocyclic or polycyclic 3- to 12-membered heterocycle, wherein the cycloalkyl or heterocycle is optionally substituted with oxo;
R⁵ and R⁶ are each independently, at each occurrence, selected from the group consisting of —H, -D, —C₁-C₆ alkyl, —C₂-C₆ alkenyl, —C₄-C₈ cycloalkenyl, —C₂-C₆ alkynyl, —C₃-C₈ cycloalkyl, a monocyclic or polycyclic 3- to 12-membered heterocycle, —OR⁷, —SR⁷, halogen, —NR⁷R⁸, —NO₂, and —CN;
R⁷ and R⁸ are independently, at each occurrence, —H, -D, —C₁-C₆ alkyl, —C₂-C₆ alkenyl, —C₄-C₈ cycloalkenyl, —C₂-C₆ alkynyl, —C₃-C₈ cycloalkyl, a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN;
m is independently 1, 2, 3, 4, 5 or 6; and
n is independently 0, 1, 2, 3, 4, 5, 6, 7, 8, 9, or 10.

The disclosure provides compounds of Formula IV-Y:

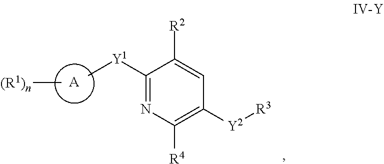

or a pharmaceutically acceptable salt, prodrug, solvate, hydrate, tautomer, or isomer thereof, wherein:
A is selected from the group consisting of 5- to 12-membered monocyclic or polycyclic cycloalkyl, heterocycloalkyl, aryl, or heteroaryl;
Y¹ is —S— or a direct bond;
Y² is selected from the group consisting of: —NR^{a}—, —(CR^{a}₂)ₘ—, —C(O)—, —C(R^{a})₂NH—, —(CR^{a}₂)ₘO—, —C(O)N(R^{a})—, —N(R^{a})C(O)—, —S(O)₂N(R^{a})—, —N(R^{a})S(O)₂—, —N(R^{a})C(O)N(R^{a})—, —N(R^{a})C(S)N(R^{a})—, —C(O)O—, —OC(O)—, —OC(O)N(R^{a})—, —N(R^{a})C(O)O—, —C(O)N(R^{a})O—, —N(R^{a})C(S)—, —C(S)N(R^{a})—, and —OC(O)O—; wherein the bond on the left side of Y², as drawn, is bound to the pyridine ring and the bond on the right side of the Y² moiety, as drawn, is bound to R³;
R¹ is independently, at each occurrence, —H, -D, —C₁-C₆ alkyl, —C₂-C₆ alkenyl, —C₄-C₈ cycloalkenyl, —C₂-C₆ alkynyl, —C₃-C₈ cycloalkyl, —OH, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)R⁵, or —CO₂R⁵, wherein each alkyl, alkenyl, cycloalkenyl, alkynyl, or cycloalkyl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; R² is independently —OR^{b}, —CN, —C₁-C₆ alkyl, —C₂-C₆ alkenyl, —C₄-C₈ cycloalkenyl, —C₂-C₆ alkynyl, —C₃-C₈ cycloalkyl, aryl, heterocyclyl containing 1-5 heteroatoms selected from the group consisting of N, S, P, or O, or heteroaryl containing 1-5 heteroatoms selected from the group consisting of N, S, P, or O; wherein each alkyl, alkenyl, cycloalkenyl, alkynyl, cycloalkyl, heterocyclyl, aryl, or heteroaryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; and wherein the heterocyclyl or heteroaryl is not attached via a nitrogen atom;
R^{a} is independently, at each occurrence, selected from the group consisting of —H, -D, —OH, —C₃-C₈ cycloalkyl, and —C₁-C₆ alkyl, wherein each alkyl or cycloalkyl is optionally substituted with one or more —NH₂, wherein 2 R^{a}, together with the carbon atom to which they are both attached, can combine to form a 3- to 8-membered cycloalkyl;
R^{b} is independently —H, -D, —C₁-C₆ alkyl, —C₁-C₆ cycloalkyl, —C₂-C₆ alkenyl, or heterocyclyl containing 1-5 heteroatoms selected from the group consisting of N, S, P, or O; wherein each alkyl, cycloalkyl, alkenyl, or heterocycle is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, heteroaryl, —(CH₂)ₙOH, —C₁-C₆ alkyl, CF₃, CHF₂, or CH₂F;
R³ is independently, at each occurrence, selected from the group consisting of —H, —C₁-C₆ alkyl, a 3- to 12-membered monocyclic or polycyclic heterocycle, C₃-C₈ cycloalkyl, or —(CH₂)ₙ—R^{b}, wherein each alkyl, heterocycle, or cycloalkyl is optionally substituted with one or more —C₁-C₆ alkyl, —OH, —NH₂, —OR^{a}, —NHR^{a}—(CH₂)ₙOH, heterocyclyl, or spiroheterocyclyl; or
R³ can combine with R^{a} to form a 3- to 12-membered monocyclic or polycyclic heterocycle, or a 5- to 12-membered spiroheterocycle, wherein each heterocycle or spiroheterocycle is optionally substituted with —C₁-C₆ alkyl, —OH, —NH₂, heteroaryl, heterocyclyl, —(CH₂)ₙNH₂, —COOR^{a}, —CONHR^{b}, —CONH(CH₂)ₙCOOR^{a}, —NHCOOR^{a}, —CF₃, CHF₂, or CH₂F;
R⁴ is independently, at each occurrence, —H, -D, —C₁-C₆ alkyl, —NH—NHR⁵, —NH—OR⁵, —O—NR⁵R⁶, —NHR⁵, —OR⁵, —NHC(O)R⁵, —NHC(O)NHR⁵, —NHS(O)₂R⁵, —NHS(O)₂NHR⁵, —S(O)₂OH, —C(O)OR⁵, —NH(CH₂)ₙOH, —C(O)NH(CH₂)ₙOH, —C(O)NH(CH₂)ₙR^{b}, —C(O)R^{b}, NH₂, —OH, —CN, —C(O)NR⁵R⁶, —S(O)₂NR⁵R⁶, C₃-C₈ cycloalkyl, aryl, heterocyclyl containing 1-5 heteroatoms selected from the group consisting of N, S, P, or O, heteroaryl containing 1-5 heteroatoms selected from the group consisting of N, S, P, or O, wherein each alkyl, cycloalkyl, or heterocyclyl is optionally substituted with one or more —OH, —NH₂, halogen, or oxo; wherein each aryl or heteroaryl is optionally substituted with one or more —OH, —NH₂, or halogen; or
R^{a} and R⁴, together with the atom or atoms to which they are attached, can combine to form a monocyclic or polycyclic C₃-C₁₂ cycloalkyl, or a monocyclic or polycyclic 3- to 12-membered heterocycle, wherein the cycloalkyl or heterocycle is optionally substituted with oxo; wherein the heterocycle optionally comprises —S(O)₂— in the heterocycle;
R⁵ and R⁶ are each independently, at each occurrence, selected from the group consisting of —H, -D, —C₁-C₆ alkyl, —C₂-C₆ alkenyl, —C₄-C₈ cycloalkenyl, —C₂-C₆ alkynyl, —C₃-C₈ cycloalkyl, a monocyclic or polycyclic 3- to 12-membered heterocycle, —OR⁷, —SR⁷, halogen, —NR⁷R⁸, —NO₂, and —CN;
R⁷ and R⁸ are independently, at each occurrence, —H, -D, —C₁-C₆ alkyl, —C₂-C₆ alkenyl, —C₄-C₈ cycloalkenyl, —C₂-C₆ alkynyl, —C₃-C₈ cycloalkyl, a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN;
m is independently 1, 2, 3, 4, 5 or 6; and
n is independently 0, 1, 2, 3, 4, 5, 6, 7, 8, 9, or 10.

The disclosure provides compounds of Formula IV-Z:

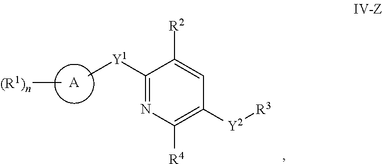

or a pharmaceutically acceptable salt, prodrug, solvate, hydrate, tautomer, or isomer thereof, wherein:
A is selected from the group consisting of 5- to 12-membered monocyclic or polycyclic cycloalkyl, heterocycloalkyl, aryl, or heteroaryl;
Y¹ is —S—, a direct bond, —NH—, —S(O)₂—, —S(O)₂—NH—, —C(═CH₂)—, —CH—, or —S(O)—;
Y² is selected from the group consisting of: —NR^{a}—, —(CR^{a}₂)ₘ—, —C(O)—, —C(R^{a})₂NH—, —(CR^{a}₂)ₘO—, —C(O)N(R^{a})—, —N(R^{a})C(O)—, —S(O)₂N(R^{a})—, —N(R^{a})S(O)₂—, —N(R^{a})C(O)N(R^{a})—, —N(R^{a})C(S)N(R^{a})—, —C(O)O—, —OC(O)—, —OC(O)N(R^{a})—, —N(R^{a})C(O)O—, —C(O)N(R^{a})O—, —N(R^{a})C(S)—, —C(S)N(R^{a})—, and —OC(O)O—; wherein the bond on the left side of Y², as drawn, is bound to the pyridine ring and the bond on the right side of the Y² moiety, as drawn, is bound to R³;
R¹ is independently, at each occurrence, —H, -D, —C₁-C₆ alkyl, —C₂-C₆ alkenyl, —C₄-C₈ cycloalkenyl, —C₂-C₆ alkynyl, —C₃-C₈ cycloalkyl, —OH, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)R⁵, or —CO₂R⁵, wherein each alkyl, alkenyl, cycloalkenyl, alkynyl, or cycloalkyl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;
R² is independently —OR^{b}, —CN, —C₁-C₆ alkyl, —C₂-C₆ alkenyl, —C₄-C₈ cycloalkenyl, —C₂-C₆ alkynyl, —NH₂, halogen, —C(O)OR^{a}, —C₃-C₈ cycloalkyl, aryl, heterocyclyl containing 1-5 heteroatoms selected from the group consisting of N, S, P, or O, or heteroaryl containing 1-5 heteroatoms selected from the group consisting of N, S, P, or O; wherein each alkyl, alkenyl, cycloalkenyl, alkynyl, cycloalkyl, heterocyclyl, aryl, or heteroaryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; and wherein the heterocyclyl or heteroaryl is not attached via a nitrogen atom;
R^{a} is independently, at each occurrence, selected from the group consisting of —H, -D, —OH, —C₃-C₈ cycloalkyl, and —C₁-C₆ alkyl, wherein each alkyl or cycloalkyl is optionally substituted with one or more —NH₂, wherein 2 R^{a}, together with the carbon atom to which they are both attached, can combine to form a 3- to 8-membered cycloalkyl;
R^{b} is independently —H, -D, —C₁-C₆ alkyl, —C₁-C₆ cycloalkyl, —C₂-C₆ alkenyl, or heterocyclyl containing 1-5 heteroatoms selected from the group consisting of N, S, P, or O; wherein each alkyl, cycloalkyl, alkenyl, or heterocycle is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, heteroaryl, —(CH₂)ₙOH, —C₁-C₆ alkyl, CF₃, CHF₂, or CH₂F;
R³ is independently, at each occurrence, selected from the group consisting of —H, —C₁-C₆ alkyl, a 3- to 12-membered monocyclic or polycyclic heterocycle, C₃-C₈ cycloalkyl, or —(CH₂)ₙ—R^{b}, wherein each alkyl, heterocycle, or cycloalkyl is optionally substituted with one or more —C₁-C₆ alkyl, —OH, —NH₂, —OR^{a}, —NHR^{a}—(CH₂)ₙOH, heterocyclyl, or spiroheterocyclyl; or
R³ can combine with R^{a} to form a 3- to 12-membered monocyclic or polycyclic heterocycle, or a 5- to 12-membered spiroheterocycle, wherein each heterocycle or spiroheterocycle is optionally substituted with —C₁-C₆ alkyl, —OH, —NH₂, heteroaryl, heterocyclyl, —(CH₂)ₙNH₂, —COOR^{a}, —CONHR^{b}, —CONH(CH₂)ₙCOOR^{a}, —NHCOOR^{a}, —CF₃, CHF₂, or CH₂F;
R⁴ is independently, at each occurrence, —H, -D, —C₁-C₆ alkyl, —NH—NHR⁵, —NH—OR⁵, —O—NR⁵R⁶, —NHR⁵, —OR⁵, —NHC(O)R⁵, —NHC(O)NHR⁵, —NHS(O)₂R⁵, —NHS(O)₂NHR⁵, —S(O)₂OH, —C(O)OR⁵, —NH(CH₂)ₙOH, —C(O)NH(CH₂)ₙOH, —C(O)NH(CH₂)ₙR^{b}, —C(O)R^{b}, NH₂, —OH, —CN, —C(O)NR⁵R⁶, —S(O)₂NR⁵R⁶, C₃-C₈ cycloalkyl, aryl, heterocyclyl containing 1-5 heteroatoms selected from the group consisting of N, S, P, or O, heteroaryl containing 1-5 heteroatoms selected from the group consisting of N, S, P, or O, wherein each alkyl, cycloalkyl, or heterocyclyl is optionally substituted with one or more —OH, —NH₂, halogen, or oxo; wherein each aryl or heteroaryl is optionally substituted with one or more —OH, —NH₂, or halogen; or
R^{a} and R⁴, together with the atom or atoms to which they are attached, can combine to form a monocyclic or polycyclic C₃-C₁₂ cycloalkyl, or a monocyclic or polycyclic 3- to 12-membered heterocycle, wherein the cycloalkyl or heterocycle is optionally substituted with oxo; wherein the heterocycle optionally comprises —S(O)₂— in the heterocycle;
R⁵ and R⁶ are each independently, at each occurrence, selected from the group consisting of —H, -D, —C₁-C₆ alkyl, —C₂-C₆ alkenyl, —C₄-C₈ cycloalkenyl, —C₂-C₆ alkynyl, —C₃-C₈ cycloalkyl, a monocyclic or polycyclic 3- to 12-membered heterocycle, —OR⁷, —SR⁷, halogen, —NR⁷R⁸, —NO₂, and —CN;
R⁷ and R⁸ are independently, at each occurrence, —H, -D, —C₁-C₆ alkyl, —C₂-C₆ alkenyl, —C₄-C₈ cycloalkenyl, —C₂-C₆ alkynyl, —C₃-C₈ cycloalkyl, a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN;
m is independently 1, 2, 3, 4, 5 or 6; and
n is independently 0, 1, 2, 3, 4, 5, 6, 7, 8, 9, or 10.

The disclosure provides compounds of Formula VII:

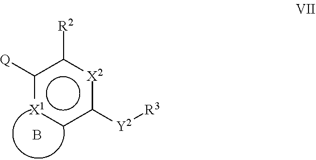

and pharmaceutically acceptable salts, prodrugs, solvates, hydrates, tautomers, or isomers thereof, wherein:
Q is H or

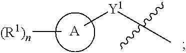

A is selected from the group consisting of 5- to 12-membered monocyclic or polycyclic cycloalkyl, heterocycloalkyl, aryl, or heteroaryl;
R¹ is independently, at each occurrence, —H, -D, —C₁-C₆ alkyl, —C₂-C₆ alkenyl, —C₄-C₈ cycloalkenyl, —C₂-C₆ alkynyl, —C₃-C₈ cycloalkyl, —OH, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)R⁵, or —CO₂R⁵, wherein each alkyl, alkenyl, cycloalkenyl, alkynyl, or cycloalkyl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; Y¹ is —S—, a direct bond, —NH—, —S(O)₂—, —S(O)₂—NH—, —C(═CH₂)—, —CH—, or —S(O)—;
X¹ is N or C;
X² is N or CH;
B, including the atoms at the points of attachment, is a monocyclic or polycyclic 5- to 12-membered heterocycle or a monocyclic or polycyclic 5- to 12-membered heteroaryl;
R² is independently H, —OR^{b}, —NR⁵R⁶, —CN, —C₁-C₆ alkyl, —C₂-C₆ alkenyl, —C₄-C₈ cycloalkenyl, —C₂-C₆ alkynyl, —NH₂, halogen, —C(O)OR^{a}, —C₃-C₈ cycloalkyl, heterocyclyl containing 1-5 heteroatoms selected from the group consisting of N, S, P, or O, or heteroaryl containing 1-5 heteroatoms selected from the group consisting of N, S, P, or O; wherein each alkyl, alkenyl, cycloalkenyl, alkynyl, cycloalkyl, heterocyclyl, or heteroaryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; and wherein the heterocyclyl or heteroaryl is not attached via a nitrogen atom;
Y² is selected from the group consisting of: —NR^{a}—, —(CR^{a}₂)ₘ—, —C(O)—, —C(R^{a})₂NH—, —(CR^{a}₂)ₘO—, —C(O)N(R^{a})—, —N(R^{a})C(O)—, —S(O)₂N(R^{a})—, —N(R^{a})S(O)₂—, —N(R^{a})C(O)N(R^{a})—, —N(R^{a})C(S)N(R^{a})—, —C(O)O—, —OC(O)—, —OC(O)N(R^{a})—, —N(R^{a})C(O)O—, —C(O)N(R^{a})O—, —N(R^{a})C(S)—, —C(S)N(R^{a})—, and —OC(O)O—; wherein the bond on the left side of Y², as drawn, is bound to the ring and the bond on the right side of the Y² moiety, as drawn, is bound to R³;
R^{a} is independently, at each occurrence, selected from the group consisting of —H, -D, —OH, —C₃-C₈ cycloalkyl, and —C₁-C₆ alkyl, wherein each alkyl or cycloalkyl is optionally substituted with one or more —NH₂, wherein 2 R^{a}, together with the carbon atom to which they are both attached, can combine to form a 3- to 8-membered cycloalkyl;
R^{b} is independently —H, -D, —C₁-C₆ alkyl, —C₁-C₆ cycloalkyl, —C₂-C₆ alkenyl, or heterocyclyl containing 1-5 heteroatoms selected from the group consisting of N, S, P, or O; wherein each alkyl, cycloalkyl, alkenyl, or heterocycle is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, heteroaryl, —(CH₂)ₙOH, —C₁-C₆ alkyl, CF₃, CHF₂, or CH₂F;
R³ is independently, at each occurrence, selected from the group consisting of —H, —C₁-C₆ alkyl, a 3- to 12-membered monocyclic or polycyclic heterocycle, C₃-C₈ cycloalkyl, or —(CH₂)ₙ—R^{b}, wherein each alkyl, heterocycle, or cycloalkyl is optionally substituted with one or more —C₁-C₆ alkyl, —OH, —NH₂, —OR^{a}, —NHR^{a}, —(CH₂)ₙOH, heterocyclyl, or spiroheterocyclyl; or
R³ can combine with R^{a} to form a 3- to 12-membered monocyclic or polycyclic heterocycle, or a 5- to 12-membered spiroheterocycle, wherein each heterocycle or spiroheterocycle is optionally substituted with —C₁-C₆ alkyl, —OH, —NH₂, heteroaryl, heterocyclyl, —(CH₂)ₙNH₂, —COOR^{a}, —CONHR^{b}, —CONH(CH₂)ₙCOOR^{a}, —NHCOOR^{a}, —CF₃, CHF₂, or CH₂F;
R⁵ and R⁶ are each independently, at each occurrence, selected from the group consisting of —H, -D, —C₁-C₆ alkyl, —C₂-C₆ alkenyl, —C₄-C₈ cycloalkenyl, —C₂-C₆ alkynyl, —C₃-C₈ cycloalkyl, a monocyclic or polycyclic 3- to 12-membered heterocycle, —OR⁷, —SR⁷, halogen, —NR⁷R⁸, —NO₂, and —CN;
R⁷ and R⁸ are independently, at each occurrence, —H, -D, —C₁-C₆ alkyl, —C₂-C₆ alkenyl, —C₄-C₈ cycloalkenyl, —C₂-C₆ alkynyl, —C₃-C₈ cycloalkyl, a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN;
m is independently 1, 2, 3, 4, 5 or 6; and
n is independently 0, 1, 2, 3, 4, 5, 6, 7, 8, 9, or 10.

The disclosure provides compounds of Formula VIII:

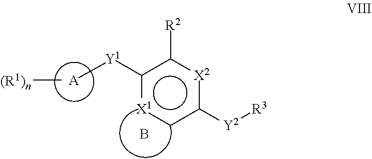

and pharmaceutically acceptable salts, prodrugs, solvates, hydrates, tautomers, or isomers thereof, wherein:
A is selected from the group consisting of 5- to 12-membered monocyclic or polycyclic cycloalkyl, heterocycloalkyl, aryl, or heteroaryl;
R¹ is independently, at each occurrence, —H, -D, —C₁-C₆ alkyl, —C₂-C₆ alkenyl, —C₄-C₈ cycloalkenyl, —C₂-C₆ alkynyl, —C₃-C₈ cycloalkyl, —OH, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)R⁵, or —CO₂R⁵, wherein each alkyl, alkenyl, cycloalkenyl, alkynyl, or cycloalkyl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;
Y¹ is —S—, a direct bond, —NH—, —S(O)₂—, —S(O)₂—NH—, —C(═CH₂)—, —CH—, or —S(O)—;
X¹ is N or C;
X² is N or CH;
B, including the atoms at the points of attachment, is a monocyclic or polycyclic 5- to 12-membered heterocycle or a monocyclic or polycyclic 5- to 12-membered heteroaryl;
R² is independently H, —OR^{b}, —NR⁵R⁶, —CN, —C₁-C₆ alkyl, —C₂-C₆ alkenyl, —C₄-C₈ cycloalkenyl, —C₂-C₆ alkynyl, —NH₂, halogen, —C(O)OR^{a}, —C₃-C₈ cycloalkyl, heterocyclyl containing 1-5 heteroatoms selected from the group consisting of N, S, P, or O, or heteroaryl containing 1-5 heteroatoms selected from the group consisting of N, S, P, or O; wherein each alkyl, alkenyl, cycloalkenyl, alkynyl, cycloalkyl, heterocyclyl, or heteroaryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; and wherein the heterocyclyl or heteroaryl is not attached via a nitrogen atom;
Y² is selected from the group consisting of: —NR^{a}—, —(CR^{a}₂)ₘ—, —C(O)—, —C(R^{a})₂NH—, —(CR^{a}₂)ₘO—, —C(O)N(R^{a})—, —N(R^{a})C(O)—, —S(O)₂N(R^{a})—, —N(R^{a})S(O)₂—, —N(R^{a})C(O)N(R^{a})—, —N(R^{a})C(S)N(R^{a})—, —C(O)O—, —OC(O)—, —OC(O)N(R^{a})—, —N(R^{a})C(O)O—, —C(O)N(R^{a})O—, —N(R^{a})C(S)—, —C(S)N(R^{a})—, and —OC(O)O—; wherein the bond on the left side of Y², as drawn, is bound to the ring and the bond on the right side of the Y² moiety, as drawn, is bound to R³;
R^{a} is independently, at each occurrence, selected from the group consisting of —H, -D, —OH, —C₃-C₈ cycloalkyl, and —C₁-C₆ alkyl, wherein each alkyl or cycloalkyl is optionally substituted with one or more —NH₂, wherein 2 R^{a}, together with the carbon atom to which they are both attached, can combine to form a 3- to 8-membered cycloalkyl;
R^{b} is independently —H, -D, —C₁-C₆ alkyl, —C₁-C₆ cycloalkyl, —C₂-C₆ alkenyl, or heterocyclyl containing 1-5 heteroatoms selected from the group consisting of N, S, P, or O; wherein each alkyl, cycloalkyl, alkenyl, or heterocycle is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, heteroaryl, —(CH₂)ₙOH, —C₁-C₆ alkyl, CF₃, CHF₂, or CH₂F;
R³ is independently, at each occurrence, selected from the group consisting of —H, —C₁-C₆ alkyl, a 3- to 12-membered monocyclic or polycyclic heterocycle, C₃-C₈ cycloalkyl, or —(CH₂)ₙ—R^{b}, wherein each alkyl, heterocycle, or cycloalkyl is optionally substituted with one or more —C₁-C₆ alkyl, —OH, —NH₂, —OR^{a}, —NHR^{a}, —(CH₂)ₙOH, heterocyclyl, or spiroheterocyclyl; or
R³ can combine with R^{a} to form a 3- to 12-membered monocyclic or polycyclic heterocycle, or a 5- to 12-membered spiroheterocycle, wherein each heterocycle or spiroheterocycle is optionally substituted with —C₁-C₆ alkyl, —OH, —NH₂, heteroaryl, heterocyclyl, —(CH₂)ₙNH₂, —COOR^{a}, —CONHR^{b}, —CONH(CH₂)ₙCOOR^{a}, —NHCOOR^{a}, —CF₃, CHF₂, or CH₂F; R⁵ and R⁶ are each independently, at each occurrence, selected from the group consisting of —H, -D, —C₁-C₆ alkyl, —C₂-C₆ alkenyl, —C₄-C₈ cycloalkenyl, —C₂-C₆ alkynyl, —C₃-C₈ cycloalkyl, a monocyclic or polycyclic 3- to 12-membered heterocycle, —OR⁷, —SR⁷, halogen, —NR⁷R⁸, —NO₂, and —CN;
R⁷ and R⁸ are independently, at each occurrence, —H, -D, —C₁-C₆ alkyl, —C₂-C₆ alkenyl, —C₄-C₈ cycloalkenyl, —C₂-C₆ alkynyl, —C₃-C₈ cycloalkyl, a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN;
m is independently 1, 2, 3, 4, 5 or 6; and
n is independently 0, 1, 2, 3, 4, 5, 6, 7, 8, 9, or 10.

The disclosure provides compounds of Formula IX:

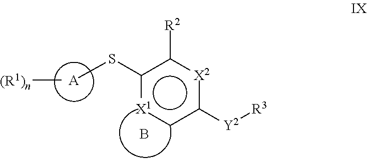

and pharmaceutically acceptable salts, prodrugs, solvates, hydrates, tautomers, or isomers thereof, wherein:
A is selected from the group consisting of 5- to 12-membered monocyclic or polycyclic cycloalkyl, heterocycloalkyl, aryl, or heteroaryl;
R¹ is independently, at each occurrence, —H, -D, —C₁-C₆ alkyl, —C₂-C₆ alkenyl, —C₄-C₈ cycloalkenyl, —C₂-C₆ alkynyl, —C₃-C₈ cycloalkyl, —OH, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)R⁵, or —CO₂R⁵, wherein each alkyl, alkenyl, cycloalkenyl, alkynyl, or cycloalkyl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;
X¹ is N or C;
X² is N or CH;
B, including the atoms at the points of attachment, is a monocyclic or polycyclic 5- to 12-membered heterocycle or a monocyclic or polycyclic 5- to 12-membered heteroaryl;
R² is independently H, —OR^{b}, —NR⁵R⁶, —CN, —C₁-C₆ alkyl, —C₂-C₆ alkenyl, —C₄-C₈ cycloalkenyl, —C₂-C₆ alkynyl, —NH₂, halogen, —C(O)OR^{a}, —C₃-C₈ cycloalkyl, aryl, heterocyclyl containing 1-5 heteroatoms selected from the group consisting of N, S, P, or O, or heteroaryl containing 1-5 heteroatoms selected from the group consisting of N, S, P, or O; wherein each alkyl, alkenyl, cycloalkenyl, alkynyl, cycloalkyl, heterocyclyl, aryl, or heteroaryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; and wherein the heterocyclyl or heteroaryl is not attached via a nitrogen atom;
Y² is selected from the group consisting of: —NR^{a}—, —(CR^{a}₂)ₘ—, —C(O)—, —C(R^{a})₂NH—, —(CR^{a}₂)ₘO—, —C(O)N(R^{a})—, —N(R^{a})C(O)—, —S(O)₂N(R^{a})—, —N(R^{a})S(O)₂—, —N(R^{a})C(O)N(R^{a})—, —N(R^{a})C(S)N(R^{a})—, —C(O)O—, —OC(O)—, —OC(O)N(R^{a})—, —N(R^{a})C(O)O—, —C(O)N(R^{a})O—, —N(R^{a})C(S)—, —C(S)N(R^{a})—, and —OC(O)O—; wherein the bond on the left side of Y², as drawn, is bound to the ring and the bond on the right side of the Y² moiety, as drawn, is bound to R³;
R^{a} is independently, at each occurrence, selected from the group consisting of —H, -D, —OH, —C₃-C₈ cycloalkyl, and —C₁-C₆ alkyl, wherein each alkyl or cycloalkyl is optionally substituted with one or more —NH₂, wherein 2 R^{a}, together with the carbon atom to which they are both attached, can combine to form a 3- to 8-membered cycloalkyl;
R^{b} is independently —H, -D, —C₁-C₆ alkyl, —C₁-C₆ cycloalkyl, —C₂-C₆ alkenyl, or heterocyclyl containing 1-5 heteroatoms selected from the group consisting of N, S, P, or O; wherein each alkyl, cycloalkyl, alkenyl, or heterocycle is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, heteroaryl, —(CH₂)ₙOH, —C₁-C₆ alkyl, CF₃, CHF₂, or CH₂F;
R³ is independently, at each occurrence, selected from the group consisting of —H, —C₁-C₆ alkyl, a 3- to 12-membered monocyclic or polycyclic heterocycle, C₃-C₈ cycloalkyl, or —(CH₂)ₙ—R^{b}, wherein each alkyl, heterocycle, or cycloalkyl is optionally substituted with one or more —C₁-C₆ alkyl, —OH, —NH₂, —OR^{a}, —NHR^{a}, —(CH₂)ₙOH, heterocyclyl, or spiroheterocyclyl; or
R³ can combine with R^{a} to form a 3- to 12-membered monocyclic or polycyclic heterocycle, or a 5- to 12-membered spiroheterocycle, wherein each heterocycle or spiroheterocycle is optionally substituted with —C₁-C₆ alkyl, —OH, —NH₂, heteroaryl, heterocyclyl, —(CH₂)ₙNH₂, —COOR^{a}, —CONHR^{b}, —CONH(CH₂)ₙCOOR^{a}, —NHCOOR^{a}, —CF₃, CHF₂, or CH₂F;
R⁵ and R⁶ are each independently, at each occurrence, selected from the group consisting of —H, -D, —C₁-C₆ alkyl, —C₂-C₆ alkenyl, —C₄-C₈ cycloalkenyl, —C₂-C₆ alkynyl, —C₃-C₈ cycloalkyl, a monocyclic or polycyclic 3- to 12-membered heterocycle, —OR⁷, —SR⁷, halogen, —NR⁷R⁸, —NO₂, and —CN;
R⁷ and R⁸ are independently, at each occurrence, —H, -D, —C₁-C₆ alkyl, —C₂-C₆ alkenyl, —C₄-C₈ cycloalkenyl, —C₂-C₆ alkynyl, —C₃-C₈ cycloalkyl, a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN;
m is independently 1, 2, 3, 4, 5 or 6; and
n is independently 0, 1, 2, 3, 4, 5, 6, 7, 8, 9, or 10.

The disclosure provides compounds of Formula X:

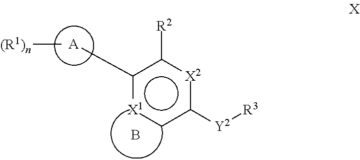

and pharmaceutically acceptable salts, prodrugs, solvates, hydrates, tautomers, or isomers thereof, wherein:
A is selected from the group consisting of 5- to 12-membered monocyclic or polycyclic cycloalkyl, heterocycloalkyl, aryl, or heteroaryl;
R¹ is independently, at each occurrence, —H, -D, —C₁-C₆ alkyl, —C₂-C₆ alkenyl, —C₄-C₈ cycloalkenyl, —C₂-C₆ alkynyl, —C₃-C₈ cycloalkyl, —OH, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)R⁵, or —CO₂R⁵, wherein each alkyl, alkenyl, cycloalkenyl, alkynyl, or cycloalkyl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;
X¹ is N or C;
X² is N or CH;
B, including the atoms at the points of attachment, is a monocyclic or polycyclic 5- to 12-membered heterocycle or a monocyclic or polycyclic 5- to 12-membered heteroaryl;
R² is independently H, —OR^{b}, —NR⁵R⁶, —CN, —C₁-C₆ alkyl, —C₂-C₆ alkenyl, —C₄-C₈ cycloalkenyl, —C₂-C₆ alkynyl, —NH₂, halogen, —C(O)OR^{a}, —C₃-C₈ cycloalkyl, heterocyclyl containing 1-5 heteroatoms selected from the group consisting of N, S, P, or O, or heteroaryl containing 1-5 heteroatoms selected from the group consisting of N, S, P, or O; wherein each alkyl, alkenyl, cycloalkenyl, alkynyl, cycloalkyl, heterocyclyl, or heteroaryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; and wherein the heterocyclyl or heteroaryl is not attached via a nitrogen atom;
Y² is selected from the group consisting of: —NR^{a}—, —(CR^{a}₂)ₘ—, —C(O)—, —C(R^{a})₂NH—, —(CR^{a}₂)ₘO—, —C(O)N(R^{a})—, —N(R^{a})C(O)—, —S(O)₂N(R^{a})—, —N(R^{a})S(O)₂—, —N(R^{a})C(O)N(R^{a})—, —N(R^{a})C(S)N(R^{a})—, —C(O)O—, —OC(O)—, —OC(O)N(R^{a})—, —N(R^{a})C(O)O—, —C(O)N(R^{a})O—, —N(R^{a})C(S)—, —C(S)N(R^{a})—, and —OC(O)O—; wherein the bond on the left side of Y², as drawn, is bound to the ring and the bond on the right side of the Y² moiety, as drawn, is bound to R³;
R^{a} is independently, at each occurrence, selected from the group consisting of —H, -D, —OH, —C₃-C₈ cycloalkyl, and —C₁-C₆ alkyl, wherein each alkyl or cycloalkyl is optionally substituted with one or more —NH₂, wherein 2 R^{a}, together with the carbon atom to which they are both attached, can combine to form a 3- to 8-membered cycloalkyl;
R^{b} is independently —H, -D, —C₁-C₆ alkyl, —C₁-C₆ cycloalkyl, —C₂-C₆ alkenyl, or heterocyclyl containing 1-5 heteroatoms selected from the group consisting of N, S, P, or O; wherein each alkyl, cycloalkyl, alkenyl, or heterocycle is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, heteroaryl, —(CH₂)ₙOH, —C₁-C₆ alkyl, CF₃, CHF₂, or CH₂F;
R³ is independently, at each occurrence, selected from the group consisting of —H, —C₁-C₆ alkyl, a 3- to 12-membered monocyclic or polycyclic heterocycle, C₃-C₈ cycloalkyl, or —(CH₂)ₙ—R^{b}, wherein each alkyl, heterocycle, or cycloalkyl is optionally substituted with one or more —C₁-C₆ alkyl, —OH, —NH₂, —OR^{a}, —NHR^{a}, —(CH₂)ₙOH, heterocyclyl, or spiroheterocyclyl; or
R³ can combine with R^{a} to form a 3- to 12-membered monocyclic or polycyclic heterocycle, or a 5- to 12-membered spiroheterocycle, wherein each heterocycle or spiroheterocycle is optionally substituted with —C₁-C₆ alkyl, —OH, —NH₂, heteroaryl, heterocyclyl, —(CH₂)ₙNH₂, —COOR^{a}, —CONHR^{b}, —CONH(CH₂)ₙCOOR^{a}, —NHCOOR^{a}, —CF₃, CHF₂, or CH₂F;
R⁵ and R⁶ are each independently, at each occurrence, selected from the group consisting of —H, -D, —C₁-C₆ alkyl, —C₂-C₆ alkenyl, —C₄-C₈ cycloalkenyl, —C₂-C₆ alkynyl, —C₃-C₈ cycloalkyl, a monocyclic or polycyclic 3- to 12-membered heterocycle, —OR⁷, —SR⁷, halogen, —NR⁷R⁸, —NO₂, and —CN;
R⁷ and R⁸ are independently, at each occurrence, —H, -D, —C₁-C₆ alkyl, —C₂-C₆ alkenyl, —C₄-C₈ cycloalkenyl, —C₂-C₆ alkynyl, —C₃-C₈ cycloalkyl, a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN;
m is independently 1, 2, 3, 4, 5 or 6; and
n is independently 0, 1, 2, 3, 4, 5, 6, 7, 8, 9, or 10.

The disclosure provides compounds, and pharmaceutically acceptable salts, prodrugs, solvates, hydrates, tautomers, or isomers thereof, in Table 1.

TABLE 1

| Cmpd | |
|---|---|
| # | Structure |
| 1 | 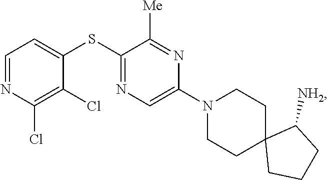 [SMILES: Cc1nc(N2CCC3(CCC[C@H]3N)CC2)cnc1Sc1ccnc(Cl)c1Cl] |
| 2 | 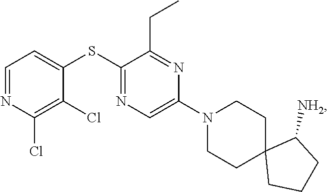 [SMILES: CCc1nc(N2CCC3(CCC[C@H]3N)CC2)cnc1Sc1ccnc(Cl)c1Cl] |
| 3 | 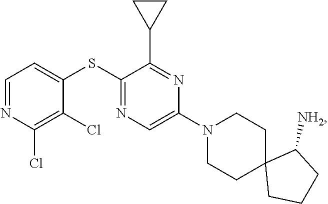 [SMILES: N[C@@H]1CCCC12CCN(c1cnc(Sc3ccnc(Cl)c3Cl)c(C3CC3)n1)CC2] |
| 4 | 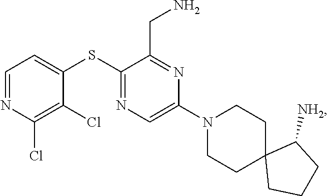 [SMILES: NCc1nc(N2CCC3(CCC[C@H]3N)CC2)cnc1Sc1ccnc(Cl)c1Cl] |
| 5 | 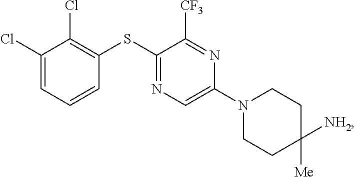 [SMILES: CC1(N)CCN(c2cnc(Sc3cccc(Cl)c3Cl)c(C(F)(F)F)n2)CC1] |
| 6 | 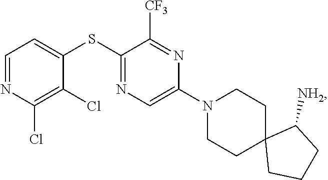 [SMILES: N[C@@H]1CCCC12CCN(c1cnc(Sc3ccnc(Cl)c3Cl)c(C(F)(F)F)n1)CC2] |
| 7 | 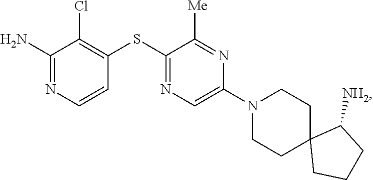 [SMILES: Cc1nc(N2CCC3(CCC[C@H]3N)CC2)cnc1Sc1ccnc(N)c1Cl] |
| 8 | 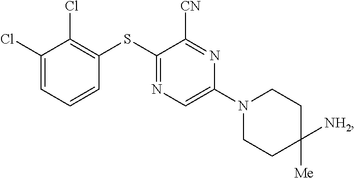 [SMILES: CC1(N)CCN(c2cnc(Sc3cccc(Cl)c3Cl)c(C#N)n2)CC1] |
| 9 | 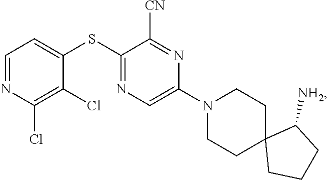 [SMILES: N#Cc1nc(N2CCC3(CCC[C@H]3N)CC2)cnc1Sc1ccnc(Cl)c1Cl] |
| 10 | 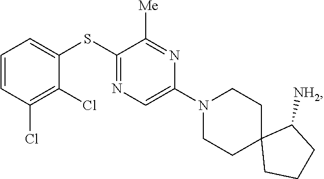 [SMILES: Cc1nc(N2CCC3(CCC[C@H]3N)CC2)cnc1Sc1cccc(Cl)c1Cl] |
| 11 | 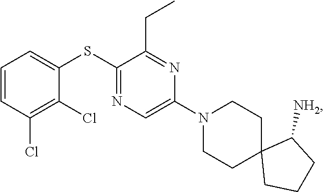 [SMILES: CCc1nc(N2CCC3(CCC[C@H]3N)CC2)cnc1Sc1cccc(Cl)c1Cl] |
| 12 | 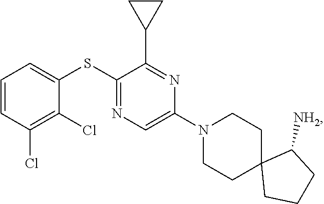 [SMILES: N[C@@H]1CCCC12CCN(c1cnc(Sc3cccc(Cl)c3Cl)c(C3CC3)n1)CC2] |
| 13 | 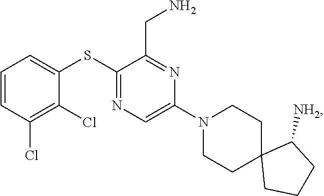 [SMILES: NCc1nc(N2CCC3(CCC[C@H]3N)CC2)cnc1Sc1cccc(Cl)c1Cl] |
| 14 | 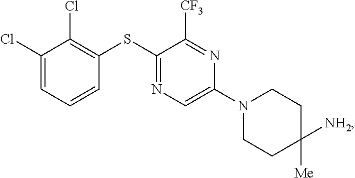 [SMILES: CC1(N)CCN(c2cnc(Sc3cccc(Cl)c3Cl)c(C(F)(F)F)n2)CC1] |
| 15 | 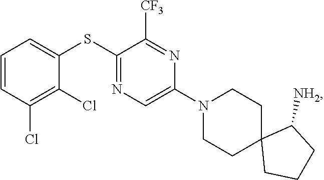 [SMILES: N[C@@H]1CCCC12CCN(c1cnc(Sc3cccc(Cl)c3Cl)c(C(F)(F)F)n1)CC2] |
| 16 | 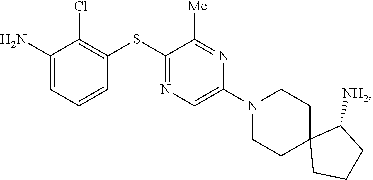 [SMILES: Cc1nc(N2CCC3(CCC[C@H]3N)CC2)cnc1Sc1cccc(N)c1Cl] |
| 17 | 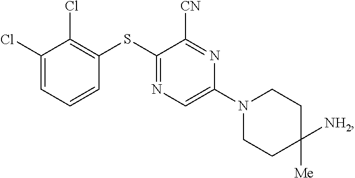 [SMILES: CC1(N)CCN(c2cnc(Sc3cccc(Cl)c3Cl)c(C#N)n2)CC1] |
| 18 | 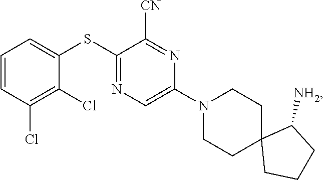 [SMILES: N#Cc1nc(N2CCC3(CCC[C@H]3N)CC2)cnc1Sc1cccc(Cl)c1Cl] |
| 19 | 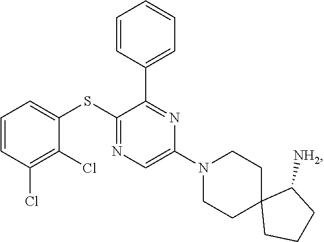 [SMILES: N[C@@H]1CCCC12CCN(c1cnc(Sc3cccc(Cl)c3Cl)c(-c3ccccc3)n1)CC2] |
| 20 | 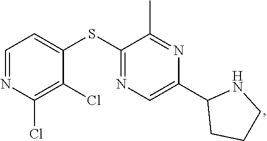 [SMILES: Cc1nc(C2CCCN2)cnc1Sc1ccnc(Cl)c1Cl] |
| 21 | 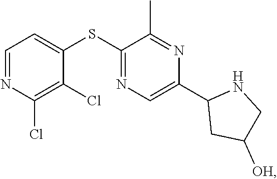 [SMILES: Cc1nc(C2CC(O)CN2)cnc1Sc1ccnc(Cl)c1Cl] |
| 22 | 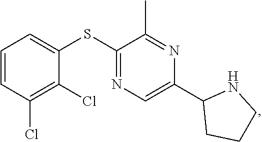 [SMILES: Cc1nc(C2CCCN2)cnc1Sc1cccc(Cl)c1Cl] |
| 23 | 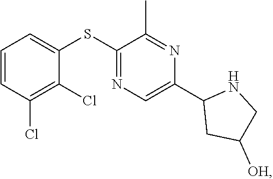 [SMILES: Cc1nc(C2CC(O)CN2)cnc1Sc1cccc(Cl)c1Cl] |
| 24 | 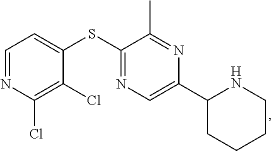 [SMILES: Cc1nc(C2CCCCN2)cnc1Sc1ccnc(Cl)c1Cl] |
| 25 | 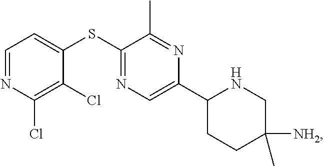 [SMILES: Cc1nc(C2CCC(C)(N)CN2)cnc1Sc1ccnc(Cl)c1Cl] |
| 26 | 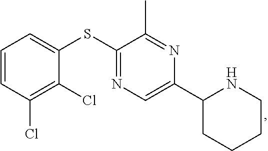 [SMILES: Cc1nc(C2CCCCN2)cnc1Sc1cccc(Cl)c1Cl] |
| 27 | 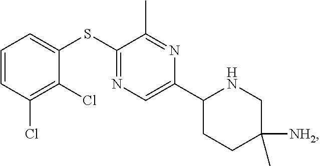 [SMILES: Cc1nc(C2CCC(C)(N)CN2)cnc1Sc1cccc(Cl)c1Cl] |
| 28 | 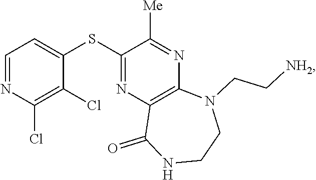 [SMILES: Cc1nc2c(nc1Sc1ccnc(Cl)c1Cl)C(=O)NCCN2CCN] |
| 29 | 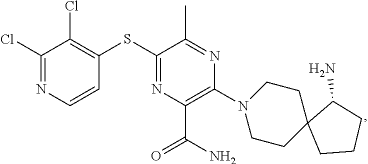 [SMILES: Cc1nc(N2CCC3(CCC[C@H]3N)CC2)c(C(N)=O)nc1Sc1ccnc(Cl)c1Cl] |
| 30 | 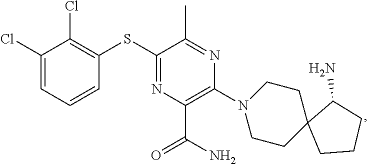 [SMILES: Cc1nc(N2CCC3(CCC[C@H]3N)CC2)c(C(N)=O)nc1Sc1cccc(Cl)c1Cl] |
| 31 | 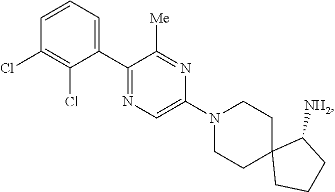 [SMILES: Cc1nc(N2CCC3(CCC[C@H]3N)CC2)cnc1-c1cccc(Cl)c1Cl] |
| 32 | 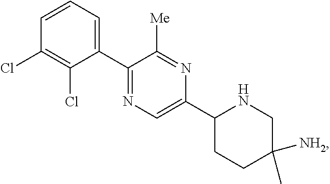 [SMILES: Cc1nc(C2CCC(C)(N)CN2)cnc1-c1cccc(Cl)c1Cl] |
| 33 | 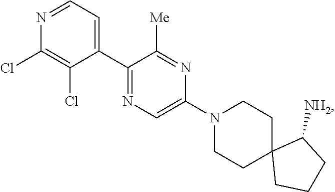 [SMILES: Cc1nc(N2CCC3(CCC[C@H]3N)CC2)cnc1-c1ccnc(Cl)c1Cl] |
| 34 | 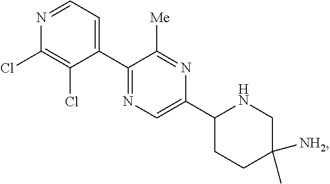 [SMILES: Cc1nc(C2CCC(C)(N)CN2)cnc1-c1ccnc(Cl)c1Cl] |
| 35 | 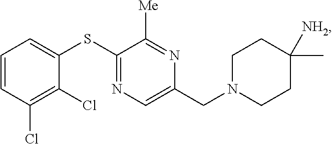 [SMILES: Cc1nc(CN2CCC(C)(N)CC2)cnc1Sc1cccc(Cl)c1Cl] |
| 36 | 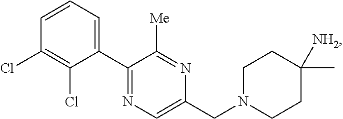 [SMILES: Cc1nc(CN2CCC(C)(N)CC2)cnc1-c1cccc(Cl)c1Cl] |
| 37 | 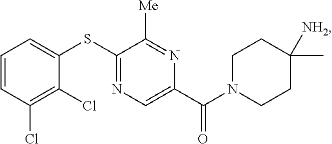 [SMILES: Cc1nc(C(=O)N2CCC(C)(N)CC2)cnc1Sc1cccc(Cl)c1Cl] |
| 38 | 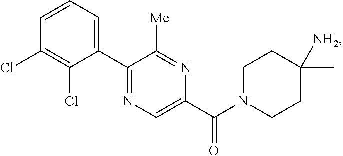 [SMILES: Cc1nc(C(=O)N2CCC(C)(N)CC2)cnc1-c1cccc(Cl)c1Cl] |
| 39 | 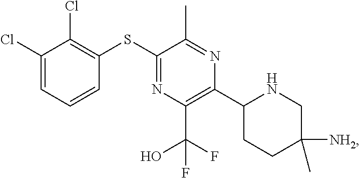 [SMILES: Cc1nc(C2CCC(C)(N)CN2)c(C(O)(F)F)nc1Sc1cccc(Cl)c1Cl] |
| 40 | 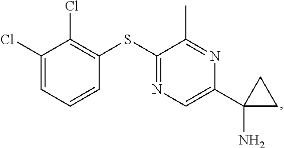 [SMILES: Cc1nc(C2(N)CC2)cnc1Sc1cccc(Cl)c1Cl] |
| 41 | 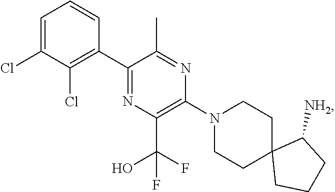 [SMILES: Cc1nc(N2CCC3(CCC[C@H]3N)CC2)c(C(O)(F)F)nc1-c1cccc(Cl)c1Cl] |
| A-1 | 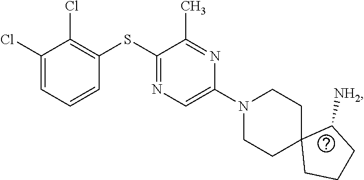 [SMILES: Cc1nc(N2CCC3(CCC[C@H]3N)CC2)cnc1Sc1cccc(Cl)c1Cl] |
| A-2 | 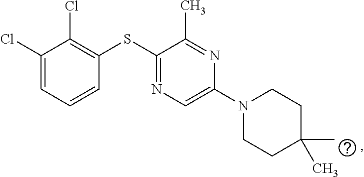 [SMILES: Cc1nc(N2CCC(C)(C)CC2)cnc1Sc1cccc(Cl)c1Cl] |
| A-3 | 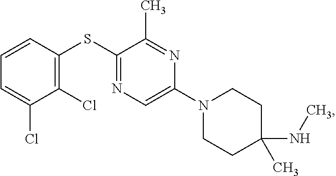 [SMILES: CNC1(C)CCN(c2cnc(Sc3cccc(Cl)c3Cl)c(C)n2)CC1] |
| A-4 | 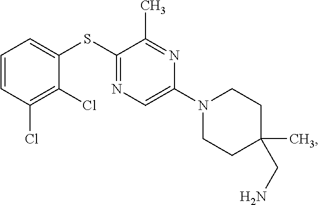 [SMILES: Cc1nc(N2CCC(C)(CN)CC2)cnc1Sc1cccc(Cl)c1Cl] |
| A-5 | 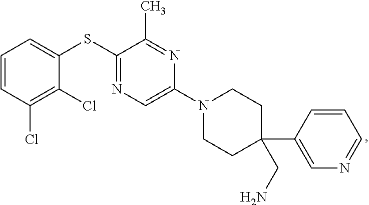 [SMILES: Cc1nc(N2CCC(CN)(c3cccnc3)CC2)cnc1Sc1cccc(Cl)c1Cl] |
| A-6 | 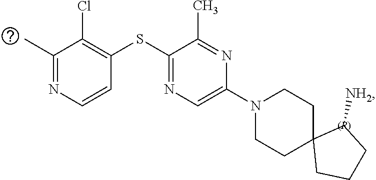 |
| A-7 | 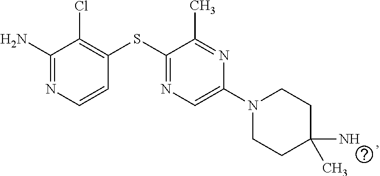 [SMILES: Cc1nc(N2CCC(C)(N)CC2)cnc1Sc1ccnc(N)c1Cl] |
| A-8 | 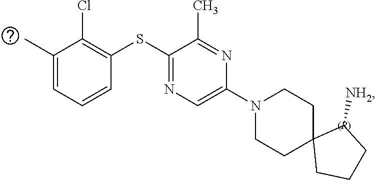 |
| A-9 | 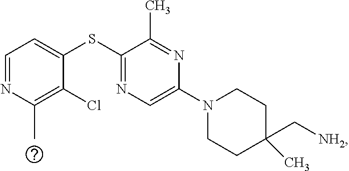 [SMILES: Cc1nc(N2CCC(C)(CN)CC2)cnc1Sc1ccnc(C)c1Cl] |
| A-10 | 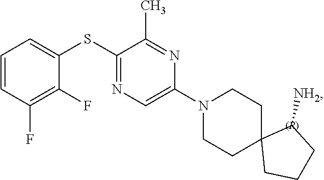 |
| A-11 | 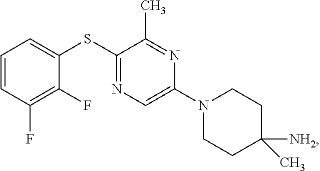 [SMILES: Cc1nc(N2CCC(C)(N)CC2)cnc1Sc1cccc(F)c1F] |
| A-12 | 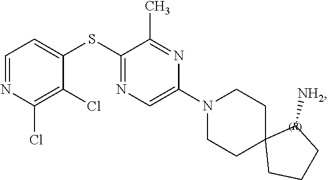 |
| A-13 | 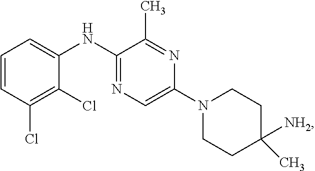 [SMILES: Cc1nc(N2CCC(C)(N)CC2)cnc1Nc1cccc(Cl)c1Cl] |
| A-14 | 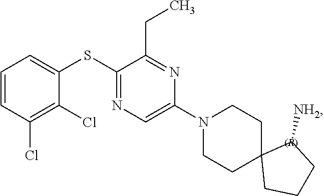 |
| A-15 | 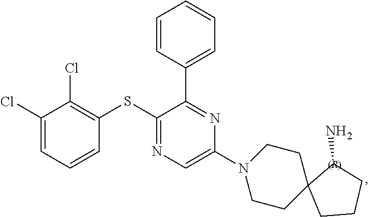 |
| A-16 | 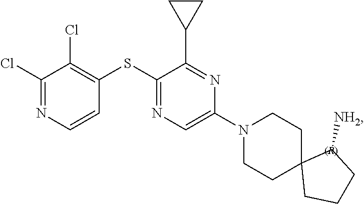 |
| A-17 | 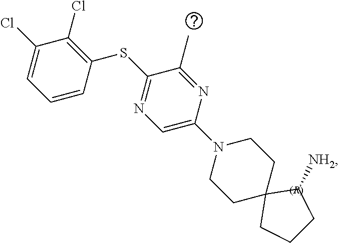 |
| A-18 | 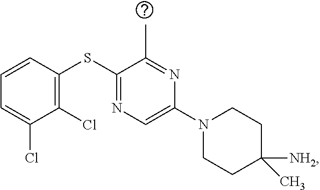 [SMILES: Cc1nc(N2CCC(C)(N)CC2)cnc1Sc1cccc(Cl)c1Cl] |
| A-19 | 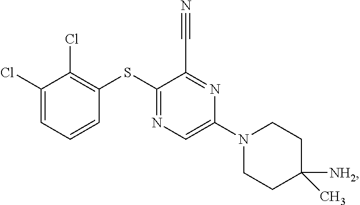 [SMILES: CC1(N)CCN(c2cnc(Sc3cccc(Cl)c3Cl)c(C#N)n2)CC1] |
| A-20 | 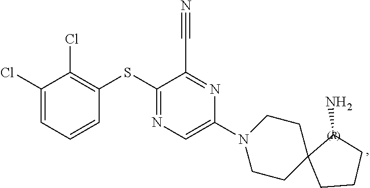 |
| A-21 | 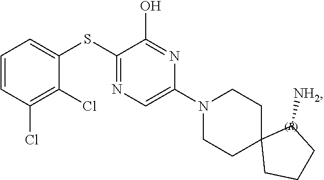 |
| A-22 | 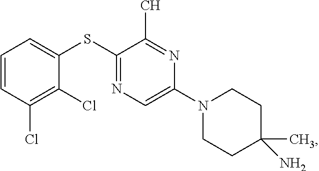 [SMILES: Cc1nc(N2CCC(C)(N)CC2)cnc1Sc1cccc(Cl)c1Cl] |
| A-23 | 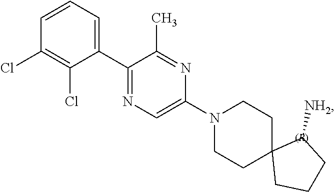 |
| A-24 | 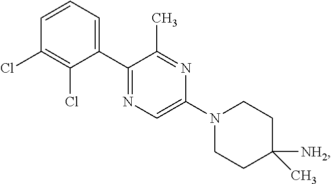 [SMILES: Cc1nc(N2CCC(C)(N)CC2)cnc1-c1cccc(Cl)c1Cl] |
| A-25 | 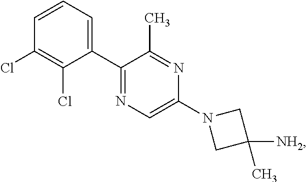 [SMILES: Cc1nc(N2CC(C)(N)C2)cnc1-c1cccc(Cl)c1Cl] |
| A-26 | 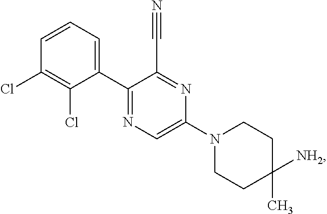 [SMILES: CC1(N)CCN(c2cnc(-c3cccc(Cl)c3Cl)c(C#N)n2)CC1] |
| A-27 | 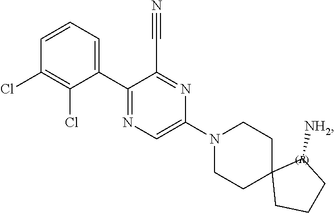 |
| A-28 | 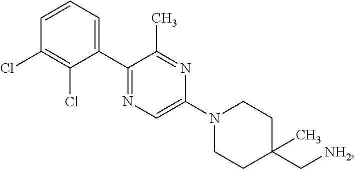 [SMILES: Cc1nc(N2CCC(C)(CN)CC2)cnc1-c1cccc(Cl)c1Cl] |
| A-29 | 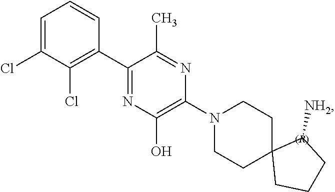 |
| A-30 | 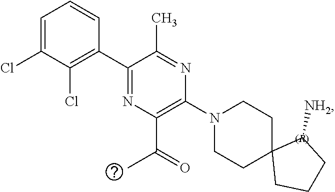 |
| A-31 | 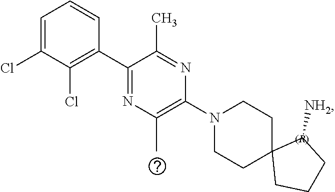 |
| A-32 | 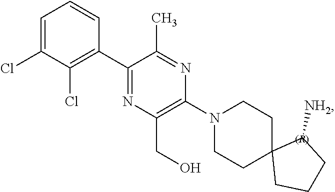 |
| A-33 | 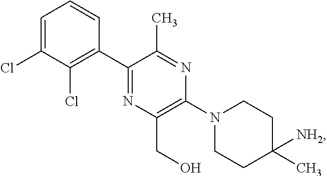 [SMILES: Cc1nc(N2CCC(C)(N)CC2)c(CO)nc1-c1cccc(Cl)c1Cl] |
| A-34 | 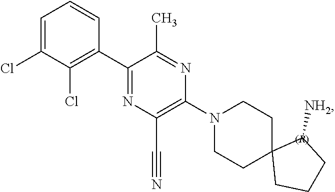 |
| A-35 | 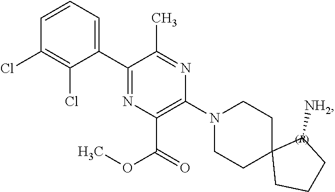 |
| A-36 | 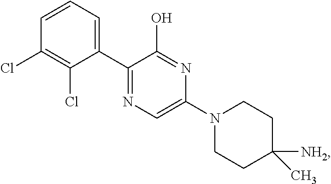 [SMILES: CC1(N)CCN(c2cnc(-c3cccc(Cl)c3Cl)c(O)n2)CC1] |
| A-37 | 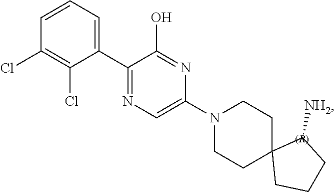 |
| A-38 | 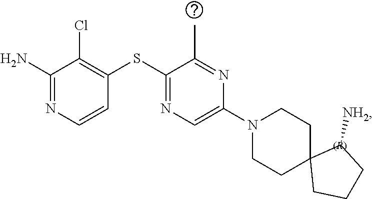 |
| A-39 | 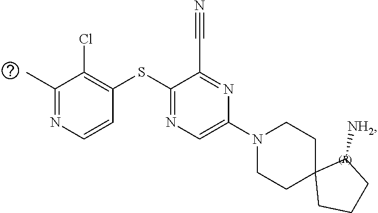 |
| A-40 | 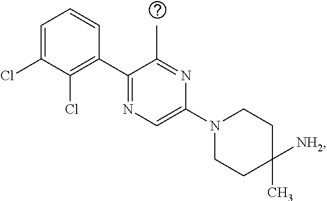 [SMILES: Cc1nc(N2CCC(C)(N)CC2)cnc1-c1cccc(Cl)c1Cl] |
| A-41 | 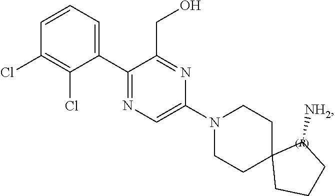 |
| A-42 | 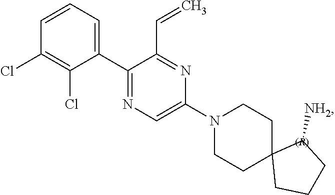 |
| A-43 | 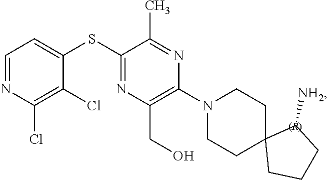 |
| A-44 | 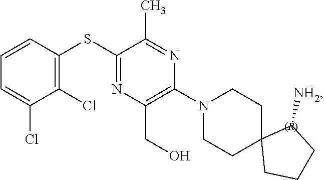 |
| A-45 | 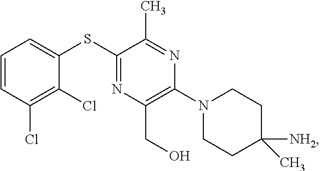 [SMILES: Cc1nc(N2CCC(C)(N)CC2)c(CO)nc1Sc1cccc(Cl)c1Cl] |
| A-46 | 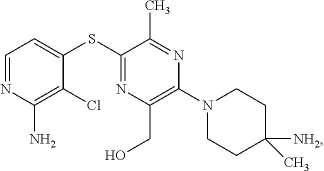 [SMILES: Cc1nc(N2CCC(C)(N)CC2)c(CO)nc1Sc1ccnc(N)c1Cl] |
| A-47 | 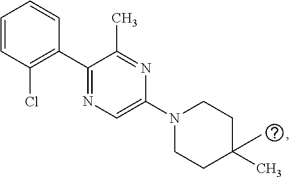 [SMILES: Cc1nc(N2CCC(C)(C)CC2)cnc1-c1ccccc1Cl] |
| A-48 | 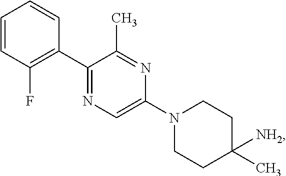 [SMILES: Cc1nc(N2CCC(C)(N)CC2)cnc1-c1ccccc1F] |
| A-49 | 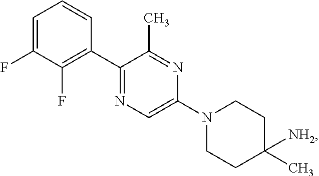 [SMILES: Cc1nc(N2CCC(C)(N)CC2)cnc1-c1cccc(F)c1F] |
| A-50 | 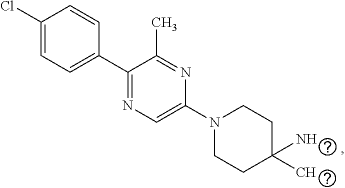 [SMILES: Cc1nc(N2CCC(C)(N)CC2)cnc1-c1ccc(Cl)cc1] |
| A-51 | 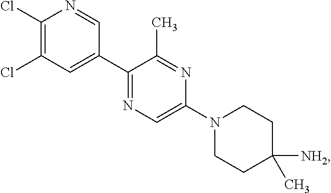 [SMILES: Cc1nc(N2CCC(C)(N)CC2)cnc1-c1cnc(Cl)c(Cl)c1] |
| A-52 | 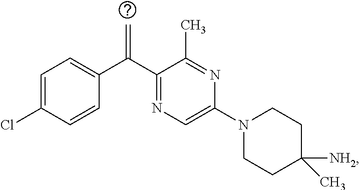 [SMILES: C=C(c1ccc(Cl)cc1)c1ncc(N2CCC(C)(N)CC2)nc1C] |
| A-53 | 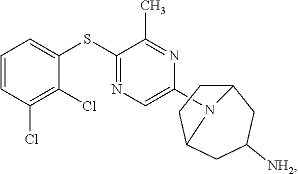 [SMILES: Cc1nc(N2C3CCC2CC(N)C3)cnc1Sc1cccc(Cl)c1Cl] |
| A-54 | 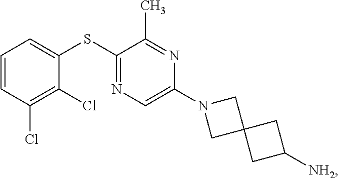 [SMILES: Cc1nc(N2CC3(CC(N)C3)C2)cnc1Sc1cccc(Cl)c1Cl] |
| A-55 | 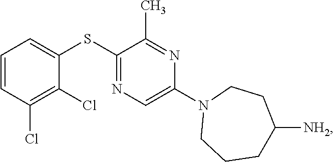 [SMILES: Cc1nc(N2CCCC(N)CC2)cnc1Sc1cccc(Cl)c1Cl] |
| A-56 | 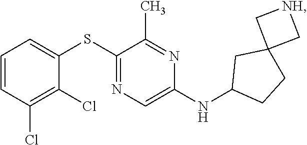 [SMILES: Cc1nc(NC2CCC3(CNC3)C2)cnc1Sc1cccc(Cl)c1Cl] |
| A-57 | 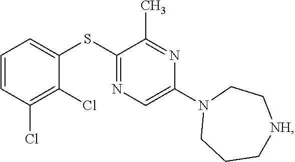 [SMILES: Cc1nc(N2CCCNCC2)cnc1Sc1cccc(Cl)c1Cl] |
| A-58 | 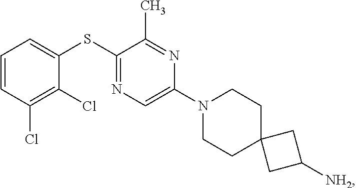 [SMILES: Cc1nc(N2CCC3(CC2)CC(N)C3)cnc1Sc1cccc(Cl)c1Cl] |
| A-59 | 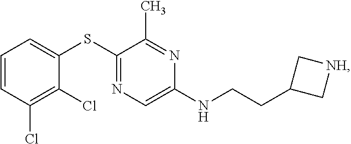 [SMILES: Cc1nc(NCCC2CNC2)cnc1Sc1cccc(Cl)c1Cl] |
| A-60 | 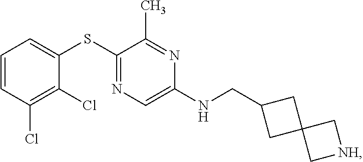 [SMILES: Cc1nc(NCC2CC3(CNC3)C2)cnc1Sc1cccc(Cl)c1Cl] |
| A-61 | 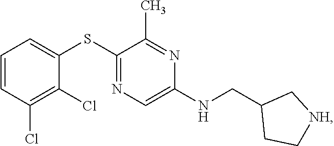 [SMILES: Cc1nc(NCC2CCNC2)cnc1Sc1cccc(Cl)c1Cl] |
| A-62 | 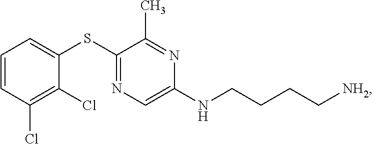 [SMILES: Cc1nc(NCCCCN)cnc1Sc1cccc(Cl)c1Cl] |
| A-63 | 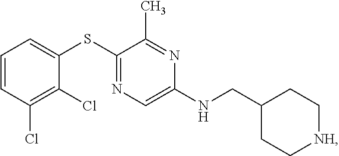 [SMILES: Cc1nc(NCC2CCNCC2)cnc1Sc1cccc(Cl)c1Cl] |
| A-64 | 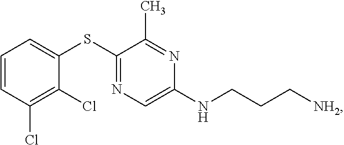 [SMILES: Cc1nc(NCCCN)cnc1Sc1cccc(Cl)c1Cl] |
| A-65 | 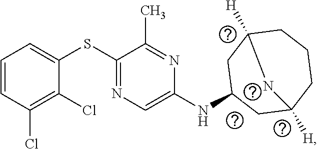 |
| A-66 | 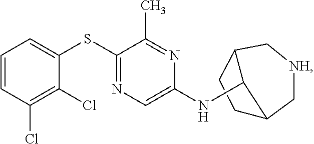 [SMILES: Cc1nc(NC2C3CCC2CNC3)cnc1Sc1cccc(Cl)c1Cl] |
| A-67 | 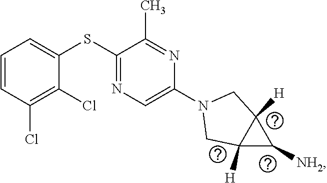 [SMILES: Cc1nc(N2C[C@@H]3[C@@H](N)[C@@H]3C2)cnc1Sc1cccc(Cl)c1Cl] |
| A-68 | 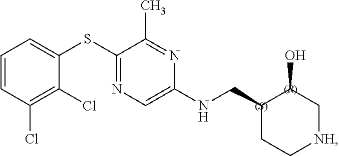 |
| A-69 | 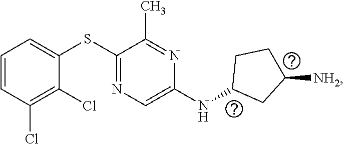 |
| A-70 | 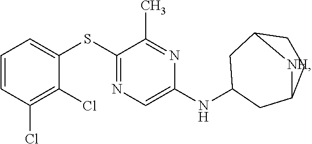 [SMILES: Cc1nc(NC2CC3CCC(C2)N3)cnc1Sc1cccc(Cl)c1Cl] |
| A-71 | 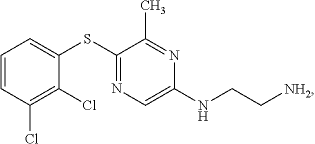 [SMILES: Cc1nc(NCCN)cnc1Sc1cccc(Cl)c1Cl] |
| A-72 | 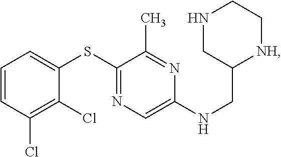 [SMILES: Cc1nc(NCC2CNCCN2)cnc1Sc1cccc(Cl)c1Cl] |
| A-73 | 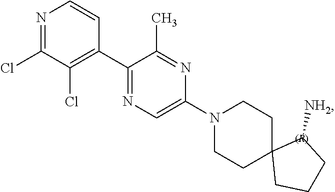 |
| A-74 | 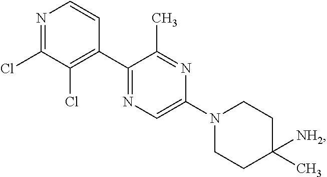 [SMILES: Cc1nc(N2CCC(C)(N)CC2)cnc1-c1ccnc(Cl)c1Cl] |
| A-75 | 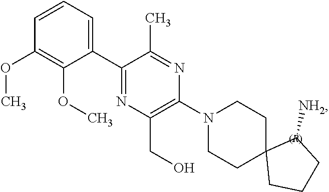 |
| A-76 | 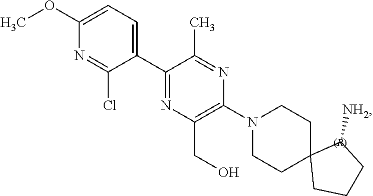 |
| A-77 | 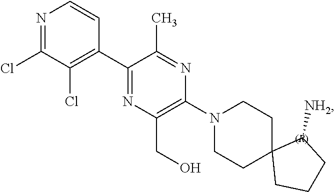 |
| A-78 | 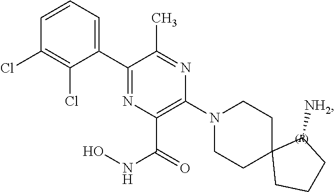 |
| A-79 | 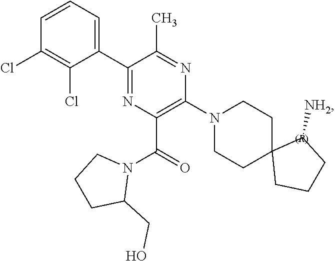 |
| A-80 | 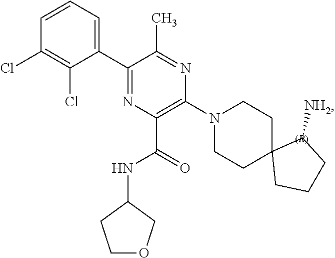 |
| A-81 | 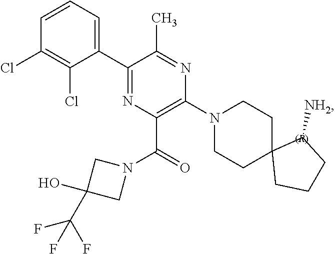 |
| A-82 | 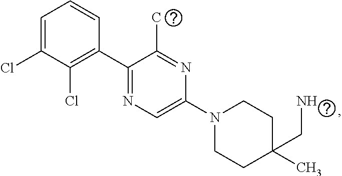 [SMILES: Cc1nc(N2CCC(C)(CN)CC2)cnc1-c1cccc(Cl)c1Cl] |
| A-83 | 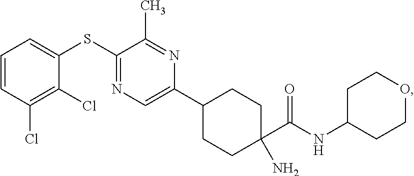 [SMILES: Cc1nc(C2CCC(N)(C(=O)NC3CCOCC3)CC2)cnc1Sc1cccc(Cl)c1Cl] |
| A-84 | 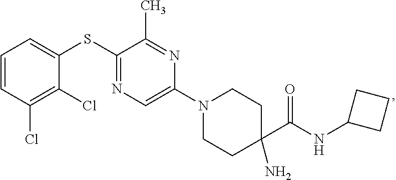 [SMILES: Cc1nc(N2CCC(N)(C(=O)NC3CCC3)CC2)cnc1Sc1cccc(Cl)c1Cl] |
| A-85 | 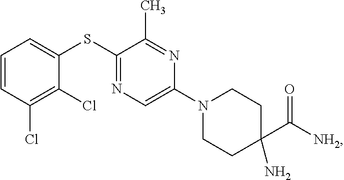 [SMILES: Cc1nc(N2CCC(N)(C(N)=O)CC2)cnc1Sc1cccc(Cl)c1Cl] |
| A-86 | 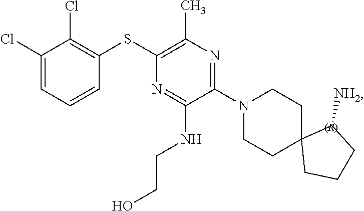 |
| A-87 | 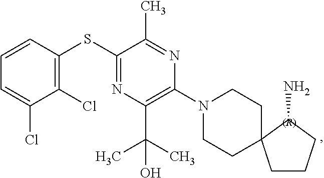 |
| A-88 | 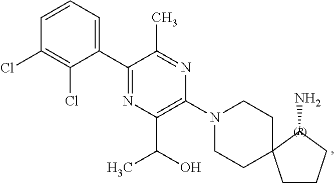 |
| A-89 | 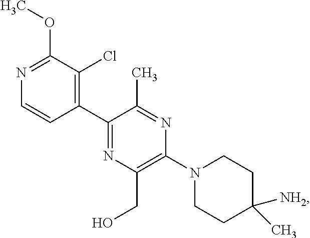 [SMILES: COc1nccc(-c2nc(CO)c(N3CCC(C)(N)CC3)nc2C)c1Cl] |
| A-90 | 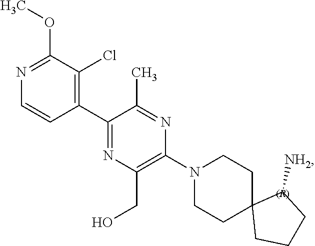 |
| A-91 | 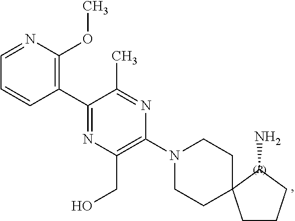 |
| A-92 | 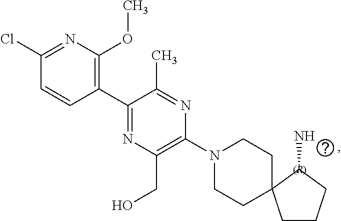 |
| A-93 | 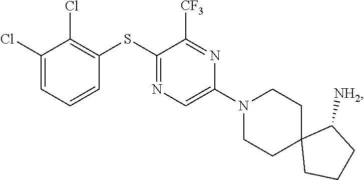 [SMILES: N[C@@H]1CCCC12CCN(c1cnc(Sc3cccc(Cl)c3Cl)c(C(F)(F)F)n1)CC2] |
| A-94 | 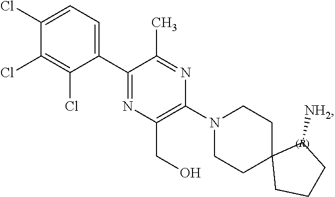 |
| A-95 | 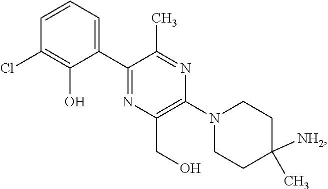 [SMILES: Cc1nc(N2CCC(C)(N)CC2)c(CO)nc1-c1cccc(Cl)c1O] |
| A-96 | 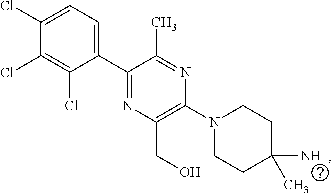 [SMILES: Cc1nc(N2CCC(C)(N)CC2)c(CO)nc1-c1ccc(Cl)c(Cl)c1Cl] |
| A-97 | 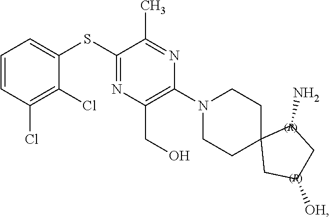 |
| A-98 | 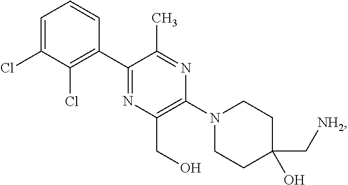 [SMILES: Cc1nc(N2CCC(O)(CN)CC2)c(CO)nc1-c1cccc(Cl)c1Cl] |
| A-99 | 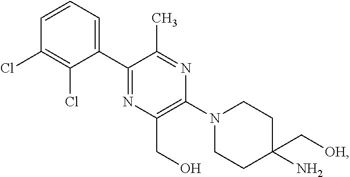 [SMILES: Cc1nc(N2CCC(N)(CO)CC2)c(CO)nc1-c1cccc(Cl)c1Cl] |
| A-100 | 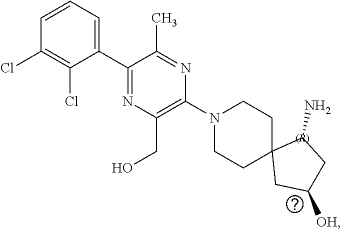 |
| A-101 | 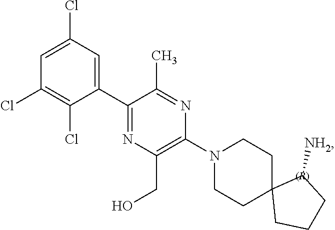 |
| A-102 | 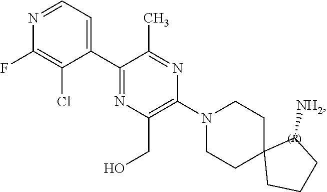 |
| A-103 | 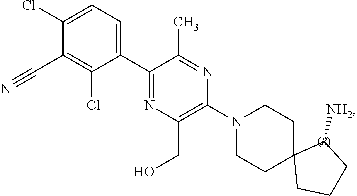 |
| A-104 | 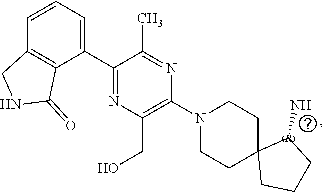 |
| A-105 | 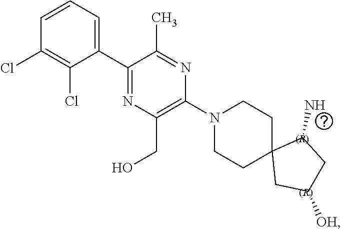 |
| A-106 | 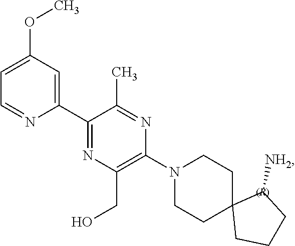 |
| A-107 | 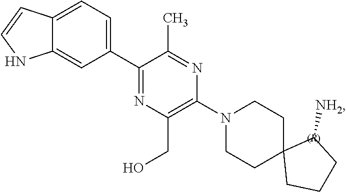 |
| A-108 | 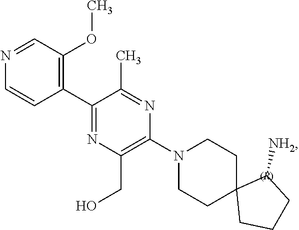 |
| A-109 | 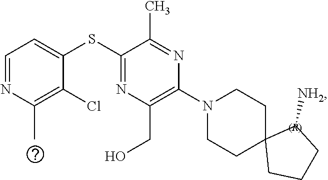 |
| A-110 | 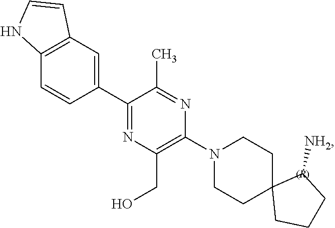 |
| A-111 | 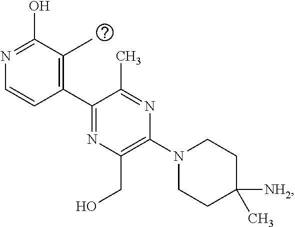 [SMILES: Cc1nc(N2CCC(C)(N)CC2)c(CO)nc1-c1ccnc(O)c1C] |
| A-112 | 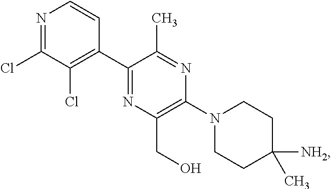 [SMILES: Cc1nc(N2CCC(C)(N)CC2)c(CO)nc1-c1ccnc(Cl)c1Cl] |
| A-113 | 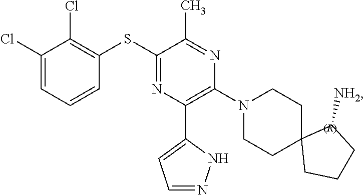 |
| A-114 | 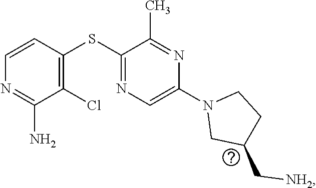 [SMILES: Cc1nc(N2CC[C@@H](CN)C2)cnc1Sc1ccnc(N)c1Cl] |
| A-115 | 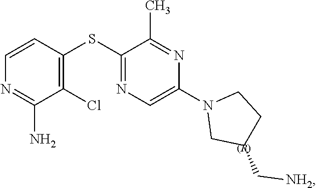 |
| A-116 | 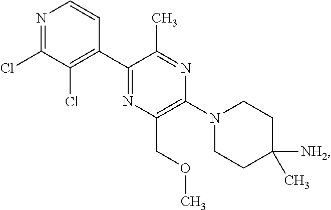 [SMILES: COCc1nc(-c2ccnc(Cl)c2Cl)c(C)nc1N1CCC(C)(N)CC1] |
| A-117 | 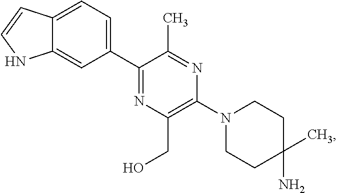 [SMILES: Cc1nc(N2CCC(C)(N)CC2)c(CO)nc1-c1ccc2cc[nH]c2c1] |
| A-118 | 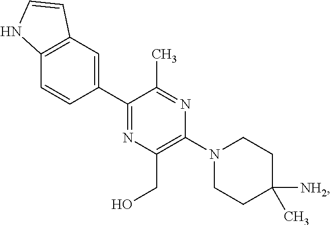 [SMILES: Cc1nc(N2CCC(C)(N)CC2)c(CO)nc1-c1ccc2[nH]ccc2c1] |
| A-119 | 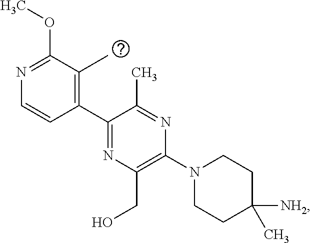 [SMILES: COc1nccc(-c2nc(CO)c(N3CCC(C)(N)CC3)nc2C)c1C] |
| A-120 | 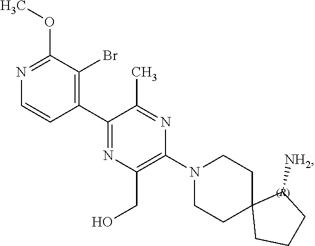 |
| A-121 | 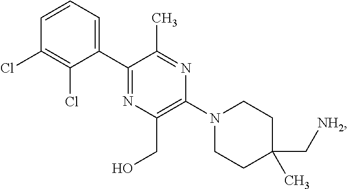 [SMILES: Cc1nc(N2CCC(C)(CN)CC2)c(CO)nc1-c1cccc(Cl)c1Cl] |
| A-122 | 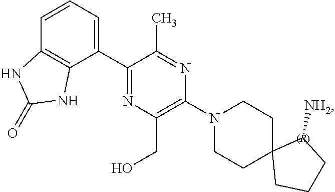 |
| A-123 | 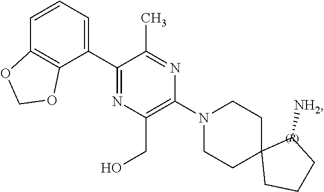 |
| A-124 | 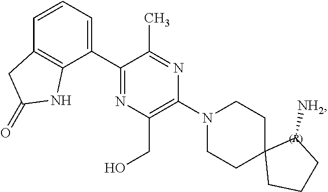 |
| A-125 | 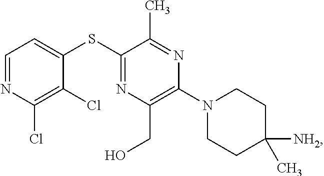 [SMILES: Cc1nc(N2CCC(C)(N)CC2)c(CO)nc1Sc1ccnc(Cl)c1Cl] |
| A-126 | 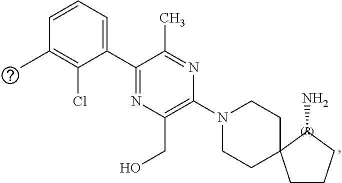 |
| A-127 | 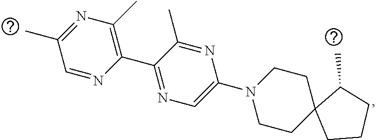 [SMILES: Cc1cnc(-c2ncc(N3CCC4(CCC[C@H]4C)CC3)nc2C)c(C)n1] |
| A-128 | 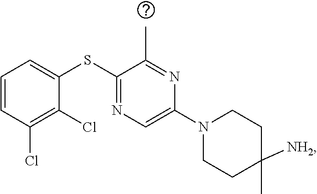 [SMILES: Cc1nc(N2CCC(C)(N)CC2)cnc1Sc1cccc(Cl)c1Cl] |
| A-129 | 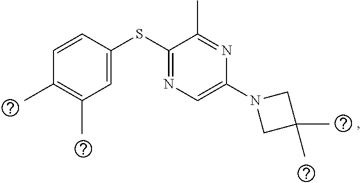 |
| A-130 | 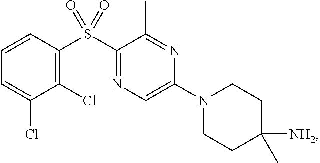 [SMILES: Cc1nc(N2CCC(C)(N)CC2)cnc1S(=O)(=O)c1cccc(Cl)c1Cl] |
| A-131 | 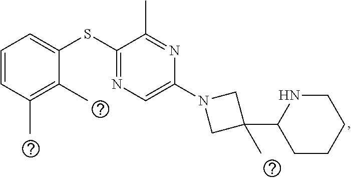 [SMILES: Cc1cccc(Sc2ncc(N3CC(C)(C4CCCCN4)C3)nc2C)c1C] |
| A-132 | 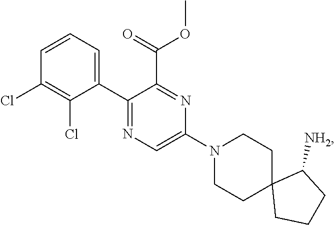 [SMILES: COC(=O)c1nc(N2CCC3(CCC[C@H]3N)CC2)cnc1-c1cccc(Cl)c1Cl] |
| A-133 | 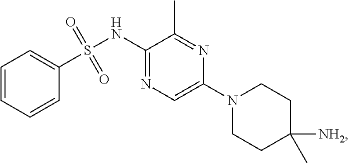 [SMILES: Cc1nc(N2CCC(C)(N)CC2)cnc1NS(=O)(=O)c1ccccc1] |
| A-134 | 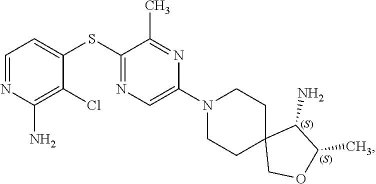 |
| A-135 | 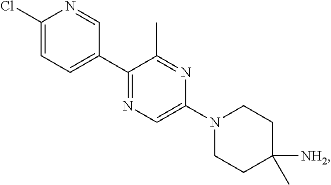 [SMILES: Cc1nc(N2CCC(C)(N)CC2)cnc1-c1ccc(Cl)nc1] |
| A-136 | 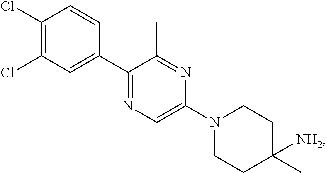 [SMILES: Cc1nc(N2CCC(C)(N)CC2)cnc1-c1ccc(Cl)c(Cl)c1] |
| A-137 | 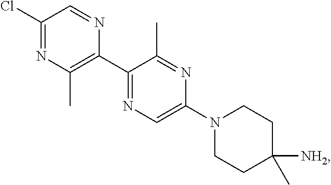 [SMILES: Cc1nc(Cl)cnc1-c1ncc(N2CCC(C)(N)CC2)nc1C] |
| A-138 | 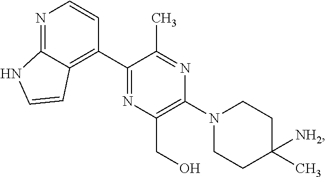 [SMILES: Cc1nc(N2CCC(C)(N)CC2)c(CO)nc1-c1ccnc2[nH]ccc12] |
| A-139 | 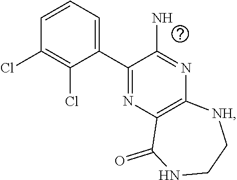 [SMILES: Nc1nc2c(nc1-c1cccc(Cl)c1Cl)C(=O)NCCN2] |
| A-140 | 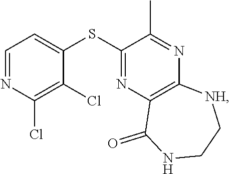 [SMILES: Cc1nc2c(nc1Sc1ccnc(Cl)c1Cl)C(=O)NCCN2] |
| A-141 | 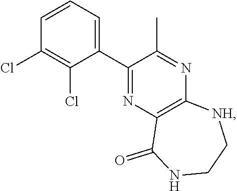 [SMILES: Cc1nc2c(nc1-c1cccc(Cl)c1Cl)C(=O)NCCN2] |
| A-142 | 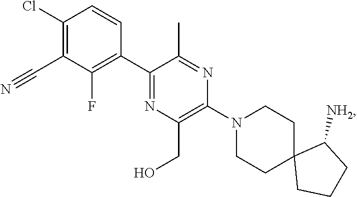 [SMILES: Cc1nc(N2CCC3(CCC[C@H]3N)CC2)c(CO)nc1-c1ccc(Cl)c(C#N)c1F] |
| A-143 | 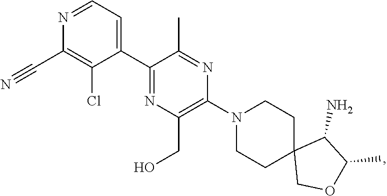 [SMILES: Cc1nc(N2CCC3(CC2)CO[C@@H](C)[C@H]3N)c(CO)nc1-c1ccnc(C#N)c1Cl] |
| A-144 | 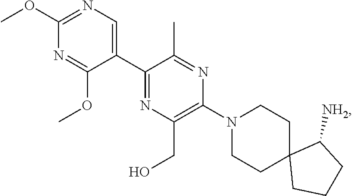 [SMILES: COc1ncc(-c2nc(CO)c(N3CCC4(CCC[C@H]4N)CC3)nc2C)c(OC)n1] |
| A-145 | 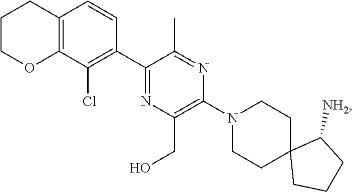 [SMILES: Cc1nc(N2CCC3(CCC[C@H]3N)CC2)c(CO)nc1-c1ccc2c(c1Cl)OCCC2] |
| A-146 | 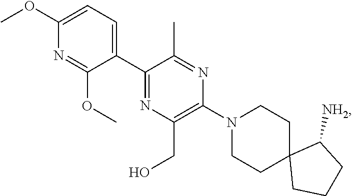 [SMILES: COc1ccc(-c2nc(CO)c(N3CCC4(CCC[C@H]4N)CC3)nc2C)c(OC)n1] |
| A-147 | 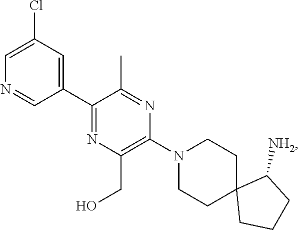 [SMILES: Cc1nc(N2CCC3(CCC[C@H]3N)CC2)c(CO)nc1-c1cncc(Cl)c1] |
| A-148 | 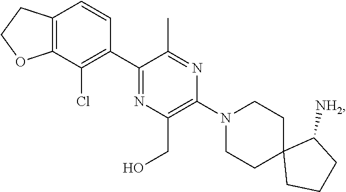 [SMILES: Cc1nc(N2CCC3(CCC[C@H]3N)CC2)c(CO)nc1-c1ccc2c(c1Cl)OCC2] |
| A-149 | 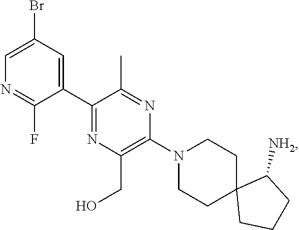 [SMILES: Cc1nc(N2CCC3(CCC[C@H]3N)CC2)c(CO)nc1-c1cc(Br)cnc1F] |
| A-150 | 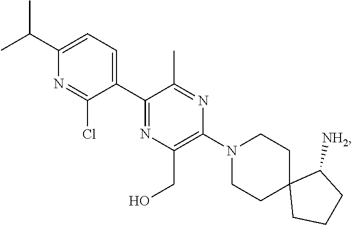 [SMILES: Cc1nc(N2CCC3(CCC[C@H]3N)CC2)c(CO)nc1-c1ccc(C(C)C)nc1Cl] |
| A-151 | 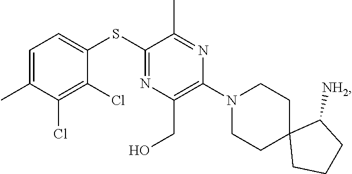 [SMILES: Cc1ccc(Sc2nc(CO)c(N3CCC4(CCC[C@H]4N)CC3)nc2C)c(Cl)c1Cl] |
| A-152 | 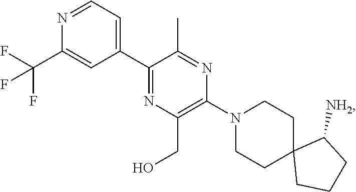 [SMILES: Cc1nc(N2CCC3(CCC[C@H]3N)CC2)c(CO)nc1-c1ccnc(C(F)(F)F)c1] |
| A-153 | 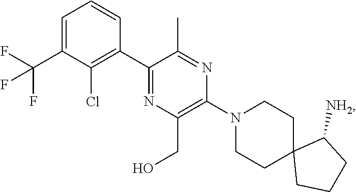 [SMILES: Cc1nc(N2CCC3(CCC[C@H]3N)CC2)c(CO)nc1-c1cccc(C(F)(F)F)c1Cl] |
| A-154 | 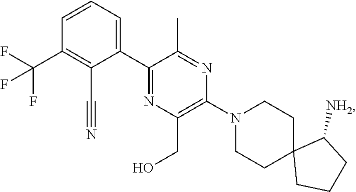 [SMILES: Cc1nc(N2CCC3(CCC[C@H]3N)CC2)c(CO)nc1-c1cccc(C(F)(F)F)c1C#N] |
| A-155 | 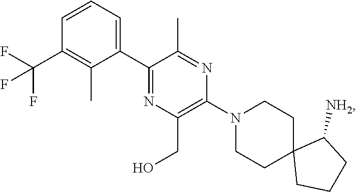 [SMILES: Cc1nc(N2CCC3(CCC[C@H]3N)CC2)c(CO)nc1-c1cccc(C(F)(F)F)c1C] |
| A-156 | 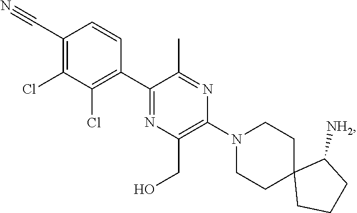 [SMILES: Cc1nc(N2CCC3(CCC[C@H]3N)CC2)c(CO)nc1-c1ccc(C#N)c(Cl)c1Cl] |
| A-157 | 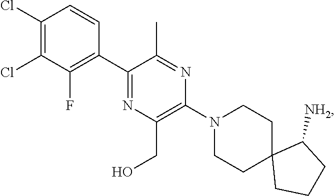 [SMILES: Cc1nc(N2CCC3(CCC[C@H]3N)CC2)c(CO)nc1-c1ccc(Cl)c(Cl)c1F] |
| A-158 | 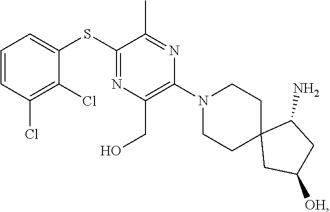 [SMILES: Cc1nc(N2CCC3(CC2)C[C@H](O)C[C@H]3N)c(CO)nc1Sc1cccc(Cl)c1Cl] |
| A-159 | 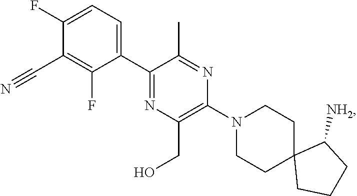 [SMILES: Cc1nc(N2CCC3(CCC[C@H]3N)CC2)c(CO)nc1-c1ccc(F)c(C#N)c1F] |
| A-160 | 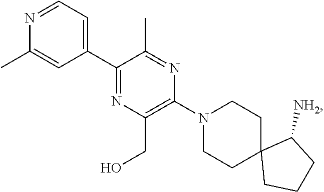 [SMILES: Cc1cc(-c2nc(CO)c(N3CCC4(CCC[C@H]4N)CC3)nc2C)ccn1] |
| A-161 | 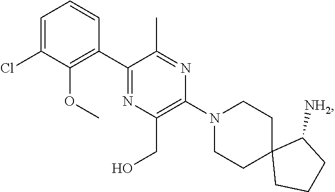 [SMILES: COc1c(Cl)cccc1-c1nc(CO)c(N2CCC3(CCC[C@H]3N)CC2)nc1C] |
| A-162 | 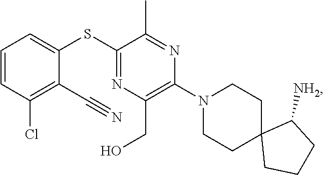 [SMILES: Cc1nc(N2CCC3(CCC[C@H]3N)CC2)c(CO)nc1Sc1cccc(Cl)c1C#N] |
| A-163 | 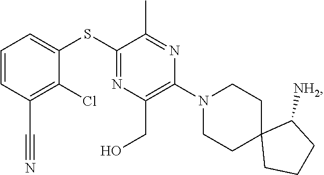 [SMILES: Cc1nc(N2CCC3(CCC[C@H]3N)CC2)c(CO)nc1Sc1cccc(C#N)c1Cl] |
| A-164 | 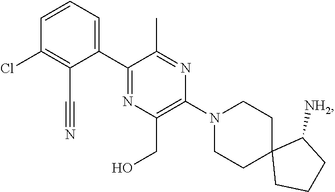 [SMILES: Cc1nc(N2CCC3(CCC[C@H]3N)CC2)c(CO)nc1-c1cccc(Cl)c1C#N] |
| A-165 | 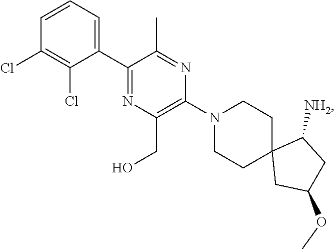 [SMILES: CO[C@@H]1C[C@@H](N)C2(CCN(c3nc(C)c(-c4cccc(Cl)c4Cl)nc3CO)CC2)C1] |
| A-166 | 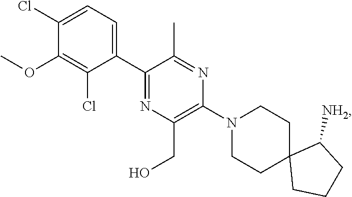 [SMILES: COc1c(Cl)ccc(-c2nc(CO)c(N3CCC4(CCC[C@H]4N)CC3)nc2C)c1Cl] |
| A-167 | 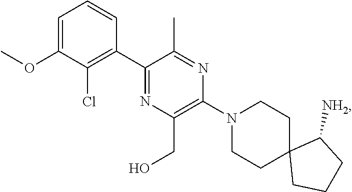 [SMILES: COc1cccc(-c2nc(CO)c(N3CCC4(CCC[C@H]4N)CC3)nc2C)c1Cl] |
| A-168 | 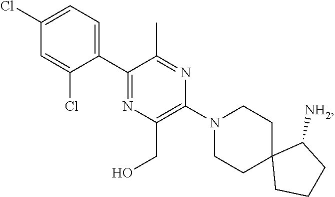 [SMILES: Cc1nc(N2CCC3(CCC[C@H]3N)CC2)c(CO)nc1-c1ccc(Cl)cc1Cl] |
| A-169 | 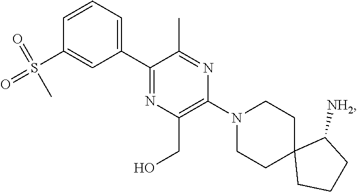 [SMILES: Cc1nc(N2CCC3(CCC[C@H]3N)CC2)c(CO)nc1-c1cccc(S(C)(=O)=O)c1] |
| A-170 | 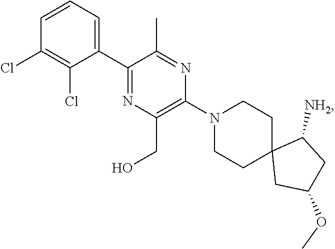 [SMILES: CO[C@H]1C[C@@H](N)C2(CCN(c3nc(C)c(-c4cccc(Cl)c4Cl)nc3CO)CC2)C1] |
| A-171 | 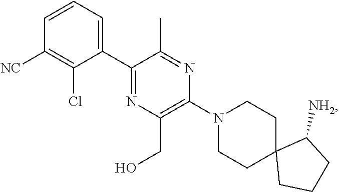 [SMILES: Cc1nc(N2CCC3(CCC[C@H]3N)CC2)c(CO)nc1-c1cccc(C#N)c1Cl] |
| A-172 | 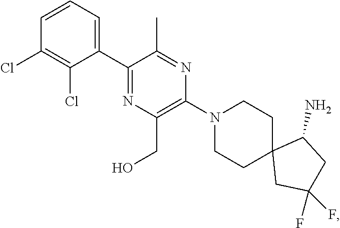 [SMILES: Cc1nc(N2CCC3(CC2)CC(F)(F)C[C@H]3N)c(CO)nc1-c1cccc(Cl)c1Cl] |
| A-173 | 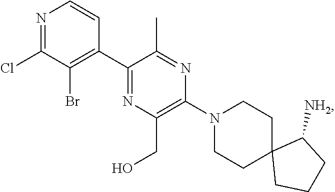 [SMILES: Cc1nc(N2CCC3(CCC[C@H]3N)CC2)c(CO)nc1-c1ccnc(Cl)c1Br] |
| A-174 | 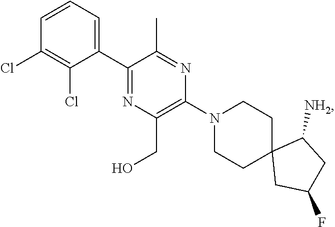 [SMILES: Cc1nc(N2CCC3(CC2)C[C@H](F)C[C@H]3N)c(CO)nc1-c1cccc(Cl)c1Cl] |
| A-175 | 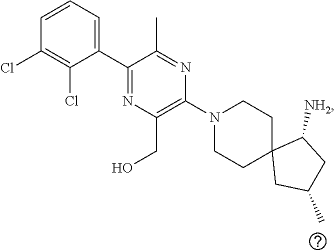 [SMILES: Cc1nc(N2CCC3(CC2)C[C@@H](C)C[C@H]3N)c(CO)nc1-c1cccc(Cl)c1Cl] |
| A-176 | 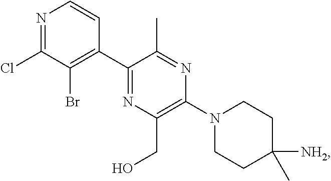 [SMILES: Cc1nc(N2CCC(C)(N)CC2)c(CO)nc1-c1ccnc(Cl)c1Br] |
| A-177 | 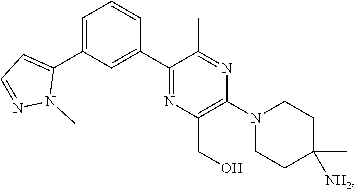 [SMILES: Cc1nc(N2CCC(C)(N)CC2)c(CO)nc1-c1cccc(-c2ccnn2C)c1] |
| A-178 | 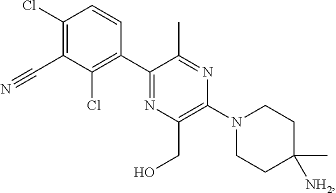 [SMILES: Cc1nc(N2CCC(C)(N)CC2)c(CO)nc1-c1ccc(Cl)c(C#N)c1Cl] |
| A-179 | 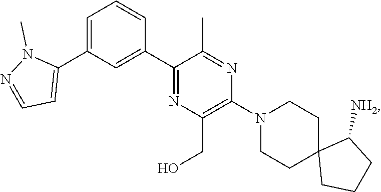 [SMILES: Cc1nc(N2CCC3(CCC[C@H]3N)CC2)c(CO)nc1-c1cccc(-c2ccnn2C)c1] |
| A-180 | 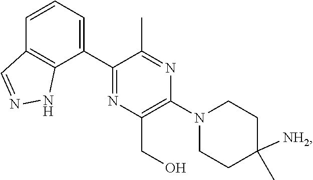 [SMILES: Cc1nc(N2CCC(C)(N)CC2)c(CO)nc1-c1cccc2cn[nH]c12] |
| A-181 | 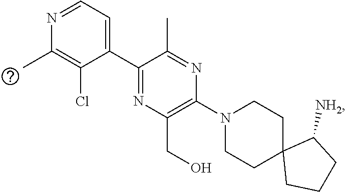 [SMILES: Cc1nc(N2CCC3(CCC[C@H]3N)CC2)c(CO)nc1-c1ccnc(C)c1Cl] |
| A-182 | 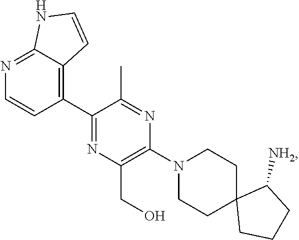 [SMILES: Cc1nc(N2CCC3(CCC[C@H]3N)CC2)c(CO)nc1-c1ccnc2[nH]ccc12] |
| A-183 | 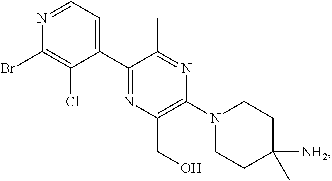 [SMILES: Cc1nc(N2CCC(C)(N)CC2)c(CO)nc1-c1ccnc(Br)c1Cl] |
| A-184 | 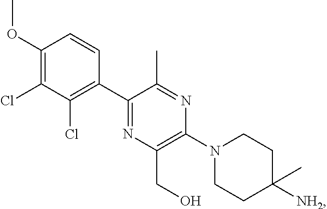 [SMILES: COc1ccc(-c2nc(CO)c(N3CCC(C)(N)CC3)nc2C)c(Cl)c1Cl] |
| A-185 | 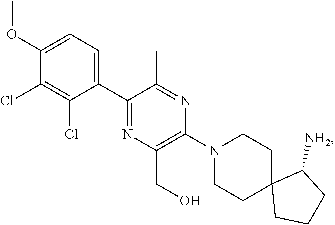 [SMILES: COc1ccc(-c2nc(CO)c(N3CCC4(CCC[C@H]4N)CC3)nc2C)c(Cl)c1Cl] |
| A-186 | 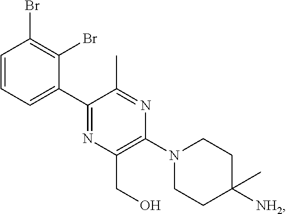 [SMILES: Cc1nc(N2CCC(C)(N)CC2)c(CO)nc1-c1cccc(Br)c1Br] |
| A-187 | 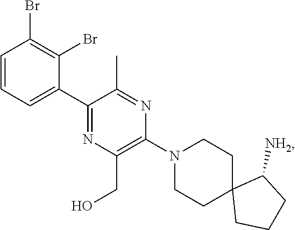 [SMILES: Cc1nc(N2CCC3(CCC[C@H]3N)CC2)c(CO)nc1-c1cccc(Br)c1Br] |
| A-188 | 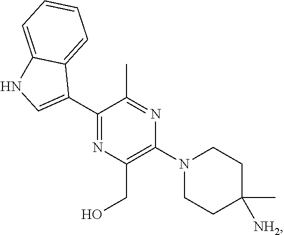 [SMILES: Cc1nc(N2CCC(C)(N)CC2)c(CO)nc1-c1c[nH]c2ccccc12] |
| A-189 | 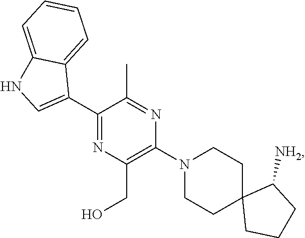 [SMILES: Cc1nc(N2CCC3(CCC[C@H]3N)CC2)c(CO)nc1-c1c[nH]c2ccccc12] |
| A-190 | 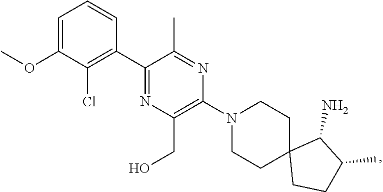 |
| A-191 | 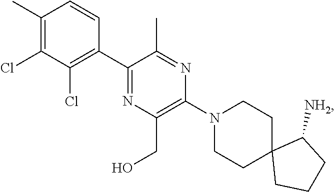 [SMILES: Cc1ccc(-c2nc(CO)c(N3CCC4(CCC[C@H]4N)CC3)nc2C)c(Cl)c1Cl] |
| A-192 | 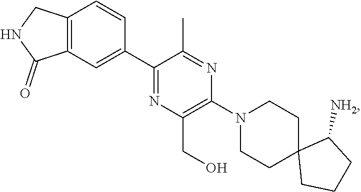 [SMILES: Cc1nc(N2CCC3(CCC[C@H]3N)CC2)c(CO)nc1-c1ccc2c(c1)C(=O)NC2] |
| A-193 | 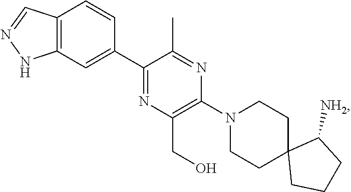 [SMILES: Cc1nc(N2CCC3(CCC[C@H]3N)CC2)c(CO)nc1-c1ccc2cn[nH]c2c1] |
| A-194 | 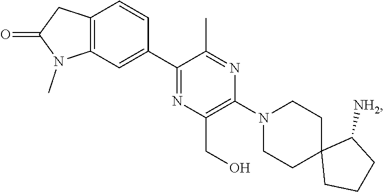 [SMILES: Cc1nc(N2CCC3(CCC[C@H]3N)CC2)c(CO)nc1-c1ccc2c(c1)N(C)C(=O)C2] |
| A-195 | 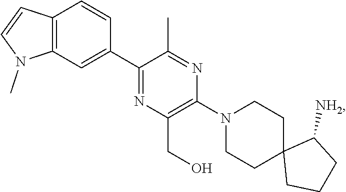 [SMILES: Cc1nc(N2CCC3(CCC[C@H]3N)CC2)c(CO)nc1-c1ccc2ccn(C)c2c1] |
| A-196 | 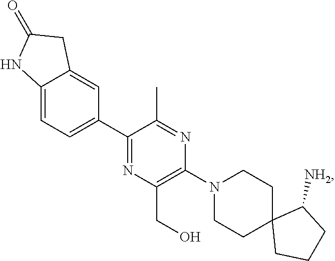 [SMILES: Cc1nc(N2CCC3(CCC[C@H]3N)CC2)c(CO)nc1-c1ccc2c(c1)CC(=O)N2] |
| A-197 | 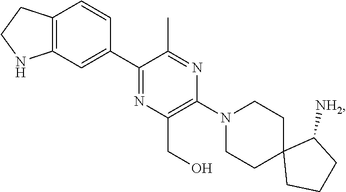 [SMILES: Cc1nc(N2CCC3(CCC[C@H]3N)CC2)c(CO)nc1-c1ccc2c(c1)NCC2] |
| A-198 | 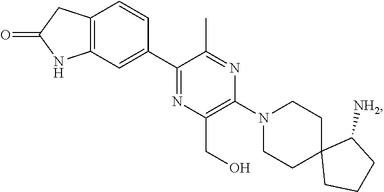 [SMILES: Cc1nc(N2CCC3(CCC[C@H]3N)CC2)c(CO)nc1-c1ccc2c(c1)NC(=O)C2] |
| A-199 | 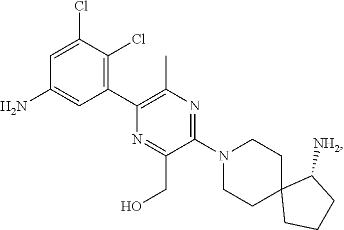 [SMILES: Cc1nc(N2CCC3(CCC[C@H]3N)CC2)c(CO)nc1-c1cc(N)cc(Cl)c1Cl] |
| A-200 | 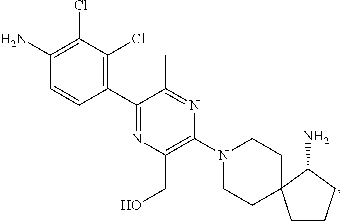 [SMILES: Cc1nc(N2CCC3(CCC[C@H]3N)CC2)c(CO)nc1-c1ccc(N)c(Cl)c1Cl] |
| A-201 | 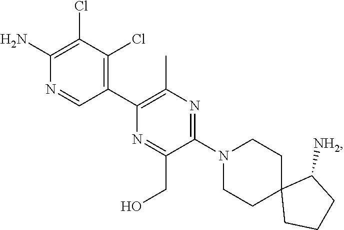 [SMILES: Cc1nc(N2CCC3(CCC[C@H]3N)CC2)c(CO)nc1-c1cnc(N)c(Cl)c1Cl] |
| A-202 | 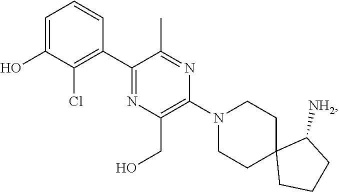 [SMILES: Cc1nc(N2CCC3(CCC[C@H]3N)CC2)c(CO)nc1-c1cccc(O)c1Cl] |
| A-203 | 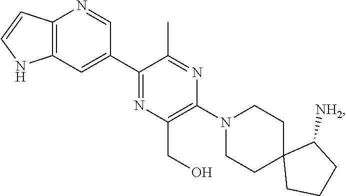 [SMILES: Cc1nc(N2CCC3(CCC[C@H]3N)CC2)c(CO)nc1-c1cnc2cc[nH]c2c1] |
| A-204 | 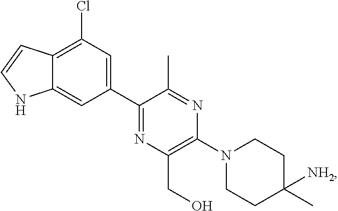 [SMILES: Cc1nc(N2CCC(C)(N)CC2)c(CO)nc1-c1cc(Cl)c2cc[nH]c2c1] |
| A-205 | 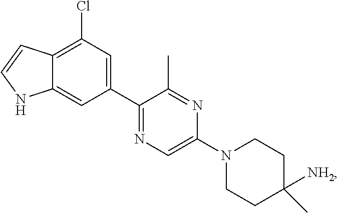 [SMILES: Cc1nc(N2CCC(C)(N)CC2)cnc1-c1cc(Cl)c2cc[nH]c2c1] |
| A-206 | 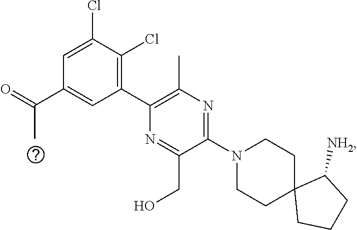 [SMILES: CC(=O)c1cc(Cl)c(Cl)c(-c2nc(CO)c(N3CCC4(CCC[C@H]4N)CC3)nc2C)c1] |
| A-207 | 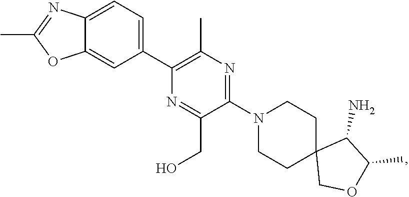 |
| A-208 | 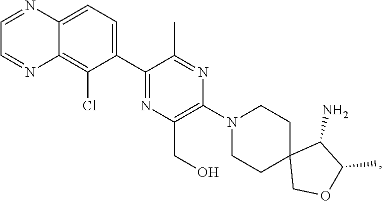 [SMILES: Cc1nc(N2CCC3(CC2)CO[C@@H](C)[C@H]3N)c(CO)nc1-c1ccc2nccnc2c1Cl] |
| A-209 | 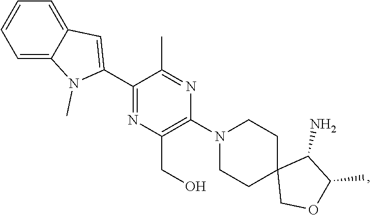 [SMILES: Cc1nc(N2CCC3(CC2)CO[C@@H](C)[C@H]3N)c(CO)nc1-c1cc2ccccc2n1C] |
| A-210 | 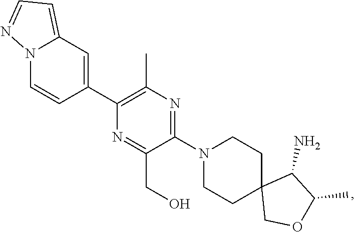 [SMILES: Cc1nc(N2CCC3(CC2)CO[C@@H](C)[C@H]3N)c(CO)nc1-c1ccn2nccc2c1] |
| A-211 | 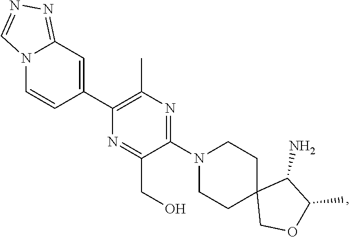 [SMILES: Cc1nc(N2CCC3(CC2)CO[C@@H](C)[C@H]3N)c(CO)nc1-c1ccn2cnnc2c1] |
| A-212 | 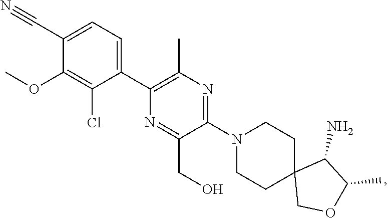 [SMILES: COc1c(C#N)ccc(-c2nc(CO)c(N3CCC4(CC3)CO[C@@H](C)[C@H]4N)nc2C)c1Cl] |
| A-213 | 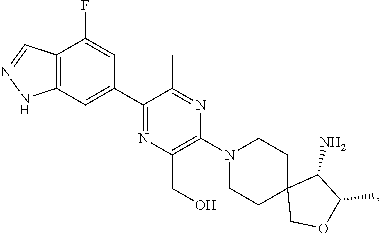 [SMILES: Cc1nc(N2CCC3(CC2)CO[C@@H](C)[C@H]3N)c(CO)nc1-c1cc(F)c2cn[nH]c2c1] |
| A-214 | 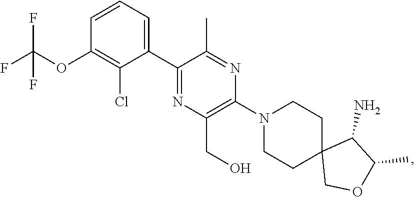 [SMILES: Cc1nc(N2CCC3(CC2)CO[C@@H](C)[C@H]3N)c(CO)nc1-c1cccc(OC(F)(F)F)c1Cl] |
| A-215 | 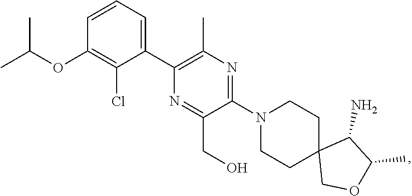 [SMILES: Cc1nc(N2CCC3(CC2)CO[C@@H](C)[C@H]3N)c(CO)nc1-c1cccc(OC(C)C)c1Cl] |
| A-216 | 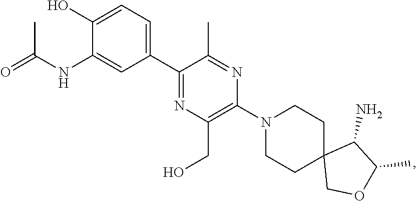 [SMILES: CC(=O)Nc1cc(-c2nc(CO)c(N3CCC4(CC3)CO[C@@H](C)[C@H]4N)nc2C)ccc1O] |
| A-217 | 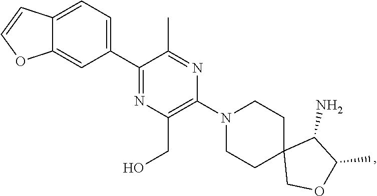 [SMILES: Cc1nc(N2CCC3(CC2)CO[C@@H](C)[C@H]3N)c(CO)nc1-c1ccc2ccoc2c1] |
| A-218 | 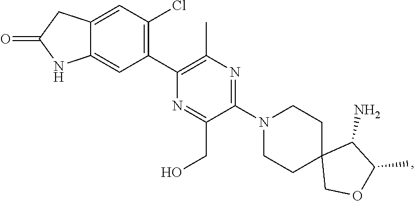 [SMILES: Cc1nc(N2CCC3(CC2)CO[C@@H](C)[C@H]3N)c(CO)nc1-c1cc2c(cc1Cl)CC(=O)N2] |
| A-219 | 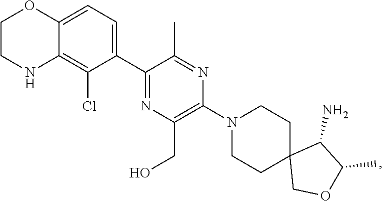 [SMILES: Cc1nc(N2CCC3(CC2)CO[C@@H](C)[C@H]3N)c(CO)nc1-c1ccc2c(c1Cl)NCCO2] |
| A-220 | 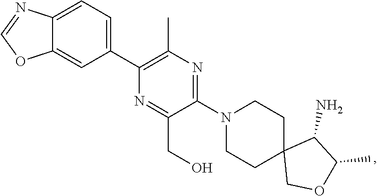 [SMILES: Cc1nc(N2CCC3(CC2)CO[C@@H](C)[C@H]3N)c(CO)nc1-c1ccc2ncoc2c1] |
| A-221 | 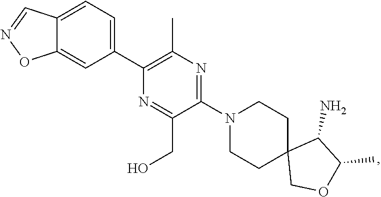 [SMILES: Cc1nc(N2CCC3(CC2)CO[C@@H](C)[C@H]3N)c(CO)nc1-c1ccc2cnoc2c1] |
| A-222 | 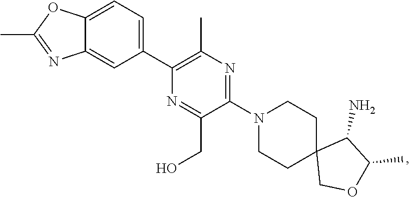 |
| A-223 | 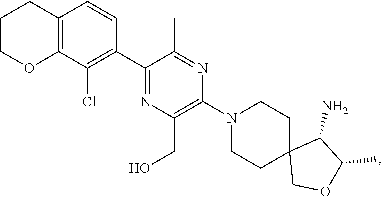 [SMILES: Cc1nc(N2CCC3(CC2)CO[C@@H](C)[C@H]3N)c(CO)nc1-c1ccc2c(c1Cl)OCCC2] |
| A-224 | 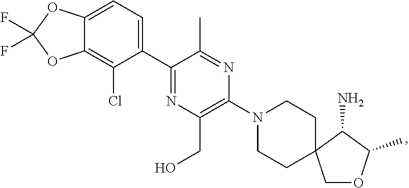 [SMILES: Cc1nc(N2CCC3(CC2)CO[C@@H](C)[C@H]3N)c(CO)nc1-c1ccc2c(c1Cl)OC(F)(F)O2] |
| A-225 | 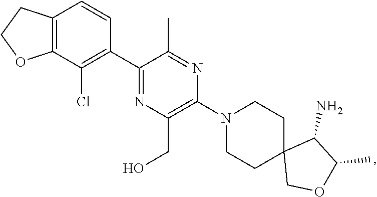 [SMILES: Cc1nc(N2CCC3(CC2)CO[C@@H](C)[C@H]3N)c(CO)nc1-c1ccc2c(c1Cl)OCC2] |
| A-226 | 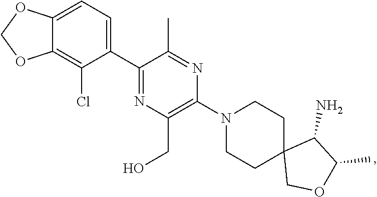 [SMILES: Cc1nc(N2CCC3(CC2)CO[C@@H](C)[C@H]3N)c(CO)nc1-c1ccc2c(c1Cl)OCO2] |
| A-227 | 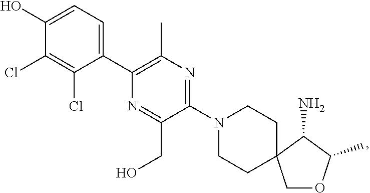 [SMILES: Cc1nc(N2CCC3(CC2)CO[C@@H](C)[C@H]3N)c(CO)nc1-c1ccc(O)c(Cl)c1Cl] |
| A-228 | 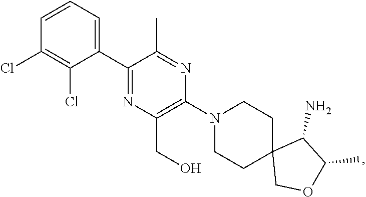 [SMILES: Cc1nc(N2CCC3(CC2)CO[C@@H](C)[C@H]3N)c(CO)nc1-c1cccc(Cl)c1Cl] |
| A-229 | 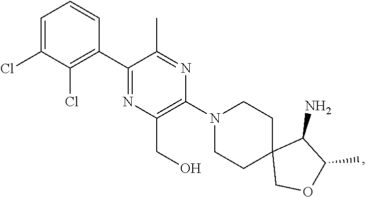 [SMILES: Cc1nc(N2CCC3(CC2)CO[C@@H](C)[C@@H]3N)c(CO)nc1-c1cccc(Cl)c1Cl] |
| A-230 | 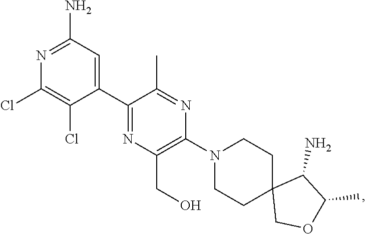 [SMILES: Cc1nc(N2CCC3(CC2)CO[C@@H](C)[C@H]3N)c(CO)nc1-c1cc(N)nc(Cl)c1Cl] |
| A-231 | 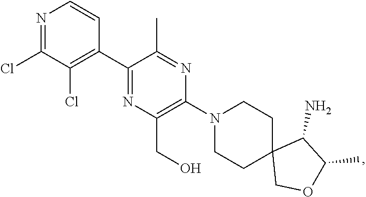 [SMILES: Cc1nc(N2CCC3(CC2)CO[C@@H](C)[C@H]3N)c(CO)nc1-c1ccnc(Cl)c1Cl] |
| A-232 | 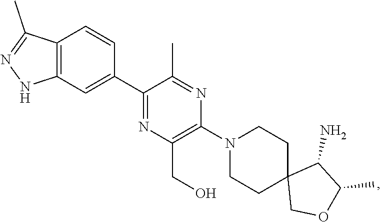 [SMILES: Cc1nc(N2CCC3(CC2)CO[C@@H](C)[C@H]3N)c(CO)nc1-c1ccc2c(C)n[nH]c2c1] |
| A-233 | 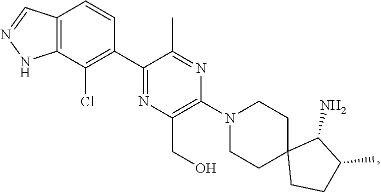 [SMILES: Cc1nc(N2CCC3(CC[C@@H](C)[C@H]3N)CC2)c(CO)nc1-c1ccc2cn[nH]c2c1Cl] |
| A-234 | 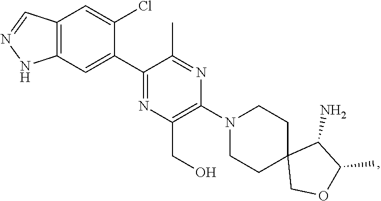 [SMILES: Cc1nc(N2CCC3(CC2)CO[C@@H](C)[C@H]3N)c(CO)nc1-c1cc2[nH]ncc2cc1Cl] |
| A-235 | 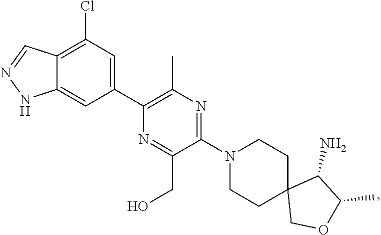 [SMILES: Cc1nc(N2CCC3(CC2)CO[C@@H](C)[C@H]3N)c(CO)nc1-c1cc(Cl)c2cn[nH]c2c1] |
| A-236 | 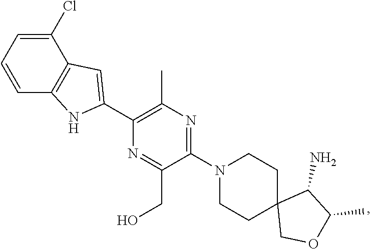 [SMILES: Cc1nc(N2CCC3(CC2)CO[C@@H](C)[C@H]3N)c(CO)nc1-c1cc2c(Cl)cccc2[nH]1] |
| A-237 | 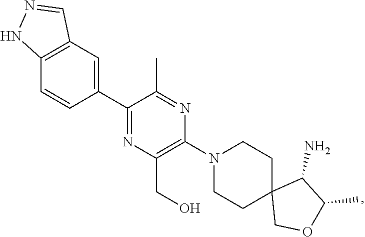 [SMILES: Cc1nc(N2CCC3(CC2)CO[C@@H](C)[C@H]3N)c(CO)nc1-c1ccc2[nH]ncc2c1] |
| A-238 | 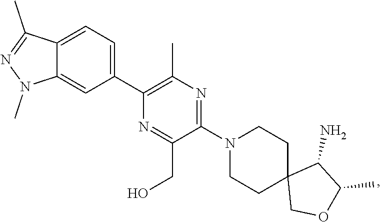 [SMILES: Cc1nc(N2CCC3(CC2)CO[C@@H](C)[C@H]3N)c(CO)nc1-c1ccc2c(C)nn(C)c2c1] |
| A-239 | 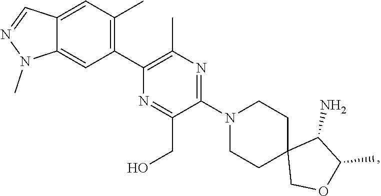 [SMILES: Cc1cc2cnn(C)c2cc1-c1nc(CO)c(N2CCC3(CC2)CO[C@@H](C)[C@H]3N)nc1C] |
| A-240 | 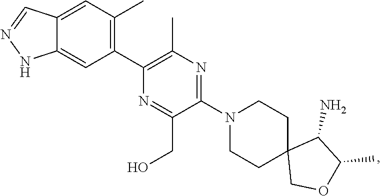 [SMILES: Cc1cc2cn[nH]c2cc1-c1nc(CO)c(N2CCC3(CC2)CO[C@@H](C)[C@H]3N)nc1C] |
| A-241 | 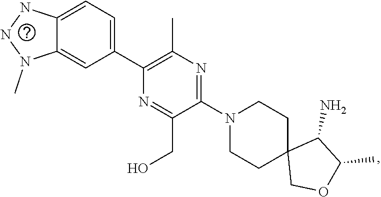 [SMILES: Cc1nc(N2CCC3(CC2)CO[C@@H](C)[C@H]3N)c(CO)nc1-c1ccc2c(c1)N(C)NN2] |
| A-242 | 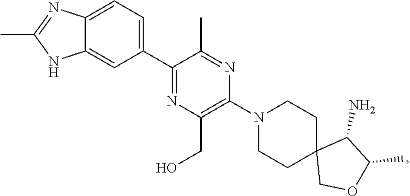 |
| A-243 | 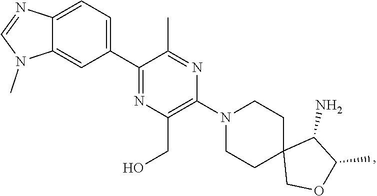 [SMILES: Cc1nc(N2CCC3(CC2)CO[C@@H](C)[C@H]3N)c(CO)nc1-c1ccc2ncn(C)c2c1] |
| A-244 | 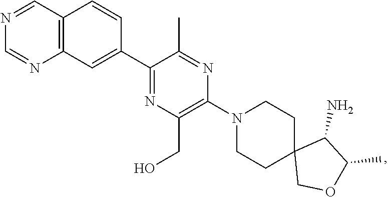 [SMILES: Cc1nc(N2CCC3(CC2)CO[C@@H](C)[C@H]3N)c(CO)nc1-c1ccc2cncnc2c1] |
| A-245 | 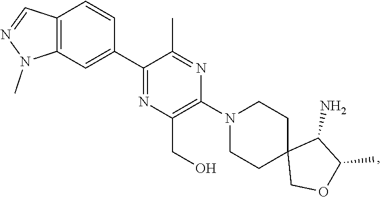 [SMILES: Cc1nc(N2CCC3(CC2)CO[C@@H](C)[C@H]3N)c(CO)nc1-c1ccc2cnn(C)c2c1] |
| A-246 | 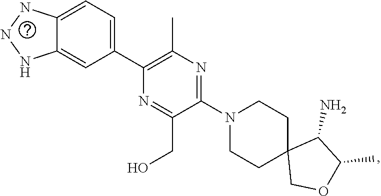 [SMILES: Cc1nc(N2CCC3(CC2)CO[C@@H](C)[C@H]3N)c(CO)nc1-c1ccc2c(c1)NNN2] |
| A-247 | 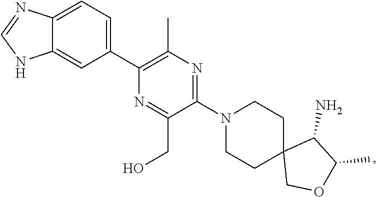 [SMILES: Cc1nc(N2CCC3(CC2)CO[C@@H](C)[C@H]3N)c(CO)nc1-c1ccc2nc[nH]c2c1] |
| A-248 | 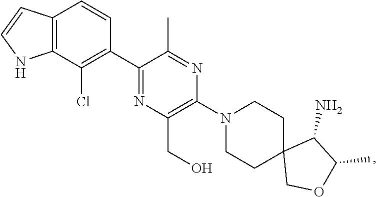 [SMILES: Cc1nc(N2CCC3(CC2)CO[C@@H](C)[C@H]3N)c(CO)nc1-c1ccc2cc[nH]c2c1Cl] |
| A-249 | 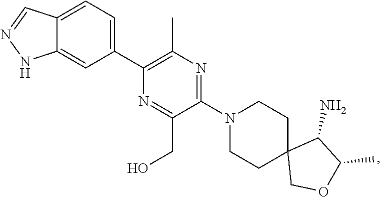 [SMILES: Cc1nc(N2CCC3(CC2)CO[C@@H](C)[C@H]3N)c(CO)nc1-c1ccc2cn[nH]c2c1] |
| A-250 | 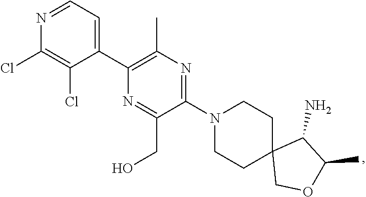 [SMILES: Cc1nc(N2CCC3(CC2)CO[C@H](C)[C@H]3N)c(CO)nc1-c1ccnc(Cl)c1Cl] |
| A-251 | 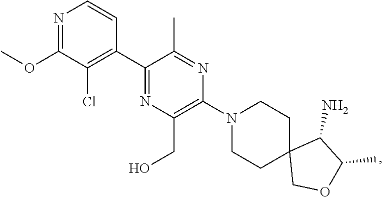 |
| A-252 | 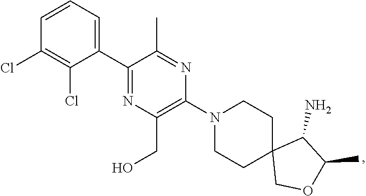 [SMILES: Cc1nc(N2CCC3(CC2)CO[C@H](C)[C@H]3N)c(CO)nc1-c1cccc(Cl)c1Cl] |
| A-253 | 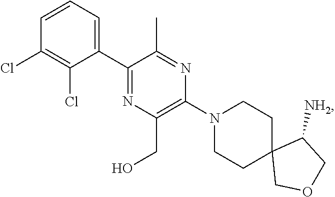 [SMILES: Cc1nc(N2CCC3(CC2)COC[C@H]3N)c(CO)nc1-c1cccc(Cl)c1Cl] |
| A-254 | 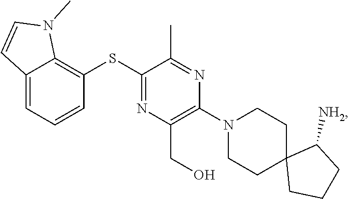 [SMILES: Cc1nc(N2CCC3(CCC[C@H]3N)CC2)c(CO)nc1Sc1cccc2ccn(C)c12] |
| A-255 | 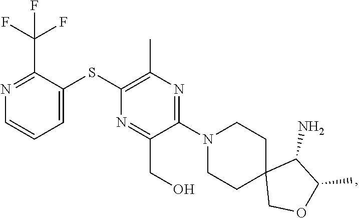 [SMILES: Cc1nc(N2CCC3(CC2)CO[C@@H](C)[C@H]3N)c(CO)nc1Sc1cccnc1C(F)(F)F] |
| A-256 | 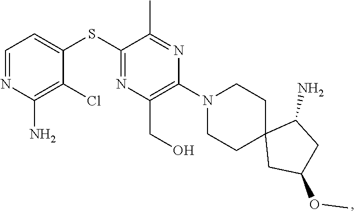 [SMILES: CO[C@@H]1C[C@@H](N)C2(CCN(c3nc(C)c(Sc4ccnc(N)c4Cl)nc3CO)CC2)C1] |
| A-257 | 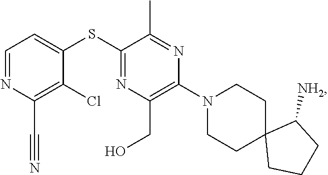 [SMILES: Cc1nc(N2CCC3(CCC[C@H]3N)CC2)c(CO)nc1Sc1ccnc(C#N)c1Cl] |
| A-258 | 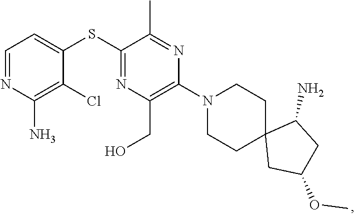 [SMILES: CO[C@H]1C[C@@H](N)C2(CCN(c3nc(C)c(Sc4ccnc(N)c4Cl)nc3CO)CC2)C1] |
| A-259 | 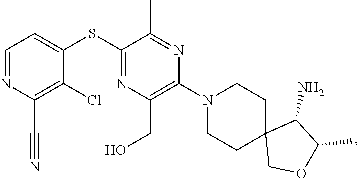 [SMILES: Cc1nc(N2CCC3(CC2)CO[C@@H](C)[C@H]3N)c(CO)nc1Sc1ccnc(C#N)c1Cl] |
| A-260 | 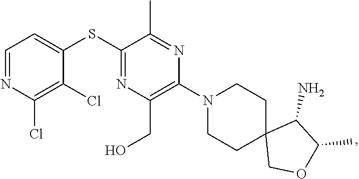 [SMILES: Cc1nc(N2CCC3(CC2)CO[C@@H](C)[C@H]3N)c(CO)nc1Sc1ccnc(Cl)c1Cl] |
| A-261 | 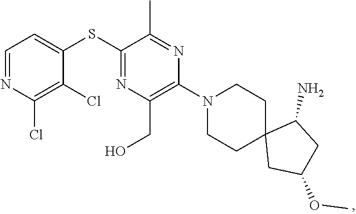 [SMILES: CO[C@H]1C[C@@H](N)C2(CCN(c3nc(C)c(Sc4ccnc(Cl)c4Cl)nc3CO)CC2)C1] |
| A-262 | 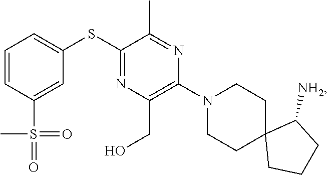 [SMILES: Cc1nc(N2CCC3(CCC[C@H]3N)CC2)c(CO)nc1Sc1cccc(S(C)(=O)=O)c1] |
| A-263 | 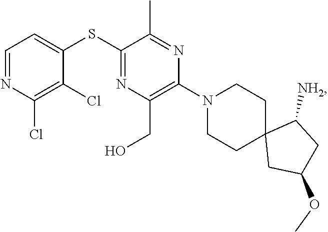 [SMILES: CO[C@@H]1C[C@@H](N)C2(CCN(c3nc(C)c(Sc4ccnc(Cl)c4Cl)nc3CO)CC2)C1] |
| A-264 | 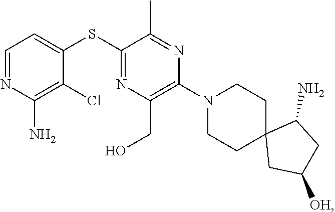 [SMILES: Cc1nc(N2CCC3(CC2)C[C@H](O)C[C@H]3N)c(CO)nc1Sc1ccnc(N)c1Cl] |
| A-265 | 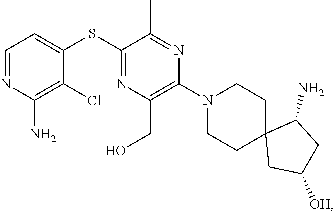 [SMILES: Cc1nc(N2CCC3(CC2)C[C@@H](O)C[C@H]3N)c(CO)nc1Sc1ccnc(N)c1Cl] |
| A-266 | 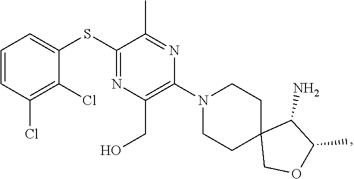 [SMILES: Cc1nc(N2CCC3(CC2)CO[C@@H](C)[C@H]3N)c(CO)nc1Sc1cccc(Cl)c1Cl] |
| A-267 | 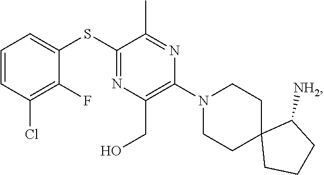 [SMILES: Cc1nc(N2CCC3(CCC[C@H]3N)CC2)c(CO)nc1Sc1cccc(Cl)c1F] |
| A-268 | 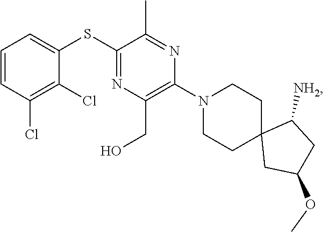 [SMILES: CO[C@@H]1C[C@@H](N)C2(CCN(c3nc(C)c(Sc4cccc(Cl)c4Cl)nc3CO)CC2)C1] |
| A-269 | 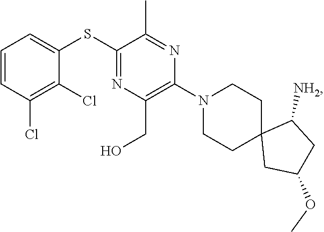 [SMILES: CO[C@H]1C[C@@H](N)C2(CCN(c3nc(C)c(Sc4cccc(Cl)c4Cl)nc3CO)CC2)C1] |
| A-270 | 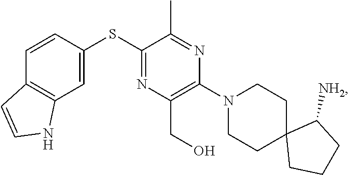 [SMILES: Cc1nc(N2CCC3(CCC[C@H]3N)CC2)c(CO)nc1Sc1ccc2cc[nH]c2c1] |
| A-271 | 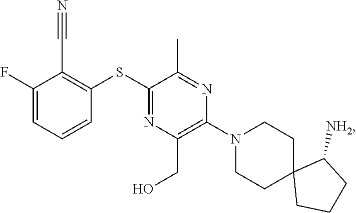 [SMILES: Cc1nc(N2CCC3(CCC[C@H]3N)CC2)c(CO)nc1Sc1cccc(F)c1C#N] |
| A-272 | 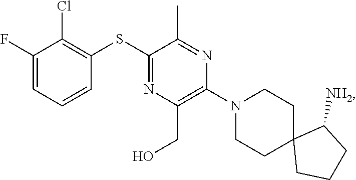 [SMILES: Cc1nc(N2CCC3(CCC[C@H]3N)CC2)c(CO)nc1Sc1cccc(F)c1Cl] |
| A-273 | 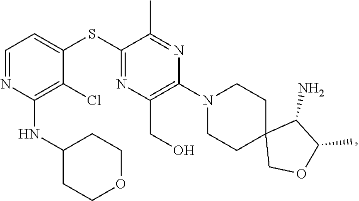 [SMILES: Cc1nc(N2CCC3(CC2)CO[C@@H](C)[C@H]3N)c(CO)nc1Sc1ccnc(NC2CCOCC2)c1Cl] |
| A-274 | 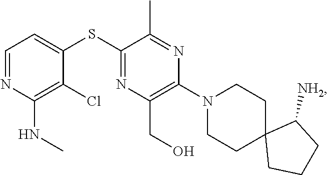 [SMILES: CNc1nccc(Sc2nc(CO)c(N3CCC4(CCC[C@H]4N)CC3)nc2C)c1Cl] |
| A-275 | 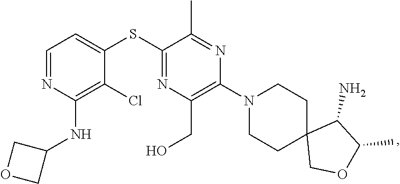 [SMILES: Cc1nc(N2CCC3(CC2)CO[C@@H](C)[C@H]3N)c(CO)nc1Sc1ccnc(NC2COC2)c1Cl] |
| A-276 | 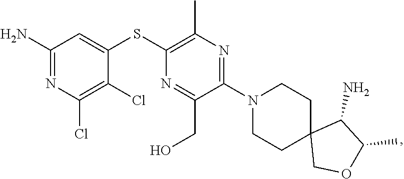 [SMILES: Cc1nc(N2CCC3(CC2)CO[C@@H](C)[C@H]3N)c(CO)nc1Sc1cc(N)nc(Cl)c1Cl] |
| A-277 | 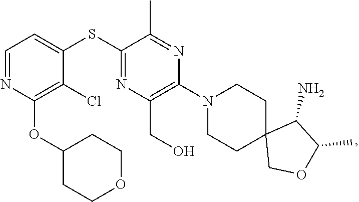 [SMILES: Cc1nc(N2CCC3(CC2)CO[C@@H](C)[C@H]3N)c(CO)nc1Sc1ccnc(OC2CCOCC2)c1Cl] |
| A-278 | 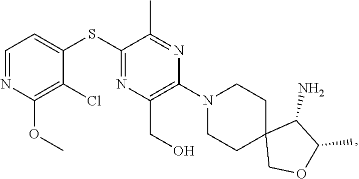 [SMILES: COc1nccc(Sc2nc(CO)c(N3CCC4(CC3)CO[C@@H](C)[C@H]4N)nc2C)c1Cl] |
| A-279 | 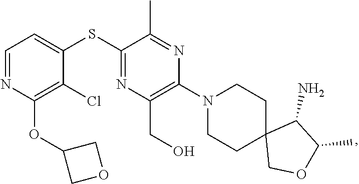 [SMILES: Cc1nc(N2CCC3(CC2)CO[C@@H](C)[C@H]3N)c(CO)nc1Sc1ccnc(OC2COC2)c1Cl] |
| A-280 | 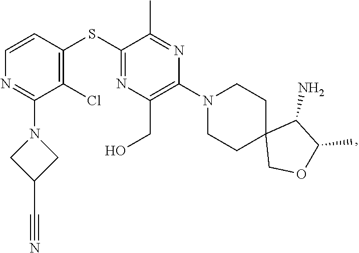 [SMILES: Cc1nc(N2CCC3(CC2)CO[C@@H](C)[C@H]3N)c(CO)nc1Sc1ccnc(N2CC(C#N)C2)c1Cl] |
| A-281 | 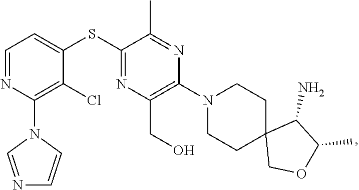 [SMILES: Cc1nc(N2CCC3(CC2)CO[C@@H](C)[C@H]3N)c(CO)nc1Sc1ccnc(-n2ccnc2)c1Cl] |
| A-282 | 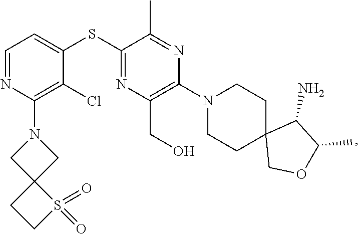 [SMILES: Cc1nc(N2CCC3(CC2)CO[C@@H](C)[C@H]3N)c(CO)nc1Sc1ccnc(N2CC3(CCS3(=O)=O)C2)c1Cl] |
| A-283 | 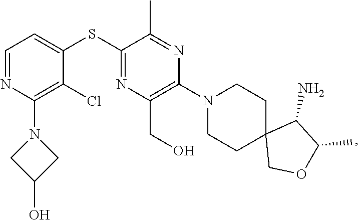 [SMILES: Cc1nc(N2CCC3(CC2)CO[C@@H](C)[C@H]3N)c(CO)nc1Sc1ccnc(N2CC(O)C2)c1Cl] |
| A-284 | 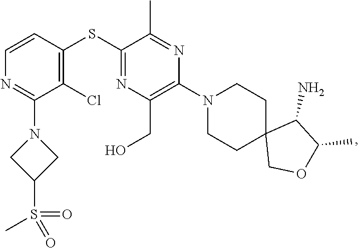 [SMILES: Cc1nc(N2CCC3(CC2)CO[C@@H](C)[C@H]3N)c(CO)nc1Sc1ccnc(N2CC(S(C)(=O)=O)C2)c1Cl] |
| A-285 | 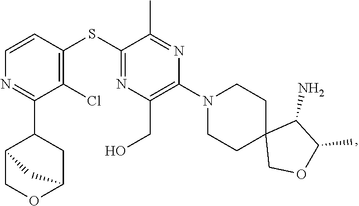 [SMILES: Cc1nc(N2CCC3(CC2)CO[C@@H](C)[C@H]3N)c(CO)nc1Sc1ccnc(C2C[C@@H]3C[C@H]2CO3)c1Cl] |
| A-286 | 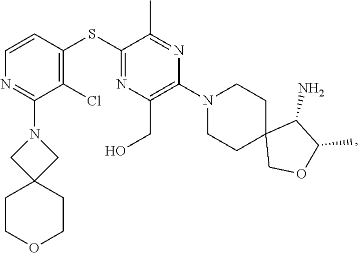 [SMILES: Cc1nc(N2CCC3(CC2)CO[C@@H](C)[C@H]3N)c(CO)nc1Sc1ccnc(N2CC3(CCOCC3)C2)c1Cl] |
| A-287 | 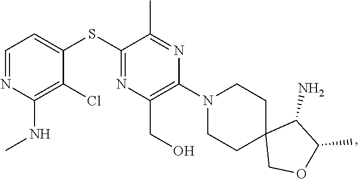 [SMILES: CNc1nccc(Sc2nc(CO)c(N3CCC4(CC3)CO[C@@H](C)[C@H]4N)nc2C)c1Cl] |
| A-288 | 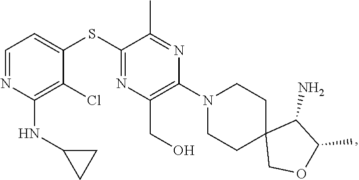 [SMILES: Cc1nc(N2CCC3(CC2)CO[C@@H](C)[C@H]3N)c(CO)nc1Sc1ccnc(NC2CC2)c1Cl] |
| A-289 | 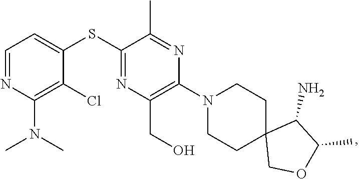 [SMILES: Cc1nc(N2CCC3(CC2)CO[C@@H](C)[C@H]3N)c(CO)nc1Sc1ccnc(N(C)C)c1Cl] |
| A-290 | 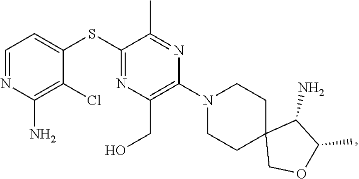 [SMILES: Cc1nc(N2CCC3(CC2)CO[C@@H](C)[C@H]3N)c(CO)nc1Sc1ccnc(N)c1Cl] |
| A-291 | 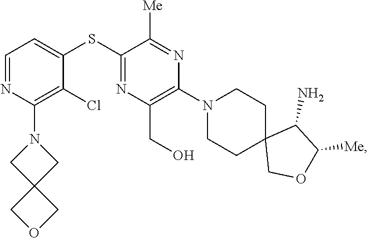 [SMILES: Cc1nc(N2CCC3(CC2)CO[C@@H](C)[C@H]3N)c(CO)nc1Sc1ccnc(N2CC3(COC3)C2)c1Cl] |
| A-292 | 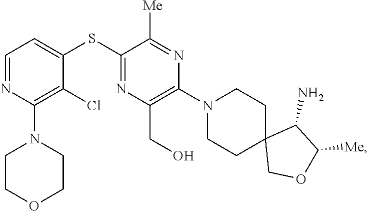 [SMILES: Cc1nc(N2CCC3(CC2)CO[C@@H](C)[C@H]3N)c(CO)nc1Sc1ccnc(N2CCOCC2)c1Cl] |
| A-293 | 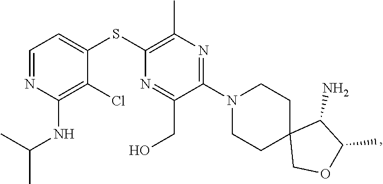 [SMILES: Cc1nc(N2CCC3(CC2)CO[C@@H](C)[C@H]3N)c(CO)nc1Sc1ccnc(NC(C)C)c1Cl] |
| A-294 | 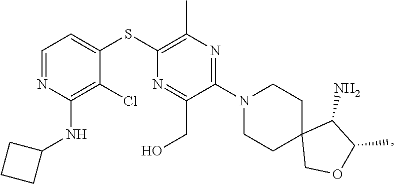 [SMILES: Cc1nc(N2CCC3(CC2)CO[C@@H](C)[C@H]3N)c(CO)nc1Sc1ccnc(NC2CCC2)c1Cl] |
| A-295 | 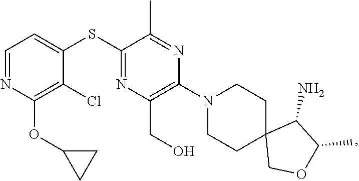 [SMILES: Cc1nc(N2CCC3(CC2)CO[C@@H](C)[C@H]3N)c(CO)nc1Sc1ccnc(OC2CC2)c1Cl] |
| A-296 | 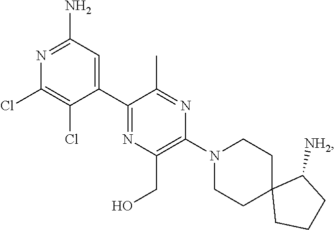 [SMILES: Cc1nc(N2CCC3(CCC[C@H]3N)CC2)c(CO)nc1-c1cc(N)nc(Cl)c1Cl] |
| A-297 | 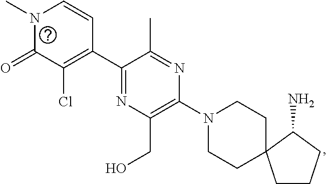 [SMILES: Cc1nc(N2CCC3(CCC[C@H]3N)CC2)c(CO)nc1-c1ccn(C)c(=O)c1Cl] |
| A-298 | 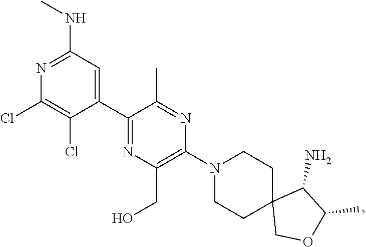 [SMILES: CNc1cc(-c2nc(CO)c(N3CCC4(CC3)CO[C@@H](C)[C@H]4N)nc2C)c(Cl)c(Cl)n1] |
| A-299 | 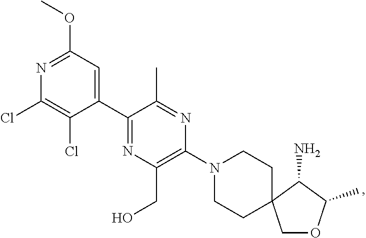 [SMILES: COc1cc(-c2nc(CO)c(N3CCC4(CC3)CO[C@@H](C)[C@H]4N)nc2C)c(Cl)c(Cl)n1] |
| A-300 | 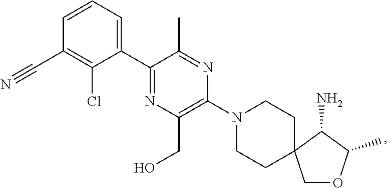 [SMILES: Cc1nc(N2CCC3(CC2)CO[C@@H](C)[C@H]3N)c(CO)nc1-c1cccc(C#N)c1Cl] |
| A-301 | 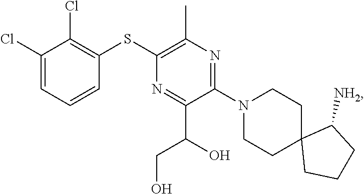 [SMILES: Cc1nc(N2CCC3(CCC[C@H]3N)CC2)c(C(O)CO)nc1Sc1cccc(Cl)c1Cl] |
| A-302 | 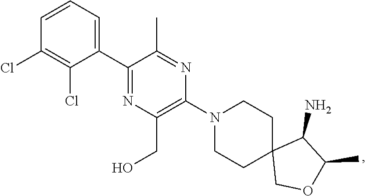 [SMILES: Cc1nc(N2CCC3(CC2)CO[C@H](C)[C@@H]3N)c(CO)nc1-c1cccc(Cl)c1Cl] |
| A-303 | 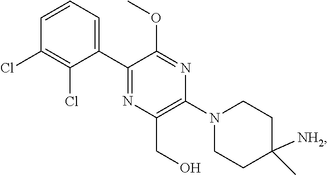 [SMILES: COc1nc(N2CCC(C)(N)CC2)c(CO)nc1-c1cccc(Cl)c1Cl] |
| A-304 | 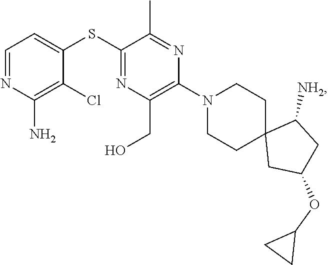 [SMILES: Cc1nc(N2CCC3(CC2)C[C@@H](OC2CC2)C[C@H]3N)c(CO)nc1Sc1ccnc(N)c1Cl] |
| A-305 | 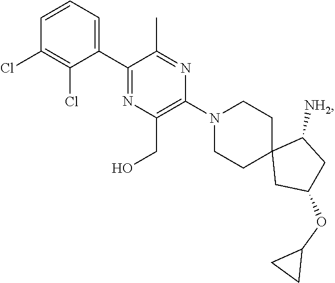 [SMILES: Cc1nc(N2CCC3(CC2)C[C@@H](OC2CC2)C[C@H]3N)c(CO)nc1-c1cccc(Cl)c1Cl] |
| A-306 | 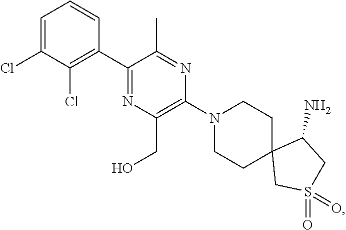 [SMILES: Cc1nc(N2CCC3(CC2)CS(=O)(=O)C[C@H]3N)c(CO)nc1-c1cccc(Cl)c1Cl] |
| A-307 | 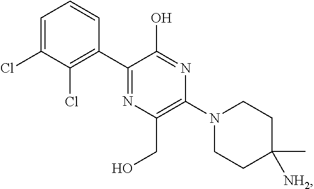 [SMILES: CC1(N)CCN(c2nc(O)c(-c3cccc(Cl)c3Cl)nc2CO)CC1] |
| A-308 | 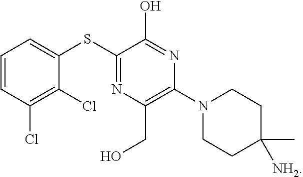 [SMILES: CC1(N)CCN(c2nc(O)c(Sc3cccc(Cl)c3Cl)nc2CO)CC1] |

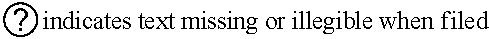

The disclosure provides compounds, and pharmaceutically acceptable salts, prodrugs, solvates, hydrates, tautomers, or isomers thereof, in Table 2.

TABLE 2
Structure

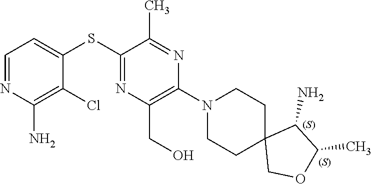

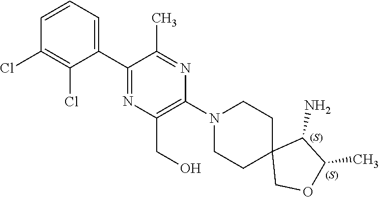

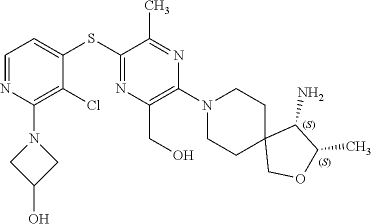

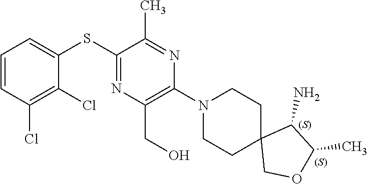

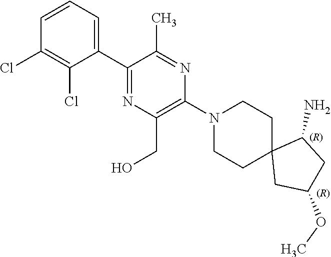

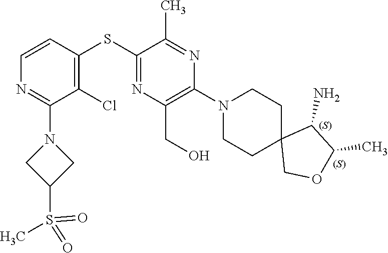

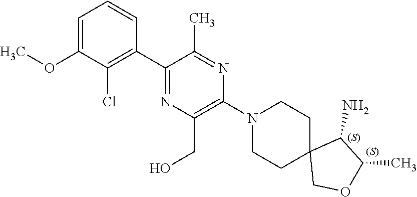

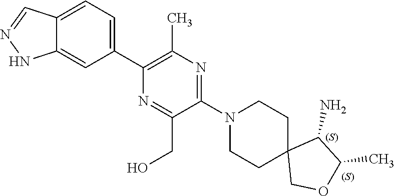

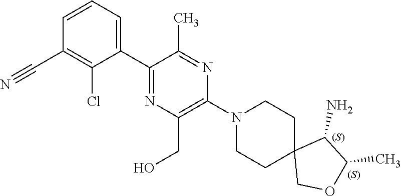

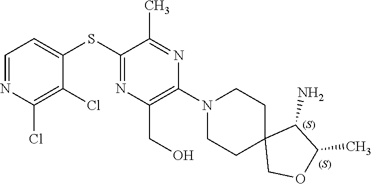

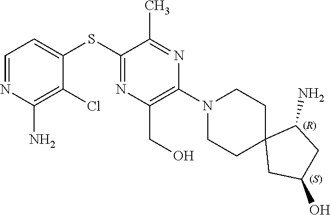

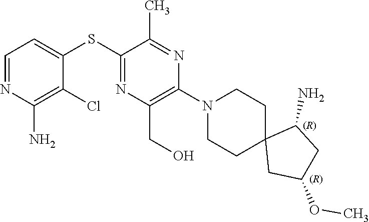

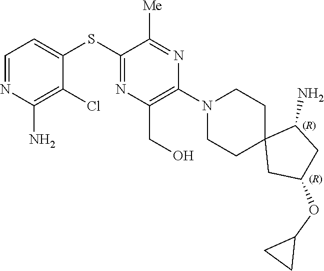

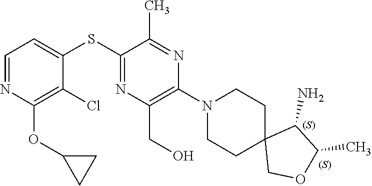

The term “aryl” refers to cyclic, aromatic hydrocarbon groups that have 1 to 2 aromatic rings, including monocyclic or bicyclic groups such as phenyl, biphenyl or naphthyl. Where containing two aromatic rings (bicyclic, etc.), the aromatic rings of the aryl group may be joined at a single point (e.g., biphenyl), or fused (e.g., naphthyl). The aryl group may be optionally substituted by one or more substituents, e.g., 1 to 5 substituents, at any point of attachment. Exemplary substituents include, but are not limited to, —H, halogen, —O—C₁-C₆ alkyl, —C₁-C₆ alkyl, —OC₂-C₆ alkenyl, —OC₂-C₆ alkynyl, —C₂-C₆ alkenyl, —C₂-C₆ alkynyl, —OH, —OP(O)(OH)₂, —OC(O)C₁-C₆ alkyl, —C(O)C₁-C₆ alkyl, —OC(O)OC₁-C₆ alkyl, —NH₂, —NH(C₁-C₆ alkyl), —N(C₁-C₆ alkyl)₂, —S(O)₂—C₁-C₆ alkyl, —S(O)NHC₁-C₆ alkyl, and —S(O)N(C₁-C₆ alkyl)₂. The substituents can themselves be optionally substituted.

Unless otherwise specifically defined, “heteroaryl” means a monovalent or multivalent monocyclic aromatic radical or a polycyclic aromatic radical of 5 to 24 ring atoms, containing one or more ring heteroatoms selected from N, S, P, and O, the remaining ring atoms being C. Heteroaryl as herein defined also means a bicyclic heteroaromatic group wherein the heteroatom is selected from N, S, P, and O. The aromatic radical is optionally substituted independently with one or more substituents described herein. Examples include, but are not limited to, furyl, thienyl, pyrrolyl, pyridyl, pyrazolyl, pyrimidinyl, imidazolyl, isoxazolyl, oxazolyl, oxadiazolyl, pyrazinyl, indolyl, thiophen-2-yl, quinolyl, benzopyranyl, isothiazolyl, thiazolyl, thiadiazolyl, benzo[d]imidazolyl, thieno[3,2-b]thiophene, triazolyl, triazinyl, imidazo[1,2-b]pyrazolyl, furo[2,3-c]pyridinyl, imidazo[1,2-a]pyridinyl, indazolyl, 1-methyl-1H-indazolyl, pyrrolo[2,3-c]pyridinyl, pyrrolo[3,2-c]pyridinyl, pyrazolo[3,4-c]pyridinyl, thieno[3,2-c]pyridinyl, thieno[2,3-c]pyridinyl, thieno[2,3-b]pyridinyl, benzothiazolyl, indolyl, indolinyl, indolinonyl, dihydrobenzothiophenyl, dihydrobenzofuranyl, benzofuran, chromanyl, thiochromanyl, tetrahydroquinolinyl, dihydrobenzothiazine, dihydrobenzoxanyl, quinolinyl, isoquinolinyl, 1,6-naphthyridinyl, benzo[de]isoquinolinyl, pyrido[4,3-b][1,6]naphthyridinyl, thieno[2,3-b]pyrazinyl, quinazolinyl, tetrazolo[1,5-a]pyridinyl, [1,2,4]triazolo[4,3-a]pyridinyl, isoindolyl, isoindolin-1-one, indolin-2-one, pyrrolo[2,3-b]pyridinyl, pyrrolo[3,4-b]pyridinyl, pyrrolo[3,2-b]pyridinyl, imidazo[5,4-b]pyridinyl, pyrrolo[1,2-a]pyrimidinyl, tetrahydropyrrolo[1,2-a]pyrimidinyl, 3,4-dihydro-2H-1□²-pyrrolo[2,1-b]pyrimidine, dibenzo[b,d]thiophene, pyridin-2-one, furo[3,2-c]pyridinyl, furo[2,3-c]pyridinyl, 1H-pyrido[3,4-b][1,4]thiazinyl, 2-methylbenzo[d]oxazolyl, 1,2,3,4-tetrahydropyrrolo[1,2-a]pyrimidyl, 2,3-dihydrobenzofuranyl, benzooxazolyl, benzoisoxazolyl, benzo[d]isoxazolyl, benzo[d]oxazolyl, furo[2,3-b]pyridinyl, benzothiophenyl, 1,5-naphthyridinyl, furo[3,2-b]pyridinyl, [1,2,4]triazolo[1,5-a]pyridinyl, benzo[1,2,3]triazolyl, 1-methyl-1H-benzo[d][1,2,3]triazolyl, imidazo[1,2-a]pyrimidinyl, [1,2,4]triazolo[4,3-b]pyridazinyl, quinoxalinyl, benzo[c][1,2,5]thiadiazolyl, benzo[c][1,2,5]oxadiazolyl, 1,3-dihydro-2H-benzo[d]imidazol-2-one, 3,4-dihydro-2H-pyrazolo[1,5-b][1,2]oxazinyl, 3,4-dihydro-2H-benzo[b][1,4]oxazinyl, 4,5,6,7-tetrahydropyrazolo[1,5-a]pyridinyl, thiazolo[5,4-d]thiazolyl, imidazo[2,1-b][1,3,4]thiadiazolyl, thieno[2,3-b]pyrrolyl, 3H-indolyl, benzo[d][1,3]dioxolyl, pyrazolo[1,5-a]pyridinyl, and derivatives thereof.

“Alkyl” refers to a straight or branched chain saturated hydrocarbon. C₁-C₆ alkyl groups contain 1 to 6 carbon atoms. Examples of a C₁-C₆ alkyl group include, but are not limited to, methyl, ethyl, propyl, butyl, pentyl, isopropyl, isobutyl, sec-butyl and tert-butyl, isopentyl and neopentyl.

The term “alkenyl” means an aliphatic hydrocarbon group containing a carbon carbon double bond and which may be straight or branched having about 2 to about 6 carbon atoms in the chain. Certain alkenyl groups have 2 to about 4 carbon atoms in the chain.

Branched means that one or more lower alkyl groups such as methyl, ethyl, or propyl are attached to a linear alkenyl chain. Exemplary alkenyl groups include ethenyl, propenyl, n-butenyl, and i-butenyl. A C₂-C₆ alkenyl group is an alkenyl group containing between 2 and 6 carbon atoms.

The term “alkynyl” means an aliphatic hydrocarbon group containing a carbon carbon triple bond and which may be straight or branched having about 2 to about 6 carbon atoms in the chain. Certain alkynyl groups have 2 to about 4 carbon atoms in the chain. Branched means that one or more lower alkyl groups such as methyl, ethyl, or propyl are attached to a linear alkynyl chain. Exemplary alkynyl groups include ethynyl, propynyl, n-butynyl, 2-butynyl, 3-methylbutynyl, and n-pentynyl. A C₂-C₆ alkynyl group is an alkynyl group containing between 2 and 6 carbon atoms.

The term “cycloalkyl” means monocyclic or polycyclic saturated carbon rings containing 3-18 carbon atoms. Examples of cycloalkyl groups include, without limitations, cyclopropyl, cyclobutyl, cyclopentyl, cyclohexyl, cycloheptanyl, cyclooctanyl, norbornanyl, norbornenyl, bicyclo[2.2.2]octanoyl, or bicyclo[2.2.2]octenyl. A C₃-C₈ cycloalkyl is a cycloalkyl group containing between 3 and 8 carbon atoms. A cycloalkyl group can be fused (e.g., decalin) or bridged (e.g., norbornane).

The term “cycloalkenyl” means monocyclic, non-aromatic unsaturated carbon rings containing 4-18 carbon atoms. Examples of cycloalkenyl groups include, without limitation, cyclopentenyl, cyclohexenyl, cycloheptenyl, cyclooctenyl, and norbornenyl. A C₄-C₈ cycloalkenyl is a cycloalkenyl group containing between 4 and 8 carbon atoms.

In some embodiments, the terms “heterocyclyl” or “heterocycloalkyl” or “heterocycle” refer to monocyclic or polycyclic 3 to 24-membered rings containing carbon and heteroatoms selected from oxygen, phosphorus, nitrogen, and sulfur and wherein there are no delocalized π electrons (aromaticity) shared among the ring carbon or heteroatoms. Heterocyclyl rings include, but are not limited to, oxetanyl, azetidinyl, tetrahydrofuranyl, pyrrolidinyl, oxazolinyl, oxazolidinyl, thiazolinyl, thiazolidinyl, pyranyl, thiopyranyl, tetrahydropyranyl, dioxalinyl, piperidinyl, morpholinyl, thiomorpholinyl, thiomorpholinyl S-oxide, thiomorpholinyl S-dioxide, piperazinyl, azepinyl, oxepinyl, diazepinyl, tropanyl, and homotropanyl. A heteroycyclyl or heterocycloalkyl ring can also be fused or bridged, e.g., can be a bicyclic ring.

In some embodiments “heterocyclyl” or “heterocycloalkyl” or “heterocycle” is a saturated, partially saturated or unsaturated, mono or bicyclic ring containing 3-24 atoms of which at least one atom is chosen from nitrogen, sulfur or oxygen, which may, unless otherwise specified, be carbon or nitrogen linked, wherein a —CH₂— group can optionally be replaced by a —C(O)— or a ring sulfur atom may be optionally oxidised to form the S-oxides. “Heterocyclyl” can be a saturated, partially saturated or unsaturated, mono or bicyclic ring containing 5 or 6 atoms of which at least one atom is chosen from nitrogen, sulfur or oxygen, which may, unless otherwise specified, be carbon or nitrogen linked, wherein a —CH₂— group can optionally be replaced by a —C(O)— or a ring sulfur atom may be optionally oxidised to form S-oxide(s). Non-limiting examples and suitable values of the term “heterocyclyl” are thiazolidinyl, pyrrolidinyl, pyrrolinyl, 2-pyrrolidonyl, 2,5-dioxopyrrolidinyl, 2-benzoxazolinonyl, 1,1-dioxotetrahydro thienyl, 2,4-dioxoimidazolidinyl, 2-oxo-1,3,4-(4-triazolinyl), 2-oxazolidinonyl, 5,6-dihydro uracilyl, 1,3-benzodioxolyl, 1,2,4-oxadiazolyl, 2-azabicyclo[2.2.1]heptyl, 4-thiazolidonyl, morpholino, 2-oxotetrahydrofuranyl, tetrahydrofuranyl, 2,3-dihydrobenzofuranyl, benzothienyl, tetrahydropyranyl, piperidyl, 1-oxo-1,3-dihydroisoindolyl, piperazinyl, thiomorpholino, 1,1-dioxothiomorpholino, tetrahydropyranyl, 1,3-dioxolanyl, homopiperazinyl, thienyl, isoxazolyl, imidazolyl, pyrrolyl, thiadiazolyl, isothiazolyl, 1,2,4-triazolyl, 1,3,4-triazolyl, pyranyl, indolyl, pyrimidyl, thiazolyl, pyrazinyl, pyridazinyl, pyridyl, 4-pyridonyl, quinolyl and 1-isoquinolonyl.

As used herein, the term “halo” or “halogen” means a fluoro, chloro, bromo, or iodo group.

The term “carbonyl” refers to a functional group comprising a carbon atom double-bonded to an oxygen atom. It can be abbreviated herein as “oxo,” as C(O), or as C═O.

“Spirocycle” or “spirocyclic” means carbogenic bicyclic ring systems with both rings connected through a single atom. The ring can be different in size and nature, or identical in size and nature. Examples include spiropentane, spirohexane, spiroheptane, spirooctane, spirononane, or spirodecane. One or both of the rings in a spirocycle can be fused to another carbocyclic, heterocyclic, aromatic, or heteroaromatic ring. One or more of the carbon atoms in the spirocycle can be substituted with a heteroatom (e.g., O, N, S, or P). A C₅-C₁₂ spirocycle is a spirocycle containing between 5 and 12 carbon atoms. In some embodiments, a C₅-C₁₂ spirocycle is a spirocycle containing from 5 to 12 carbon atoms. One or more of the carbon atoms can be substituted with a heteroatom.

The term “spirocyclic heterocycle,” “spiroheterocyclyl,” or “spiroheterocycle” is understood to mean a spirocycle wherein at least one of the rings is a heterocycle (e.g., at least one of the rings is furanyl, morpholinyl, or piperadinyl). A spirocyclic heterocycle can contain between 5 and 12 atoms, at least one of which is a heteroatom selected from N, O, S and P. In some embodiments, a spirocyclic heterocycle can contain from 5 to 12 atoms, at least one of which is a heteroatom selected from N, O, S and P.

The term “tautomers” refers to a set of compounds that have the same number and type of atoms, but differ in bond connectivity and are in equilibrium with one another. A “tautomer” is a single member of this set of compounds. A single tautomer may be drawn but it is understood that this single structure is meant to represent all possible tautomers that might exist. Examples include enol-ketone tautomerism. When a ketone is drawn it is understood that both the enol and ketone forms are part of the disclosure.

The SHP2 inhibitor may be administered alone as a monotherapy or in combination with one or more other therapeutic agent (e.g., an inhibitor of a MAP kinase pathway or an anti-cancer therapeutic agent) as a combination therapy. The SHP2 inhibitor may be administered as a pharmaceutical composition. The SHP2 inhibitor may be administered before, after, and/or concurrently with the one or more other therapeutic agent (e.g., an inhibitor of a MAP kinase pathway or an anti-cancer therapeutic agent). If administered concurrently with the one or more other therapeutic agent, such administration may be simultaneous (e.g., in a single composition) or may be via two or more separate compositions, optionally via the same or different modes of administration (e.g., local, systemic, oral, intravenous, etc.). In some embodiments, the SHP2 inhibitor may be administered in combination with a cancer immunotherapy, radiation therapy, and/or with surgical tumor resection and additionally or alternatively with one or more other therapeutic agent (e.g., an inhibitor of a MAP kinase pathway or an anti-cancer therapeutic agent).

### Therapeutic Methods

In some embodiments of the methods of the disclosure, administration of the disclosed compositions and compounds (e.g., SHP2 inhibitors and/or other therapeutic agents) can be accomplished via any mode of administration for therapeutic agents. These modes include systemic or local administration such as oral, nasal, parenteral, transdermal, subcutaneous, vaginal, buccal, rectal or topical administration modes.

Depending on the intended mode of administration, the disclosed compounds or pharmaceutical compositions can be in solid, semi-solid or liquid dosage form, such as, for example, injectables, tablets, suppositories, pills, time-release capsules, elixirs, tinctures, emulsions, syrups, powders, liquids, suspensions, or the like, sometimes in unit dosages and consistent with conventional pharmaceutical practices. Likewise, they can also be administered in intravenous (both bolus and infusion), intraperitoneal, subcutaneous or intramuscular form, and all using forms well known to those skilled in the pharmaceutical arts. Pharmaceutical compositions suitable for the delivery of a SHP2 inhibitor (alone or, e.g., in combination with another therapeutic agent according to the present disclosure) and methods for their preparation will be readily apparent to those skilled in the art. Such compositions and methods for their preparation may be found, e.g., in Remington's Pharmaceutical Sciences, 19th Edition (Mack Publishing Company, 1995), incorporated herein in its entirety.

Illustrative pharmaceutical compositions are tablets and gelatin capsules comprising a SHP2 inhibitor alone or in combination with another therapeutic agent according to the disclosure and a pharmaceutically acceptable carrier, such as: a) a diluent, e.g., purified water, triglyceride oils, such as hydrogenated or partially hydrogenated vegetable oil, or mixtures thereof, corn oil, olive oil, sunflower oil, safflower oil, fish oils, such as EPA or DHA, or their esters or triglycerides or mixtures thereof, omega-3 fatty acids or derivatives thereof, lactose, dextrose, sucrose, mannitol, sorbitol, cellulose, sodium, saccharin, glucose and/or glycine; b) a lubricant, e.g., silica, talcum, stearic acid, its magnesium or calcium salt, sodium oleate, sodium stearate, magnesium stearate, sodium benzoate, sodium acetate, sodium chloride and/or polyethylene glycol; for tablets also; c) a binder, e.g., magnesium aluminum silicate, starch paste, gelatin, tragacanth, methylcellulose, sodium carboxymethylcellulose, magnesium carbonate, natural sugars such as glucose or beta-lactose, corn sweeteners, natural and synthetic gums such as acacia, tragacanth or sodium alginate, waxes and/or polyvinylpyrrolidone, if desired; d) a disintegrant, e.g., starches, agar, methyl cellulose, bentonite, xanthan gum, alginic acid or its sodium salt, or effervescent mixtures; e) absorbent, colorant, flavorant and sweetener; f) an emulsifier or dispersing agent, such as Tween® 80, Labrasol®, HPMC, DOSS, caproyl 909, labrafac, labrafil, peceol, transcutol, capmul MCM, capmul PG-12, captex 355, gelucire, vitamin E TGPS or other acceptable emulsifier; and/or g) an agent that enhances absorption of the compound such as cyclodextrin, hydroxypropyl-cyclodextrin, PEG400, PEG200.

Liquid, particularly injectable, compositions can be prepared by dissolution, dispersion, etc. For example, a SHP2 inhibitor (alone or in combination with another therapeutic agent according to the disclosure) is dissolved in or mixed with a pharmaceutically acceptable solvent such as, for example, water, saline, aqueous dextrose, glycerol, ethanol, and the like, to thereby form an injectable isotonic solution or suspension. Proteins such as albumin, chylomicron particles, or serum proteins can be used to solubilize the SHP2 inhibitor (alone or in combination with another therapeutic agent according to the disclosure).

The SHP2 inhibitor can be also formulated as a suppository, alone or in combination with another therapeutic agent according to the disclosure, which can be prepared from fatty emulsions or suspensions; using polyalkylene glycols such as propylene glycol, as the carrier.

The SHP2 inhibitor can also be administered in the form of liposome delivery systems, such as small unilamellar vesicles, large unilamellar vesicles and multilamellar vesicles, either alone or in combination with another therapeutic agent according to the disclosure. Liposomes can be formed from a variety of phospholipids, containing cholesterol, stearylamine or phosphatidylcholines. In some embodiments, a film of lipid components is hydrated with an aqueous solution of drug to a form lipid layer encapsulating the drug, as described for instance in U.S. Pat. No. 5,262,564, the contents of which are hereby incorporated by reference.

SHP2 inhibitors can also be delivered by the use of monoclonal antibodies as individual carriers to which the disclosed compounds are coupled. SHP2 inhibitors can also be coupled with soluble polymers as targetable drug carriers. Such polymers can include polyvinylpyrrolidone, pyran copolymer, polyhydroxypropylmethacrylamide-phenol, polyhydroxyethylaspanamidephenol, or polyethyleneoxidepolylysine substituted with palmitoyl residues. Furthermore, a SHP2 inhibitor can be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels. In one embodiment, disclosed compounds are not covalently bound to a polymer, e.g., a polycarboxylic acid polymer, or a polyacrylate.

Parental injectable administration is generally used for subcutaneous, intramuscular or intravenous injections and infusions. Injectables can be prepared in conventional forms, either as liquid solutions or suspensions or solid forms suitable for dissolving in liquid prior to injection.

### Pharmaceutical Formulations

Another aspect of the invention relates to a pharmaceutical composition comprising a SHP2 inhibitor (alone or in combination with another therapeutic agent according to the present disclosure) and a pharmaceutically acceptable carrier. The pharmaceutically acceptable carrier can further include an excipient, diluent, or surfactant.

Thus, the present disclosure provides compositions (e.g., pharmaceutical compositions) comprising one or more SHP2 inhibitor for use in a method disclosed herein, e.g., a SHP2 monotherapy. Such compositions may comprise a SHP2 inhibitor and, e.g., one or more carrier, excipient, diluent, and/or surfactant.

The disclosure provides compositions (e.g., pharmaceutical compositions) comprising one or more SHP2 inhibitor and one or more additional therapeutic agent for use in a method disclosed herein, e.g., a SHP2 combination therapy. Such compositions may comprise a SHP2 inhibitor, an additional therapeutic agent (e.g., a TKI, a MAPK pathway inhibitor, an EGFR inhibitor, an ALK inhibitor, a MEK inhibitor) and, e.g., one or more carrier, excipient, diluent, and/or surfactant.

The present disclosure provides compositions (e.g., pharmaceutical compositions) comprising one or more SHP2 inhibitors and one or more MEK inhibitors for use in a method disclosed herein, e.g., a SHP2 combination therapy. Such compositions may comprise a SHP2 inhibitor, a MEK inhibitor and, e.g., one or more carrier, excipient, diluent, and/or surfactant. Such compositions may consist essentially of a SHP2 inhibitor, a MEK inhibitor and, e.g., one or more carrier, excipient, diluent, and/or surfactant. Such compositions may consist of a SHP2 inhibitor, a MEK inhibitor and, e.g., one or more carrier, excipient, diluent, and/or surfactant. For example, one non-limiting example of a composition of the present disclosure may comprise, consist essentially of, or consist of (a) a SHP2 inhibitor; (b) a MEK inhibitor selected from one or more of Trametinib (GSK1120212); Selumetinib (AZD6244); Cobimetinib (GDC-0973/XL581), Binimetinib, Vemurafenib, Pimasertib, TAK733, RO4987655 (CH4987655); CI-1040; PD-0325901; Refametinib (RDEA 119/BAY 86-9766); RO5126766, AZD8330 (ARRY-424704/ARRY-704); and GSK1120212; and (c) one or more carrier, excipient, diluent, and/or surfactant. Another non-limiting example of a composition of the present disclosure may comprise, consist essentially of, or consist of (a) a MEK inhibitor; (b) a SHP2 inhibitor selected from (i) RMC-3943; (ii) RMC-4550; (iii) SHP099; (iv) a SHP2 inhibitor compound of any one of Formula I, of Formula II, of Formula III, of Formula I-V1, of Formula I-V2, of Formula I-W, of Formula I-X, of Formula I-Y, of Formula I-Z, of Formula IV, of Formula V, of Formula VI, of Formula IV-X, of Formula IV-Y, of Formula IV-Z, of Formula VII, of Formula VIII, of Formula IX, and of Formula X disclosed herein; (v) TNO155; (vi) a compound from Table 1, disclosed herein; (vii) a compound from Table 2, disclosed herein, (viii) RLY-1971; and (ix) a combination thereof, and (c) one or more carrier, excipient, diluent, and/or surfactant.

Compositions can be prepared according to conventional mixing, granulating or coating methods, respectively, and the present pharmaceutical compositions can contain from about 0.1% to about 99%, from about 5% to about 90%, or from about 1% to about 20% of the disclosed therapeutic agent by weight or volume. Accordingly, such compositions may contain from about 0.1% to about 99%, from about 5% to about 90%, or from about 1% to about 20% of a SHP2 inhibitor by weight or volume. Compositions may contain from about 0.1% to about 99%, from about 5% to about 90%, or from about 1% to about 20% of a SHP2 inhibitor compound listed in Table 1 by weight or volume. Compositions may contain from about 0.1% to about 99%, from about 5% to about 90%, or from about 1% to about 20% of a SHP2 inhibitor compound listed in Table 2 by weight or volume. Compositions may contain from about 0.1% to about 99%, from about 5% to about 90%, or from about 1% to about 20% of a combination of two or more SHP2 inhibitors by weight or volume, e.g., of a SHP2 inhibitor and one or more additional SHP2 inhibitor that may be the same or different by weight or by volume.

The dosage regimen utilizing the disclosed compound is selected in accordance with a variety of factors including type, species, age, weight, sex and medical condition of the patient; the severity of the condition to be treated; the route of administration; the renal or hepatic function of the patient; and the particular disclosed compound employed. A physician or veterinarian of ordinary skill in the art can readily determine and prescribe the effective amount of the drug required to prevent, counter or arrest the progress of the condition.

Effective dosage amounts of a SHP2 inhibitor, when used for the indicated effects, range from about 0.5 mg to about 5000 mg as needed to treat the condition. Compositions for in vivo or in vitro use can contain about 0.5, 5, 20, 50, 75, 100, 150, 250, 500, 750, 1000, 1250, 2500, 3500, or 5000 mg of the disclosed compound, or, in a range of from one amount to another amount in the list of doses. In one embodiment, the compositions are in the form of a tablet that can be scored.

### Kits

The disclosure provides kits for treating a disease or disorder with a SHP2 inhibitor, one or more carrier, excipient, diluent, and/or surfactant, and a means for determining whether a sample from a subject (e.g., a tumor sample) is likely to be sensitive to SHP2 treatment. In some embodiments, the means for determining comprises a means for determining whether the sample comprises an RTK fusion. In some embodiments, the means for determining comprises a means for determining whether the sample comprises and RTK fusion that activates the MAPK pathway. In some embodiments, the means for determining comprises a means for determining whether the sample comprises any of the RTK fusion mutations described herein. Such means include, but are not limited to direct sequencing, and utilization of a high-sensitivity diagnostic assay (with CE-IVD mark), e.g., as described in Domagala, et al., Pol J Pathol 3: 145-164 (2012), incorporated herein by reference in its entirety, including TheraScreen® PCR; AmoyDx; PNAClamp; RealQuality; EntroGen; LightMix; StripAssay®; Hybcell plexA; Devyser; Surveyor; Cobas; and TheraScreen Pyro. In some embodiments, the means for determining comprises a means for determining whether a sample that comprises an RTK fusion mutations described herein activates the MAPK pathway. Thus, the means may be an immunoblot; immunofluorescence; or ELISA.

pERK Assay

SHP2 inhibition with RMC-4630 inhibits ERK phosphorylation (pERK) and proliferation in vitro. Inhibition of pERK may be used as an assay monitoring or determining efficacy of treatment with a SHP2 inhibitor of the disclosure.

Without wishing to be bound by theory, SHP may be allosterically activated through binding of bis-tyrosyl-phosphorylated peptides to its Src Homology 2 (SH2) domains. The latter activation step leads to the release of the auto-inhibitory interface of SHP2, which in turn renders the SHP2 protein tyrosine phosphatase (PTP) active and available for substrate recognition and reaction catalysis. The catalytic activity of SHP2 was monitored using the surrogate substrate DiFMUP in a prompt fluorescence assay format.

The phosphatase reactions were performed at room temperature in 96-well black polystyrene plate, flat bottom, non-binding surface (Corning, Cat #3650) using a final reaction volume of 100 μL and the following assay buffer conditions: 50 mM HEPES, pH 7.2, 100 mM NaCl, 0.5 mM EDTA, 0.05% P-20, 1 mM DTT.

The inhibition of SHP2 by RMC-4630 was monitored using an assay in which 0.2 nM of SHP2 was incubated with 0.5 μM of Activating Peptide 1 (sequence: H2N-LN(pY)IDLDLV(dPEG8)LST(pY)ASINFQK-amide)(SEQ ID NO: 1) or Activating Peptide 2 (sequence: H2N-LN(pY)AQLWHA(dPEG8)LTI(pY)ATIRRF-amide) (SEQ ID NO: 2). After 30-60 minutes incubation at 25° C., the surrogate substrate DiFMUP (Invitrogen, Cat #D6567) was added to the reaction and activity was determined by a kinetic read using a microplate reader (Envision, Perkin-Elmer or Spectramax M5, Molecular Devices). The excitation and emission wavelengths were 340 nm and 450 nm, respectively. Initial rates were determined from a linear fit of the data, and the inhibitor dose response curves were analyzed using normalized IC₅₀ regression curve fitting with control based normalization. Using this exemplary and non-limiting protocol, SHP2 inhibition by a SHP2 inhibitor of the disclosure, including RMC-4630, may be determined.

### Methods and Definitions

The practice of the methods of the disclosure may employ, unless otherwise indicated, techniques of cell culturing, molecular biology (including recombinant techniques), microbiology, cell biology, biochemistry and immunology, which are explained in at least one embodiment in the literature, such as, Molecular Cloning: A Laboratory Manual, third edition (Sambrook et al., 2001) Cold Spring Harbor Press; Oligonucleotide Synthesis (P. Herdewijn, ed., 2004); Animal Cell Culture (R. I. Freshney), ed., 1987); Methods in Enzymology (Academic Press, Inc.); Handbook of Experimental Immunology (D. M. Weir & C. C. Blackwell, eds.); Gene Transfer Vectors for Mammalian Cells (J. M. Miller & M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Manual of Clinical Laboratory Immunology (B. Detrick, N. R. Rose, and J. D. Folds eds., 2006); Immunochemical Protocols (J. Pound, ed., 2003); Lab Manual in Biochemistry: Immunology and Biotechnology (A. Nigam and A. Ayyagari, eds. 2007); Immunology Methods Manual: The Comprehensive Sourcebook of Techniques (Ivan Lefkovits, ed., 1996); Using Antibodies: A Laboratory Manual (E. Harlow and D. Lane, eds., 1988); and others.

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, particular methods and materials are described. For the purposes of the present invention, the following terms are defined below.

The articles “a” and “an” are used in this disclosure to refer to one or more than one (i.e., to at least one) of the grammatical object of the article. By way of example, “an element” means one element or more than one element.

The term “and/or” is used in this disclosure to mean either “and” or “or” unless indicated otherwise.

By “optional” or “optionally,” it is meant that the subsequently described event or circumstance may or may not occur, and that the description includes instances where the event or circumstance occurs and instances in which it does not. For example, “optionally substituted aryl” encompasses both “aryl” and “substituted aryl” as defined herein. It will be understood by those ordinarily skilled in the art, with respect to any group containing one or more substituents, that such groups are not intended to introduce any substitution or substitution patterns that are sterically impractical, synthetically non-feasible, and/or inherently unstable.

The term “administer”, “administering”, or “administration” as used in this disclosure refers to either directly administering a disclosed compound or pharmaceutically acceptable salt of the disclosed compound or a composition to a subject, or administering a prodrug derivative or analog of the compound or pharmaceutically acceptable salt of the compound or composition to the subject, which can form an equivalent amount of active compound within the subject's body.

The term “Sample” or “biological sample,” as used herein, refers to a sample obtained from a subject, e.g., a human subject or a patient, which may be tested for an abundance or an activity of a particular molecule. Samples may include, but are not limited to, biopsies, tissues, cells, buccal swab sample, body fluids, including blood, serum, plasma, urine, saliva, cerebral spinal fluid, tears, pleural fluid and the like. In some embodiments, the samples that are suitable for use in the methods described herein contain genetic material, e.g., genomic DNA (gDNA). In some embodiments, the samples contain nucleotides, e.g., RNA (e.g., mRNA) or cDNA derived from mRNA. In some embodiments, the samples contain protein. Methods and reagents are known in the art for obtaining, processing, and analyzing samples. The sample may be further processed before the detecting step. For example, DNA or protein in a cell or tissue sample can be separated from other components of the sample. The sample can be concentrated and/or purified to isolate DNA and/or protein. Cells can be harvested from a biological sample using standard techniques known in the art. For example, cells can be harvested by centrifuging a cell sample and resuspending the pelleted cells. The cells can be resuspended in a buffered solution such as phosphate-buffered saline (PBS). After centrifuging the cell suspension to obtain a cell pellet, the cells can be lysed to extract DNA, e.g., genomic DNA, and/or protein. All samples obtained from a subject, including those subjected to any sort of further processing, are considered to be obtained from the subject.

The term “carrier”, as used in this disclosure, encompasses carriers, excipients, and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body of a subject.

The term SHP099 refers to a SHP2 inhibitor having the following structure:

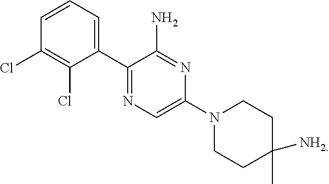 [SMILES: CC1(N)CCN(c2cnc(-c3cccc(Cl)c3Cl)c(N)n2)CC1]

The term “disorder” is used in this disclosure to mean, and is used interchangeably with, the terms disease, condition, or illness, unless otherwise indicated.

An “effective amount,” when used in connection with a compound, is an amount of the compound, e.g., a SHP2 inhibitor, needed to elicit a desired response. In some embodiments, the desired response is a biological response, e.g., in a subject. In some embodiments, the compound (e.g., a SHP2 inhibitor) may be administered to a subject in an effective amount to effect a biological response in the subject. In some embodiments, the effective amount is a “therapeutically effective amount.”

The term “inhibitor” means a compound that prevents a biomolecule, (e.g., a protein, nucleic acid) from completing or initiating a reaction. An inhibitor can inhibit a reaction by competitive, uncompetitive, or non-competitive means. Exemplary inhibitors include, but are not limited to, nucleic acids, DNA, RNA, shRNA, siRNA, proteins, protein mimetics, peptides, peptidomimetics, antibodies, small molecules, chemicals, analogs that mimic the binding site of an enzyme, receptor, or other protein, e.g., that is involved in signal transduction, therapeutic agents, pharmaceutical compositions, drugs, and combinations of these. In some embodiments, the inhibitor can be nucleic acid molecules including, but not limited to, siRNA that reduce the amount of functional protein in a cell. Accordingly, compounds said to be “capable of inhibiting” a particular protein, e.g., SHP2, comprise any such inhibitor.

The term “inhibiting” or any variation thereof, includes any measurable decrease or complete inhibition to achieve a desired result. For example, there may be a decrease of about, at most about, or at least about 5%, 10%, 15%, 20%, 25%, 30%, 35%, 40%, 45%, 50%, 55%, 60%, 65%, 70%, 75%, 80%, 85%, 90%, 95%, 99%, or more, or any range derivable therein, reduction of activity (e.g., SHP2 activity) compared to normal.

The term “allosteric SHP2 inhibitor” means a small-molecule compound capable of inhibiting SHP2 through binding to SHP2 at a site other than the active site of the enzyme. Exemplary allosteric SHP2 inhibitors disclosed herein include, without limitation: (i) RMC-3943; (ii) RMC-4550; (iii) SHP099; (iv) an allosteric SHP2 inhibitor compound of any one of Formula I, of Formula II, of Formula III, of Formula I-V1, of Formula I-V2, of Formula I-W, of Formula I-X, of Formula I-Y, of Formula I-Z, of Formula IV, of Formula V, of Formula VI, of Formula IV-X, of Formula IV-Y, of Formula IV-Z, of Formula VII, of Formula VIII, of Formula IX, and of Formula X, disclosed herein; (v) TNO155, (vi) JAB-3068, (vii) a compound from Table 1, disclosed herein; (viii) a compound from Table 2, disclosed herein; (ix) RLY-1971; or (x) combinations thereof.

The term “mutation” as used herein indicates any modification of a nucleic acid and/or polypeptide, which results in an altered nucleic acid or polypeptide. The term “mutation” may include, for example, point mutations, deletions or insertions of single or multiple residues in a polynucleotide, which includes alterations arising within a protein-encoding region of a gene as well as alterations in regions outside of a protein-encoding sequence, such as, but not limited to, regulatory or promoter sequences, as well as amplifications and/or chromosomal breaks or translocations.

A “patient” or “subject” is a mammal, e.g., a human, mouse, rat, guinea pig, dog, cat, horse, cow, pig, or non-human primate, such as a monkey, chimpanzee, baboon or rhesus.

The term “prevent” or “preventing” with regard to a subject refers to keeping a disease or disorder from afflicting the subject. Preventing includes prophylactic treatment. For instance, preventing can include administering to the subject a compound disclosed herein before a subject is afflicted with a disease and the administration will keep the subject from being afflicted with the disease.

The term “providing to a/the subject” a therapeutic agent, e.g., a SHP2 inhibitor, includes administering such an agent.

The terms “RAS pathway” and “RAS/MAPK pathway” are used interchangeably herein to refer to a signal transduction cascade downstream of various cell surface growth factor receptors in which activation of RAS (and its various isoforms and alleotypes) is a central event that drives a variety of cellular effector events that determine the proliferation, activation, differentiation, mobilization, and other functional properties of the cell. SHP2 conveys positive signals from growth factor receptors to the RAS activation/deactivation cycle, which is modulated by guanine nucleotide exchange factors (GEFs, such as SOS1) that load GTP onto RAS to produce functionally active GTP-bound RAS as well as GTP-accelerating proteins (GAPs, such as NF1) that facilitate termination of the signals by conversion of GTP to GDP. GTP-bound RAS produced by this cycle conveys essential positive signals to a series of serine/threonine kinases including RAF and MAP kinases, from which emanate additional signals to various cellular effector functions.

The term “SHP2” means “Src Homology 2 domain-containing protein tyrosine phosphatase 2” and is also known as SH-PTP2, SH-PTP3, Syp, PTP1D, PTP2C, SAP-2 or PTPN11. Numbering of SHP2 mutations in the present disclosure is according to Uniprot Isoform 2 (accession number Q06124-2), also provided herein (SEQ ID NO: 3):

| | |
|---|---|
| 1 | MTSRRWFHPN ITGVEAENLL LTRGVDGSFL ARPSKSNPGD FTLSVRRNGA VTHIKIQNTG |
| 61 | DYYDLYGGEK FATLAELVQY YMEHHGQLKE KNGDVIELKY PLNCADPTSE RWFHGHLSGK |
| 121 | EAEKLLTEKG KHGSFLVRES QSHPGDFVLS VRTGDDKGES NDGKSKVTHV MIRCQELKYD |
| 181 | VGGGERFDSL TDLVEHYKKN PMVETLGTVL QLKQPLNTTR INAAEIESRV RELSKLAETT |
| 241 | DKVKQGFWEE FETLQQQECK LLYSRKEGQR QENKNKNRYK NILPFDHTRV VLHDGDPNEP |
| 301 | VSDYINANII MPEFETKCNN SKPKKSYIAT QGCLQNTVND FWRMVFQENS RVIVMTTKEV |
| 361 | ERGKSKCVKY WPDEYALKEY GVMRVRNVKE SAAHDYTLRE LKLSKVGQGN TERTVWQYHF |
| 421 | RTWPDHGVPS DPGGVLDFLE EVHHKQESIM DAGPVVVHCS AGIGRTGTFI VIDILIDIIR |
| 481 | EKGVDCDIDV PKTIQMVRSQ RSGMVQTEAQ YRFIYMAVQH YIETLQRRIE EEQKSKRKGH |
| 541 | EYTNIKYSLA DQTSGDQSPL PPCTPTPPCA EMREDSARVY ENVGLMQQQK SFR. |

A “therapeutic agent” is any substance, e.g., a compound or composition, capable of treating a disease or disorder. In some embodiments, therapeutic agents that are useful in connection with the present disclosure include without limitation SHP2 inhibitors, ALK inhibitors, MEK inhibitors, RTK inhibitors (TKIs), and cancer chemotherapeutics.

The terms “therapeutically effective amount” and “therapeutic dose” are used interchangeably herein to refer to an amount of a compound, e.g., a SHP2 inhibitor, which is effective following administration to a subject for treating a disease or disorder in the subject as described herein.

The term “prophylactically effective amount” is used herein to refer to an amount of a compound, e.g., a SHP2 inhibitor, which is effective following administration to a subject, for preventing or delaying the onset of a disease or disorder in the subject as described herein.

The term “treatment” or “treating” with regard to a subject, refers to improving at least one symptom, pathology or marker of the subject's disease or disorder, either directly or by enhancing the effect of another treatment. Treating includes curing, improving, or at least partially ameliorating the disorder, and may include even minimal changes or improvements in one or more measurable markers of the disease or condition being treated. “Treatment” or “treating” does not necessarily indicate complete eradication or cure of the disease or condition, or associated symptoms thereof. The subject receiving this treatment is any subject in need thereof. Exemplary markers of clinical improvement will be apparent to persons skilled in the art.

All of the U.S. patents, U.S. patent application publications, U.S. patent applications, PCT patent application, PCT patent application publications, foreign patents, foreign patent applications and non-patent publications referred to in this specification or listed in any Application Data Sheet are incorporated herein by reference in their entirety. From the foregoing it will be appreciated that, although specific embodiments of the invention have been described herein for purposes of illustration, various modifications may be made without deviating from the spirit and scope of the invention.

Every document cited herein, including any cross-referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the disclosure have been illustrated and described, various other changes and modifications can be made without departing from the spirit and scope of the disclosure. The scope of the appended claims includes all such changes and modifications that are within the scope of this disclosure.

### EXAMPLES

In order that the invention disclosed herein may be more efficiently understood, examples are provided below. It should be understood that these examples are for illustrative purposes only and are not to be construed as limiting the invention in any manner. Throughout these examples, molecular cloning reactions, and other standard recombinant DNA techniques, were carried out according to methods described in Maniatis et al., Molecular Cloning—A Laboratory Manual, 2nd ed., Cold Spring Harbor Press (1989), using commercially available reagents, except where otherwise noted.

### Example 1

### Clinical Data Using RMC-4630

The RMC-4630 phase 1/2 program includes two clinical trials. RMC-4630-01, a phase 1 dose escalation study of RMC-4630 as a single agent RMC-4630-02, a phase 1b/2 study of RMC-4630 in combination with the MEK inhibitor cobimetinib (Cotellic®). The disclosure provides clinical data from both the RMC-4630-01 study and RMC-4630-02 study.

RMC-4630-01 study of single agent RMC-4630 inpatients with advanced solid tumors. RMC-4630-01 is a phase 1 dose escalation study in patients with advanced cancers that evaluates the safety, pharmacokinetics and pharmacodynamic effects of RMC-4630 as a single agent under two different dose administration schedules; daily dosing and twice weekly dosing. Anti-tumor activity is also evaluated in patients who have tumors harboring mutations in the RAS-MAPK pathway.

The RMC-4630-01 study was initially designed to evaluate two different schedules: a daily dosing schedule and an intermittent dosing schedule (D1, D4 of every week). The intermittent schedule was intended to achieve intermittent target coverage, which, in preclinical models, was associated with similar or superior activity and better tolerability.

At the latest data cut-off, 63 patients had received study drug and were evaluable for safety: 14 with the intermittent schedule and 49 with the daily schedule. Dose escalation has been completed for the daily dosing schedule. Dose escalation continues using the intermittent schedule. Preliminary data suggest that the intermittent schedule is a particular schedule for RMC-4630. Safety, tolerability and PK data for patients treated with the intermittent schedule are provided here separately from patients treated with the daily schedule.

RMC-4630 Interim safety and tolerability of an intermittent schedule. Fourteen patients dosed with the D1, D4 schedule have been evaluated for safety after a median follow-up of 2 months. Demographic information is shown in FIG. 10.

The emerging safety profile is consistent with the mechanistic effects of the drug candidate on SHP2 and hence the RAS signaling cascade, including edema, reduced red cell production (low hemoglobin concentration and worsening of pre-existing anemia), reduced platelet production (thrombocytopenia), hypertension and fatigue. This safety profile was largely predictable from non-clinical studies and clinical studies of other well-known inhibitors of this pathway. Treatment-related and emergent adverse events (AEs) occurring in greater than 15% of patients are provided in FIG. 11. No related grade 4 or grade 5 AEs have been reported for this schedule. One related SAE has been reported in a patient with pancreatic cancer receiving 200 mg twice weekly who was hospitalized with grade 3 abdominal distension; the AE was unresolved at the time the patient withdrew from the study to transfer to hospice care.

RMC-4630 Pharmacokinetics with Intermittent Schedule. The pharmacokinetic profile of RMC-4630 after dosing on D1, D4 schedule is shown in FIGS. 12 and 13. Plasma levels of RMC-4630 after oral administration to patients were similar to those predicted from preclinical studies in rats and dogs. No accumulation from day 1 to day 15 was observed. Plasma exposure at both dose levels was within the range anticipated to be biologically active from preclinical models. After a single dose of 140 mg the plasma concentration of RMC-4630 remains above the in vivo EC₅₀ for pERK for 72 hours. The half-life of RMC-4630 is estimated to be 25 hrs.

Interim safety and tolerability of RMC-4630 by a daily schedule. Forty-nine patients have been treated with the daily schedule. Median follow-up is 2 months (range 1-14 m). Demographic information is shown in FIG. 14.

Daily dosing has been associated with more frequent and severe AEs than the intermittent schedule. As with the intermittent schedule the emerging safety profile from the daily dosing schedule is consistent with the mechanistic effects of the drug on SHP2 and the RAS signaling pathways. The maximal tolerated dose (MTD) for daily dosing has not been formally determined, although dose escalation will not continue beyond the 80 mg daily level already evaluated. Were further development with this schedule to be pursued, the recommended phase 2 dose for this daily schedule would be in the range of 60 mg.

Related grade 3 and grade 4 AEs are shown in FIG. 15. No toxicities consistent with ‘off-target’ effects have been reported. No deaths (grade 5 AEs) have been ascribed to daily administration of RMC-4630. Increases in liver enzymes such as alanine transaminase and aspartate transaminase have been observed at all grades. These have been attributed, wholly or in part, to RMC-4630 in 10% or 16% of patients treated with the daily schedule respectively. In two patients (4%) the increase in alanine transaminase or aspartate transaminase was either grade 3 or grade 4.

Eight patients (16%) treated with the daily schedule have experienced toxicities involving the lungs or respiratory system that were attributed by the treating investigator in part to RMC-4630. These were generally moderate or mild. Two additional cases of grade 4 respiratory failure are discussed in more detail below in the description of serious adverse events (SAEs). There has been little evidence of systemic activation of the immune system in subjects treated with RMC-4630. There have been no reports of pneumonitis. Related adverse events involving other important organs such as the heart, brain, kidneys have been either uncommon and mild to moderate in severity, or not reported.

There have been three (6%) serious adverse events thought to be possibly or probably related to study drug as assessed by the Sponsor (FIG. 16). Three additional SAEs have occurred in which the investigator was unable to rule out an association with study drug, but where the evidence for causality by RMC-4630 was absent or considered unlikely by the Sponsor. One patient with extensive metastases of tumor in the lungs developed grade 4 shortness of breath and was hospitalized and treated with oxygen. The adverse event was ongoing when the patient was withdrawn from the study. A second patient with fever and radiologic evidence of infectious pneumonia developed grade 4 respiratory failure and was treated with oxygen, systemic antibiotics and corticosteroids. The event was ongoing when the patient died due to progression of underlying cancer. A third patient developed a single reading of grade 3 prolongation of QTc. This patient had been receiving 60 mg daily of RMC-4630 but had not received any dose for three days at the time of the reading. The patient had a previous history of prolonged QTc, underlying systemic lupus, and was taking ondansetron. QTc was prolonged (grade 1) at baseline. Five hours after the prolonged QTc reading the patient had two follow-up ECGs that showed normal QTc interval.

Pharmacokinetics of RMC-4630 with daily schedule. With daily dosing plasma concentrations of RMC-4630 reached a steady state by day 22 (FIGS. 17 and 18). Plasma concentrations of RMC-4630 in the blood at all daily dose levels were consistently higher than the in vivo EC₅₀ for pERK in tumor models. Exposure increased approximately proportionally with increasing dose. The total exposure to RMC-4630 over a 24 hour period at the putative MTD of 60 mg daily was 14.6 uM·hr. This is more than twice the exposure that is required to see anti-tumor effects, particularly tumor stasis, in animal models (6.44 uM·hr).

Pharmacodynamic effects of RMC-4630, comparison of daily and intermittent schedules. Activation of the protein ERK, which is an important protein in the RAS signaling pathway and a substrate for MEK, is a good surrogate for the inhibition of pathway activity by a SHP2 inhibitor. The pharmacodynamic effects of RMC-4630 on activation of ERK were studied in the blood cells of patients being treated with RMC-4630. Despite considerable assay variability and inter-patient variability, which is common for these types of dynamic assays in patients, there was a trend in favor of inhibition of activated ERK in peripheral blood cells at all dose levels tested. These effects are consistent with engagement and inhibition of the SHP2 target and downstream RAS signaling by RMC-4630.

Phosphorylation of ERK has been assessed in tumor before, and while receiving, RMC-4630 (FIG. 7). In three cases there was a reduction in phosphorylation of cytoplasmic and nuclear ERK in the tumor while RMC-4630 was at steady state. One patient's tumor showed no reduction in tumor pERK, but this tumor showed very little phosphorylation in the pre-treatment sample and had not received any RMC-4630 for eight days prior to the second tumor biopsy.

Allelic burden of circulating KRAS^{G12C} tumor DNA (ctDNA) has been assessed prior to study and at least once on study in seven patients with tumors harboring KRAS^{G12C} (FIG. 19). KRAS^{G12C} DNA was detected in four of seven patients prior to study. In three patients with NSCLC and either PR or SD as best response there was a reduction in circulating KRAS^{G12C} In one patient with colon cancer who had PD the allelic frequency of KRAS^{G12C} increased.

Interim evidence of clinical activity of RMC-4630 on daily and intermittent schedules. There is preliminary evidence that RMC-4630 has single agent anti-tumor activity in KRAS mutant NSCLC. One patient with KRAS^{G12C} NSCLC treated at 60 mg daily had a confirmed PR, with a 49% reduction in tumor volume as measured by CT imaging. A second NSCLC patient with KRAS^{G12D}+SHP2^{V428M} treated with 140 mg D1, D4 had an unconfirmed PR. Disease control rate (DCR, the sum of best response of PR and SD cases) for patients with KRAS^{G12C} NSCLC thus far is 6/8 (75%).

Five patients with KRAS^{G12C} NSCLC have had follow-up CT scans of target lesions and have had either PR or SD (FIG. 20); three patients have not reported follow-up measurements of target lesions, of which one has been recorded as best response of SD and two of PD. For all patients with KRAS mutant NSCLC disease, DCR thus far is 12/18 (67%) (FIG. 21). One patient with KRAS^{G12V} NSCLC has been on treatment for over 14 months with stable disease (˜15% reduction in tumor volume). In histotypes other than NSCLC the best response thus far has been SD.

RMC-4630-02 study of RMC-4630 in combination with cobimetinib (Cotellic®) patients with advanced solid tumors. RMC-4630-02 is a phase 1b/2 dose escalation study of RMC-4630 in combination with the MEK inhibitor cobimetinib in patients with advanced cancers that harbor mutations in the RAS signaling pathway. The study evaluates the safety, tolerability and pharmacokinetics of RMC-4630 and cobimetinib under two different dose administration schedules in order to determine a recommended phase 2 dose and schedule for further clinical testing. Initially the study assesses twice weekly RMC-4630 (D1, D4) with daily cobimetinib (21 days on, 7 off). In the second schedule, both RMC-4630 and cobimetinib are dosed intermittently. A preliminary evaluation of anti-tumor activity is also being made.

At the latest data cut-off, eight patients had received study medication at the first dose level and were evaluable for safety. Dose escalation to the next highest dose level has occurred and enrollment is ongoing.

Interim safety and tolerability. Eight patients have been evaluated for safety after a median follow-up of less than 2 months. Demographic information is shown in FIG. 22.

The emerging safety profile is consistent with the mechanistic effects of both SHP2 inhibition and MEK inhibition, including edema, diarrhea and other gastrointestinal toxicity, anemia and rash. This safety profile was largely predictable from single agent clinical studies of both agents.

Treatment-related and emergent adverse events (AEs) are listed in FIGS. 23 and 24. There have been no grade 4 or grade 5 AEs or related serious AEs (SAEs) reported.

Pharmacokinetics. The pharmacokinetic profiles of RMC-4630 and cobimetinib are shown in FIGS. 25 and 26. Plasma levels of RMC-4630 are continuously greater than the predicted EC₅₀ for pERK inhibition in preclinical tumor models.

PD and Clinical activity. Only three patients have been evaluated for efficacy in this study. No efficacy data or ctDNA data are available in the electronic database at the time of reporting.

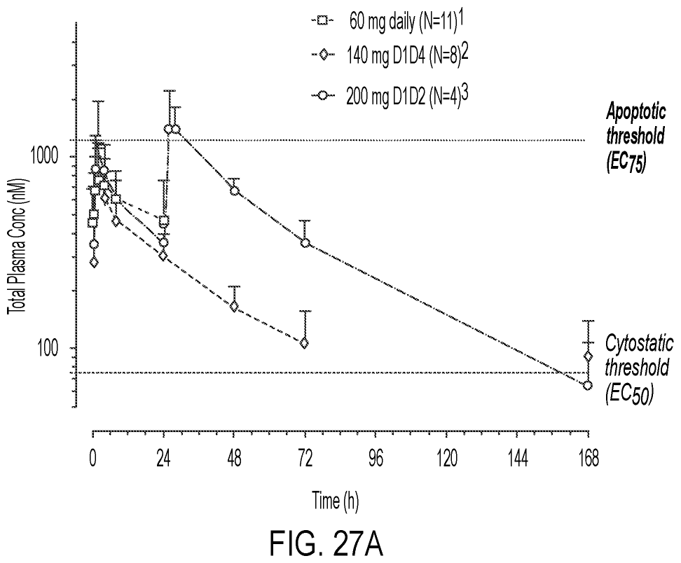

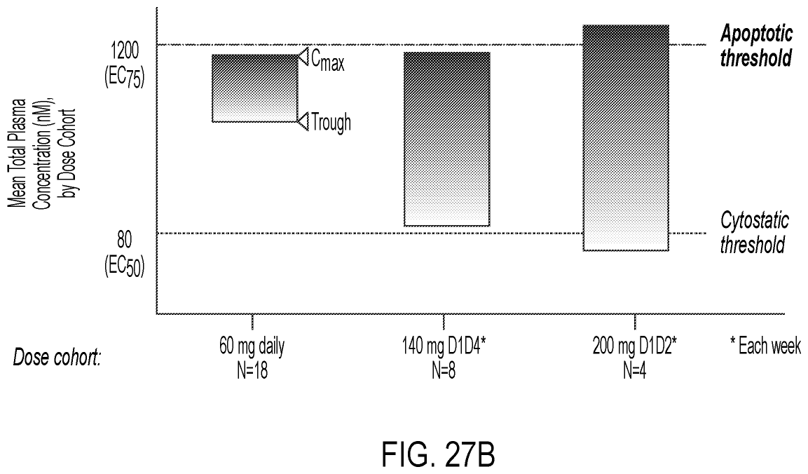

The pharmacokinetic profile of RMC-4630 after dosing on the intermittent schedule is shown in Table 3 and FIG. 27a.

The median half-life of RMC-4630 was approximately 28 and 33 hours following a single dose at 140 and 200 mg, respectively. No accumulation from day 1 to day 15 was observed with either D1, D4 dosing or D1, D2 dosing schedules. Plasma exposure at all dose translated well from preclinical modeling. At 200 mg D1, D2, the Cmax concentrations were generally above those thought to represent the ‘apoptotic threshold’ or plasma concentration at which RMC-4630 can best induce tumor cell death (FIG. 27a). In addition, trough concentrations towards the end of the week were below those thought to be required for normal tissue recovery. This is consistent with the improved safety/tolerability of the D1, D2 schedule. The pharmacokinetic profile of the 200 mg D1, D2 schedule seems to represent the one closest to that associated with an optimal therapeutic index in preclinical models, compared with the maximum tolerated dose at the alternative schedules (60 mg daily or 140 mg D1, D4).

FIG. 27b provides a schematic representation of RMC-4630 pharmacokinetics at three tolerated dose schedules with peak and trough concentrations of RMC-4630 derived from the data from FIG. 27a and Table 3.

Single agent activity of RMC-4630 has also been reported in two patients with tumors harboring NF1 LOF mutations. One patient, an adult female with a poorly differentiated uterine carcinosarcoma, had a complete response. This patient was diagnosed with a tumor harboring two NF1 LOF mutations, a POLE (DNA repair) mutation, and ultra-high tumor mutational burden. The patient had received two treatment regimens prior to starting RMC-4630. She started RMC-4630 200 mg D1D4 and was subsequently reduced to 140 mg D1D4 due to gastrointestinal toxicity. At two months, her tumor dimension had reduced from 1.7 cm to undetectable. CR was subsequently confirmed and she continues in CR at five months on study therapy.

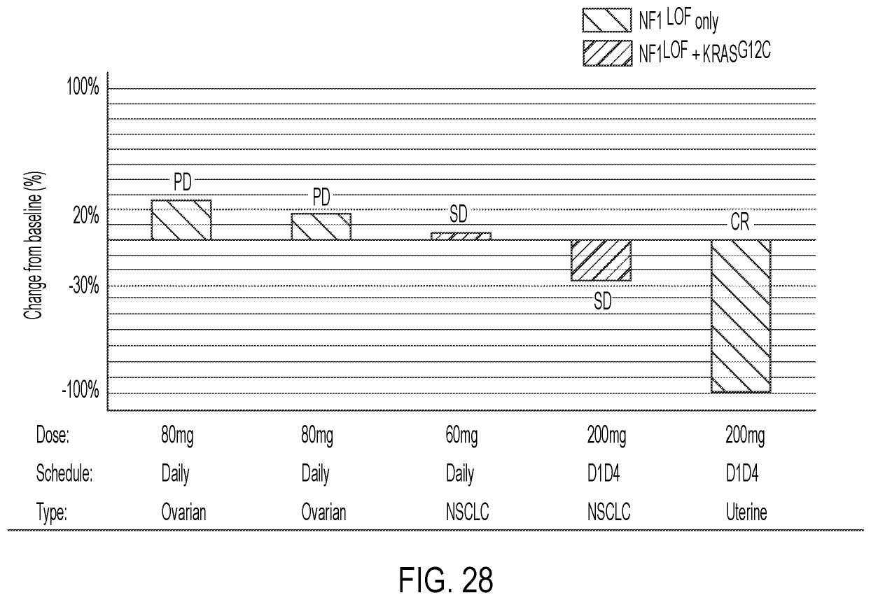

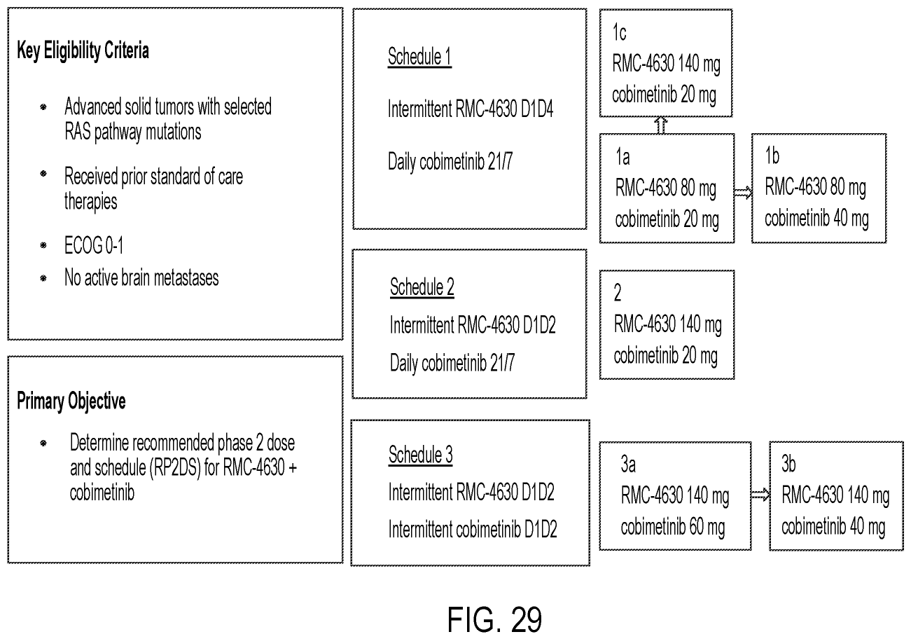

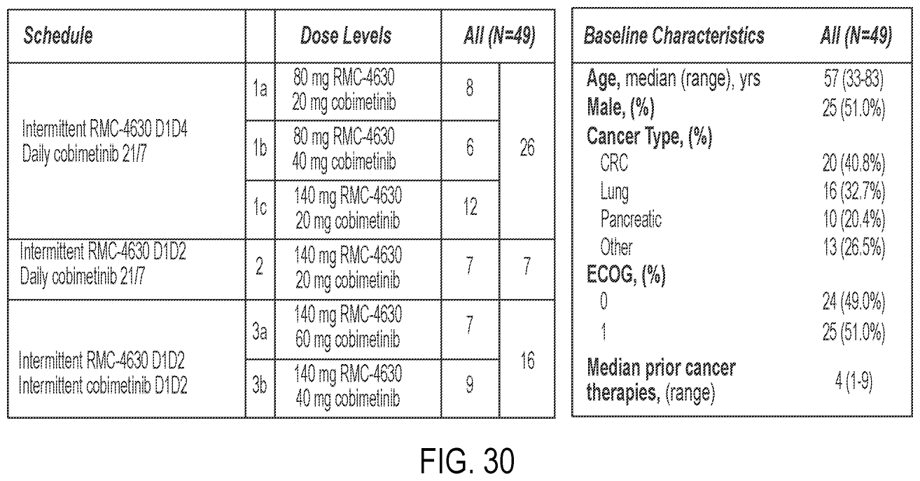

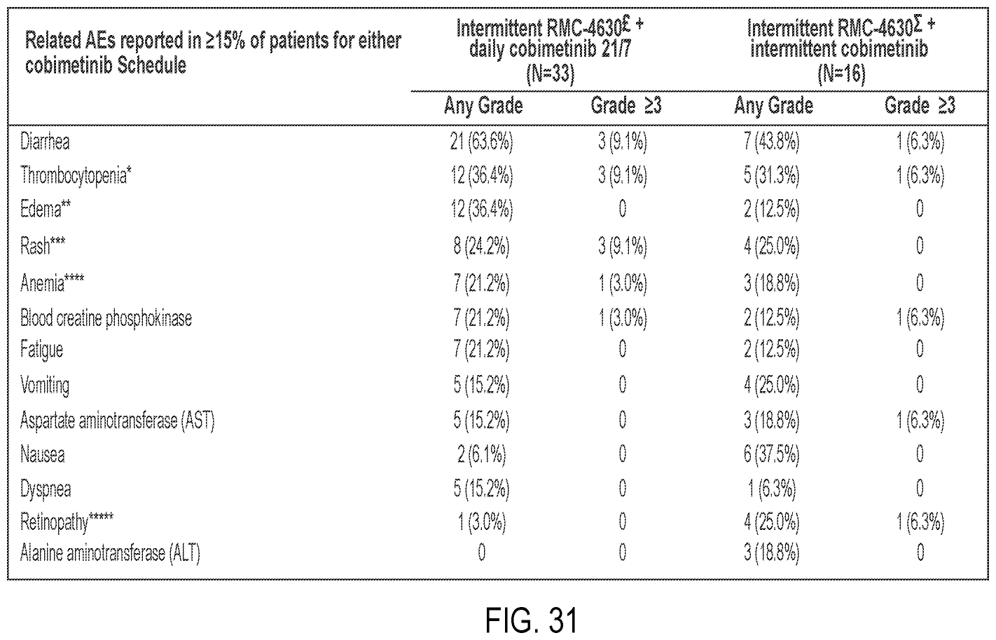

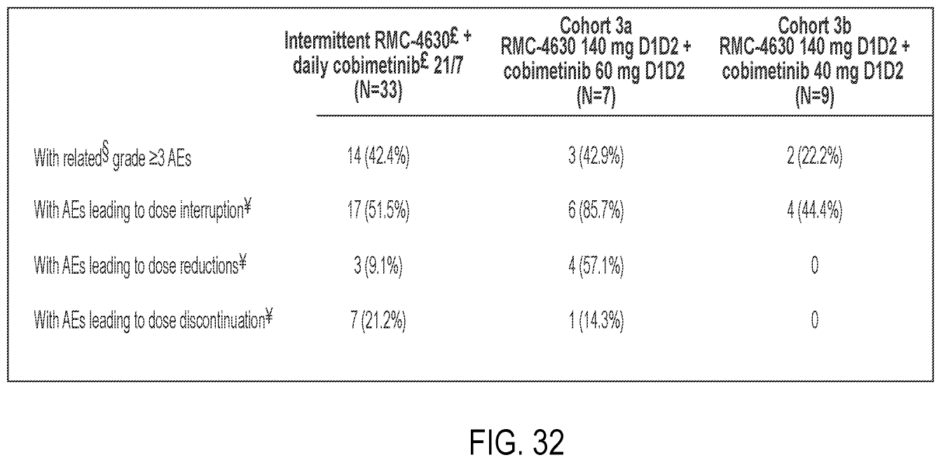

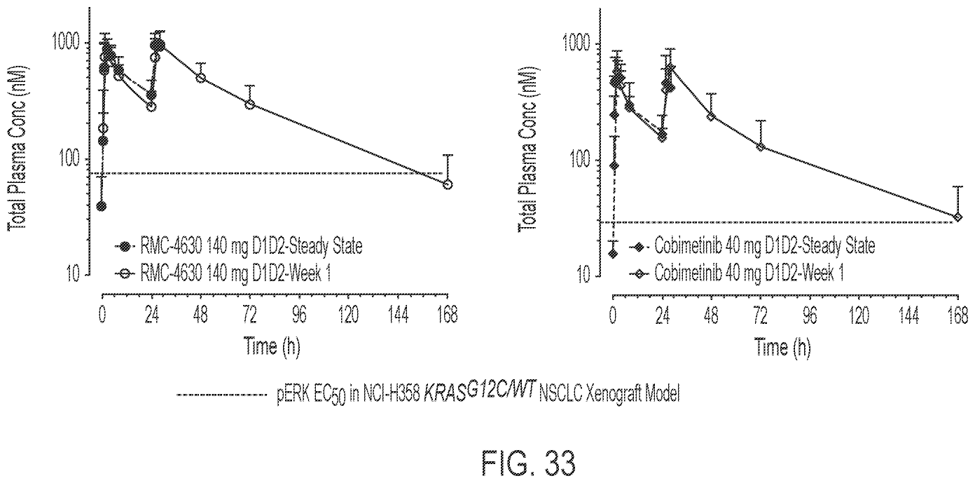

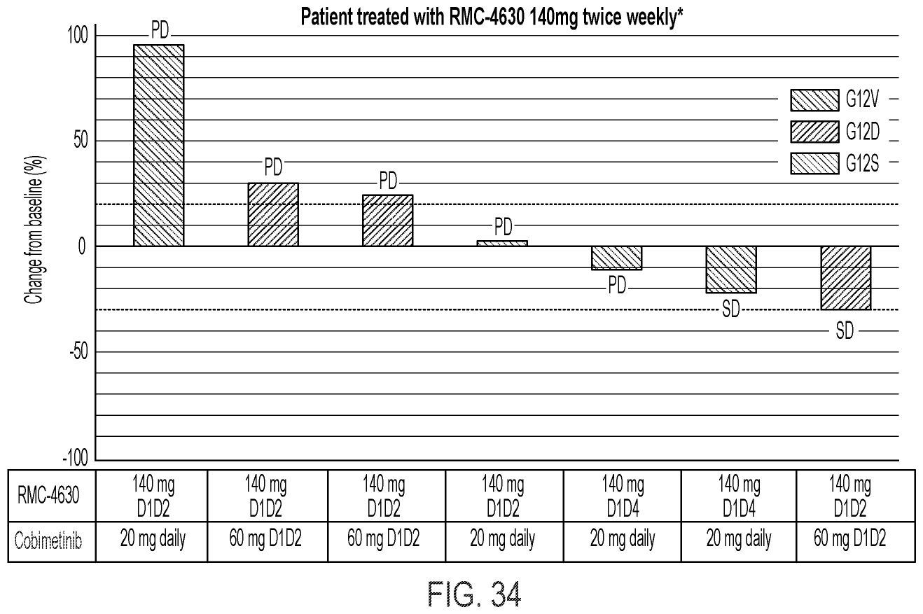

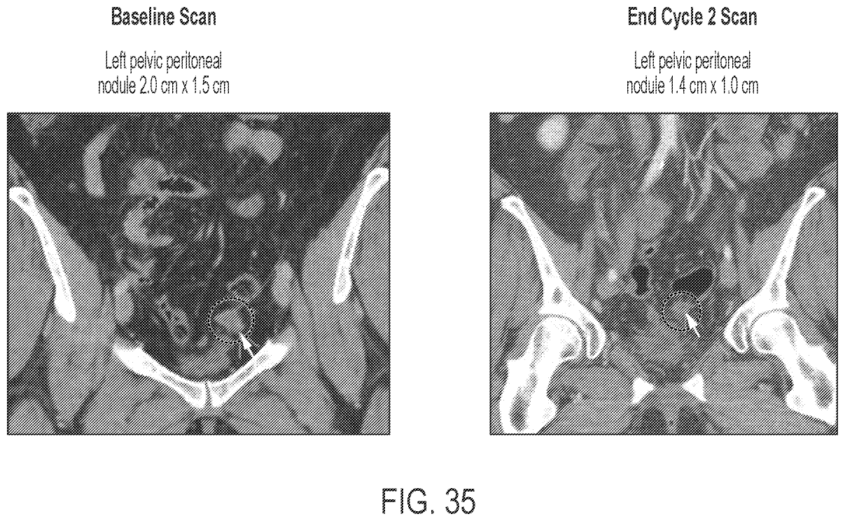

A second patient with NSCLC harboring a co-existing NF1 LOF and KRASG12C had tumor shrinkage (FIG. 28). Data are presented for the efficacy evaluable population (N=6) defined as participants with baseline and at least one post-baseline scan or who died or had clinical progression prior to first post-baseline scan. One patient (NSCLC) with death due to clinical PD prior to first scan is not represented in this figure. NF1 LOF is loss, or significant reduction, in neurofibromin protein function which is presumed from the nature of the mutation in the neurofibromin 1 gene.

TABLE 3
Pharmacokinetics-intermittent schedule in RMC-4630-01 study.

| | | | | | | PK parameters [Mean(CV %)] | | | | | |
|---|---|---|---|---|---|---|---|---|---|---|---|
| | | | | | | | Median | | Mean | | |
| | | | | | | | Tₘₐₓ | | accumulation | | Median t_{1/2} |
| | | | | Cycle/ | N(Cₘₐₓ/ | Cₘₐₓ | (range) | AUC₀₋₂₄ | (AUC₀₋₂₄ | AUC₀₋₇₂ | (range) |
| Study | Schedule | Dose | | Day | AUC) | μM | h | μM*h | ratio) | μM*h | H |
| Mouse | QD | 10 | mg/kg | | | 0.98 | | 6.44 | | NA | NA |
| efficacy | steady | 20 | mg/kg | | | 3.4 | | 11.7 | | | |
| | state | | | | | | | | | | |
| RMC- | Twice | 140 | mg | 1/1 | 8/8 | 0.915 (50) | 2 (1-8) | 10.8 (35) | | 19.7 (31) | 28 (23-33) |
| 4630-01 | weekly | | | 1/15 | 8/8 | 0.935 (35) | 2 (2-4) | 14.0 (41) | 1.3 | NA | NA |
| | (D 1, D 4) | 200 | mg | 1/1 | 18/18 | 1.38 (41) | 2 (0.5-8) | 17.5 (38) | | 39.0 (40) | 33 (20-40) |
| | | | | 1/15 | 12/12 | 1.23 (32) | 3 (1-24) | 18.6 (31) | 1.1 | NA | NA |
| | Twice | 200 | mg | 1/1 | 4/4 | 1.58 (45) | 3 (2-4)* | 13.9 (33) | | NA | NA |
| | weekly | | | 1/15 | 3/3 | 1.63 (25) | 2 (2-2) | 14.5 (18) | 1.0 | NA | NA |
| | (D 1, D 2) | | | | | | | | | | |

*Tmax value was time post D 2 or D 16 dosing for 200 mg (D 1, D 2)

1. A method of treating a disease or disorder, comprising administering to a subject in need thereof a first dose of a first Src homology region 2 (SH2)-containing protein tyrosine phosphatase 2 (SHP2) inhibitor and a second dose of a second SHP2 inhibitor, wherein the first dose and the second dose are administered on an intermittent schedule, and wherein the first SHP2 inhibitor and the second SHP2 inhibitor are identical.

2-4. (canceled)

5. The method of claim 1, wherein the first dose is administered on a first day (D1) of the intermittent schedule and the second dose is administered on a fourth day (D4) of the intermittent schedule.

6. The method of claim 1, wherein the first dose is administered on a first day (D1) of the intermittent schedule and the second dose is administered on a second day (D2) of the intermittent schedule.

7-15. (canceled)

16. The method of claim 1, wherein an iteration of the intermittent schedule is 7 days.

17-22. (canceled)

23. The method of claim 16, wherein a subsequent dose is administered on an eighth day (D8).

24-28. (canceled)

29. The method of claim 23, wherein a first iteration comprises the first dose and the second dose and wherein the subsequent dose is the first dose of a second or subsequent iteration.

30-32. (canceled)

33. The method of claim 1, wherein the method further comprises administering a second therapeutic agent.

34-36. (canceled)

37. The method of claim 33, wherein the second therapeutic agent comprises a rat sarcoma (RAS) inhibitor.

38-53. (canceled)

54. The method of claim 33, wherein the first SHP2 inhibitor or the first dose of a SHP2 inhibitor and the second therapeutic agent are administered simultaneously.

55. (canceled)

56. The method of claim 33, wherein the second SHP2 inhibitor or the second dose of a SHP2 inhibitor and the second therapeutic agent are administered simultaneously.

57-67. (canceled)

68. The method of claim 33, wherein the second therapeutic agent is administered before the second SHP2 inhibitor or the second dose of a SHP2 inhibitor.

69-73. (canceled)

74. The method of claim 33, wherein the second therapeutic agent is administered before the subsequent SHP2 inhibitor or the subsequent dose of a SHP2 inhibitor.

75. The method of claim 33, wherein the first dose of the first SHP2 inhibitor and a first dose of the second therapeutic agent are administered on D1 of the intermittent schedule and wherein the second dose of the second SHP2 inhibitor and a second dose of the second therapeutic agent are administered on different days of the intermittent schedule.

76-90. (canceled)

91. The method of claim 33, wherein an iteration of the intermittent schedule is 7 days.

92-93. (canceled)

94. The method of claim 1, wherein the SHP2 inhibitor is an allosteric SHP2 inhibitor.

95-107. (canceled)

108. The method of claim 1, wherein the SHP2 inhibitor comprises
(i) SHP099;
(ii) an allosteric SHP2 inhibitor compound of any one of Formula I, of Formula II, of Formula III, of Formula 1-VI, of Formula I-V2, of Formula I-W, of Formula i-X, of Formula I-Y, of Formula I-Z, of Formula IV, of Formula V, of Formula VI, of Formula IV-X, of Formula IV-Y, of Formula 1V-Z, of Formula VII, of Formula VIII, of Formula IX, and of Formula X;
(iii) TNO155;
(iv) JAB-3068;
(v) a compound from Table 1, disclosed herein;
(vi) a compound from Table 2, disclosed herein;
(vii) RLY-1971; or
(viii) a combination thereof.

109-118. (canceled)

119. The method of claim 1, wherein the SHP2 inhibitor comprises

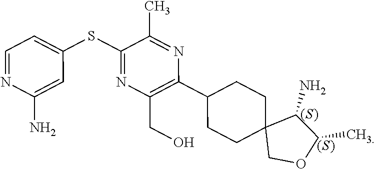

120-122. (canceled)

123. The method of claim 1, wherein the subject further comprises a mutation in a component of a rat sarcoma (RAS) signaling pathway.

124. The method of claim 123, wherein the mutation in the component of the RAS signaling pathway occurs in KRAS, neurofibromin 1 (NF1), or serine/threonine-protein kinase B-raf (BRAF).

125. The method of claim 123, wherein the mutation in the component of the RAS signaling pathway comprises:
i) a substitution of a cysteine (C) for a glycine (G) at position 12 of KRAS (KRAS^{G12C});
ii) a KRAS amplification (KRAS^{amp});
iii) a loss of function (LOF) mutation of NF1 (NF1^{LOF}); or
iv) a class 3 mutant of BRAF (BRAF^{class3}).

126-129. (canceled)

130. The method of claim 123, wherein the disease or disorder is cancer.

131-136. (canceled)

137. The method of claim 130, wherein the cancer:
(i) is non-small cell lung cancer;
(ii) presents a brain metastasis in the subject;
(iii) has a primary presentation in a pancreas of the subject;
(iv) has a secondary presentation in a pancreas of the subject;
(v) has a primary presentation in one or more of a large intestine, a small intestine, a stomach, a bladder, a kidney, a colon or a rectum of the subject;
(vi) has a secondary presentation in one or more of a large intestine, a small intestine, a stomach, a bladder, a kidney, a colon or a rectum of the subject;
(vii) has a primary presentation as a sarcoma in the subject; or
(viii) has a secondary presentation as a sarcoma in the subject.

138-188. (canceled)

189. A method of treating a disease or disorder in a subject in need thereof, comprising administering to the subject a first dose of a first Src homology region 2 (SH2)-containing protein tyrosine phosphatase 2 (SHP2) inhibitor and a second dose of a second SHP2 inhibitor, wherein the first dose and the second dose are administered on an intermittent schedule, and wherein the subject has a mutation of SHP2.

190. A method of decreasing activation of a component of a RAS signaling pathway in a subject in need thereof, comprising administering to the subject a first dose of a first Src homology region 2 (SH2)-containing protein tyrosine phosphatase 2 (SHP2) inhibitor and a second dose of a second SHP2 inhibitor, wherein the first dose and the second dose are administered on an intermittent schedule.
